(19) United States
(12) Patent Application Publication
(10) Pub. No.: US 2025/0074890 A1
(43) Pub. Date: Mar. 6, 2025

(54) CYANOPYRIDINE AND CYANOPYRIMIDINE BCL6 DEGRADERS

(71) Applicant: DANA-FARBER CANCER INSTITUTE, INC., Boston, MA (US)

(72) Inventors: Lyn Howard Jones, Winchester, MA (US); Jianwei Che, Sharon, MA (US); Huang Huang, Belmont, MA (US); Nikki Kong, Boston, MA (US); Silas Ferrao, Boston, MA (US); Yingpeng Liu, Boston, MA (US); Justin Cruite, Boston, MA (US)

(73) Assignee: DANA-FARBER CANCER INSTITUTE, INC., Boston, MA (US)

(21) Appl. No.: 18/293,978

(22) PCT Filed: Aug. 1, 2022
(86) PCT No.: PCT/US2022/074387

§ 371 Date: Jan. 31, 2024

Related U.S. Application Data
(60) Provisional application No. 63/352,063, filed on Jun. 14, 2022.
(60) Provisional application No. 63/228,303, filed on Aug. 2, 2021.

(51) Int. Cl. C07D 401/14; A61K 31/4545; A61K 31/4709; A61K 31/506; A61K 31/5377; A61K 31/553; C07D 405/14; C07D 413/14; C07D 498/04

(52) U.S. Cl. CPC C07D 401/14; A61K 31/4545; A61K 31/4709; A61K 31/506; A61K 31/5377; A61K 31/553; C07D 405/14; C07D 413/14; C07D 498/04

(57) ABSTRACT

Described are the compounds, compositions and methods of treating a disease or disorder characterized by aberrant B-cell lymphoma 6 (BCL6) activity.

### RELATED APPLICATIONS

This application claims the benefit of priority under 35 U.S.C. § 119(e) to U.S. Provisional Application No. 63/228,303, filed Aug. 2, 2021, and U.S. Provisional Application No. 63/352,063, filed Jun. 14, 2022, each of which are incorporated herein by reference in their entireties.

### BACKGROUND OF THE INVENTION

As demonstrated by the clinical efficacy of thalidomide analogs for the treatment of hematologic malignancies, small molecule-induced protein degradation has emerged as a powerful therapeutic strategy. Thalidomide analogs, including lenalidomide and pomalidomide, modulate the activity of the Cullin Really Interesting New Gene (RING) ligase 4-cereblon (CRBN) (CRL4^{CRBN}) E3 ubiquitin ligase to recruit and ubiquitinate neo-substrates including Ikaros family zinc finger 1 (IKZF1), IKZF3, and casein kinase 1-alpha (CK1α), which leads to their proteasomal degradation (Kronke et al., Science 343:301-305 (2014); Lu et al., Science 343:305-309 (2014); Kronke et al., Nature 523:183-188 (2015)). Other small molecules that induce protein degradation include aryl sulfonamides, which promote the destruction of RNA binding motif protein 39 (RBM39) in a CRL4-DNA damage binding protein 1 (DDB1) and CUL4 associated factor 15 (DCAF15) (CRL4^{DCAF15})-dependent manner (Han et al., Science 356:eaal3755 (2017).

Other types of small molecules include hetero-bifunctional degraders (also known as PROTACs) (Toure et al., Angew. Chem. Int. Ed. Engl. 55:1966-1973 (2016)) have been developed for a wide range of targets including kinases (Huang et al., Cell Chem. Biol. 25:88-99 (2018)), nuclear receptors (Bondeson et al., Nat. Chem. Biol. 11:611-617 (2015)), and epigenetic enzymes (Winter et al., Science 348:1376-1381 (2015)). These small molecule degraders engage both the E3 ligase and the target protein substrate, promoting formation of a substrate-drug-ligase ternary complex (Nowak et al., Nat. Chem. Biol. 14:706-714 (2018); Petzold et al., Nature 532:127-130 (2016); Sievers et al., Science 362:aat0572 (2018)).

While degraders have shown remarkable efficacy and sustained depletion for some target proteins, other proteins have proven recalcitrant to this approach. One such example is the B cell lymphoma 6 (BCL6) protein, for which hetero-bifunctional degraders have shown insufficient target modulation to induce growth inhibition (McCoull et al., ACS. Chem. Biol. 13:3131-3141 (2018)).

BCL6 was first identified as a locus affected by chromosomal translocations in diffuse large B-cell lymphomas (DLBCL). It is now known to be broadly expressed in many lymphomas. Its role in lymphomagenesis stems from its function in the humoral immune system, where upregulation of BCL6 is required for the formation of germinal centers (GC) during the humoral immune response (Ye et al., Nat. Genet. 16:161-170 (1997); Dent et al., Science 276:89-92 (1997)). GCs are transient structures that form in response to antigen stimulation. Within GCs, B cells tolerate massive proliferation and the mutagenic effect of the DNA-editing enzyme AICDA to undergo immunoglobulin affinity maturation (Klein et al., Nat. Rev. Immunol. 8:22-33 (2008)). These activities are orchestrated by and dependent on BCL6, a powerful transcriptional repressor that silences hundreds of genes. Some of these target genes control DNA damage sensing (i.e., ATR, CHEK1, TP53, ARF) and proliferation checkpoints (i.e., CDKN1A, CDKN1B, CDKN2A, CDKN2B, PTEN) (Hatzi et al., Trends Mol. Med. 20:343-352 (2014)). BCL6 also represses genes required for exit from the GC reaction and plasma cell differentiation (e.g., IRF4, PRDM1). This ensures that GC B cells have sufficient time to acquire somatic hyper-mutation of their immunoglobulin genes. Thus, deregulated suppression of these target genes could result in malignant transformation of B cells.

BCL6 also represses numerous oncogenes in GC B cells, including MYC, BCL2, BMI1, and CCND1 (Ci et al., Blood 113:5536-5548 (2009)). Through this function, BCL6 may mitigate its own pro-oncogenic checkpoint repression effect and thus reduce the potential for malignant transformation of GC B cells. This effect is abrogated in the presence of BCL2 or MYC translocations, which drive expression of these oncogenes through aberrant regulatory elements. The presence of both MYC and/or BCL2 together with BCL6 (regardless of translocations) is clearly deleterious because it provides B cells with simultaneous suppression of checkpoints through BCL6, along with the pro-growth and survival effects of MYC and BCL6 (Cardenas et al., Clin. Cancer Res. 23:885-893 (2017)). In the normal immune response, BCL6 function is terminated by the disruption of BCL6 transcriptional complexes through CD40-induced ERK signaling and downregulation of BCL6 mRNA by IRF4 and PRDM1 (Polo et al., Blood 112:644-651 (2008)). Termination of BCL6 function is required for B cells to exit the GC reaction.

BCL6 is a promising drug target for non-Hodgkin lymphomas such as diffuse large B cell lymphoma (DLBCL) (Cerchietti et al., Cancer Cell 17:400-411 (2010); Cardenas et al., J. Clin. Invest. 126:3351-3362 (2016)) and follicular lymphoma (Bosga-Bouwer et al., Genes Chromosomes Cancer 44:301-304 (2005)). Pathologically increased BCL6 expression, as a result of somatic BCL6 translocation, exonic mutation, promoter mutation, or mutations in regulatory pathways, is a common driver of B cell malignancies (Hatzi et al., Trends Mol. Med. 20:343-352 (2014)). In genetically engineered mice, overexpression of BCL6 is sufficient to drive lymphoma development (Cattoretti et al., Cancer Cell 7:445-455 (2005)). BCL6 acts as a master transcriptional repressor enabling rapid expression of germinal center (GC) B cells and tolerance to genomic instability caused by hypermutation of the immunoglobulin genes and class switch recombination (Hatzi et al., Trends Mol. Med. 20:343-352 (2014)). BCL6 represses a broad range of genes involved in the DNA damage response (Ranuncolo et al., Blood Cells Mol. Dis. 41:95-99 (2008)), cell cycle checkpoints (Tunyaplin et al., J. Immunol. 173:1158-1165 (2004)), and differentiation (Phan et al., Nat. Immunol. 6:1054-1060 (2005)). As expected, knock-out of BCL6 in lymphoma cells results in tumor stasis (Schlager et al., Oncotarget 11:875-890 (2020)). Several peptide and small molecule inhibitors targeting BCL6 have shown efficacy in vivo, but only at high concentrations, which has limited their translation into clinical therapeutic agents (Cerchietti et al., Cancer Cell 17:400-411 (2010); Cardenas et al., J. Clin. Invest. 126:3351-3362 (2016)).

Broad complex/Tramtrack/Bric-a-brac (BTB) proteins are a diverse family of proteins that are characterized by the presence of a common protein-protein interaction domain, known as the BTB domain. BTB proteins have diverse functions ranging from transcriptional regulation and chromatin remodeling to protein degradation and cytoskeletal regulation. Specificity of function is determined in part by additional domains present in a given BTB protein, as well as by interaction partners. Studies of BTB proteins in Drosophila and mammalian systems have revealed the importance of these proteins in multiple developmental contexts, as well as in cancer and neurological and musculoskeletal diseases. BTB proteins play critical roles in transcriptional regulation and chromatin remodeling (Chaharbakhshi et al., Genesis 54:505-518 (2016)).

The BTB domain mediates various functions of BCL6, such as homodimerization and interaction with co-repressor proteins (Ghetu et al., Mol. Cell 29:384-391 (2008); Ahmad et al., Mol. Cell 12:1551-1564 (2003)). Techniques that disrupt the protein-protein interaction between the BTB domain of BCL6 and its co-repressors may be useful to combat BCL6-related diseases.

### SUMMARY OF THE INVENTION

A first aspect of the present invention is directed to a compound having a structure represented by formula (I):

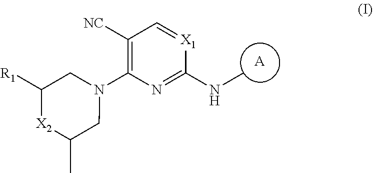

wherein A, X₁, X₂, and R₁ are as defined herein, or a pharmaceutically acceptable salt or stereoisomer thereof.

Another aspect of the present invention is directed to a pharmaceutical composition that includes a therapeutically effective amount of a compound of formula (I) or a pharmaceutically acceptable salt or stereoisomer thereof, and a pharmaceutically acceptable carrier.

A further aspect of the present invention is directed to a method of treating a disease or disorder that is characterized or mediated by aberrant BCL6 activity that entails administering to a subject in need thereof a therapeutically effective amount of a compound of formula (I) or a pharmaceutically acceptable salt or stereoisomer thereof.

In some embodiments, the disease or disorder is a lymphoid malignancy. In some embodiments, the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), cutaneous T-cell lymphoma, chronic myeloid leukemia, or B-cell non-Hodgkin's lymphoma. In some embodiments, the disease or disorder is cancer.

### BRIEF DESCRIPTION OF THE DRAWINGS

FIG. 1A is a graph showing the anti-proliferative effects of Tazemetostat (Taz) treatment. FIG. 1B is a graph showing anti-proliferative effects of Lirametostat (Lira) treatment. FIG. 1C is a heatmap showing excess over Bliss (eob) scores that were calculated for Tazemetostat treatment. FIG. 1D is a heatmap showing excess over Bliss (eob) scores that were calculated for Lirametostat treatment. FIG. 1E is a plot showing that both Tazemetostat and Lirametostat treatments reduced the BCL6 degrader dose requirement.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₃-C₅ branched-chain alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “alkoxylene” refers to a saturated monovalent aliphatic radicals of the general formula (—O—CₙH₂ₙ—) where n represents an integer (e.g., 1, 2, 3, 4, 5, 6, or 7) and is inclusive of both straight-chain and branched-chain radicals. The alkoxylene chain may be attached to the rest of the molecule through a single bond and to the radical group through a single bond. In some embodiments, the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen that is independently optionally substituted. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

The term heteroaryl also embraces N-heteroaryl groups which as used herein refers to a heteroaryl group, as defined above, and which contains at least one nitrogen atom and where the point of attachment of the N-heteroaryl group to the rest of the molecule is through a nitrogen atom in the heteroaryl group. The term heteroaryl further embraces C-heteroaryl groups which as used herein refer to a heteroaryl group as defined above and where the point of attachment of the heteroaryl group to the rest of the molecule is through a carbon atom in the heteroaryl group. The term heteroaryl further embraces heteroarylalkyl groups which as disclosed above refer to a group of the formula —R^{c}-heteroaryl, wherein R^{c} is an alkylene chain as defined above. The term heteroaryl further embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may thus include alkyl (e.g., C₁-C₆, C₁-C₅, C₁-C₄, C₁-C₃, C₁-C₂, C₁), substituted alkyl (e.g., substituted C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., substituted C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., substituted C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., substituted C₂-C₆, C₂-C₅, C₂-C₄, C₂-C₃, C₂), cyclic (e.g., C₃-C₁₂, C₅-C₆), substituted cyclic (e.g., substituted C₃-C₁₂, C₅-C₆), carbocyclic (e.g., C₃-C₁₂, C₅-C₆), substituted carbocyclic (e.g., substituted C₃-C₁₂, C₅-C₆), heterocyclic (e.g., 3- to 12-membered, 5- to 6-membered), substituted heterocyclic (e.g., substituted 3- to 12-membered, 5- to 6-membered), aryl (e.g., benzyl and phenyl), substituted aryl (e.g., substituted benzyl or substituted phenyl), heteroaryl (e.g., pyridyl or pyrimidyl), substituted heteroaryl (e.g., substituted pyridyl or substituted pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., substituted C₆-C₁₂, C₆), alkylthio (e.g., C₁-C₆), substituted alkylthio (e.g., substituted C₁-C₆), arylthio (e.g., C₆-C₁₂, C₆), substituted arylthio (e.g., substituted C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

In one aspect, compounds of the invention are represented by formula (I):

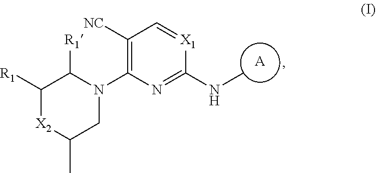

or a pharmaceutically acceptable salt or stereoisomer thereof, wherein:
X₁ is N, CH, CCl, CF, or CCN;
each X₂ is independently CH₂, S, CHF, CHCl, CHOH, or CF₂;
R₁ is hydrogen, ═O, —CN, —C≡CH, —OH, —SH, —NH₂, —COOH, halo, (C₁-C₆)alkyl, —O—(C₁-C₆)alkyl, (C₁-C₆)haloalkyl, amido, carboxy, carbamoyl, sulfamoyl, phenyl, 5- to 8-membered heterocyclyl, —NR₇R₈, —C(O)R₉, —C(O)NR₁₀R₁₁, or L₁Y₁, wherein said alkyl, phenyl, or heterocyclyl is optionally substituted with one or more groups selected from halo, —COOH, —OH, —NH₂, (C₁-C₆)alkyl, —C(O)O—(C₁-C₆)alkyl, —C(O)N(C₁-C₆ alkyl)₂, —O—(C₁-C₆)alkyl, —N(C₁-C₃ alkyl)₂, phenyl, and 4- to 6-membered heterocyclyl, optionally substituted with one or more groups selected from halo and (C₁-C₆)alkyl;
   R₇ is hydrogen, (C₁-C₄)alkyl, or (C₃-C₆)cycloalkyl;
   R₈ is hydrogen, (C₁-C₄)alkyl, (C₁-C₄)haloalkyl, (C₃-C₆)cycloalkyl, or 6-membered heterocyclyl;
   R₉ is —(C₁-C₃)alkyl-N(C₁-C₃ alkyl)₂, (C₃-C₆)cycloalkyl, or 5- to 6-membered heterocyclyl, wherein said heterocyclyl is optionally substituted with (C₁-C₃)alkyl;
   R₁₀ is hydrogen, (C₁-C₃)alkyl, or (C₃-C₆)cycloalkyl;
   R₁₁ is (C₃-C₆)cycloalkyl or (C₁-C₆)alkyl optionally substituted with —NH₂, —O—(C₁-C₆)alkyl, —O—(C₁-C₆)alkyl-NH₂, or —O—(C₁-C₆)alkyl-O—(C₁-C₆)alkyl-NH₂;
   L₁ is absent, (C₁-C₆)alkylene or (C₃-C₇)carbocyclyl; wherein said alkylene or carbocyclyl is further optionally substituted by one or more, identical or different R_{A} groups;
      each R_{A} is independently oxo, alkyl, alkenyl, alkynyl, halo, haloalkyl, carbocyclyl, heterocyclyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, or phosphinyl;
   Y₁ is —CN, —OH, halo, (C₁-C₄)alkoxy, (C₁-C₄)haloalkyl, (C₁-C₄)haloalkoxy, 4- to 7-membered heterocyclyl, (C₃-C₆)carbocyclyl, —NR_{7′}R_{8′}, —C(O)R₉, —C(O)NR₁₀R₁₁; wherein said alkyl, carbocyclyl, or heterocyclyl is further optionally substituted by one or more, identical or different groups selected from (C₁-C₄)alkyl, halo, (C₁-C₄)haloalkyl, —CN, —OH, and —NH₂;
      R_{7′} and R_{8′} are each independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl; wherein said alkyl, carbocyclyl, heterocyclyl, aryl or heteroaryl is further optionally substituted by one or more, identical or different R_{A} groups, or
      R_{7′} and R_{8′} together with the nitrogen atom to which they are attached form a 3- to 7-membered heterocyclyl, wherein said heterocyclyl is further optionally substituted by one or more, identical or different R_{A} groups, or
   L₁ is (C₂-C₄)alkylene which is bound to R₇, to form a 4- to 6-membered heterocyclyl group;
R₁′ is absent, hydrogen, —CN, —C≡CH, —OH, —SH, —NH₂, —COOH, halo, (C₁-C₆)alkyl, —O—(C₁-C₆)alkyl, (C₁-C₆)haloalkyl, amido, carboxy, carbamoyl, sulfamoyl, phenyl, 5- to 8-membered heterocyclyl, —NR₇R₈, —C(O)R₉, or —C(O)NR₁₀R₁₁; wherein said alkyl, phenyl, or heterocyclyl is further optionally substituted by one or more, identical or different R_{A} groups, or
R₁′ and L₁ together with the same carbon atom to which they are attached form a spiro (C₃-C₇)carbocyclyl group or a 4- to 7-membered heterocyclyl group; wherein said carbocyclyl or heterocyclyl, is further optionally substituted by one or more, identical or different R_{A} groups;
\textcircled{A} is

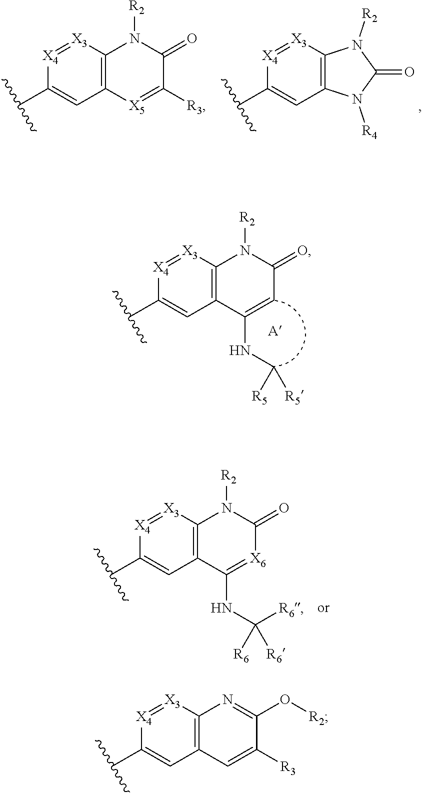

X₃ and X₄ are independently CR₁₂ or N;
X₅ is CH or N;
   R₁₂ is hydrogen, (C₁-C₄)alkyl, halo, hydroxy, amino, (C₁-C₄)alkoxy, (C₁-C₄)haloalkyl, (C₁-C₄)haloalkoxy, (C₂-C₄)alkenyl, (C₂-C₄)alkynyl, nitro, cyano, NH(C₁-C₄)alkyl, or N(C₁-C₄ alkyl)₂;
R₂ is hydrogen, (C₁-C₆)alkyl, (C₃-C₆)carbocyclyl, 4- to 7-membered heterocyclyl, (C₃-C₇)carbocyclyl(C₁-C₆)alkyl, or 4- to 7-membered heterocyclyl(C₁-C₆)alkyl; wherein said alkyl, carbocyclyl, or heterocyclyl is further optionally substituted by one or more, identical or different R₁₃ groups, wherein R₁₃ is (C₁-C₆)alkyl, (C₁-C₆)alkoxy, halo, amino, hydroxyl, haloalkyl, NH(C₁-C₆)alkyl, or N((C₁-C₆)alkyl)₂, (C₃-C₆)carbocyclyl, or 4- to 7-membered heterocyclyl, or R₂ is -L₂-Y₂—Z;
L₂ is absent or (C₁-C₅)alkylene optionally substituted by one or more substituents selected from (C₁-C₂)alkyl and oxo;
Y₂ is absent, O, S, S(O), S(O)₂, NR′, C(O), C(O)O, OC(O), C(O)N(R′), N(R′)C(O), N(R′)C(O)N(R′), N(R′)C(O)O, OC(O)N(R′), S(O)₂N(R′), or N(R′)S(O)₂;
each R′ is independently hydrogen or (C₁-C₄)alkyl;
Z is hydrogen, (C₁-C₆)alkyl, (C₂-C₆)alkenyl, (C₂-C₆)alkynyl, (C₃-C₁₀)carbocyclyl, or 3- to 10-membered heterocyclyl; wherein Z is optionally substituted by one or more substituents independently selected from (C₁-C₄)alkyl, halo, (C₁-C₄)haloalkyl, (C₁-C₄)haloalkoxy, amino, (C₁-C₄)aminoalkyl, cyano, hydroxy, carboxy, carbamoyl, sulphamoyl, mercapto, ureido, NR^{r}R^{s}, OR^{r}, C(O)R^{r}, C(O)OR^{r}, OC(O)R^{r}, C(O)NR^{r}R^{s}, N(R^{r})C(O)R^{r}, S(O)₀₋₂R^{r}, S(O)₂NR^{r}R^{s}, N(R^{r})SO₂R^{r}, Si(R^{r})(R^{s})R^{t} and (CH₂)₁₋₃NR^{r}R^{s}; wherein R^{r}, R^{s}, and R^{t} are each independently hydrogen, (C₁-C₆)alkyl, or (C₃-C₆)cycloalkyl; or R^{r} and R^{s} together with the nitrogen atom to which they are attached form a 4- to 9-membered heterocyclyl which is optionally substituted by one or more substituents selected from (C₁-C₄)alkyl, halo, (C₁-C₄)haloalkyl, (C₁-C₄)haloalkoxy, (C₁-C₄)alkoxy, (C₁-C₄)alkylamino, amino, cyano, and hydroxy;
R₃ is -L₃CR₁₄R₁₅R₁₆, or —CH═CH—R₁₆;
   L₃ is absent, O, S, (C₁-C₄)alkylene, —O—(C₁-C₄)alkylene, or —S—(C₁-C₄)alkylene;
   R₁₄ is hydrogen or (C₁-C₄)alkyl;
   R₁₅ is hydrogen or (C₁-C₄)alkyl, or
   R₁₄ and R₁₅ together with the carbon atom to which they are attached form a (C₃-C₅)carbocyclyl, 4- to 7-membered heterocyclyl, or C═O;
   R₁₆ is (C₁-C₆)alkyl, —NR₁₇R₁₈, —OR₁₇, —C(O)R₁₇, —C(O)OR₁₇, —N(R₁₈)C(O)R₁₇, —C(O)NR₁₇R₁₈, —S(O)—(C₁-C₆)alkyl, —S(O)₂—(C₁-C₆)alkyl, —P(O)—(C₁-C₆ alkyl)₂, —C(NH)NH₂, —(C₁-C₄)alkyl-NR₁₈C(O)R₁₇, or 4- to 7-membered heterocyclyl;
      R₁₇ is hydrogen, 3- to 6-membered heterocyclyl, or (C₁-C₄)alkyl optionally substituted by one or more, identical or different groups selected from OH, Cl, F, CF₃, N(C₁-C₄ alkyl)₂, (C₃-C₆)carbocyclyl, 3- to 6-membered heterocyclyl, (C₂-C₄)alkenyl, and (C₂-C₄)alkynyl;
      R₁₈ is hydrogen or (C₁-C₄)alkyl;
R₄ is hydrogen, methyl, —(CH₂)₁₋₃W₁W₂, or

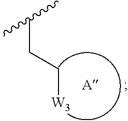

W₁ is CR₁₉R_{19′} or C(O);
   R₁₉ and R_{19′} are independently hydrogen, (C₁-C₂)alkyl, fluoro, hydroxy, cyano, nitro, (C₁-C₂)alkoxy, (C₁-C₂)haloalkyl, (C₁-C₂)haloalkoxy, amino, NH(C₁-C₂)alkyl, or N(C₁-C₂ alkyl)₂, or
R₁₉ and R_{19′} together with the carbon atom to which they are attached form C(O), (C₃-C₆)carbocyclyl or 3- to 6-membered heterocyclyl, which is optionally substituted by one or more substituents independently selected from (C₁-C₂)alkyl, halo, (C₁-C₂)haloalkyl, (C₁-C₂)haloalkoxy, (C₁-C₂)alkoxy, (C₁-C₂)alkylamino, amino, cyano, and hydroxy;
W₂ is cyano, hydroxy, 5- or 6-membered heteroaryl, phenyl, C(O)—(C₁-C₂)alkyl, S(O)₂—(C₁-C₂)alkyl, C(O)OCH₃, C(O)NHCH₃, CR₂₀R₂₁R₂₂, amino, NH(C₁-C₂)alkyl, or N(C₁-C₂ alkyl)₂:
   R₂₀ is hydrogen, (C₁-C₂)alkyl, fluoro, chloro, bromo, hydroxy, amino, cyano, nitro, (C₁-C₂)alkoxy, (C₁-C₂)haloalkyl, or (C₁-C₂)haloalkoxy;
   R₂₁ is hydrogen, (C₁-C₂)alkyl, fluoro, chloro, bromo, hydroxy, cyano, nitro, (C₁-C₂)alkoxy, (C₁-C₂)haloalkyl, (C₁-C₂)haloalkoxy, or —Y₃-L₄-Z₂;
      Y₃ is absent, O, S, S(O), S(O)₂, NR′, C(O), C(O)O, OC(O), C(O)N(R′), N(R′)C(O), S(O)₂N(R′), or N(R′)SO₂;
      L₄ is absent or (C₁-C₂)alkylene;
      Z₂ is hydrogen, (C₁-C₆)alkyl, (C₂-C₄)alkenyl, (C₂-C₄)alkynyl, phenyl, (C₃-C₆)carbocyclyl, or 4- to 6-membered heterocyclyl, wherein Z₂ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, amino, cyano, hydroxy, C(O)R′, C(O)OR′, OC(O)R′, C(O)NR′R′, and N(R′)C(O)R′, wherein each R′ is independently hydrogen or (C₁-C₄)alkyl;
or R₂₀ and R₂₁ together with the carbon atom to which they are attached form (C₃-C₆)carbocyclyl or 3- to 6-membered heterocyclyl, optionally substituted by one or more substituents selected from (C₁-C₂)alkyl, halo, (C₁-C₂)haloalkyl, (C₁-C₂)haloalkoxy, (C₁-C₂)alkoxy, (C₁-C₂)alkylamino, amino, cyano, and hydroxy;
R₂₂ is (C₁-C₂)alkyl, —C(O)OR″, OR″, —C(O)NR″, NR″R″, phenyl, or 5-membered heteroaryl, wherein each R″ is independently hydrogen or (C₁-C₂)alkyl;
A″ is (C₄-C₆)carbocyclyl or 4- to 6-membered heterocyclyl, optionally substituted with one or more substituents independently selected from (C₁-C₂)alkyl, halo, hydroxy, oxo, cyano, and (C₁-C₂)alkoxy;
W₃ is NR₂₃ or CR₂₄R_{24′};
   R₂₃ is hydrogen, (C₁-C₂)alkyl, (C₁-C₄)haloalkyl, (C₁-C₄)hydroxyalkyl, —C(O)CH₃, or —C(O)O—(C₁-C₄)alkyl;
   R₂₄ and R_{24′} are independently hydrogen, (C₁-C₂)alkyl, cyclopropyl, fluoro, chloro, bromo, hydroxy, amino, cyano, nitro, (C₁-C₂)haloalkyl, (C₁-C₂)haloalkoxy, (C₁-C₂)alkoxy, —C(O)OR″, NR″R″, phenyl, or 5-membered heteroaryl;
R₅ is hydrogen, (C₁-C₄)alkyl, (C₃-C₆)cycloalkyl, (C₁-C₄)haloalkyl, or cyano, wherein said alkyl or cycloalkyl is optionally substituted by one or more substituents selected from (C₁-C₄)alkyl, (C₃-C₆)cycloalkyl, hydroxy, (C₁-C₂)alkoxy, amino, NH(C₁-C₂)alkyl, N((C₁-C₂)alkyl)₂, (C₁-C₂)aminoalkyl, and halo;
R₅′ is hydrogen, (C₁-C₄)alkyl, cyano, (C₁-C₄)haloalkyl, or —Y₄-L₅-Z₃;
   Y₄ is absent, C(O)O, or C(O)N(R″);
   L₅ is absent or (C₁-C₂)alkylene;
   Z₃ is hydrogen, (C₁-C₆)alkyl, phenyl, (C₃-C₆)cycloalkyl, or 4- to 6-membered heterocyclyl, wherein Z₃ is optionally substituted by one or more substituents independently selected from (C₁-C₂)alkyl, halo, (C₁-C₂)haloalkyl, (C₁-C₂)haloalkoxy, (C₁-C₂)alkoxy, amino, nitro, cyano, and hydroxy, or
R₅ and R₅′, together with the carbon atom to which they are attached, form a (C₄-C₆)carbocyclyl, or 4- to 6-membered heterocyclyl;
A′ is a 6- or 7-membered heterocyclyl, which in addition to R₅ and R₅′, is optionally further substituted by one more substituents independently selected from oxo, (C₁-C₂)alkyl, cyclopropyl, spiro-cyclopropyl, halo, (C₁-C₂)haloalkyl, (C₁-C₂)haloalkoxy, (C₁-C₂)alkoxy, amino, cyano, and hydroxy;
X₆ is CR₂₅ or N;
   R₂₅ is hydrogen, fluor, chloro, or methyl;
R₆ is hydrogen, (C₁-C₂)alkyl, (C₃-C₄)cycloalkyl, (C₁-C₂)haloalkyl, cyano, (C₂-C₄)alkenyl, or (C₂-C₄)alkynyl;
R₆′ is (C₁-C₄)alkyl, cyano, (C₁-C₄)haloalkyl, or —Y₅-L₆-Z₄;
   Y₅ is absent, C(O), C(O)O, OC(O), C(O)N(R″), or S(O)₂N(R″);
   L₆ is absent or (C₁-C₂)alkylene optionally substituted by one or more substituents selected from (C₁-C₂)alkyl and oxo;
   Z₄ is hydrogen, (C₁-C₆)alkyl, (C₂-C₄)alkenyl, (C₂-C₄)alkynyl, phenyl, (C₃-C₆)carbocyclyl, (C₃-C₆)cycloalkenyl, or 4- to 6-membered heterocyclyl, wherein Z₄ is optionally substituted by one or more substituents independently selected from oxo, (C₁-C₄)alkyl, (C₃-C₆)cycloalkyl, halo, (C₁-C₄)haloalkyl, (C₁-C₄)haloalkoxy, (C₁-C₄)alkoxy, (C₁-C₄)alkylamino, amino, nitro, cyano, hydroxy, C(O)R^{u}, C(O)OR^{u}, OC(O)R^{u}, C(O)NR^{u}R^{u}, and N(R^{u})C(O)R^{u}, wherein each R^{u} is independently hydrogen, (C₁-C₄)alkyl, or (C₃-C₆)cycloalkyl, or
Z₄ is -Q-L₇-W₄, wherein
   Q is absent, O, NH, or N(C₁-C₂)alkyl;
   L₇ is absent or (C₁-C₂)alkylene optionally substituted by one or more substituents selected from oxo and (C₁-C₂)alkyl;
   W₄ is (C₁-C₄)alkyl, phenyl, (C₃-C₆)cycloalkyl, (C₃-C₆)cycloalkenyl, or 5- or 6-membered heterocyclyl, wherein W₄ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, amino, nitro, cyano, or hydroxy, or
R₆ and R₆′, together with the carbon atom to which they are attached, for a (C₃-C₁₀)carbocyclyl or a 4- to 10-membered heterocyclyl, which is optionally substituted by one or more substituents independently selected from oxo, (C₁-C₂)alkyl, halo, (C₁-C₂)haloalkyl, (C₁-C₂)haloalkoxy, (C₁-C₂)alkoxy, (C₁-C₂)alkylamino, amino, nitro, cyano, or hydroxy; or the (C₃-C₁₀)carbocyclyl or 4- to 10-membered heterocyclyl is optionally fused to a 5- or 6-membered heteroaryl or phenyl ring, and the 5- or 6-membered heteroaryl or phenyl ring is optionally substituted by (C₁-C₂)alkyl, halo, (C₁-C₂)haloalkyl, (C₁-C₂)haloalkoxy, (C₁-C₂)alkoxy, (C₁-C₂)alkylamino, amino, nitro, cyano, or hydroxy; and
R₆″ is hydrogen, (C₁-C₄)alkyl, (C₁-C₂)haloalkyl, (C₁-C₂)alkoxy, (C₁-C₂)haloalkoxy, cyano, nitro, acetylenyl, phenyl, or 5- or 6-membered heteroaryl, wherein said alkyl, phenyl, or heteroaryl is optionally substituted by one or more substituents independently selected from halo, hydroxy, and amino.

In some embodiments, R₁ is methyl, —OH, —NH₂, —COOH, —CH₂CH₂OH, —CH₂CH₂NH₂,

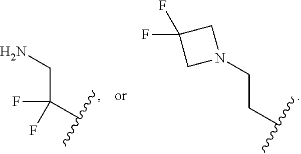 [SMILES: *C(F)(F)CN ; *CCN1CC(F)(F)C1]

In some embodiments, R₁ is methyl. In some embodiments, R₁ is —OH. In some embodiments, R₁ is —NH₂. In some embodiments, R₁ is —COOH. In some embodiments, R₁ is —CH₂CH₂OH. In some embodiments, R₁ is —CH₂CH₂NH₂.

In some embodiments, X₁ is N, CH, CCl, or CF. In some embodiments, X₁ is N. In some embodiments, X₁ is CH. In some embodiments, X₁ is CCl. In some embodiments, X₁ is CF.

In some embodiments, X₂ is CH₂, CHF, CHCl, or CF₂. In some embodiments, X₂ is CH₂. In some embodiments, X₂ is CHF. In some embodiments, X₂ is CHCl. In some embodiments, X₂ is CF₂.

In some embodiments, \textcircled{A} is

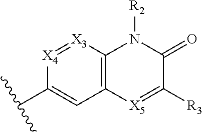

and the compound of formula (I) has the structure of formula I-1,

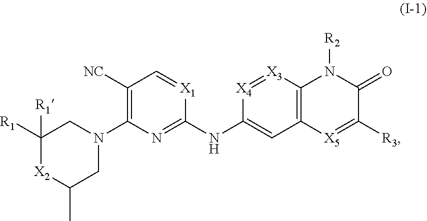

or a pharmaceutically acceptable salt or stereoisomer thereof.

In some embodiments of formula I-1, X₃ is N. In some embodiments of formula I-1, X₃ is CR₁₂. In some embodiments of formula I-1, X₃ is CH. In some embodiments of formula I-1, X₃ is CF. In some embodiments of formula I-1, X₃ is COMe.

In some embodiments of formula I-1, X₄ is N. In some embodiments of formula I-1, X₄ is CR₁₂. In some embodiments of formula I-1, X₄ is CH. In some embodiments of formula I-1, X₄ is CF. In some embodiments of formula I-1, X₄ is COMe.

In some embodiments of formula I-1, X₅ is N. In some embodiments of formula I-1, X₅ is CH.

In some embodiments of formula I-1, R₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different R₁₃ groups. In some embodiments, R₂ is methyl. In some embodiments, R₂ is 4-membered heterocyclyl, (C₃)carbocyclyl(C₁)alkyl, or 4-membered heterocyclyl(C₁)alkyl, wherein the heterocyclyl contains 1 heteroatom selected from N and O.

In some embodiments of formula I-1, R₃ is -L₃CR₁₄R₁₅R₁₆. In some embodiments of formula I-1, L₃ is (C₁-C₄)alkylene, —O—(C₁-C₄)alkylene, or —S—(C₁-C₄)alkylene. In some embodiments of formula I-1, L₃ is —O—(C₁-C₄)alkylene. In some embodiments of formula I-1, L₃ is —O—(C₁)alkylene.

In some embodiments of formula I-1, R₁₄ and R₁₅ together with the carbon atom to which they are attached form a (C₃-C₅)carbocyclyl, 4- to 7-membered heterocyclyl, or C═O. In some embodiments of formula I-1, R₁₄ and R₁₅ together with the same carbon atom to which they are attached form C═O. In some embodiments of formula I-1, R₁₄ and R₁₅ together with the same carbon atom to which they are attached form 4- to 7-membered heterocyclyl. In some embodiments of formula I-1, R₁₄ and R₁₅ together with the same carbon atom to which they are attached form an oxetane ring.

In some embodiments of formula I-1, R₁₆ is (C₁-C₆)alkyl, —NR₁₇R₁₈, or —OR₁₇. In some embodiments of formula I-1, R₁₆ is methyl, hydroxyl, amino, or NHMe. In some embodiments, R₁₆ is methyl. In some embodiments, R₁₆ is hydroxyl. In some embodiments, R₁₆ is amino. In some embodiments, R₁₆ is NHMe.

In some embodiments, the compound of formula I-1 is of formula I-1a, I-1b, I-1c, I-1d, I-1e, I-1f, I-1g, I-1h, I-1i, or I-1j:

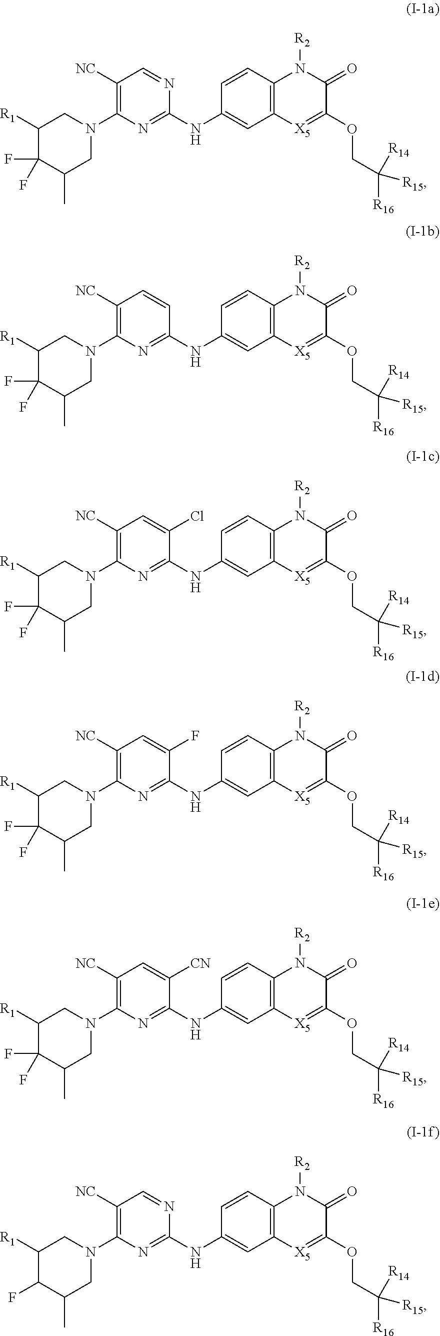

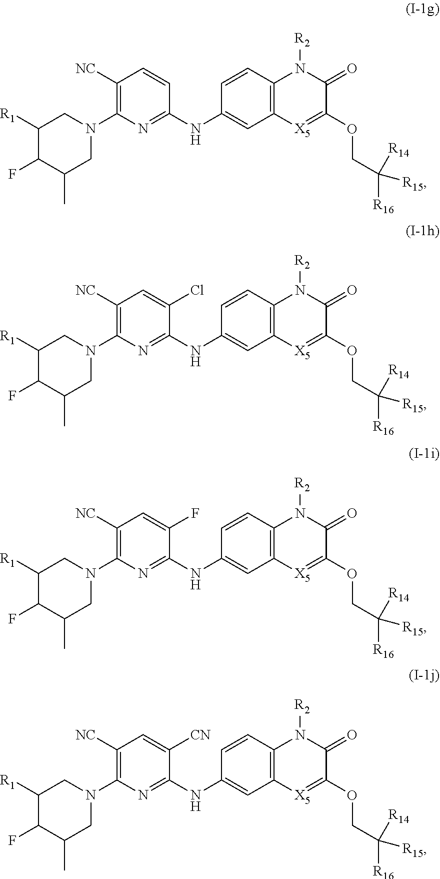

or a pharmaceutically acceptable salt or stereoisomer thereof.

In some embodiments, the compound of formula I-1 is of formula I-1a′, I-1b′, I-1c′,

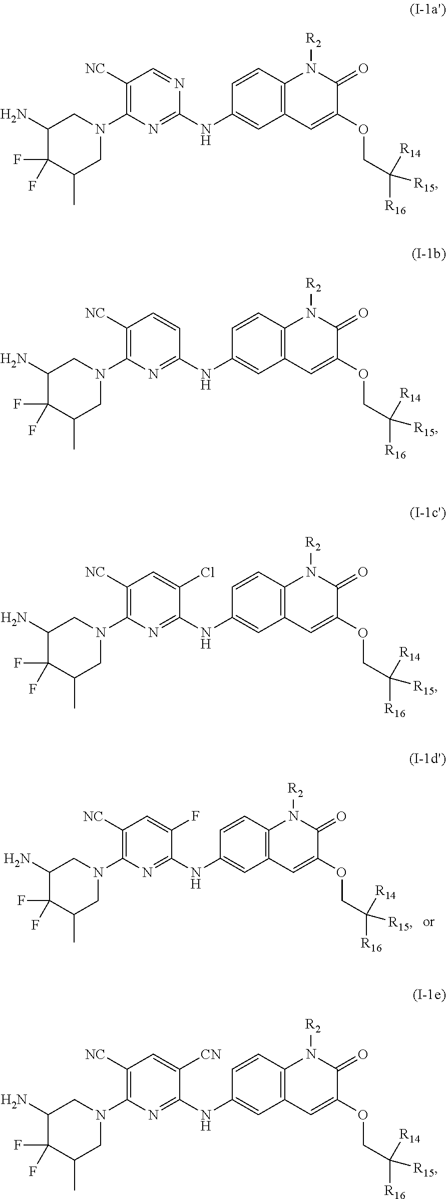

or a pharmaceutically acceptable salt or stereoisomer thereof. In some embodiments of formula I-1a′, I-1b′, I-1c′, I-1d′, and I-1e′, R₂ is methyl. In some embodiments of formula I-1a′, I-1b′, I-1c′, I-1d′, and I-1e′, R₁₄ and R₁₅ form C═O and R₁₆ is —NR₁₇R₁₈. In some embodiments of formula I-1a′, I-1b′, I-1c′, I-1d′, and I-1e′, R₁₆ is NHMe.

In some embodiments, the compound of formula I-1 is of formula I-1k, I-1l, I-1m, I-1n, I-1o, I-1p, I-1q, I-1r, I-1s, I-1t, I-1u, I-1v, I-1w, I-1x, I-1y, I-1 z, I-1 aa, I-1bb, I-1cc, or I-1dd.

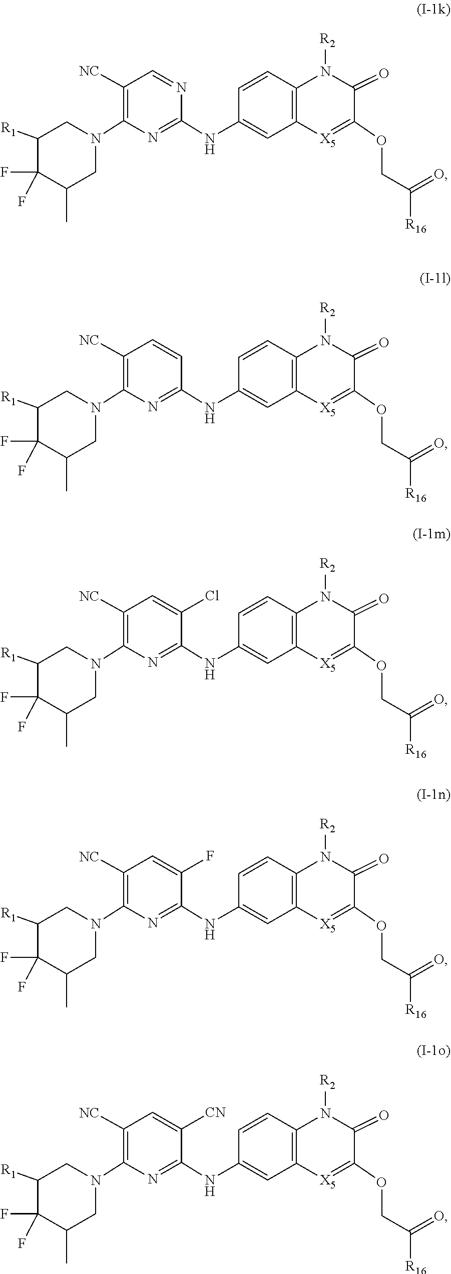

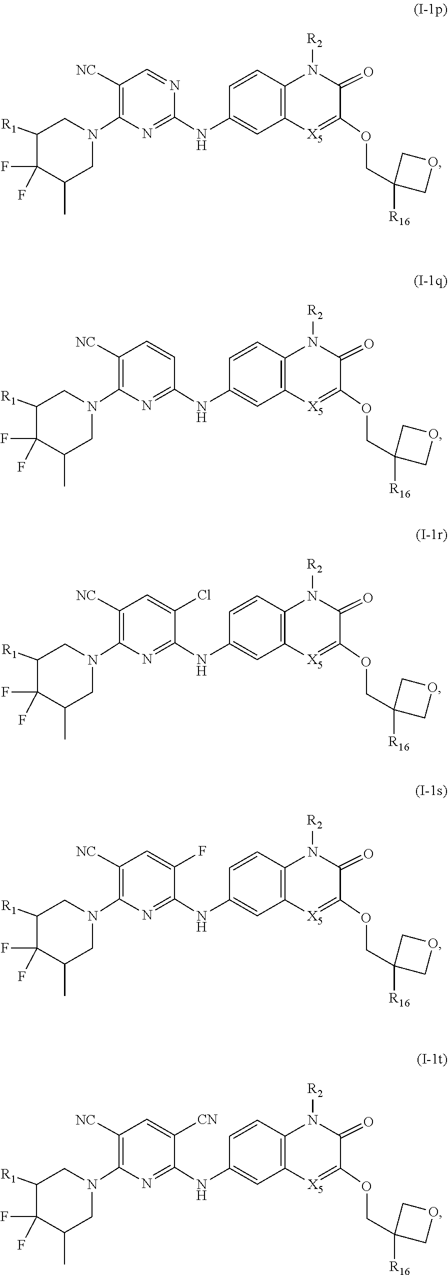

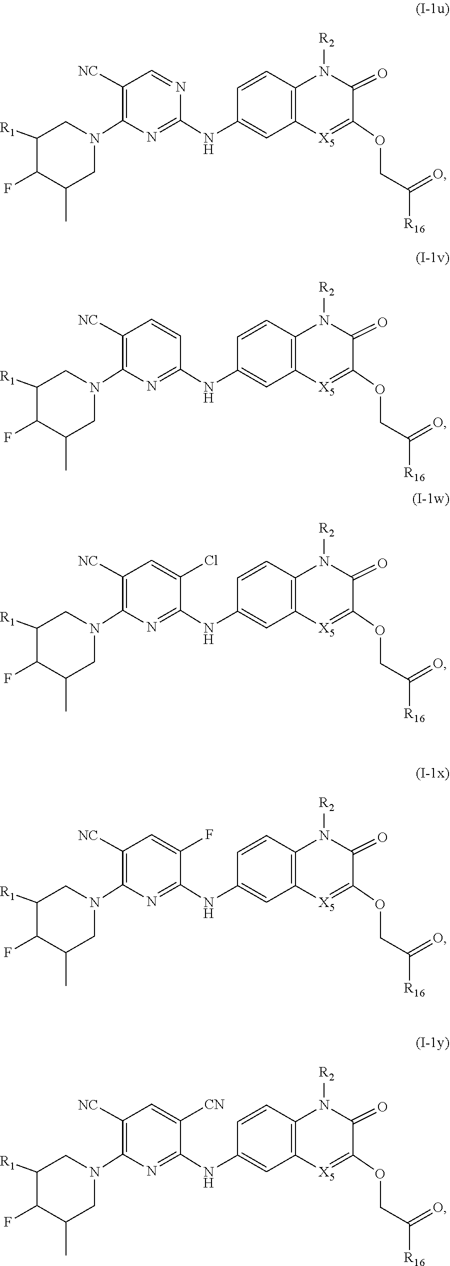

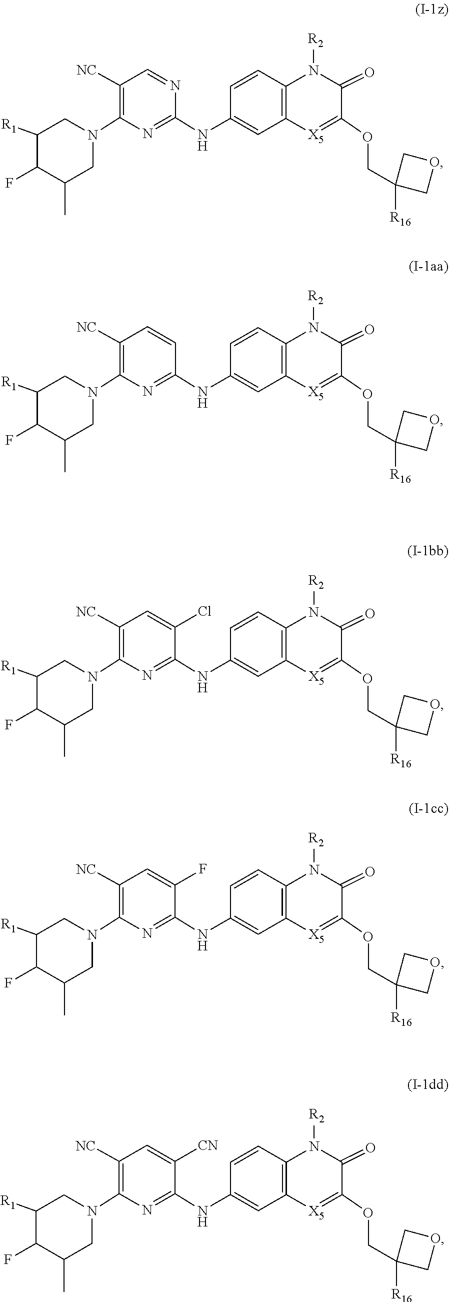

or a pharmaceutically acceptable salt or stereoisomer thereof.

In some embodiments, \textcircled{A} is

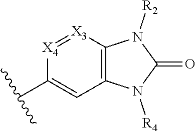

and the compound of formula (I) has the structure of formula I-2,

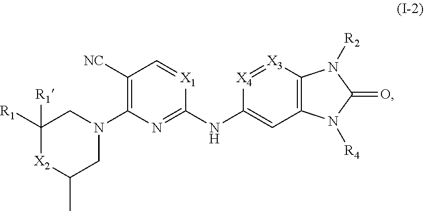

or a pharmaceutically acceptable salt or stereoisomer thereof.

In some embodiments of formula I-2, X₃ is CR₁₂. In some embodiments of formula I-2, X₃ is CH. In some embodiments of formula I-2, X₃ is N.

In some embodiments of formula I-2, X₄ is CR₁₂. In some embodiments of formula I-2, X₄ is CH. In some embodiments of formula I-2, X₄ is N.

In some embodiments of formula I-2, R₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different R₁₃ groups. In some embodiments, R₂ is methyl. In some embodiments, R₂ is 4-membered heterocyclyl, (C₃)carbocyclyl(C₁)alkyl, or 4-membered heterocyclyl(C₁)alkyl, wherein the heterocyclyl contains 1 heteroatom selected from N and O.

In some embodiments of formula I-2, R₄ is —(CH₂)₁₋₃W₁W₂. In some embodiments of formula I-2, R₄ is —(CH₂)₂W₁W₂. In some embodiments of formula I-2, R₄ is

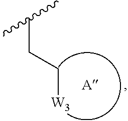

wherein W₃ is NR₂₃ and A″ is optionally substituted 4- to 6-membered heterocyclyl. In some embodiments of formula I-2, R₄ is

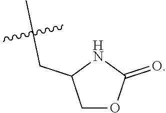 [SMILES: *CC1COC(=O)N1]

In some embodiments of formula I-2, W₁ is CR₁₉R_{19′}. In some embodiments of formula I-2, R₁₉ and R_{19′} are independently hydrogen or (C₁-C₂)alkyl. In some embodiments of formula I-2, R₁₉ and R_{19′} are both (C₁-C₂)alkyl. In some embodiments of formula I-2, R₁₉ and R_{19′} are both methyl. In some embodiments of formula I-2, R₁₉ and R_{19′} together with the carbon atom to which they are attached form (C₃-C₆)carbocyclyl. In some embodiments of formula I-2, R₁₉ and R_{19′} together with the carbon atom to which they are attached form cyclopropyl.

In some embodiments of formula I-2, W₂ is cyano, hydroxy, or amino. In some embodiments of formula I-2, W₂ is hydroxy.

In some embodiments, the compound of formula I-2 is of formula I-2a, I-2b, I-2c, I-2d, I-2e, I-2f, I-2g, I-2h, I-2i, or I-2j:

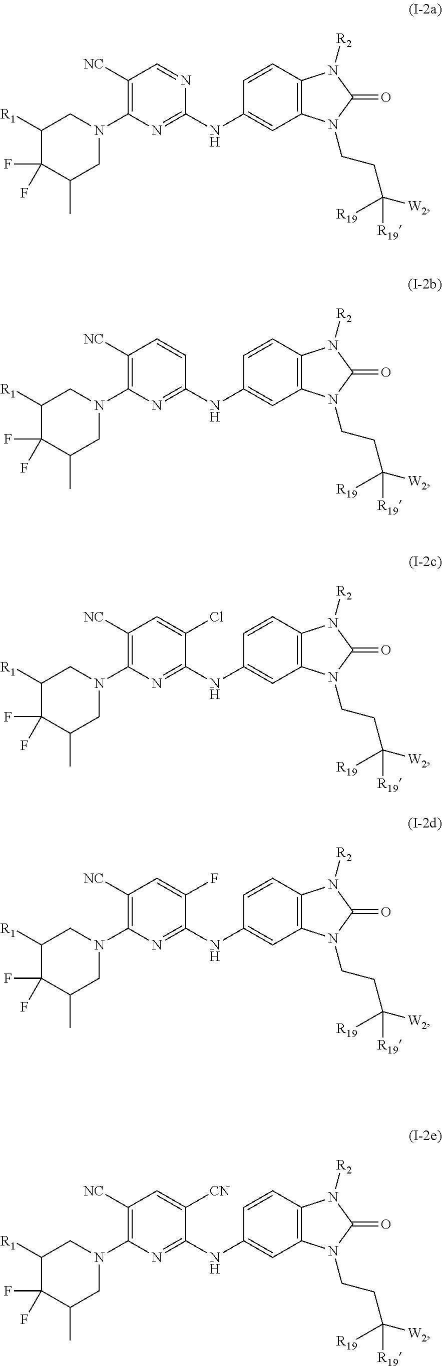

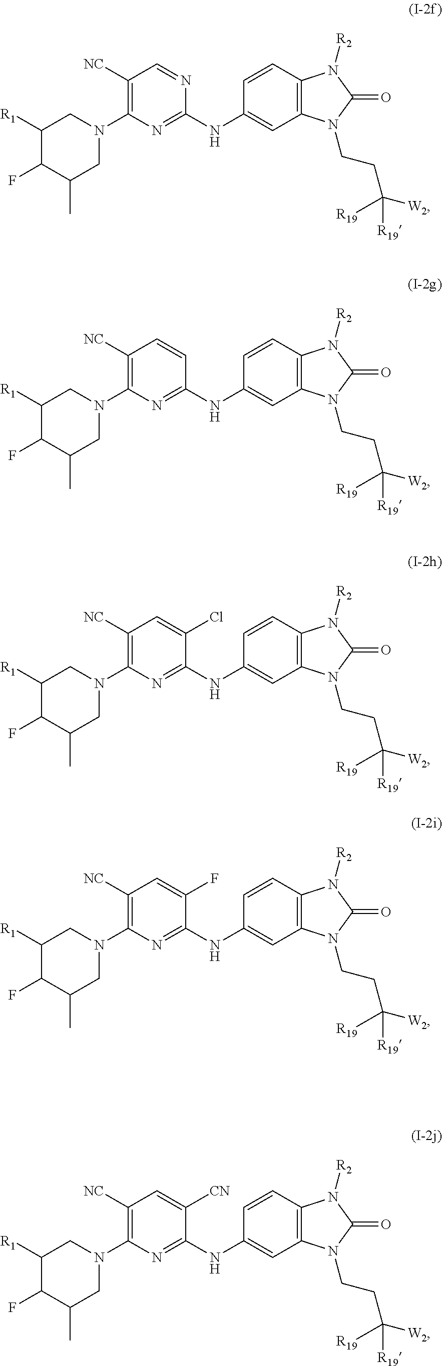

or a pharmaceutically acceptable salt or stereoisomer thereof.

In some embodiments, the compound of formula I-2 is of formula I-2k, I-2l, I-2m, I-2n, I-2o, I-2p, I-2q, I-2r, I-2s, I-2t, I-2u, I-2v, I-2w, I-2x, I-2y, I-2z, I-2aa, I-2bb, I-2cc, or I-2dd:

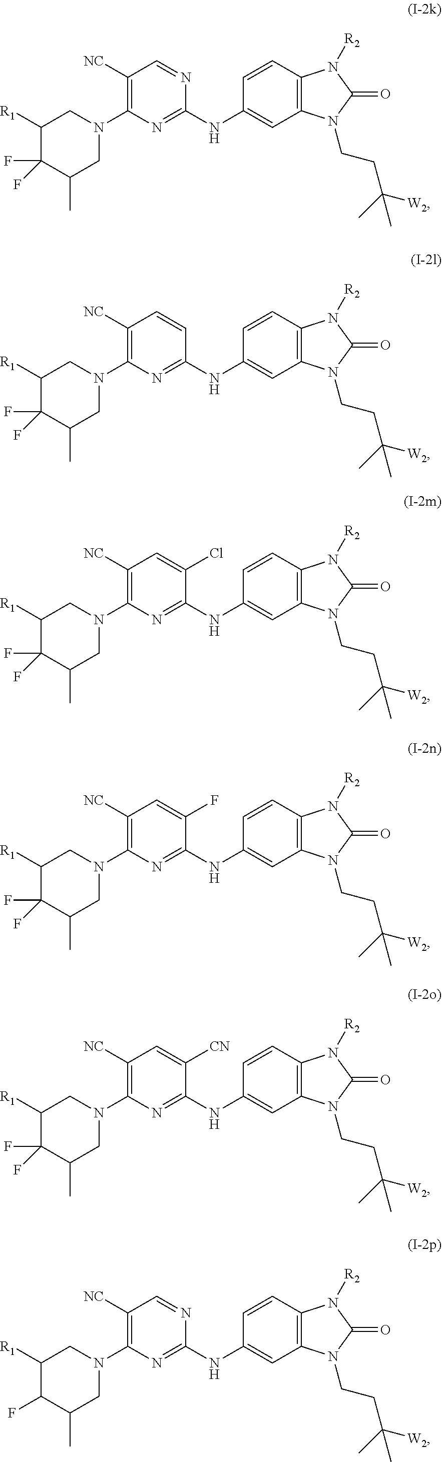

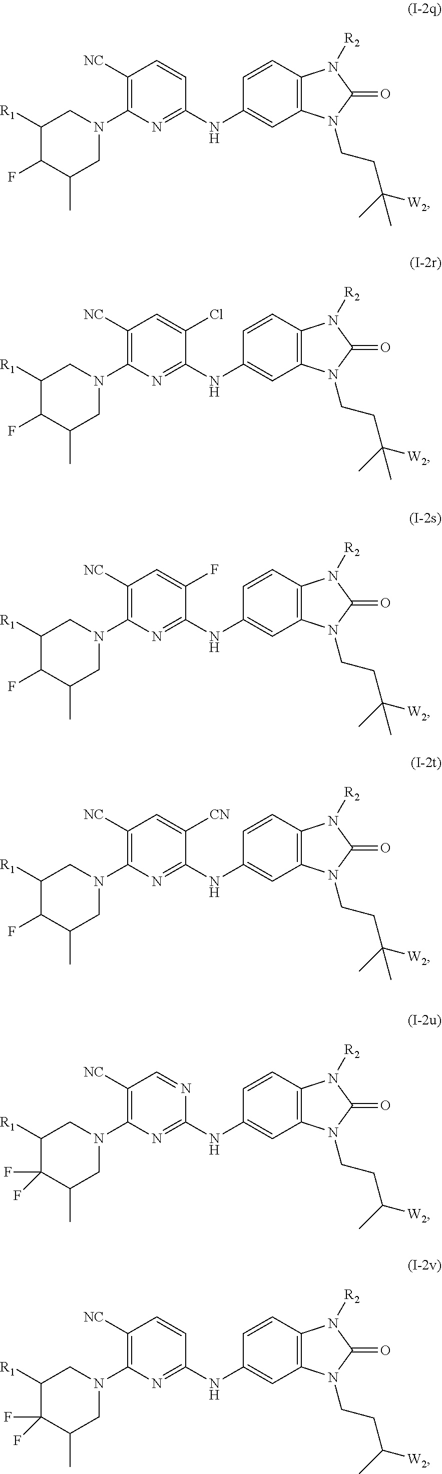

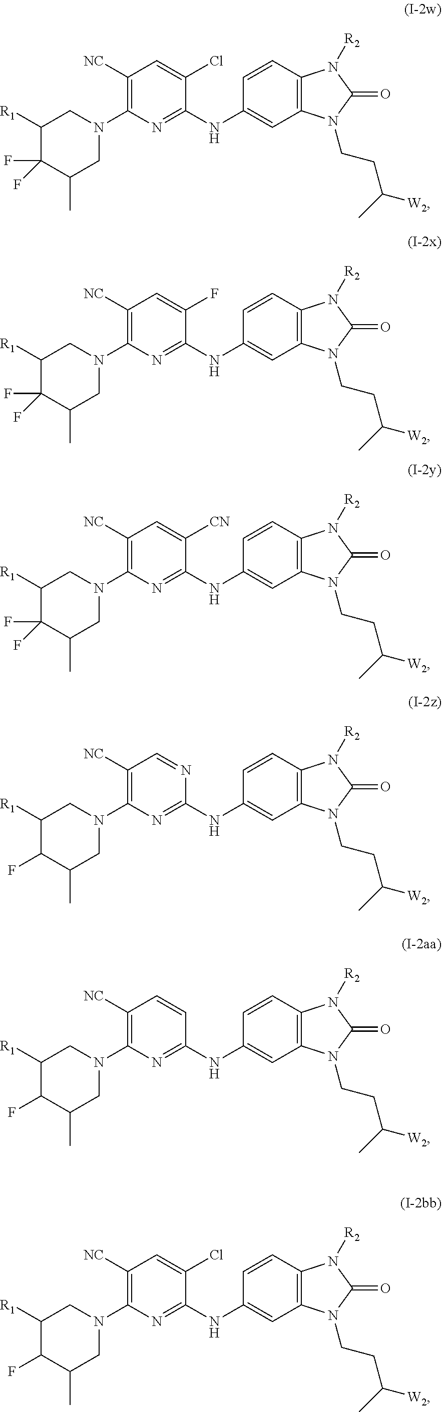

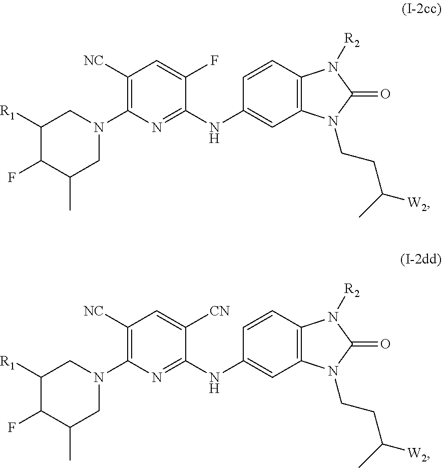

or a pharmaceutically acceptable salt or stereoisomer thereof.

In some embodiments, \textcircled{A} is

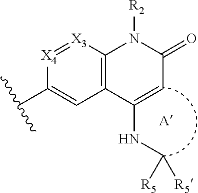

and the compound of formula (I) has the structure of formula I-3,

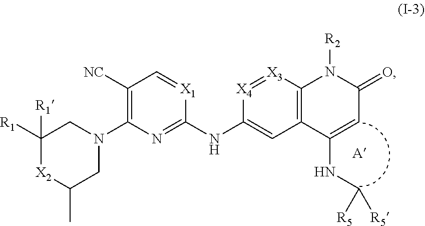

or a pharmaceutically acceptable salt or stereoisomer thereof.

In some embodiments of formula I-3, X₂ is CH₂. In some embodiments of formula I-3, X₂ is CF₂.

In some embodiments of formula I-3, X₃ is CR₁₂. In some embodiments of formula I-3, X₃ is CH. In some embodiments of formula I-3, X₃ is N.

In some embodiments of formula I-3, X₄ is CR₁₂. In some embodiments of formula I-3, X₄ is CH. In some embodiments of formula I-3, X₄ is N.

In some embodiments of formula I-3, R₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different R₁₃ groups. In some embodiments, R₂ is methyl. In some embodiments, R₂ is 4-membered heterocyclyl, (C₃)carbocyclyl(C₁)alkyl, or 4-membered heterocyclyl(C₁)alkyl, wherein the heterocyclyl contains 1 heteroatom selected from N and O.

In some embodiments of formula I-3, R₅ is (C₃-C₆)cycloalkyl and R₅′ is H. In some embodiments of formula I-3, R₅ is cyclopropyl and R₅′ is H.

In some embodiments of formula I-3, A′ is a 7-membered heterocyclyl, wherein the heterocyclyl contains 2 heteroatoms selected from N and O, and which in addition to R₅ and R₅′, is optionally further substituted by one more substituents independently selected from oxo, (C₁-C₂)alkyl, cyclopropyl, spiro-cyclopropyl, halo, (C₁-C₂)haloalkyl, (C₁-C₂)alkoxy, amino, cyano, and hydroxy.

In some embodiments, the compound of formula I-3 is of formula I-3a, I-3b, I-3c, I-3d, I-3e, I-3f, I-3g, I-3h, I-3i, I-3j, I-3k, I-3l, I-3m, I-3n, I-3o, I-3p, I-3q, I-3r, I-3s, or I-3t:

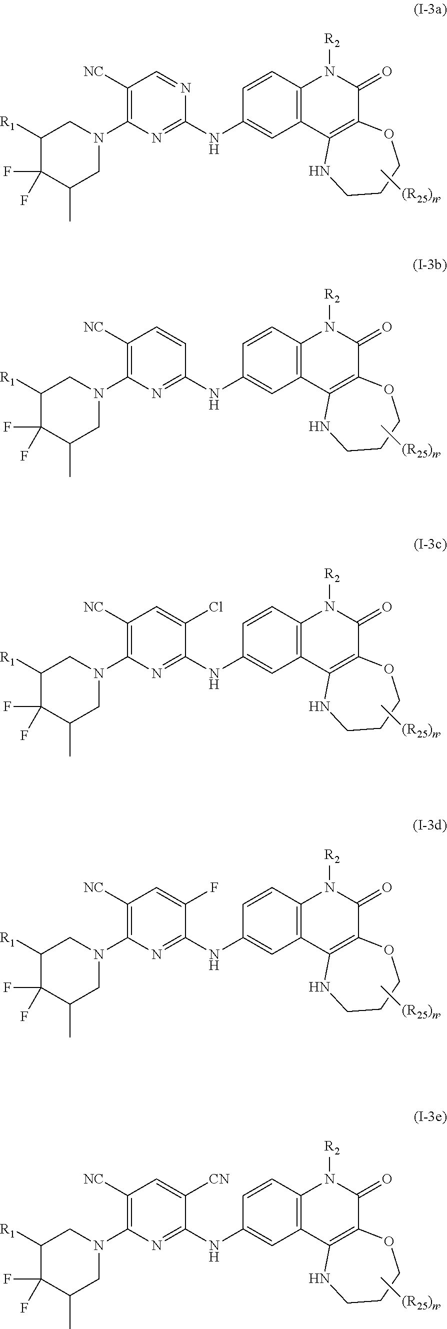

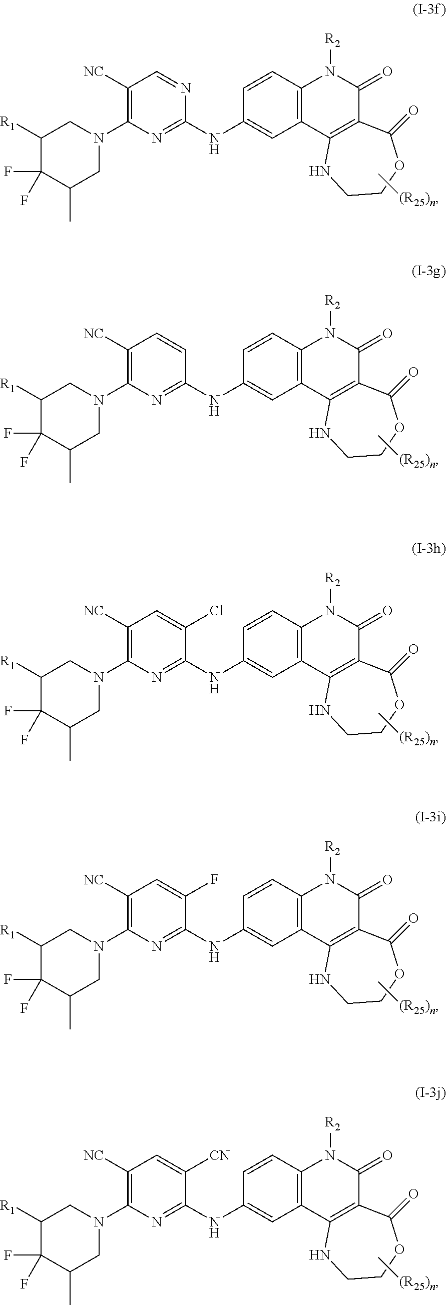

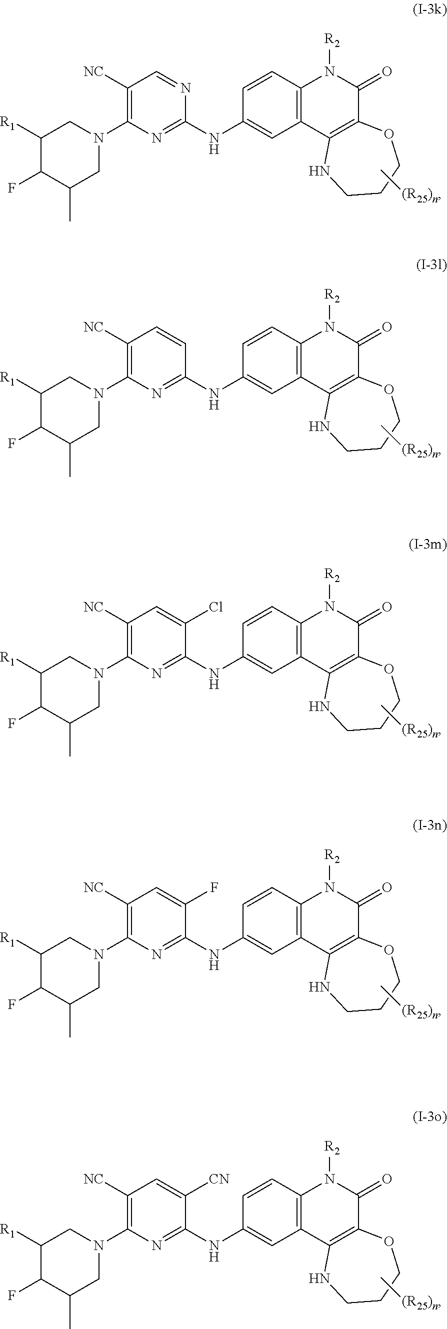

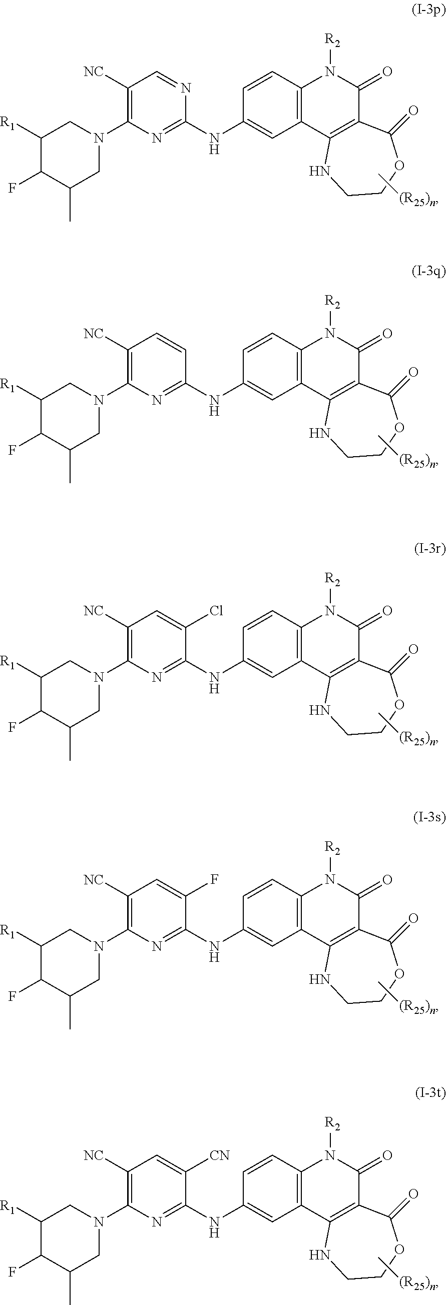

or a pharmaceutically acceptable salt or stereoisomer thereof, wherein each R₂₅ is independently oxo, (C₁-C₂)alkyl, cyclopropyl, spiro-cyclopropyl, halo, (C₁-C₂)haloalkyl, (C₁-C₂)alkoxy, amino, cyano, and hydroxy; and n is 0-3.

In some embodiments, the compound of formula I-3 is of formula I-3u or I-3v:

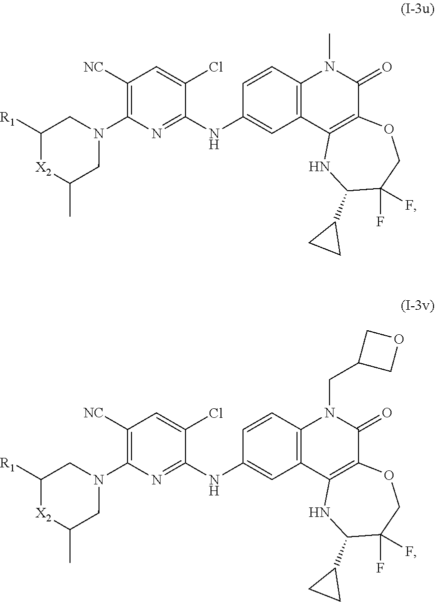

or a pharmaceutically acceptable salt or stereoisomer thereof.

In some embodiments, \textcircled{A} is

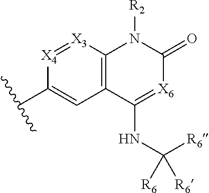

and the compound of formula (I) has the structure of formula I-4,

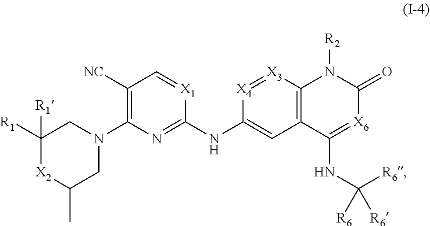

or a pharmaceutically acceptable salt or stereoisomer thereof.

In some embodiments of formula I-4, X₃ is CR₁₂. In some embodiments of formula I-4, X₃ is CH. In some embodiments of formula I-4, X₃ is N.

In some embodiments of formula I-4, X₄ is CR₁₂. In some embodiments of formula I-4, X₄ is CH. In some embodiments of formula I-4, X₄ is N.

In some embodiments of formula I-4, X₆ is N. In some embodiments of formula I-4, X₆ is CH.

In some embodiments of formula I-4, R₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different R₁₃ groups. In some embodiments, R₂ is methyl. In some embodiments, R₂ is 4-membered heterocyclyl, (C₃)carbocyclyl(C₁)alkyl, or 4-membered heterocyclyl(C₁)alkyl, wherein the heterocyclyl contains 1 heteroatom selected from N and O.

In some embodiments, the compound of formula I-4 is of formula I-4a, I-4b, I-4c, I-4d, I-4e, I-4f, I-4g, I-4h, I-4i, or I-4j:

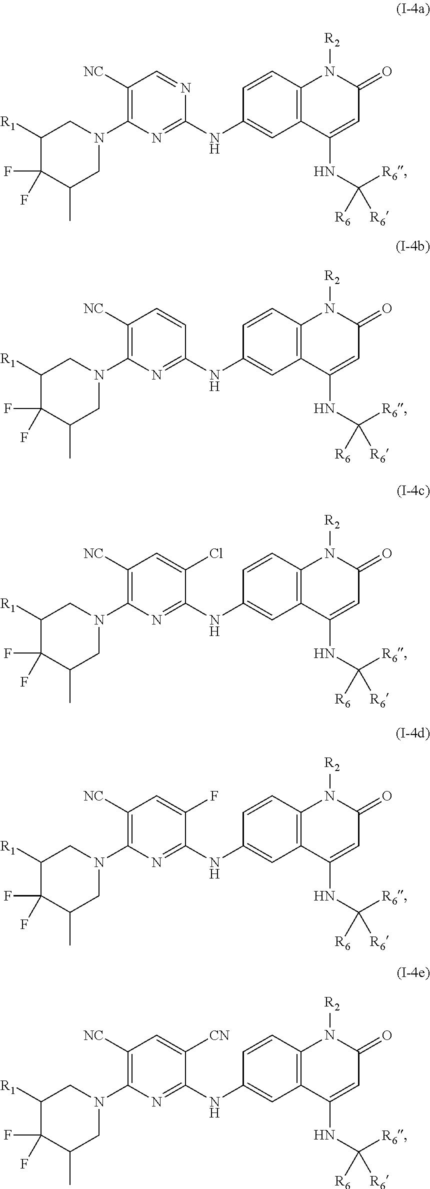

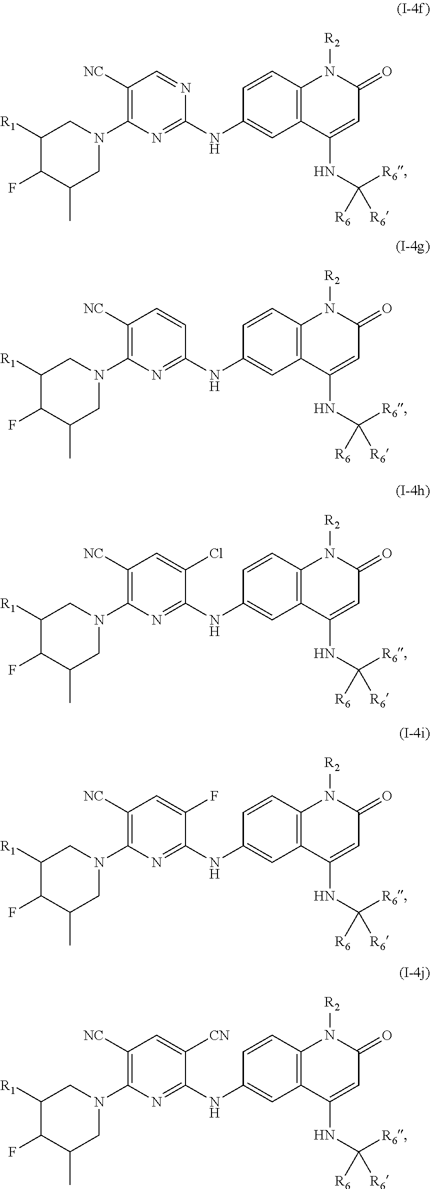

or a pharmaceutically acceptable salt or stereoisomer thereof.

In some embodiments, \textcircled{A} is

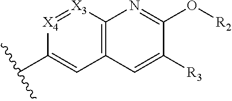

and the compound of formula (I) has the structure of formula I-5,

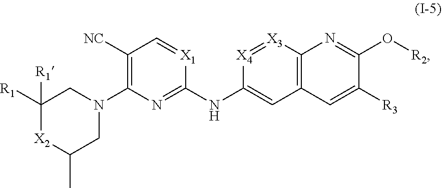

or a pharmaceutically acceptable salt or stereoisomer thereof.

In some embodiments of formula I-5, X₃ is N. In some embodiments of formula I-5, X₃ is CR₁₂. In some embodiments of formula I-5, X₃ is CH. In some embodiments of formula I-5, X₃ is CF. In some embodiments of formula I-5, X₃ is COMe.

In some embodiments of formula I-5, X₄ is N. In some embodiments of formula I-5, X₄ is CR₁₂. In some embodiments of formula I-5, X₄ is CH. In some embodiments of formula I-5, X₄ is CF. In some embodiments of formula I-5, X₄ is COMe.

In some embodiments of formula I-5, R₂ is 4-membered heterocyclyl or 4-membered heterocyclyl(C₂)alkyl; wherein said alkyl, carbocyclyl, or heterocyclyl is further optionally substituted by one or more, identical or different R₁₃ groups. In some embodiments, R₂ is 4-membered heterocyclyl or 4-membered heterocyclyl(C₂)alkyl, wherein the heterocyclyl contains 1 heteroatom selected from N and O.

In some embodiments of formula I-5, R₂ is (C₁-C₆)alkyl, (C₁-C₆)alkyl-OH, (C₁-C₆)alkyl-NH(C₁-C₆)alkyl, or (C₁-C₆)alkyl-N((C₁-C₆)alkyl)₂.

In some embodiments of formula I-5, R₃ is -L₃CR₁₄R₁₅R₁₆. In some embodiments of formula I-5, L₃ is (C₁-C₄)alkylene, —O—(C₁-C₄)alkylene, or —S—(C₁-C₄)alkylene. In some embodiments of formula I-5, L₃ is —O—(C₁-C₄)alkylene. In some embodiments of formula I-5, L₃ is —O—(C₁)alkylene.

In some embodiments of formula I-5, R₁₄ and R₁₅ together with the carbon atom to which they are attached form a (C₃-C₅)carbocyclyl, 4- to 7-membered heterocyclyl, or C═O. In some embodiments of formula I-5, R₁₄ and R₁₅ together with the same carbon atom to which they are attached form C═O. In some embodiments of formula I-5, R₁₄ and R₁₅ together with the same carbon atom to which they are attached form 4- to 7-membered heterocyclyl. In some embodiments of formula I-5, R₁₄ and R₁₅ together with the same carbon atom to which they are attached form an oxetane ring.

In some embodiments of formula I-5, R₁₆ is (C₁-C₆)alkyl, —NR₁₇R₁₈, or —OR₁₇. In some embodiments of formula I-5, R₁₆ is methyl, hydroxyl, amino, or NHMe. In some embodiments, R₁₆ is methyl. In some embodiments, R₁₆ is hydroxyl. In some embodiments, R₁₆ is amino. In some embodiments, R₁₆ is NHMe.

In some embodiments, the compound of formula I-5 is of formula I-5a, I-5b, I-5c, I-5d, I-5e, I-5f, I-5g, I-5h, I-5i, or I-5j:

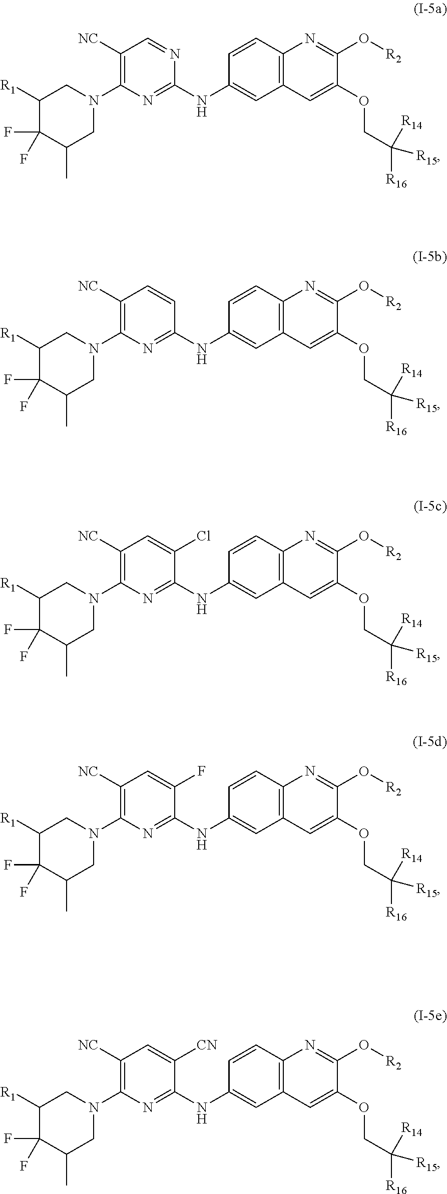

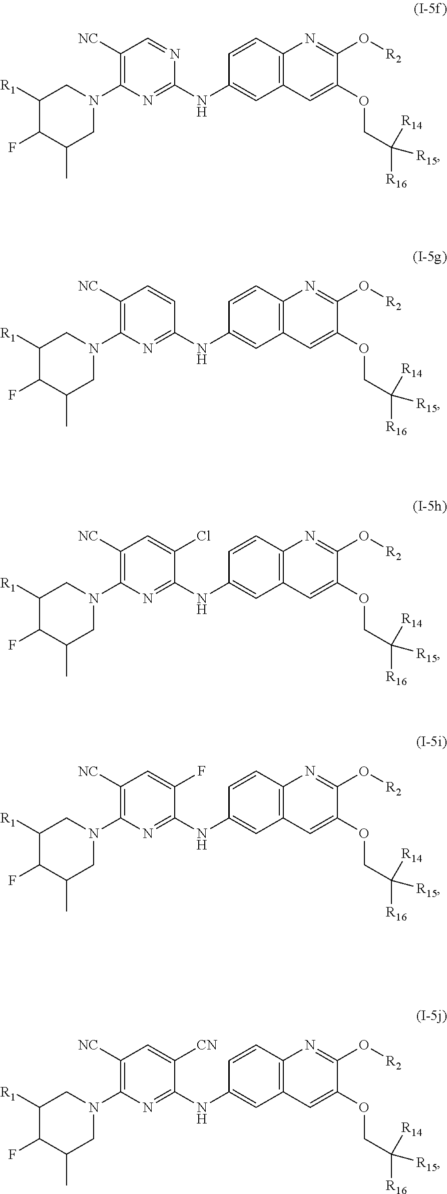

or a pharmaceutically acceptable salt or stereoisomer thereof.

Representative examples of compounds of the invention include:

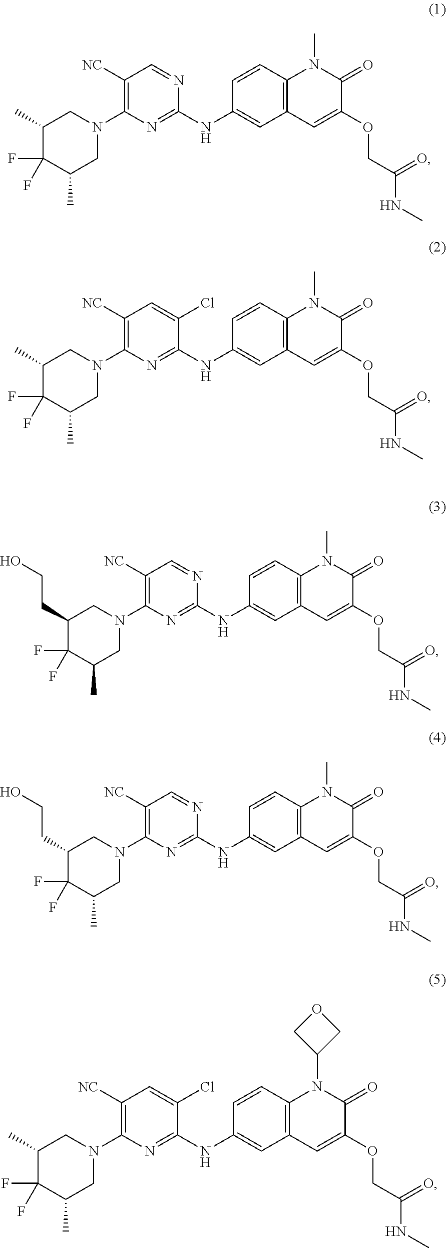 [SMILES: CNC(=O)COc1cc2cc(Nc3ncc(C#N)c(N4C[C@@H](C)C(F)(F)[C@@H](C)C4)n3)ccc2n(C)c1=O ; CNC(=O)COc1cc2cc(Nc3nc(N4C[C@@H](C)C(F)(F)[C@@H](C)C4)c(C#N)cc3Cl)ccc2n(C)c1=O ; CNC(=O)COc1cc2cc(Nc3ncc(C#N)c(N4C[C@H](CCO)C(F)(F)[C@H](C)C4)n3)ccc2n(C)c1=O ; CNC(=O)COc1cc2cc(Nc3ncc(C#N)c(N4C[C@@H](CCO)C(F)(F)[C@@H](C)C4)n3)ccc2n(C)c1=O ; CNC(=O)COc1cc2cc(Nc3nc(N4C[C@@H](C)C(F)(F)[C@@H](C)C4)c(C#N)cc3Cl)ccc2n(C2COC2)c1=O]

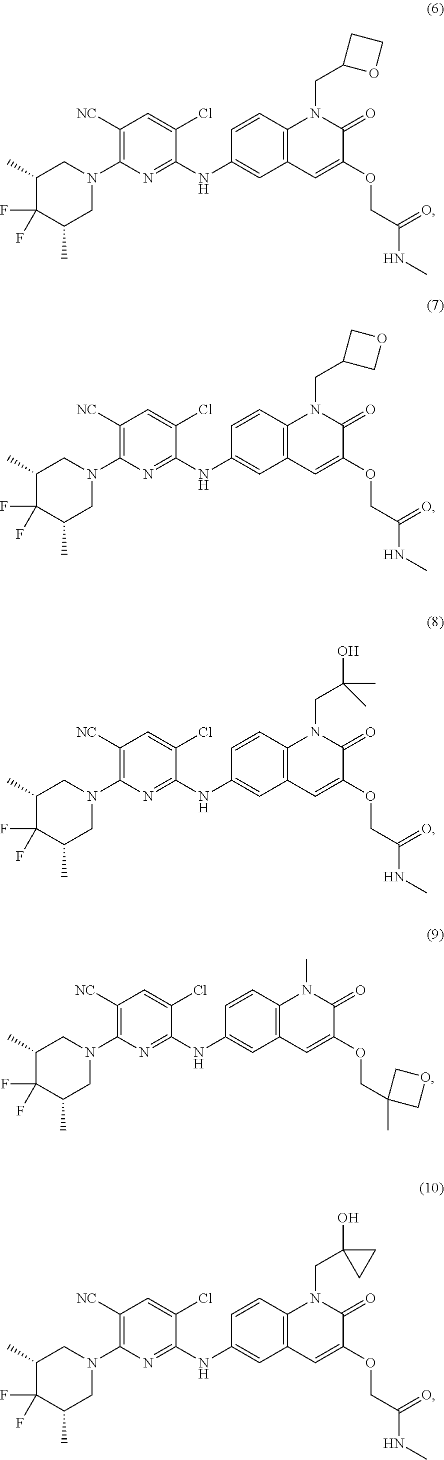 [SMILES: CNC(=O)COc1cc2cc(Nc3nc(N4C[C@@H](C)C(F)(F)[C@@H](C)C4)c(C#N)cc3Cl)ccc2n(CC2CCO2)c1=O ; CNC(=O)COc1cc2cc(Nc3nc(N4C[C@@H](C)C(F)(F)[C@@H](C)C4)c(C#N)cc3Cl)ccc2n(CC2COC2)c1=O ; CNC(=O)COc1cc2cc(Nc3nc(N4C[C@@H](C)C(F)(F)[C@@H](C)C4)c(C#N)cc3Cl)ccc2n(CC(C)(C)O)c1=O ; C[C@@H]1CN(c2nc(Nc3ccc4c(c3)cc(OCC3(C)COC3)c(=O)n4C)c(Cl)cc2C#N)C[C@H](C)C1(F)F ; CNC(=O)COc1cc2cc(Nc3nc(N4C[C@@H](C)C(F)(F)[C@@H](C)C4)c(C#N)cc3Cl)ccc2n(CC2(O)CC2)c1=O]

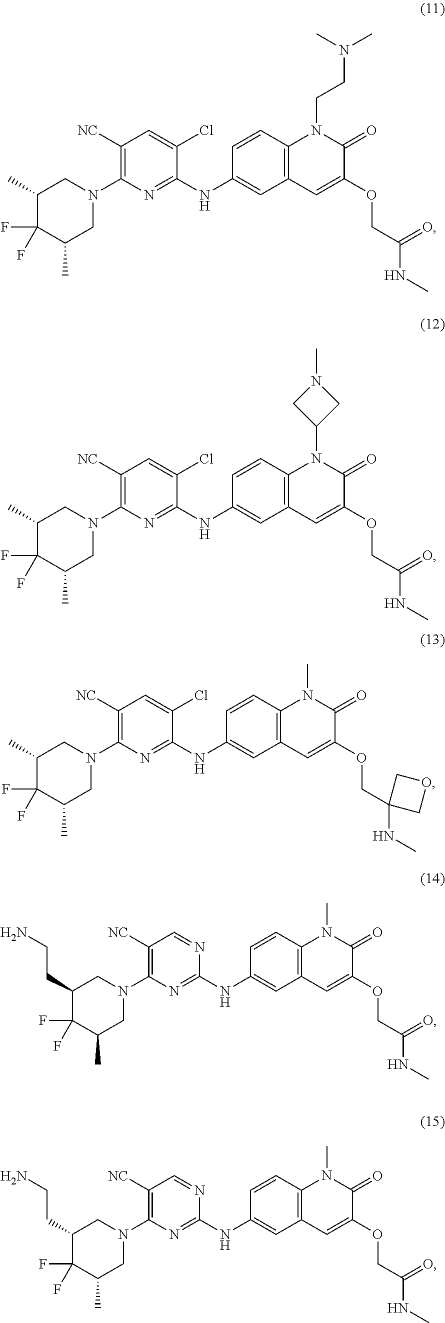 [SMILES: CNC(=O)COc1cc2cc(Nc3nc(N4C[C@@H](C)C(F)(F)[C@@H](C)C4)c(C#N)cc3Cl)ccc2n(CCN(C)C)c1=O ; CNC(=O)COc1cc2cc(Nc3nc(N4C[C@@H](C)C(F)(F)[C@@H](C)C4)c(C#N)cc3Cl)ccc2n(C2CN(C)C2)c1=O ; CNC1(COc2cc3cc(Nc4nc(N5C[C@@H](C)C(F)(F)[C@@H](C)C5)c(C#N)cc4Cl)ccc3n(C)c2=O)COC1 ; CNC(=O)COc1cc2cc(Nc3ncc(C#N)c(N4C[C@H](CCN)C(F)(F)[C@H](C)C4)n3)ccc2n(C)c1=O ; CNC(=O)COc1cc2cc(Nc3ncc(C#N)c(N4C[C@@H](CCN)C(F)(F)[C@@H](C)C4)n3)ccc2n(C)c1=O]

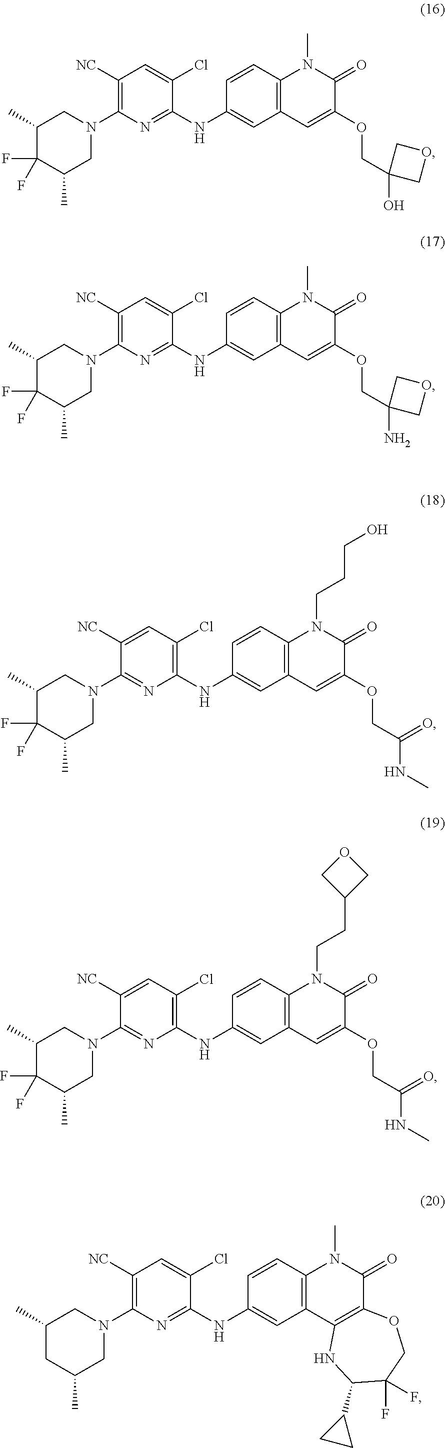 [SMILES: C[C@@H]1CN(c2nc(Nc3ccc4c(c3)cc(OCC3(O)COC3)c(=O)n4C)c(Cl)cc2C#N)C[C@H](C)C1(F)F ; C[C@@H]1CN(c2nc(Nc3ccc4c(c3)cc(OCC3(N)COC3)c(=O)n4C)c(Cl)cc2C#N)C[C@H](C)C1(F)F ; CNC(=O)COc1cc2cc(Nc3nc(N4C[C@@H](C)C(F)(F)[C@@H](C)C4)c(C#N)cc3Cl)ccc2n(CCCO)c1=O ; CNC(=O)COc1cc2cc(Nc3nc(N4C[C@@H](C)C(F)(F)[C@@H](C)C4)c(C#N)cc3Cl)ccc2n(CCC2COC2)c1=O ; C[C@@H]1C[C@H](C)CN(c2nc(Nc3ccc4c(c3)c3c(c(=O)n4C)OCC(F)(F)[C@H](C4CC4)N3)c(Cl)cc2C#N)C1]

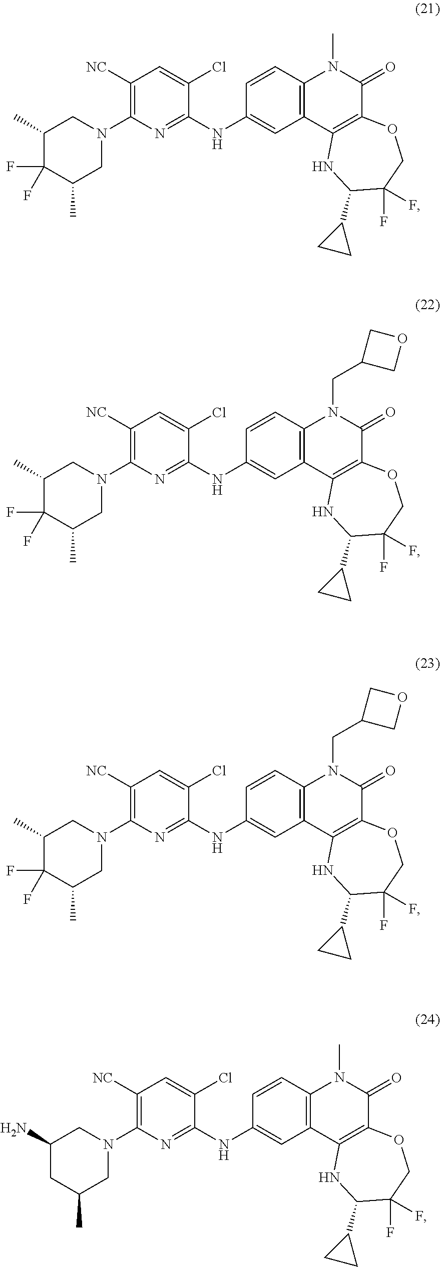 [SMILES: C[C@@H]1CN(c2nc(Nc3ccc4c(c3)c3c(c(=O)n4C)OCC(F)(F)[C@H](C4CC4)N3)c(Cl)cc2C#N)C[C@H](C)C1(F)F ; C[C@@H]1CN(c2nc(Nc3ccc4c(c3)c3c(c(=O)n4CC4COC4)OCC(F)(F)[C@H](C4CC4)N3)c(Cl)cc2C#N)C[C@H](C)C1(F)F ; C[C@@H]1CN(c2nc(Nc3ccc4c(c3)c3c(c(=O)n4CC4COC4)OCC(F)(F)[C@H](C4CC4)N3)c(Cl)cc2C#N)C[C@H](C)C1(F)F ; C[C@H]1C[C@@H](N)CN(c2nc(Nc3ccc4c(c3)c3c(c(=O)n4C)OCC(F)(F)[C@H](C4CC4)N3)c(Cl)cc2C#N)C1]

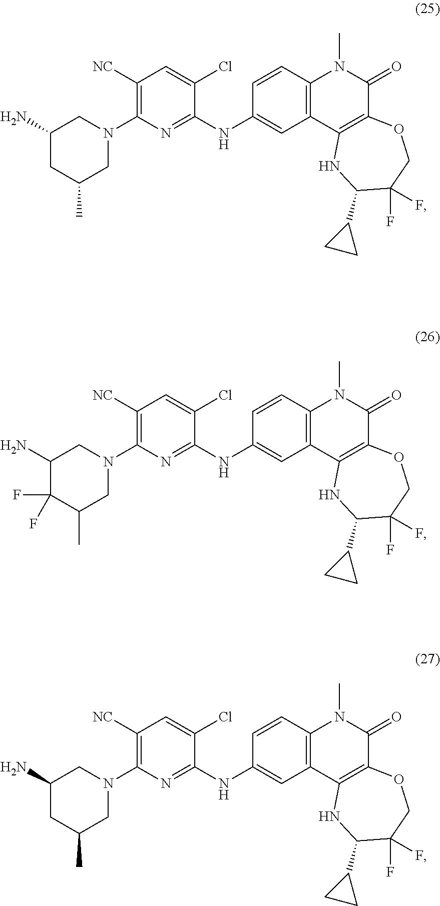 [SMILES: C[C@@H]1C[C@H](N)CN(c2nc(Nc3ccc4c(c3)c3c(c(=O)n4C)OCC(F)(F)[C@H](C4CC4)N3)c(Cl)cc2C#N)C1 ; CC1CN(c2nc(Nc3ccc4c(c3)c3c(c(=O)n4C)OCC(F)(F)[C@H](C4CC4)N3)c(Cl)cc2C#N)CC(N)C1(F)F ; C[C@H]1C[C@@H](N)CN(c2nc(Nc3ccc4c(c3)c3c(c(=O)n4C)OCC(F)(F)[C@H](C4CC4)N3)c(Cl)cc2C#N)C1]

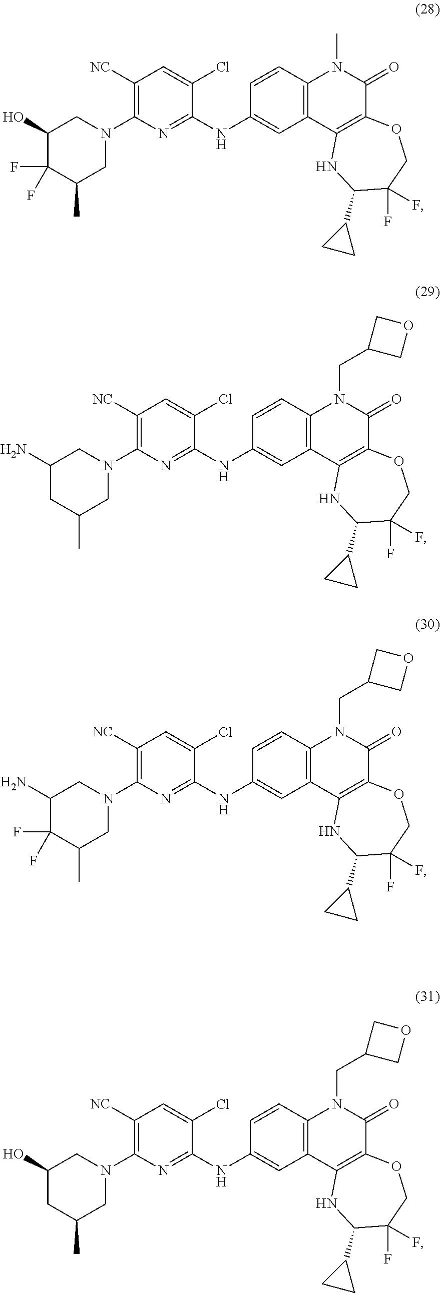 [SMILES: C[C@@H]1CN(c2nc(Nc3ccc4c(c3)c3c(c(=O)n4C)OCC(F)(F)[C@H](C4CC4)N3)c(Cl)cc2C#N)C[C@H](O)C1(F)F ; CC1CC(N)CN(c2nc(Nc3ccc4c(c3)c3c(c(=O)n4CC4COC4)OCC(F)(F)[C@H](C4CC4)N3)c(Cl)cc2C#N)C1 ; CC1CN(c2nc(Nc3ccc4c(c3)c3c(c(=O)n4CC4COC4)OCC(F)(F)[C@H](C4CC4)N3)c(Cl)cc2C#N)CC(N)C1(F)F ; C[C@H]1C[C@@H](O)CN(c2nc(Nc3ccc4c(c3)c3c(c(=O)n4CC4COC4)OCC(F)(F)[C@H](C4CC4)N3)c(Cl)cc2C#N)C1]

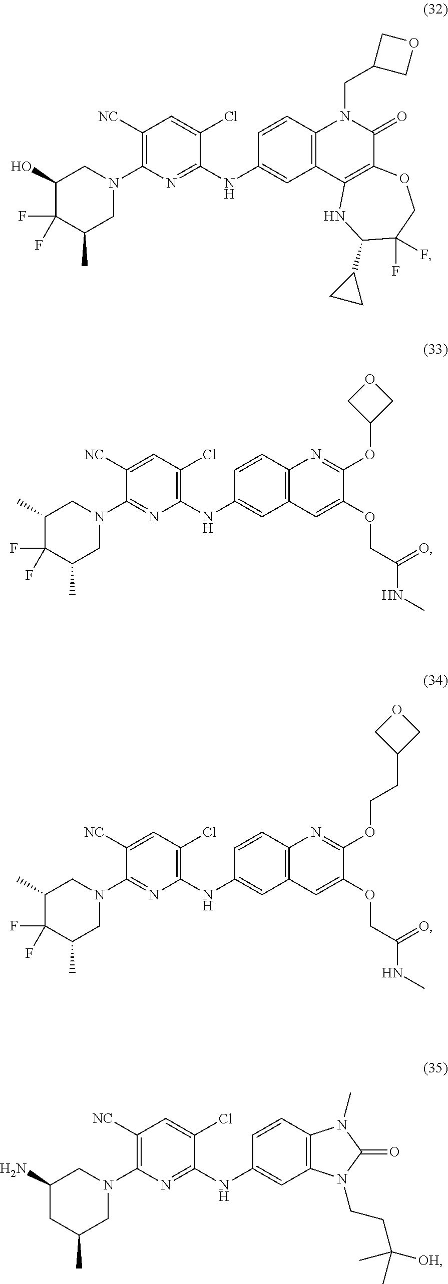 [SMILES: C[C@@H]1CN(c2nc(Nc3ccc4c(c3)c3c(c(=O)n4CC4COC4)OCC(F)(F)[C@H](C4CC4)N3)c(Cl)cc2C#N)C[C@H](O)C1(F)F ; CNC(=O)COc1cc2cc(Nc3nc(N4C[C@@H](C)C(F)(F)[C@@H](C)C4)c(C#N)cc3Cl)ccc2nc1OC1COC1 ; CNC(=O)COc1cc2cc(Nc3nc(N4C[C@@H](C)C(F)(F)[C@@H](C)C4)c(C#N)cc3Cl)ccc2nc1OCCC1COC1 ; C[C@H]1C[C@@H](N)CN(c2nc(Nc3ccc4c(c3)n(CCC(C)(C)O)c(=O)n4C)c(Cl)cc2C#N)C1]

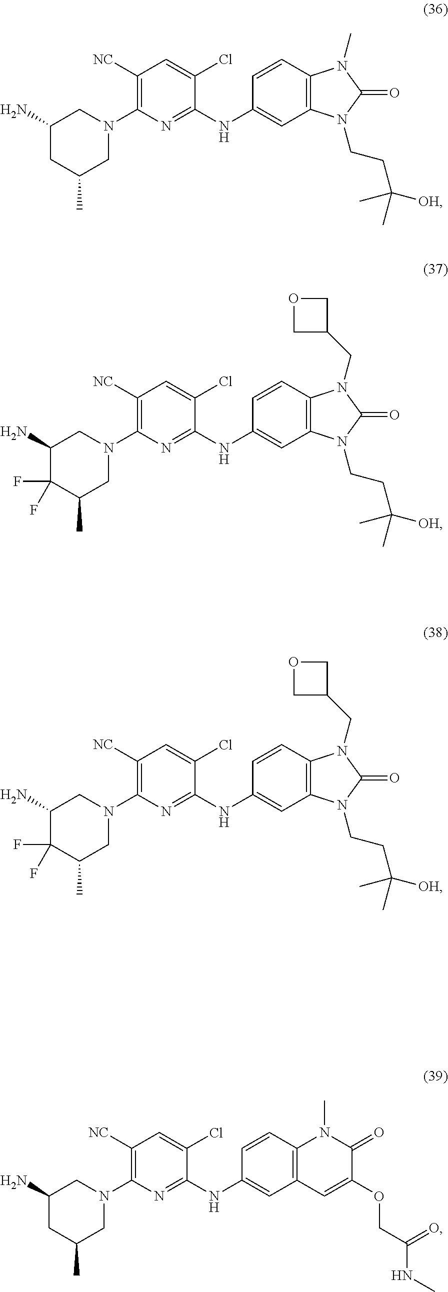 [SMILES: C[C@@H]1C[C@H](N)CN(c2nc(Nc3ccc4c(c3)n(CCC(C)(C)O)c(=O)n4C)c(Cl)cc2C#N)C1 ; C[C@@H]1CN(c2nc(Nc3ccc4c(c3)n(CCC(C)(C)O)c(=O)n4CC3COC3)c(Cl)cc2C#N)C[C@H](N)C1(F)F ; C[C@H]1CN(c2nc(Nc3ccc4c(c3)n(CCC(C)(C)O)c(=O)n4CC3COC3)c(Cl)cc2C#N)C[C@@H](N)C1(F)F ; CNC(=O)COc1cc2cc(Nc3nc(N4C[C@@H](C)C[C@@H](N)C4)c(C#N)cc3Cl)ccc2n(C)c1=O]

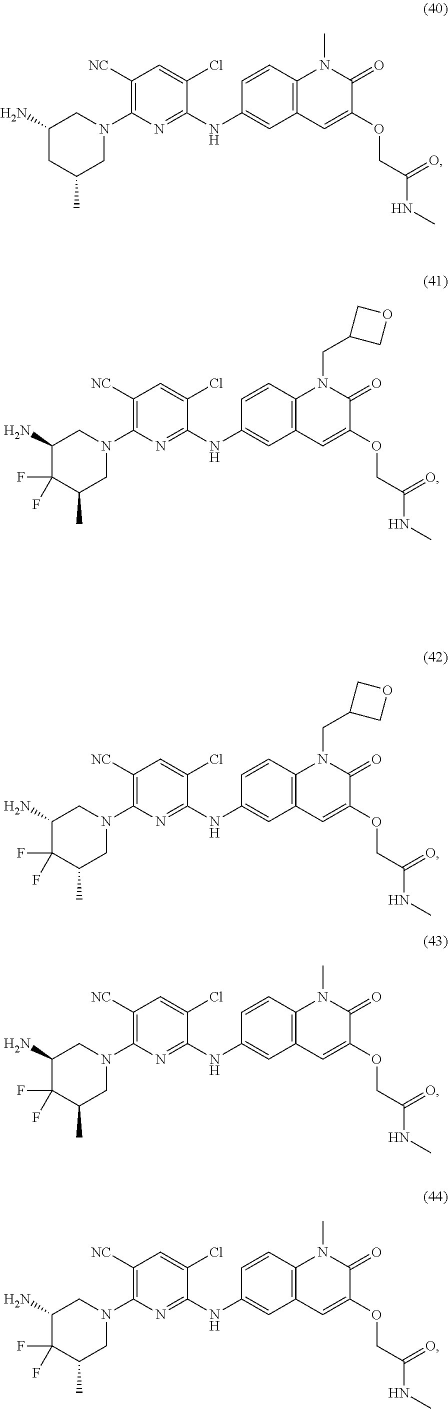 [SMILES: CNC(=O)COc1cc2cc(Nc3nc(N4C[C@H](C)C[C@H](N)C4)c(C#N)cc3Cl)ccc2n(C)c1=O ; CNC(=O)COc1cc2cc(Nc3nc(N4C[C@@H](C)C(F)(F)[C@@H](N)C4)c(C#N)cc3Cl)ccc2n(CC2COC2)c1=O ; CNC(=O)COc1cc2cc(Nc3nc(N4C[C@@H](N)C(F)(F)[C@@H](C)C4)c(C#N)cc3Cl)ccc2n(CC2COC2)c1=O ; CNC(=O)COc1cc2cc(Nc3nc(N4C[C@@H](C)C(F)(F)[C@@H](N)C4)c(C#N)cc3Cl)ccc2n(C)c1=O ; CNC(=O)COc1cc2cc(Nc3nc(N4C[C@@H](N)C(F)(F)[C@@H](C)C4)c(C#N)cc3Cl)ccc2n(C)c1=O]

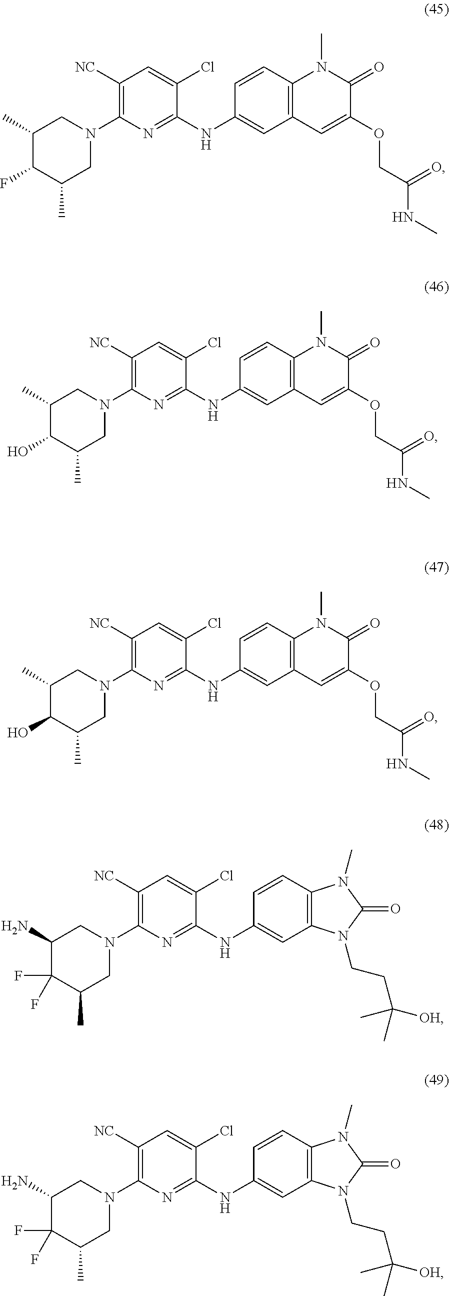

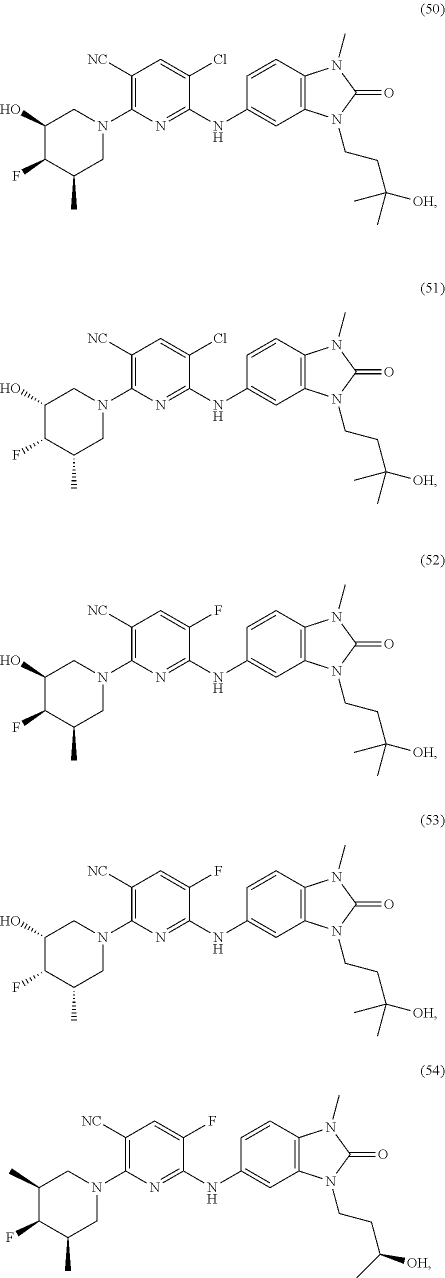 [SMILES: C[C@@H]1CN(c2nc(Nc3ccc4c(c3)n(CCC(C)(C)O)c(=O)n4C)c(Cl)cc2C#N)C[C@H](O)[C@@H]1F ; C[C@H]1CN(c2nc(Nc3ccc4c(c3)n(CCC(C)(C)O)c(=O)n4C)c(Cl)cc2C#N)C[C@@H](O)[C@H]1F ; C[C@@H]1CN(c2nc(Nc3ccc4c(c3)n(CCC(C)(C)O)c(=O)n4C)c(F)cc2C#N)C[C@H](O)[C@@H]1F ; C[C@H]1CN(c2nc(Nc3ccc4c(c3)n(CCC(C)(C)O)c(=O)n4C)c(F)cc2C#N)C[C@@H](O)[C@H]1F ; C[C@H](O)CCn1c(=O)n(C)c2ccc(Nc3nc(N4C[C@@H](C)[C@@H](F)[C@@H](C)C4)c(C#N)cc3F)cc21]

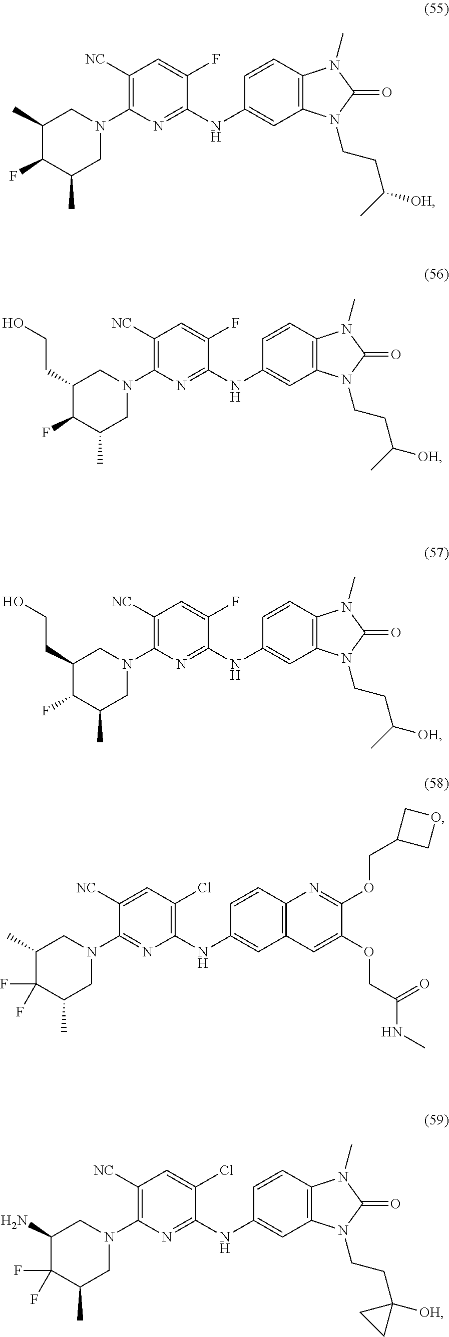 [SMILES: C[C@@H]1CN(c2nc(Nc3ccc4c(c3)n(CC[C@@H](C)O)c(=O)n4C)c(F)cc2C#N)C[C@H](C)[C@@H]1F ; CC(O)CCn1c(=O)n(C)c2ccc(Nc3nc(N4C[C@@H](CCO)[C@H](F)[C@@H](C)C4)c(C#N)cc3F)cc21 ; CC(O)CCn1c(=O)n(C)c2ccc(Nc3nc(N4C[C@H](CCO)[C@@H](F)[C@H](C)C4)c(C#N)cc3F)cc21 ; CNC(=O)COc1cc2cc(Nc3nc(N4C[C@@H](C)C(F)(F)[C@@H](C)C4)c(C#N)cc3Cl)ccc2nc1OCC1COC1 ; C[C@@H]1CN(c2nc(Nc3ccc4c(c3)n(CCC3(O)CC3)c(=O)n4C)c(Cl)cc2C#N)C[C@H](N)C1(F)F]

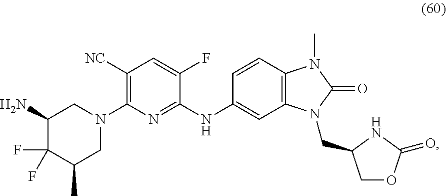 [SMILES: C[C@@H]1CN(c2nc(Nc3ccc4c(c3)n(C[C@@H]3COC(=O)N3)c(=O)n4C)c(F)cc2C#N)C[C@H](N)C1(F)F]

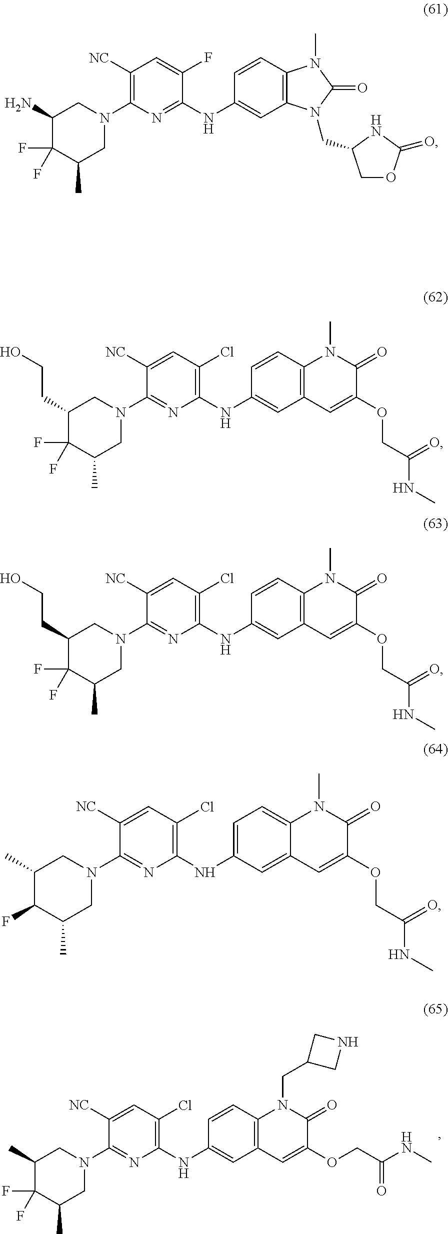 [SMILES: C[C@@H]1CN(c2nc(Nc3ccc4c(c3)n(C[C@H]3COC(=O)N3)c(=O)n4C)c(F)cc2C#N)C[C@H](N)C1(F)F ; CNC(=O)COc1cc2cc(Nc3nc(N4C[C@@H](CCO)C(F)(F)[C@@H](C)C4)c(C#N)cc3Cl)ccc2n(C)c1=O ; CNC(=O)COc1cc2cc(Nc3nc(N4C[C@H](CCO)C(F)(F)[C@H](C)C4)c(C#N)cc3Cl)ccc2n(C)c1=O ; CNC(=O)COc1cc2cc(Nc3nc(N4C[C@@H](C)[C@H](F)[C@@H](C)C4)c(C#N)cc3Cl)ccc2n(C)c1=O ; CNC(=O)COc1cc2cc(Nc3nc(N4C[C@@H](C)C(F)(F)[C@@H](C)C4)c(C#N)cc3Cl)ccc2n(CC2CNC2)c1=O]

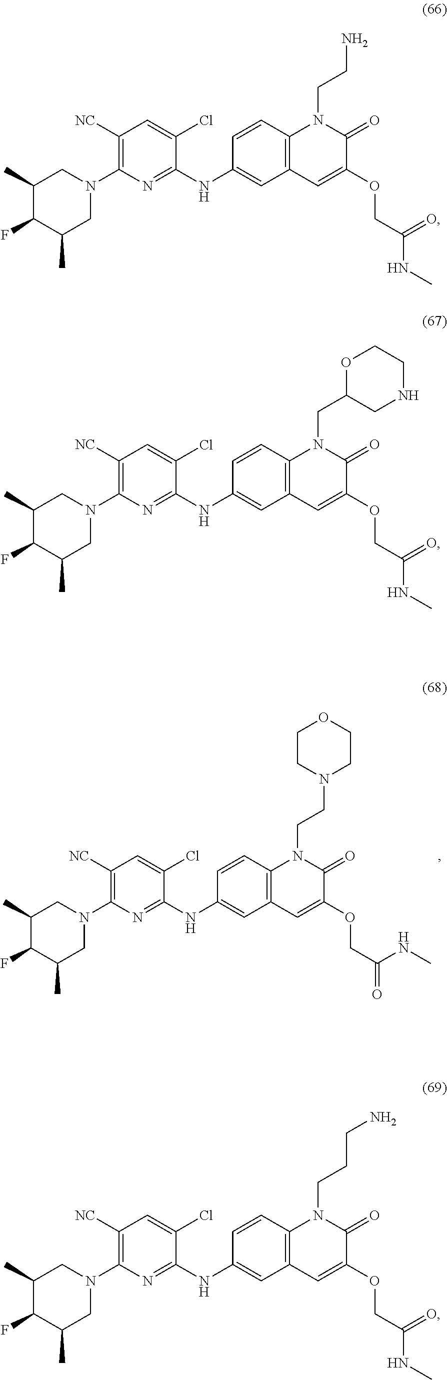 [SMILES: CNC(=O)COc1cc2cc(Nc3nc(N4C[C@@H](C)[C@@H](F)[C@@H](C)C4)c(C#N)cc3Cl)ccc2n(CCN)c1=O ; CNC(=O)COc1cc2cc(Nc3nc(N4C[C@@H](C)[C@@H](F)[C@@H](C)C4)c(C#N)cc3Cl)ccc2n(CC2CNCCO2)c1=O ; CNC(=O)COc1cc2cc(Nc3nc(N4C[C@@H](C)[C@@H](F)[C@@H](C)C4)c(C#N)cc3Cl)ccc2n(CCN2CCOCC2)c1=O ; CNC(=O)COc1cc2cc(Nc3nc(N4C[C@@H](C)[C@@H](F)[C@@H](C)C4)c(C#N)cc3Cl)ccc2n(CCCN)c1=O]

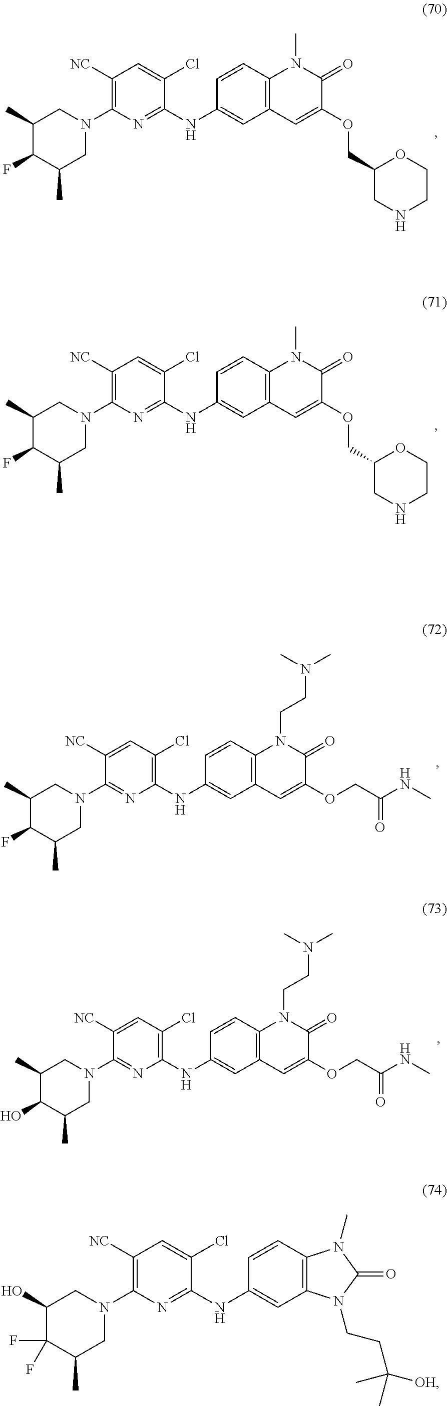 [SMILES: C[C@@H]1CN(c2nc(Nc3ccc4c(c3)cc(OC[C@@H]3CNCCO3)c(=O)n4C)c(Cl)cc2C#N)C[C@H](C)[C@@H]1F ; C[C@@H]1CN(c2nc(Nc3ccc4c(c3)cc(OC[C@H]3CNCCO3)c(=O)n4C)c(Cl)cc2C#N)C[C@H](C)[C@@H]1F ; CNC(=O)COc1cc2cc(Nc3nc(N4C[C@@H](C)[C@@H](F)[C@@H](C)C4)c(C#N)cc3Cl)ccc2n(CCN(C)C)c1=O ; CNC(=O)COc1cc2cc(Nc3nc(N4C[C@@H](C)[C@@H](O)[C@@H](C)C4)c(C#N)cc3Cl)ccc2n(CCN(C)C)c1=O ; C[C@@H]1CN(c2nc(Nc3ccc4c(c3)n(CCC(C)(C)O)c(=O)n4C)c(Cl)cc2C#N)C[C@H](O)C1(F)F]

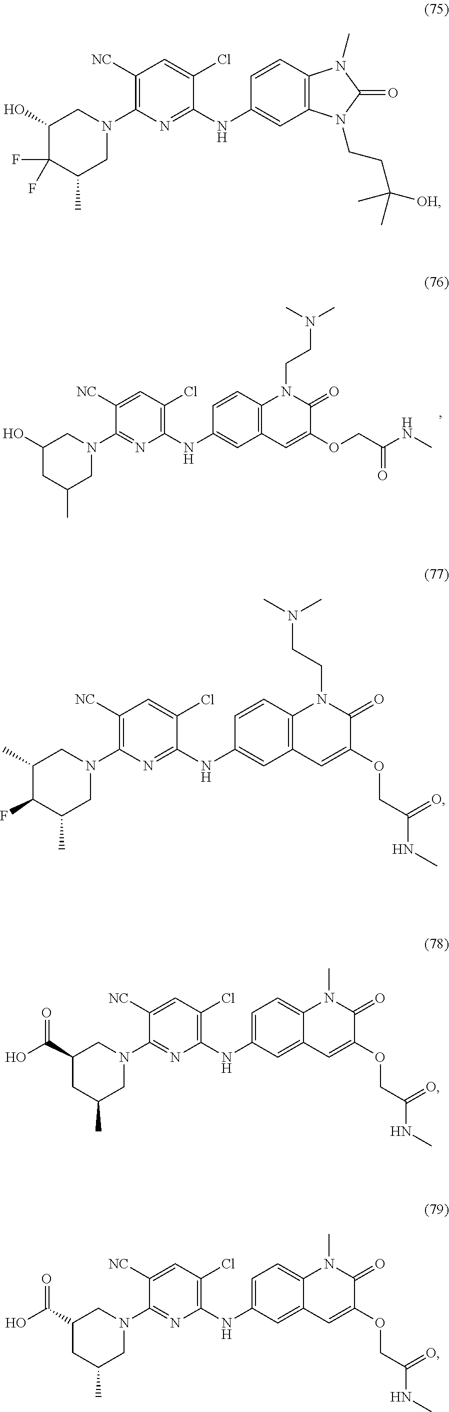

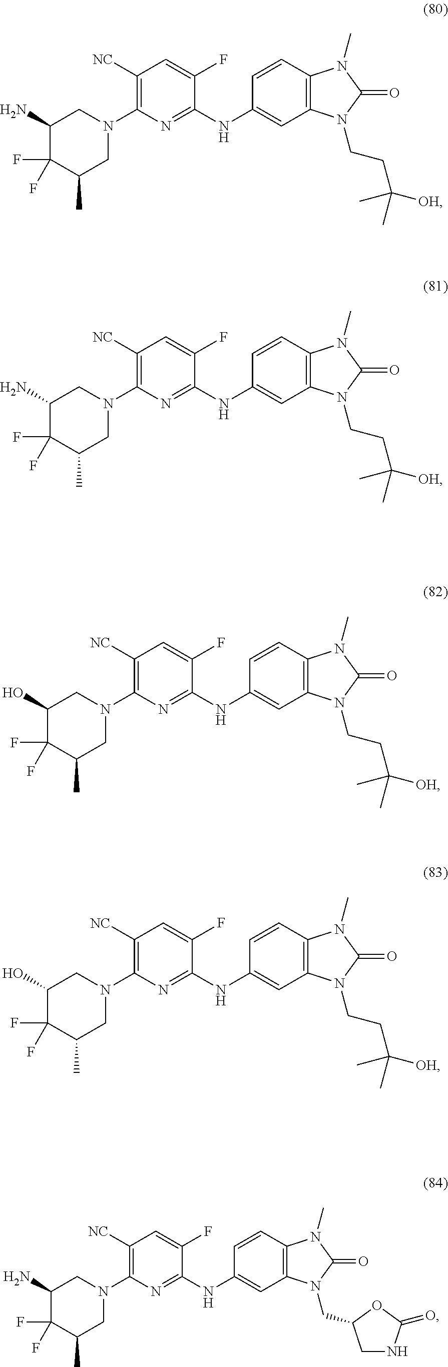 [SMILES: C[C@@H]1CN(c2nc(Nc3ccc4c(c3)n(CCC(C)(C)O)c(=O)n4C)c(F)cc2C#N)C[C@H](N)C1(F)F ; C[C@H]1CN(c2nc(Nc3ccc4c(c3)n(CCC(C)(C)O)c(=O)n4C)c(F)cc2C#N)C[C@@H](N)C1(F)F ; C[C@@H]1CN(c2nc(Nc3ccc4c(c3)n(CCC(C)(C)O)c(=O)n4C)c(F)cc2C#N)C[C@H](O)C1(F)F ; C[C@H]1CN(c2nc(Nc3ccc4c(c3)n(CCC(C)(C)O)c(=O)n4C)c(F)cc2C#N)C[C@@H](O)C1(F)F ; C[C@@H]1CN(c2nc(Nc3ccc4c(c3)n(C[C@H]3CNC(=O)O3)c(=O)n4C)c(F)cc2C#N)C[C@H](N)C1(F)F]

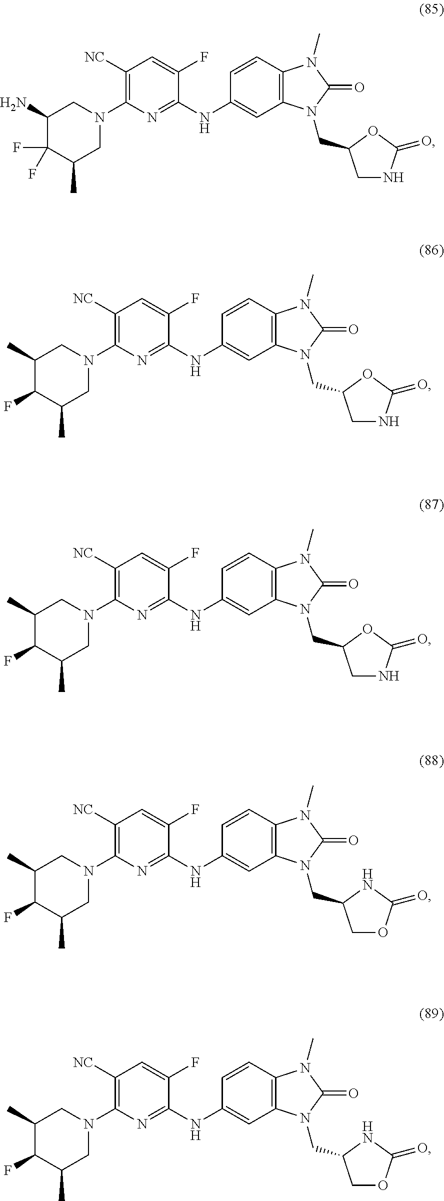 [SMILES: C[C@@H]1CN(c2nc(Nc3ccc4c(c3)n(C[C@@H]3CNC(=O)O3)c(=O)n4C)c(F)cc2C#N)C[C@H](N)C1(F)F ; C[C@@H]1CN(c2nc(Nc3ccc4c(c3)n(C[C@H]3CNC(=O)O3)c(=O)n4C)c(F)cc2C#N)C[C@H](C)[C@@H]1F ; C[C@@H]1CN(c2nc(Nc3ccc4c(c3)n(C[C@@H]3CNC(=O)O3)c(=O)n4C)c(F)cc2C#N)C[C@H](C)[C@@H]1F ; C[C@@H]1CN(c2nc(Nc3ccc4c(c3)n(C[C@@H]3COC(=O)N3)c(=O)n4C)c(F)cc2C#N)C[C@H](C)[C@@H]1F ; C[C@@H]1CN(c2nc(Nc3ccc4c(c3)n(C[C@H]3COC(=O)N3)c(=O)n4C)c(F)cc2C#N)C[C@H](C)[C@@H]1F]

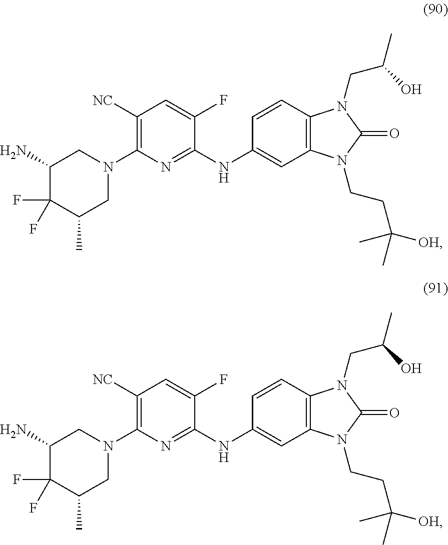 [SMILES: C[C@H](O)Cn1c(=O)n(CCC(C)(C)O)c2cc(Nc3nc(N4C[C@@H](N)C(F)(F)[C@@H](C)C4)c(C#N)cc3F)ccc21 ; C[C@@H](O)Cn1c(=O)n(CCC(C)(C)O)c2cc(Nc3nc(N4C[C@@H](N)C(F)(F)[C@@H](C)C4)c(C#N)cc3F)ccc21]

or a pharmaceutically acceptable salt or stereoisomer thereof.

Compounds of the present invention may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin. Suitable base salts include aluminum, calcium, lithium, magnesium, potassium, sodium, or zinc salts.

Compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R—) or (S—) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R—) form is considered equivalent to administration of the compound in its (S—) form. Accordingly, the compounds of the present invention may be made and used in the form of individual isomers and substantially free of other isomers, or in the form of a mixture of various isomers, e.g., racemic mixtures of stereoisomers.

In some embodiments, the compound is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

Compounds of formula (I) may also be in the form of N-oxides, crystalline forms (also known as polymorphs), active metabolites of the compounds having the same type of activity, prodrugs, tautomers, and unsolvated as well as solvated (e.g., hydrated) forms with pharmaceutically acceptable solvents such as water, ethanol, and the like, of the compounds.

The compounds of the present invention may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

In some embodiments, the pharmaceutical composition comprises a co-crystal of an inventive compound. The term “co-crystal”, as used herein, refers to a stoichiometric multi-component system comprising a compound of the invention and a co-crystal former wherein the compound of the invention and the co-crystal former are connected by non-covalent interactions. The term “co-crystal former”, as used herein, refers to compounds which can form intermolecular interactions with a compound of the invention and co-crystallize with it. Representative examples of co-crystal formers include benzoic acid, succinic acid, fumaric acid, glutaric acid, trans-cinnamic acid, 2,5-dihydroxybenzoic acid, glycolic acid, trans-2-hexanoic acid, 2-hydroxycaproic acid, lactic acid, sorbic acid, tartaric acid, ferulic acid, suberic acid, picolinic acid, salicyclic acid, maleic acid, saccharin, 4,4′-bipyridine p-aminosalicyclic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present invention is directed to a method for making a compound of formula (I), or a pharmaceutically acceptable salt or stereoisomer thereof. Broadly, the inventive compounds or pharmaceutically-acceptable salts or stereoisomers thereof may be prepared by any process known to be applicable to the preparation of chemically related compounds. The compounds of the present invention will be better understood in connection with the synthetic schemes that described in various working examples and which illustrate non-limiting methods by which the compounds of the invention may be prepared.

### Pharmaceutical Compositions

Another aspect of the present invention is directed to a pharmaceutical composition that includes a therapeutically effective amount of a compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds are formulated for oral or intravenous administration (e.g., systemic intravenous injection).

Accordingly, compounds of formula (I) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of formula (I) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of formula (I) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of formula (I) may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound of formula (I) or a pharmaceutically acceptable salt or a stereoisomer thereof that is effective in producing the desired therapeutic response in a particular patient suffering from a disease or disorder mediated by aberrant BCL6 activity. The term “therapeutically effective amount” thus includes the amount of the compound or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer) cells, or reduces the amounts of BCL6 in diseased cells.

The total daily dosage of the compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the compound; and like factors well known in the medical arts (see, for example, Goodman and Gilman's, The Pharmacological Basis of Therapeutics, 10th Edition, A. Gilman, J. Hardman and L. Limbird, eds., McGraw-Hill Press, 155-173, 2001).

Compounds of formula (I) and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, or in yet other embodiments from about 10 to about 30 mg per day. In some embodiments, the total daily dosage may range from 400 mg to 600 mg. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the compound may be administered at a dose in range from about 0.001 mg/kg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

In some embodiments, compounds of formula (I) and their pharmaceutically acceptable salts and stereoisomers may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect.

### Methods of Use

In some aspects, the present invention is directed to treating diseases or disorders characterized or mediated by aberrant (e.g., elevated levels of BCL6 or otherwise functionally abnormal e.g., dysfunctional BCL6 levels) BCL6 activity relative to a non-pathological state. The methods entail administering a therapeutically effective amount of a compound of formula (I) or a pharmaceutically acceptable salt or stereoisomer thereof, to a subject in need thereof. A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from a specific disease or disorder, and subjects suspected of suffering from a specific disease or disorder are not necessarily two distinct groups.

In some embodiments, the inventive compounds may be useful in the treatment of cell proliferative diseases and disorders (e.g., cancer or benign neoplasms). As used herein, the term “cell proliferative disease or disorder” refers to the conditions characterized by aberrant cell growth, or both, including noncancerous conditions such as neoplasms, precancerous conditions, benign tumors, and cancer.

In some embodiments, the methods are directed to treating subjects having cancer. Both adult tumors/cancers and pediatric tumors/cancers are included. The cancers may be vascularized, or not yet substantially vascularized, or non-vascularized tumors.

In some embodiments, methods of the present invention entail treatment of subjects having cell proliferative diseases or disorders of the hematological system.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

In some embodiments, the methods are directed to treating subjects having a lymphoid malignancy.

In some embodiments, the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), cutaneous T-cell lymphoma, chronic myeloid leukemia, or B-cell non-Hodgkin's lymphoma.

In some embodiments, the cancer is melanoma, breast cancer or non-small cell lung cancer.

Compounds of formula (I) may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became unresponsive or intolerant to the particular treatment. Therapy may also be given as an adjuvant treatment, i.e., to prevent reoccurrence of cancer in patients with no currently detectable disease or after surgical removal of a tumor. Thus, in some embodiments, the compounds may be administered to a patient who has received another therapy, such as chemotherapy, radioimmunotherapy, surgical therapy, immunotherapy, radiation therapy, targeted therapy or any combination thereof.

The methods of the present invention may entail administration of a compound of formula (I) or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day “off” period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of 5 days.

### Combination Therapy

The compounds of formula (I) and their pharmaceutically acceptable salts and stereoisomers may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of formula (I) in combination with one or more additional therapeutics known for use in treating a disease or condition (e.g., cancer). The dosage of the additional therapeutic may be the same or even lower than known or recommended doses. See, Hardman et al., eds., Goodman & Gilman's the Pharmacological Basis of Basis of Therapeutics, 10th ed., McGraw-Hill, New York, 2001; Physician's Desk Reference 60th ed., 2006. For example, anti-cancer agents that may be suitable for use in combination with the inventive compounds are known in the art. See, e.g., U.S. Pat. No. 9,101,622 (Section 5.2 thereof) and U.S. Pat. No. 9,345,705 B2 (Columns 12-18 thereof). Representative examples of additional anti-cancer agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy.

In some embodiments, a compound of formula (I) and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

When the active components of the combination are not administered in the same pharmaceutical composition, it is understood that they can be administered in any order to a subject in need thereof. For example, a compound of the present invention can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional therapeutic, to a subject in need thereof. In various aspects, the therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the (e.g., anticancer) therapeutics are administered within the same office visit. In another example, the combination anticancer therapeutics may be administered at 1 minute to 24 hours apart.

In some embodiments involving cancer treatment, a compound of formula (I) and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, the compound of the present invention may be used in combination with other anti-cancer agents, examples of which include Etoposide (e.g., lymphomas, and non-lymphocytic leukemia), Vincristine (e.g., leukemia), Daunorubicin (e.g., acute myeloid leukemia (AML), acute lymphocytic leukemia (ALL), chronic myelogenous leukemia (CML), and Kaposi's sarcoma), Rituximab (e.g., non-Hodgkin's lymphoma), Alemtuzumab (e.g., chronic lymphocytic leukemia (CLL), cutaneous T-cell lymphoma (CTCL) and T-cell lymphoma), Bortezomib (e.g., multiple myeloma and mantle cell lymphoma), Pegaspargase (e.g., acute lymphoblastic leukemia), Keytruda (e.g., Hodgkin lymphoma), and dexamethasone (e.g., acute multiple myeloma).

In some embodiments, the additional anti-cancer agent is an enhancer of zeste homolog 2 (EZH2) inhibitor, examples of which include tazemetostat, GSK126, lirametostat (CPI-1205), CPI-0209, PF-06821497, SHR2554, HH2853, valemetostat (DS3201), MAK-683, and FTX-6058.

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a compound of the present invention or a pharmaceutical composition which contains the compound and a pharmaceutically acceptable carrier wherein the compound and the carrier may be disposed in the same or separate containers. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

### Example 1: Synthesis of 2-((6-((5-cyano-4-((3S,5R)-4,4-difluoro-3,5-dimethylpiperidin-1-yl)pyrimidin-2-yl)amino)-1-methyl-2-oxo-1,2-dihydroquinolin-3-yl)oxy)-N-methylacetamide (1)

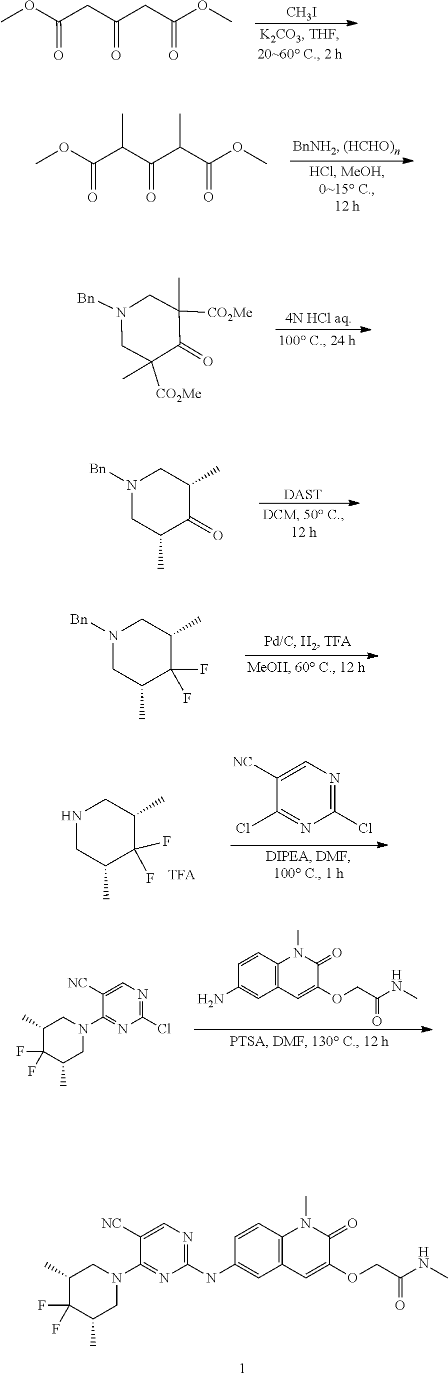

### Dimethyl 2,4-dimethyl-3-oxopentanedioate

K₂CO₃ (39.68 g, 287.11 mmol) was added to a solution of dimethyl 3-oxopentanedioate (20 g, 114.84 mmol) in THF (240 mL) at 20° C. and the resulting mixture was stirred at 45° C. for 20 minutes. CH₃I (32.60 g, 229.69 mmol) was then added and the reaction mixture was heated to 60° C. and was stirred for 100 minutes. The reaction mixture was cooled to 20° C., filtered, and the filter cake was washed with THF (400 mL). The filtrate was dried in vacuum to give the title compound as a crude yellow oil (40 g). ¹H NMR (400 MHz, CDCl₃) δ 3.80-3.71 (m, 6H), 1.44-1.34 (m, 6H).

### Dimethyl 1-benzyl-3,5-dimethyl-4-oxopiperidine-3,5-dicarboxylate

Aq. HCl (1 M, 21.76 mL), phenylmethanamine (11.66 g, 108.80 mmol) and formaldehyde (17.66 g, 217.60 mmol, 37% purity) were added to a solution of dimethyl 2,4-dimethyl-3-oxo-pentanedioate (22 g, 108.80 mmol) in MeOH (400 mL) at 0° C. The reaction mixture was stirred at 15° C. for 12 hours. The reaction mixture was concentrated under reduced pressure to give a residue that was purified by column chromatography (SiO₂, Petroleum ether/Ethyl acetate=100/1 to 20/1) to give the title compound as a yellow oil (15 g, 41%). ¹H NMR (400 MHz, CDCl₃) δ 7.37-7.27 (m, 5H), 3.67 (s, 6H), 3.65 (s, 2H), 3.53 (d, J=11.6 Hz, 2H), 1.30 (s, 6H).

### (3S,5R)-1-Benzyl-3,5-dimethylpiperidin-4-one

A solution of dimethyl 1-benzyl-3,5-dimethyl-4-oxo-piperidine-3,5-dicarboxylate (15 g, 44.99 mmol) in aq. HCl (150 mL, 4 M) was stirred at 100° C. for 24 hours. The reaction mixture was concentrated under reduced pressure and adjusted pH to 8 with 2 M NaOH and then the mixture was extracted with ethyl acetate (200 mL×3). The organic phases were combined and dried with anhydrous Na₂SO₄, filtered and concentrated under reduced pressure. The residue was purified by column chromatography (SiO₂, Petroleum ether/Ethyl acetate=20/1 to 5/1) to give the title compound as a yellow oil (7.2 g, 74%). ¹H NMR (400 MHz, CDCl₃) δ 7.33-7.27 (m, 5H), 3.57 (s, 2H), 3.12 (dd, J=8.8, 5.2 Hz 2H), 2.69 (dd, J=10.8, 5.6 Hz 2H), 2.05-1.99 (m, 2H), 0.93 (d, J=6.8 Hz, 6H).

### (3S,5R)-1-Benzyl-4,4-difluoro-3,5-dimethylpiperidine

DAST (55.63 g, 345.14 mmol) was added to a solution of (3S,5R)-1-benzyl-3,5-dimethyl-piperidin-4-one (5 g, 23.01 mmol) in DCM (50 mL) and the reaction mixture was stirred at 50° C. for 12 hours. The pH was adjusted to 7˜8 with sat. NaHCO₃ and extracted with ethyl acetate (500 mL×3). The organic phases were combined and dried with anhydrous Na₂SO₄, filtered and concentrated under reduced pressure. The residue was purified by column chromatography (SiO₂, Petroleum ether/Ethyl acetate=1/0 to 20/1) to give the title compound as a yellow oil (2.5 g, 45%). ¹H NMR (400 MHz, CDCl₃) δ 7.28-7.21 (m, 5H), 3.46 (s, 2H), 2.71 (d, J=10.8 Hz, 2H), 2.09-2.03 (m, 2H), 1.96-1.90 (m, 2H), 0.93 (d, J=6.8 Hz, 6H).

### (3S,5R)-4,4-Difluoro-3,5-dimethylpiperidine

TFA (4.76 g, 41.79 mmol) and Pd/C (0.8 g, 10% purity) were added to a solution of (3S,5R)-1-benzyl-4,4-difluoro-3,5-dimethyl-piperidine (2.5 g, 10.45 mmol) in MeOH (25 mL) under a N₂ atmosphere and the reaction mixture was stirred under H₂ (15 Psi) at 60° C. for 12 hours. The reaction mixture was filtered and concentrated under reduced pressure to give the title compound as a white solid (2.3 g, 84%, TFA salt). ¹H NMR (400 MHz, CDCl₃) δ 9.64-9.51 (m, 2H), 3.34 (d, J=12.4 Hz, 2H), 2.88-2.80 (m, 2H), 2.46-2.37 (m, 2H), 1.12 (d, J=6.8 Hz, 6H).

### 2-Chloro-4-((3S,5R)-4,4-difluoro-3,5-dimethylpiperidin-1-yl)pyrimidine-5-carbonitrile

A mixture of 2,4-dichloropyrimidine-5-carbonitrile (300 mg, 1.72 mmol), (3R,5S)-4,4-difluoro-3,5-dimethyl-piperidine (453.83 mg, 1.72 mmol, TFA salt), and DIEA (557.11 mg, 4.31 mmol) in DMF (5 mL) was stirred at 100° C. for 1 hour under a N₂ atmosphere. The reaction mixture was concentrated under reduced pressure to give a residue that was purified by reversed-phase HPLC (neutral condition: Column: 80 g Agela C18; Mobile phase: [water-ACN]; B %: 30-60% 15 min; 60% 5 min) to give 4-chloro-2-[(3R,5S)-4,4-difluoro-3,5-dimethyl-1-piperidyl]pyrimidine-5-carbonitrile as a white solid (150 mg, 30%, LCMS: [M+H⁺]=287, retention time=0.928 min) and the title compound as a white solid (60 mg, 12%, LCMS: [M+H⁺]=287, retention time=0.885 min).

### 2-((6-((5-cyano-4-((3S,5R)-4,4-difluoro-3,5-dimethylpiperidin-1-yl)pyrimidin-2-yl)amino)-1-methyl-2-oxo-1,2-dihydroquinolin-3-yl)oxy)-N-methylacetamide (1)

A mixture of 2-[(6-amino-1-methyl-2-oxo-3-quinolyl)oxy]-N-methyl-acetamide (30 mg, 114.82 μmol), 2-chloro-4-[(3R,5S)-4,4-difluoro-3,5-dimethyl-1-piperidyl]pyrimidine-5-carbonitrile (36.21 mg, 126.30 μmol), and 4-methylbenzenesulfonic acid hydrate (26.21 mg, 137.79 μmol) in DMF (1 mL) was stirred at 130° C. for 12 hours under a N₂ atmosphere. H₂O (15 mL) was added to the reaction mixture and the precipitated solids were filtered and washed with H₂O (30 mL) and ethyl acetate (30 mL). The solids were dried under reduced pressure. The solids were triturated with ethanol (5 mL) at 15° C. for 30 minutes to give the title compound as a yellow solid (21.2 mg, 35%). ¹H NMR (400 MHz, DMSO-d₆) δ 10.05 (s, 1H), 8.49 (s, 1H), 7.90-7.89 (m, 2H), 7.68 (dd, J=8.8, 2.0 Hz, 1H), 7.46 (d, J=9.2 Hz, 1H), 7.18 (s, 1H), 4.62 (d, J=13.6 Hz, 2H), 4.56 (s, 2H), 3.66 (s, 3H), 2.93 (t, J=12.8 Hz, 2H), 2.66 (d, J=4.8 Hz, 3H), 2.33-2.27 (m, 2H), 1.00 (d, J=6.8 Hz, 6H). LCMS: [M+H⁺]=512.2.

### Example 2: Synthesis of 2-((6-((3-chloro-5-cyano-6-((3S,5R)-4,4-difluoro-3,5-dimethylpiperidin-1-yl) pyridin-2-yl)amino)-1-methyl-2-oxo-1,2-dihydroquinolin-3-yl)oxy)-N-methylacetamide (2)

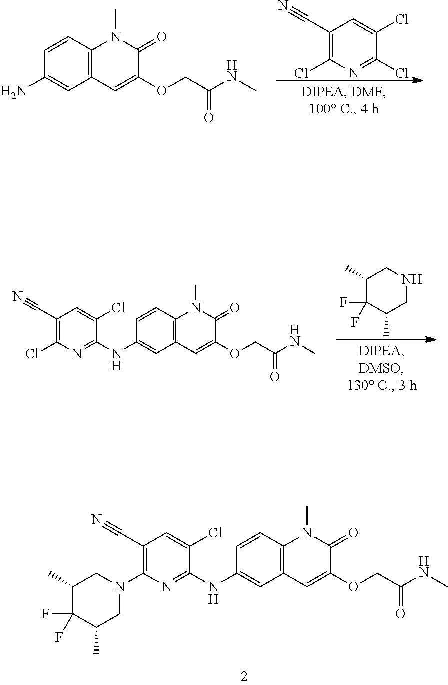

### 2-((6-((3,6-Dichloro-5-cyanopyridin-2-yl)amino)-1-methyl-2-oxo-1,2-dihydroquinolin-3-yl)oxy)-N-methylacetamide

DIEA (197.86 mg, 1.53 mmol) was added to a mixture of 2,5,6-trichloropyridine-3-carbonitrile (158.79 mg, 765.47 μmol) and 2-[(6-amino-1-methyl-2-oxo-3-quinolyl)oxy]-N-methyl-acetamide (200 mg, 765.47 μmol) in DMF (3 mL) and the reaction mixture was stirred at 100° C. for 4 hours under a N₂. The reaction mixture was cooled to 15° C., water (˜5 mL) was added and a precipitate formed. The reaction mixture was filtered and the filter cake was washed with EtOAc (˜20 mL) and concentrated in vacuum to give the title compound as a yellow solid (250 mg, 76%). ¹H NMR (400 MHz, DMSO-d₆) δ 9.64 (s, 1H), 8.39 (s, 1H), 7.93 (s, 1H), 7.73 (s, 1H), 7.66 (d, J=8.4 Hz, 1H), 7.53 (d, J=8.8 Hz, 1H), 7.22 (s, 1H), 4.58 (s, 2H), 3.69 (s, 3H), 2.67 (s, 3H).

### 2-((6-((3-Chloro-5-cyano-6-((3S,5R)-4,4-difluoro-3,5-dimethylpiperidin-1-yl)pyridin-2-yl)amino)-1-methyl-2-oxo-1,2-dihydroquinolin-3-yl)oxy)-N-methylacetamide (2)

DIPEA (298.99 mg, 2.31 mmol) was added to a solution of 2-[[6-[(3,6-dichloro-5-cyano-2-pyridyl)amino]-1-methyl-2-oxo-3-quinolyl]oxy]-N-methyl-acetamide (100 mg, 231.34 μmol) and (3R,5S)-4,4-difluoro-3,5-dimethyl-piperidine (304.45 mg, 1.16 mmol, TFA salt) in DMSO (3 mL) and the reaction mixture was stirred at 130° C. for 3 hours. The reaction mixture was then cooled to 15° C. and treated with water (8 mL) which caused a precipitate to form. The mixture was filtered and washed with water (10 mL), EtOAc (10 mL) and EtOH (20 mL), respectively. The filter cake was concentrated under reduced pressure to give the title compound as a pale brown solid (80.6 mg, 62%). ¹H NMR (400 MHz, DMSO-d₆) δ 9.14 (s, 1H), 7.99 (s, 1H), 7.92 (br s, 1H), 7.78 (s, 1H), 7.63 (br d, J=8.8 Hz, 1H), 7.47 (br d, J=9.2 Hz, 1H), 7.23 (s, 1H), 4.54 (s, 2H), 4.13 (br d, J=13.2 Hz, 2H), 3.68 (s, 3H), 2.78 (br t, J=12.8 Hz, 2H), 2.66 (br d, J=4.4 Hz, 3H), 2.15-1.98 (m, 2H), 0.84 (br d, J=6.8 Hz, 6H).

### Example 3: Synthesis of 2-((6-((5-cyano-4-((3S,5R)-4,4-difluoro-3-(2-hydroxyethyl)-5-methylpiperidin-1-yl)pyrimidin-2-yl)amino)-1-methyl-2-oxo-1,2-dihydroquinolin-3-yl)oxy)-N-methylacetamide (3) and 2-((6-((5-cyano-4-((3R,5S)-4,4-difluoro-3-(2-hydroxyethyl)-5-methylpiperidin-1-yl)pyrimidin-2-yl)amino)-1-methyl-2-oxo-1,2-dihydroquinolin-3-yl)oxy)-N-methylacetamide (4)

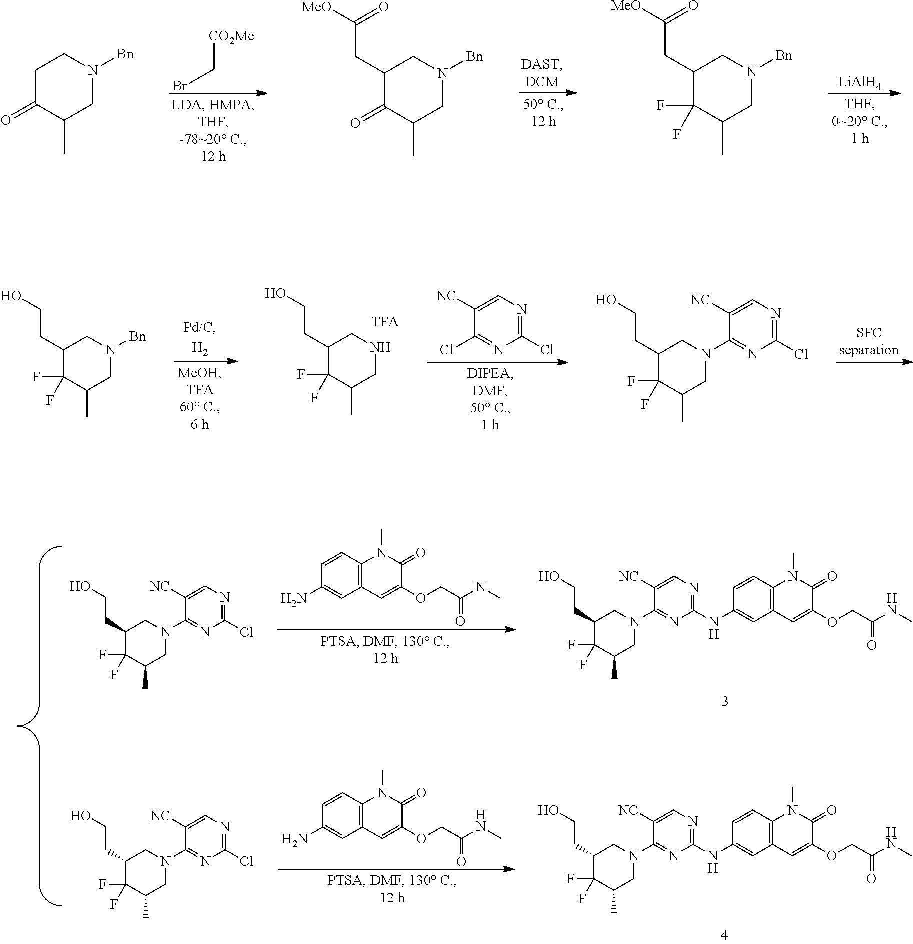

### Methyl 2-(1-benzyl-5-methyl-4-oxo-3-piperidyl)acetate

LDA (2 M, 73.80 mL) was added dropwise to a solution of 1-benzyl-3-methyl-piperidin-4-one (30 g, 147.58 mmol) in THE (300 mL) at −78° C. and the reaction was stirred at −78° C. for 30 minutes. Methyl 2-bromoacetate (33.86 g, 221.38 mmol) and HMPA (31.74 g, 177.10 mmol) were then added dropwise at −78° C. and the reaction mixture was stirred at −78° C. for 2.5 hours. The reaction mixture was allowed to warm to 20° C. and was stirred for another 9 hours. The reaction mixture was quenched with sat. NH₄Cl (100 mL), diluted with H₂O (500 mL) and then extracted with EtOAc (300 mL×3). The combined organic layers were washed with brine (200 mL), dried over Na₂SO₄, filtered and concentrated under reduced pressure to give a residue that was purified by column chromatography (SiO₂, Petroleum ether/Ethyl acetate=1/0 to 0/1) to give the title compound as a yellow oil (8.3 g, 17%). LCMS: [M+H⁺]=276.2

### Methyl 2-(1-benzyl-4,4-difluoro-5-methyl-3-piperidyl)acetate

DAST (77.74 g, 482.31 mmol) was added to a solution of methyl 2-(1-benzyl-5-methyl-4-oxo-3-piperidyl)acetate (8.3 g, 30.14 mmol) in DCM (85 mL) and the reaction mixture was stirred at 50° C. for 12 hours. The reaction mixture was diluted with DCM (60 mL) and then sat. aq. NaHCO₃ (200 mL) was added dropwise. The resulting mixture was stirred at 20° C. for 20 minutes and then extracted with EtOAc (100 mL×3). The combined organic layers were washed with brine (50 mL), dried over Na₂SO₄, filtered and concentrated under reduced pressure to give a residue that was purified by column chromatography (SiO₂, Petroleum ether/Ethyl acetate=1/0 to 0/1) to give the title compound as a yellow oil (3.7 g, 41%). ¹H NMR (400 MHz, CDCl₃) δ 7.31-7.21 (m, 5H), 3.64-3.60 (m, 3H), 3.57-3.54 (m, 1H), 3.42 (d, J=13.2 Hz, 1H), 2.97-2.87 (m, 1H), 2.78-2.66 (m, 2H), 2.65-2.48 (m, 1H), 2.25-2.05 (m, 2H), 2.04-1.87 (m, 2H), 0.92 (d, J=6.8 Hz, 3H).

### 2-(1-Benzyl-4,4-difluoro-5-methyl-3-piperidyl)ethanol

LiAlH₄ (255.29 mg, 6.73 mmol) was added slowly to a solution of methyl 2-(1-benzyl-4,4-difluoro-5-methyl-3-piperidyl)acetate (1 g, 3.36 mmol) in THE (10 mL) at 0° C. and then the reaction mixture was stirred at 20° C. for 1 hour. The reaction mixture was quenched with of sat. aq. NH₄Cl (10 mL), diluted with H₂O (10 mL) and then extracted with EtOAc (10 mL×3). The combined organic layers were washed with brine (10 mL), dried over Na₂SO₄, filtered and concentrated under reduced pressure to give the title compound as a yellow oil (700 mg, 77%). LCMS: [M+H⁺]=270.1

### 2-(4,4-Difluoro-5-methylpiperidin-3-yl)ethanol

TFA (3.39 g, 29.70 mmol) and Pd/C (0.5 g, 10% purity) were added to a solution of 2-(1-benzyl-4,4-difluoro-5-methyl-3-piperidyl)ethanol (2 g, 7.43 mmol) in MeOH (20 mL) and the reaction mixture was stirred under H₂ (15 psi) at 60° C. for 6 hours. The reaction mixture was filtered and concentrated under reduced pressure to give the title compound as a white solid (2 g, 92%, TFA salt). ¹H NMR (400 MHz, methanol-d₄) δ 3.66 (d, J=6.4 Hz, 2H), 3.62-3.60 (m, 1H), 3.45-3.39 (m, 1H), 2.97-2.88 (m, 2H), 2.51-2.26 (m, 2H), 2.11-2.03 (m, 1H), 1.53-1.44 (m, 1H), 1.11 (d, J=6.8 Hz, 3H).

### 2-Chloro-4-(4,4-difluoro-3-(2-hydroxyethyl)-5-methylpiperidin-1-yl)pyrimidine-5-carbonitrile

A mixture of 2-(4,4-difluoro-5-methyl-3-piperidyl)ethanol (1 g, 3.41 mmol, TFA salt), 2,4-dichloropyrimidine-5-carbonitrile (593.35 mg, 3.41 mmol), and DIEA (1.10 g, 8.53 mmol) in DMF (10 mL) was stirred at 50° C. for 1 hour under a N₂ atmosphere. The reaction mixture was concentrated under reduced pressure to give a residue that was purified by reversed-phase HPLC (neutral condition: Column: 80 g Agela C18; Mobile phase: [water-ACN]; B %: 35-65% 25 min; 65% 5 min) to give the title compound as a yellow solid (120 mg, 11%). LCMS: [M+H⁺]=317.0.

### 2-Chloro-4-((3S,5R)-4,4-difluoro-3-(2-hydroxyethyl)-5-methylpiperidin-1-yl)pyrimidine-5-carbonitrile & 2-chloro-4-((3R,5S)-4,4-difluoro-3-(2-hydroxyethyl)-5-methylpiperidin-1-yl)pyrimidine-5-carbonitrile

2-Chloro-4-[4,4-difluoro-3-(2-hydroxyethyl)-5-methyl-1-piperidyl]pyrimidine-5-carbonitrile (170 mg, 536.73 μmol) was separated by SFC (column: DAICEL CHIRALCEL® OJ (250 mm*30 mm, 10 μm); mobile phase: [Neu-MeOH]; B %: 10%-40%, 15 min) to give 2-chloro-4-[(3S,5R)-4,4-difluoro-3-(2-hydroxyethyl)-5-methyl-1-piperidyl]pyrimidine-5-carbonitrile as a yellow solid (70 mg, 41%, retention time=1.894 min) and 2-chloro-4-[(3R,5S)-4,4-difluoro-3-(2-hydroxyethyl)-5-methyl-1-piperidyl]pyrimidine-5-carbonitrile as a yellow solid (70 mg, 40%, retention time=2.013 min).

### 2-((6-((5-Cyano-4-((3S,5R)-4,4-difluoro-3-(2-hydroxyethyl)-5-methylpiperidin-1-yl)pyrimidin-2-yl)amino)-1-methyl-2-oxo-1,2-dihydroquinolin-3-yl)oxy)-N-methylacetamide (3)

A mixture of 2-chloro-4-[(3S,5R)-4,4-difluoro-3-(2-hydroxyethyl)-5-methyl-1-piperidyl]pyrimidine-5-carbonitrile (70 mg, 221.01 μmol), 2-[(6-amino-1-methyl-2-oxo-3-quinolyl)oxy]-N-methyl-acetamide (52.49 mg, 200.91 μmol), and 4-methylbenzenesulfonic acid monohydrate (42.04 mg, 221.01 μmol) in DMF (1 mL) was stirred at 130° C. for 12 hours under a N₂ atmosphere. The reaction mixture was concentrated under reduced pressure to give a residue that was purified by prep-HPLC (column: Waters™ Xbridge BEH C18 100*30 mm*10 μm; mobile phase: [water(10 mM NH₄HCO₃)-ACN]; B %: 25%-55%, 8 min) to give the title compound as a white solid (38 mg, 34%) as a white solid. ¹H NMR (400 MHz, DMSO-d₆) δ 10.06 (s, 1H), 8.49 (s, 1H), 7.93 (d, J=4.4 Hz, 1H), 7.85 (s, 1H), 7.71 (d, J=9.2 Hz, 1H), 7.46 (d, J=9.2 Hz, 1H), 7.20 (s, 1H), 4.83 (d, J=13.2 Hz, 1H), 4.62 (t, J=5.2 Hz, 2H), 4.56 (s, 2H), 3.66 (s, 3H), 3.52-3.47 (m, 2H), 2.96-2.87 (m, 2H), 2.66 (d, J=4.8 Hz, 3H), 2.21-2.14 (m, 2H), 1.91-1.83 (m, 1H), 1.42-1.33 (m, 1H), 1.00 (d, J=6.8 Hz, 3H). LCMS: [M+H⁺]=542.2.

### 2-((6-((5-Cyano-4-((3R,5S)-4,4-difluoro-3-(2-hydroxyethyl)-5-methylpiperidin-1-yl)pyrimidin-2-yl)amino)-1-methyl-2-oxo-1,2-dihydroquinolin-3-yl)oxy)-N-methylacetamide (4)

A mixture of 2-chloro-4-[(3R,5S)-4,4-difluoro-3-(2-hydroxyethyl)-5-methyl-1-piperidyl]pyrimidine-5-carbonitrile (70 mg, 221.01 μmol), 2-[(6-amino-1-methyl-2-oxo-3-quinolyl)oxy]-N-methyl-acetamide (52.49 mg, 200.91 μmol), and 4-methylbenzenesulfonic acid monohydrate (45.86 mg, 241.10 μmol) in DMF (2 mL) was stirred at 130° C. for 12 hours under a N₂ atmosphere. The reaction mixture was concentrated under reduced pressure to give a residue that was purified by prep-HPLC (column: Waters™ Xbridge BEH C18 100*30 mm*10 μm; mobile phase: [water(10 mM NH₄HCO₃)-ACN]; B %: 25%-55%, 8 min) to give the title compound as a white solid (16 mg, 14%). ¹H NMR (400 MHz, DMSO-d₆) δ 10.07 (s, 1H), 8.49 (s, 1H), 7.94-7.86 (m, 2H), 7.71 (d, J=9.2 Hz, 1H), 7.46 (d, J=8.8 Hz, 1H), 7.20 (s, 1H), 4.83 (d, J=11.6 Hz, 1H), 4.62 (t, J=5.2 Hz, 2H), 4.56 (s, 2H), 3.67 (s, 3H), 3.50-3.49 (m, 2H), 2.96-2.87 (m, 2H), 2.66 (d, J=4.4 Hz, 3H), 2.20 (s, 2H), 1.86 (s, 1H), 1.37 (s, 1H), 1.00 (d, J=6.8 Hz, 3H). LCMS: [M+H⁺]=542.2.

### Example 4: Synthesis of 2-((6-((4-(3-(2-aminoethyl)-4,4-difluoro-5-methylpiperidin-1-yl)-5-cyanopyrimidin-2-yl)amino)-1-methyl-2-oxo-1,2-dihydroquinolin-3-yl)oxy)-N-methylacetamide (mixture of 14 & 15)

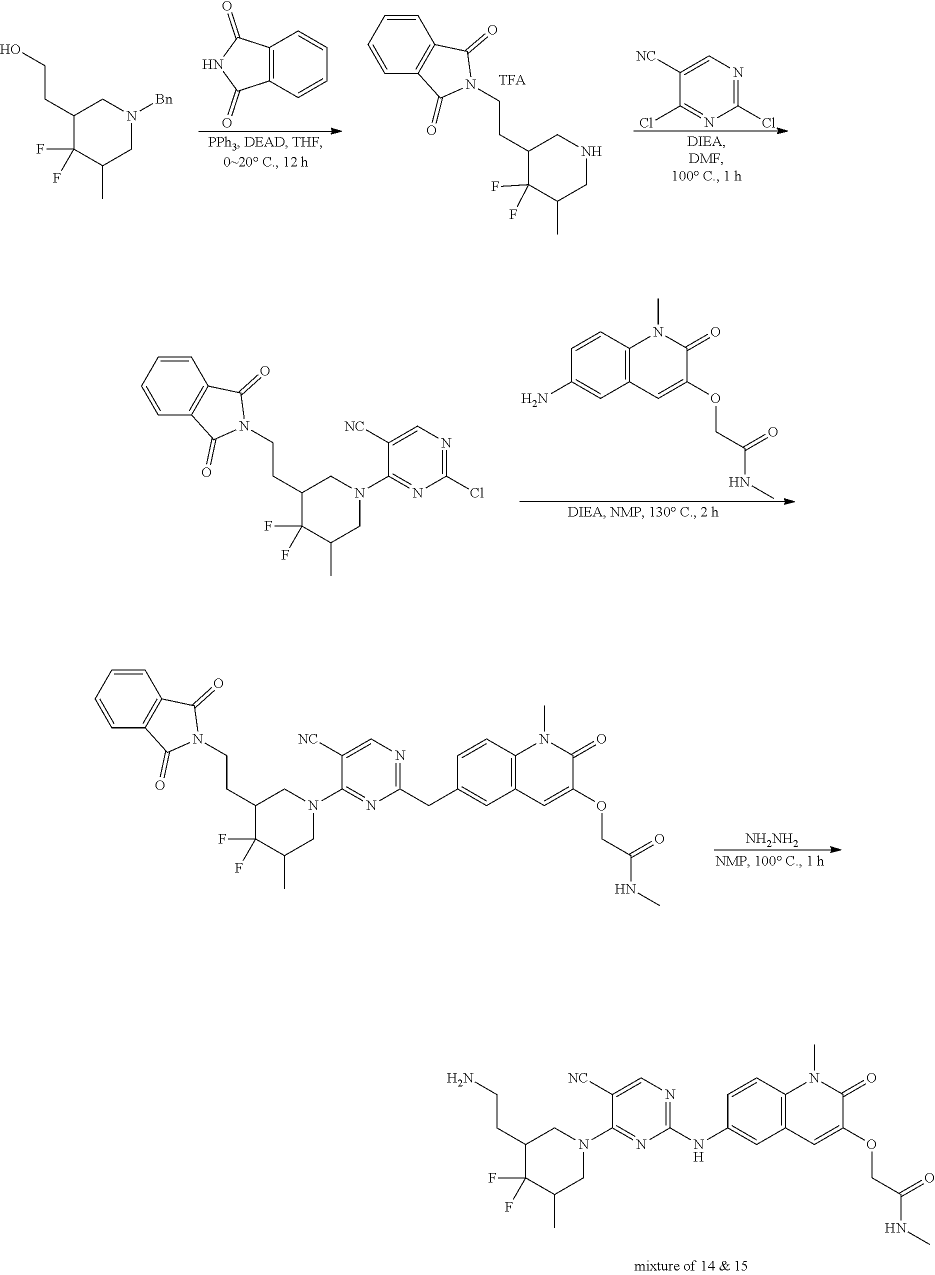

### 2-[2-(1-Benzyl-4,4-difluoro-5-methyl-3-piperidyl)ethyl]isoindoline-1,3-dione

PPh₃ (886.20 mg, 3.38 mmol) was added to a solution of 2-(1-benzyl-4,4-difluoro-5-methyl-3-piperidyl)ethanol (700 mg, 2.60 mmol) and isoindoline-1,3-dione (458.88 mg, 3.12 mmol) in DCM (7 mL) at 0° C. A solution of DEAD (588.43 mg, 3.38 mmol) in DCM (1 mL) was then added dropwise at 0° C. The reaction mixture then was stirred at 20° C. for 12 hours. The reaction mixture was concentrated under reduced pressure to give a residue that was purified by column chromatography (SiO₂, Petroleum ether/Ethyl acetate=1/0 to 0/1) to give the title compound as a colorless oil (600 mg, 58%). LCMS: [M+H⁺]=399.1

### 2-Chloro-4-(3-(2-(1,3-dioxoisoindolin-2-yl)ethyl)-4,4-difluoro-5-methylpiperidin-1-yl)pyrimidine-5-carbonitrile

A mixture of 2-[2-(4,4-difluoro-5-methyl-3-piperidyl)ethyl]isoindoline-1,3-dione (1 g, 2.37 mmol, TFA salt), 2,4-dichloropyrimidine-5-carbonitrile (494.35 mg, 2.84 mmol), and DIEA (612.03 mg, 4.74 mmol) in DMF (10 mL) was stirred at 100° C. for 1 hour under a N₂ atmosphere. The reaction mixture was concentrated under reduced pressure to give a residue that was purified by reversed-phase HPLC (neutral condition: Column: 120 g Agela C18; Mobile phase: [water-ACN]; Gradient B %: 30-60% 20 min; 60% 10 min) to give the title compound as a yellow solid (200 mg, 19%). LCMS: [M+H⁺]=446.1.

### 2-((6-((5-cyano-4-(3-(2-(1,3-dioxoisoindolin-2-yl)ethyl)-4,4-difluoro-5-methylpiperidin-1-yl)pyrimidin-2-yl)amino)-1-methyl-2-oxo-1,2-dihydroquinolin-3-yl)oxy)-N-methylacetamide

A mixture of 2-chloro-4-[3-[2-(1,3-dioxoisoindolin-2-yl)ethyl]-4,4-difluoro-5-methyl-1-piperidyl]pyrimidine-5-carbonitrile (200 mg, 448.58 μmol), 2-[(6-amino-1-methyl-2-oxo-3-quinolyl)oxy]-N-methyl-acetamide (106.55 mg, 407.80 μmol), and DIEA (105.41 mg, 815.60 μmol) in NMP (3 mL) was stirred at 130° C. for 2 hours under a N₂ atmosphere. H₂O (5 mL) was added to the reaction mixture and the precipitated solids were filtered and washed with H₂O (5 mL) and EtOAc (5 mL). The solids were dried under reduced pressure to give the title compound as a brown solid (200 mg, 64%). ¹H NMR (400 MHz, DMSO-d₆) δ 10.06 (s, 1H), 8.50 (s, 1H), 7.87-7.82 (m, 5H), 7.75-7.66 (m, 2H), 7.46 (d, J=9.2 Hz, 1H), 7.17 (s, 1H), 4.83 (d, J=12.8 Hz, 1H), 4.6 (d, J=12.4 Hz, 1H), 4.52 (s, 2H), 3.60 (s, 3H), 3.30-3.28 (m, 2H), 2.69 (s, 3H), 2.65 (d, J=4.4 Hz, 2H), 2.00-1.86 (m, 3H), 1.57-1.48 (m, 1H), 0.98 (d, J=6.4 Hz, 3H).

### 2-((6-((4-(3-(2-Aminoethyl)-4,4-difluoro-5-methylpiperidin-1-yl)-5-cyanopyrimidin-2-yl)amino)-1-methyl-2-oxo-1,2-dihydroquinolin-3-yl)oxy)-N-methylacetamide (mixture of 14 & 15)

NH₂NH₂·H₂O (279.91 mg, 5.59 mmol) was added to a mixture of 2-[[6-[[5-cyano-4-[3-[2-(1,3-dioxoisoindolin-2-yl)ethyl]-4,4-difluoro-5-methyl-1-piperidyl]pyrimidin-2-yl]amino]-1-methyl-2-oxo-3-quinolyl]oxy]-N-methyl-acetamide (250 mg, 372.76 μmol) in NMP (0.5 mL) and the reaction was heated to 100° C. and was stirred for 1 hour. The reaction mixture was concentrated under reduced pressure to give a residue that was purified by prep-HPLC (column: Phenomenex Luna® 80*30 mm*3 μm; mobile phase: [water(TFA)-ACN]; B %: 10%-40%, 8 min) to give the title compound as a yellow solid (25 mg, 12%). ¹H NMR (400 MHz, DMSO-d₆) δ 10.12 (s, 1H), 8.52 (s, 1H), 7.95 (d, J=4.8 Hz, 1H), 7.88 (s, 1H), 7.74-7.69 (m, 3H), 7.47 (d, J=9.2 Hz, 1H), 7.18 (s, 1H), 4.72-4.64 (m, 2H), 4.57 (s, 2H), 3.67 (s, 3H), 3.02 (d, J=12.8 Hz, 1H), 2.91-2.84 (m, 3H), 2.66 (d, J=4.4 Hz, 3H), 2.25-2.15 (m, 2H), 2.03-1.94 (m, 1H), 1.59-1.49 (m, 1H), 1.01 (d, J=6.8 Hz, 3H).

### Example 5: Synthesis of 5-chloro-2-((3R,5S)-4,4-difluoro-3,5-dimethylpiperidin-1-yl)-6-((1-methyl-3-((3-methyloxetan-3-yl)methoxy)-2-oxo-1,2-dihydroquinolin-6-yl)amino)nicotinonitrile (9)

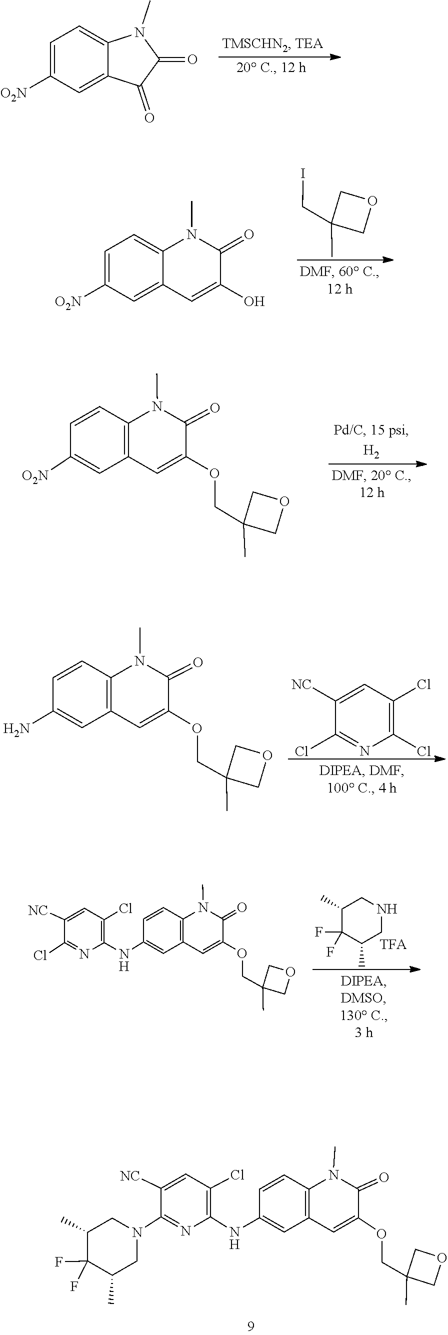

### 3-Hydroxy-1-methyl-6-nitroquinolin-2(1H)-one

Diazomethyl(trimethyl)silane (2 M, 29.10 mL) was added dropwise to a mixture of 1-methyl-5-nitro-indoline-2,3-dione (10 g, 48.51 mmol) and TEA (9.82 g, 97.01 mmol) in EtOH (300 mL) and the reaction mixture was stirred at 20° C. for 12 hours. The reaction mixture was concentrated under reduced pressure and 1 N HCl (100 mL) was added and the mixture was stirred at 20° C. for 2 hours. The mixture was filtered and washed with DMF/ethyl acetate (1/10, 50 mL) and the filter cake was dried under reduced pressure to give the title compound as a yellow solid (2 g, 19%). ¹H NMR (400 MHz, DMSO-d₆) δ 10.09 (s, 1H), 8.66 (d, J=2.4 Hz, 1H), 8.19 (dd, J=9.2, 2.4 Hz, 1H), 7.65 (d, J=9.2 Hz, 1H), 7.34 (s, 1H), 3.74 (s, 3H).

### 1-Methyl-3-((3-methyloxetan-3-yl)methoxy)-6-nitroquinolin-2(1H)-one

A mixture of 3-hydroxy-1-methyl-6-nitro-quinolin-2-one (500 mg, 2.27 mmol), 3-(iodomethyl)-3-methyl-oxetane (577.78 mg, 2.73 mmol) and Cs₂CO₃ (1.48 g, 4.54 mmol) in DMF (5 mL) was stirred at 60° C. for 12 hours under a N₂ atmosphere. H₂O (5 mL) was added and the precipitated solids were filtered and washed with H₂O (10 mL) and EtOAc (5 mL). The solids were dried under reduced pressure to give the title compound as a brown solid (350 mg, 51%). ¹H NMR (400 MHz, DMSO-d₆) δ 8.57 (d, J=2.4 Hz, 1H), 8.24 (dd, J=9.2, 2.4 Hz, 1H), 7.66 (d, J=9.2 Hz, 1H), 7.65 (s, 1H), 4.50 (d, J=6.0 Hz, 2H), 4.34 (d, J=6.0 Hz, 2H), 4.13 (s, 2H), 3.70 (s, 3H), 1.40 (s, 3H).

### 6-Amino-1-methyl-3-((3-methyloxetan-3-yl)methoxy)quinolin-2(1H)-one

Pd/C (10 mg, 10% purity) was added to a solution of 1-methyl-3-[(3-methyloxetan-3-yl)methoxy]-6-nitro-quinolin-2-one (350 mg, 1.15 mmol) in DMF (0.5 mL) under a N₂ atmosphere. The reaction mixture was stirred under H₂ (15 Psi) at 20° C. for 12 hours. The reaction mixture was filtered and concentrated under reduced pressure to give the title compound as a brown solid (250 mg, 79%). ¹H NMR (400 MHz, DMSO-d₆) δ 7.18 (d, J=9.2 Hz, 1H), 7.10 (s, 1H), 6.78 (dd, J=8.8, 2.4 Hz, 1H), 6.72 (d, J=2.4 Hz, 1H), 5.03 (s, 2H), 4.49 (d, J=5.6 Hz, 2H), 4.31 (d, J=6.0 Hz, 2H), 4.06 (s, 2H), 3.56 (s, 3H), 1.38 (s, 3H).

### 2,5-Dichloro-6-((1-methyl-3-((3-methyloxetan-3-yl)methoxy)-2-oxo-1,2-dihydroquinolin-6-yl)amino)nicotinonitrile

A mixture of 6-amino-1-methyl-3-[(3-methyloxetan-3-yl)methoxy]quinolin-2-one (250 mg, 911.36 μmol), 2,5,6-trichloropyridine-3-carbonitrile (207.96 mg, 1.00 mmol), and DIEA (235.57 mg, 1.82 mmol) in DMF (3 mL) was stirred at 100° C. for 4 hours under a N₂ atmosphere. H₂O (3 mL) was added and the precipitated solids were filtered and washed with H₂O (3 mL) and EtOAc (5 mL). The solids were dried under reduced pressure to give the title compound as a brown solid (300 mg, 74%). ¹H NMR (400 MHz, DMSO-d₆) δ 9.42 (s, 1H), 8.29 (s, 1H), 7.78 (s, 1H), 7.65 (d, J=8.8 Hz, 1H), 7.49 (d, J=9.2 Hz, 1H), 7.27 (s, 1H), 4.55 (d, J=5.6 Hz, 2H), 4.33 (d, J=6.0 Hz, 2H), 4.16 (s, 2H), 3.68 (s, 3H), 1.42 (s, 3H).

### 5-Chloro-2-((3R,5S)-4,4-difluoro-3,5-dimethylpiperidin-1-yl)-6-((1-methyl-3-((3-methyloxetan-3-yl)methoxy)-2-oxo-1,2-dihydroquinolin-6-yl)amino)nicotinonitrile (9)

A mixture of 2,5-dichloro-6-[[1-methyl-3-[(3-methyloxetan-3-yl)methoxy]-2-oxo-6-quinolyl]amino]pyridine-3-carbonitrile (100 mg, 224.57 μmol), (3S,5R)-4,4-difluoro-3,5-dimethyl-piperidine (76.84 mg, 291.94 μmol, TFA salt), and DIEA (72.56 mg, 561.42 μmol) in DMSO (1 mL) was stirred at 130° C. for 3 hours under a N₂ atmosphere. The reaction mixture was concentrated under reduced pressure to give a residue that was purified by prep-HPLC (column: Phenomenex C18 80*40 mm*3 μm; mobile phase: [water(NH₄HCO₃)-ACN]; B %: 35%-65%, 8 min) to give the title compound as a yellow solid (40 mg). ¹H NMR (400 MHz, DMSO-d₆) δ 9.16 (s, 1H), 8.0 (s, 1H), 7.84 (d, J=2.4 Hz, 1H), 7.59 (dd, J=9.2, 2.4 Hz, 1H), 7.46 (d, J=9.2 Hz, 1H), 7.29 (s, 1H), 4.48 (d, J=6.0 Hz, 2H), 4.33 (d, J=6.0 Hz, 2H), 4.13 (d, J=12.8 Hz, 2H), 4.09 (s, 2H), 3.66 (s, 3H), 2.78 (t, J=12.8 Hz, 2H), 2.11-2.04 (m, 2H), 1.39 (s, 3H), 0.85 (t, J=6.8 Hz, 6H).

### Example 6: Synthesis of 2-((3S,5R)-3-amino-4,4-difluoro-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile (48) and 2-((3R,5S)-3-amino-4,4-difluoro-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile (49)

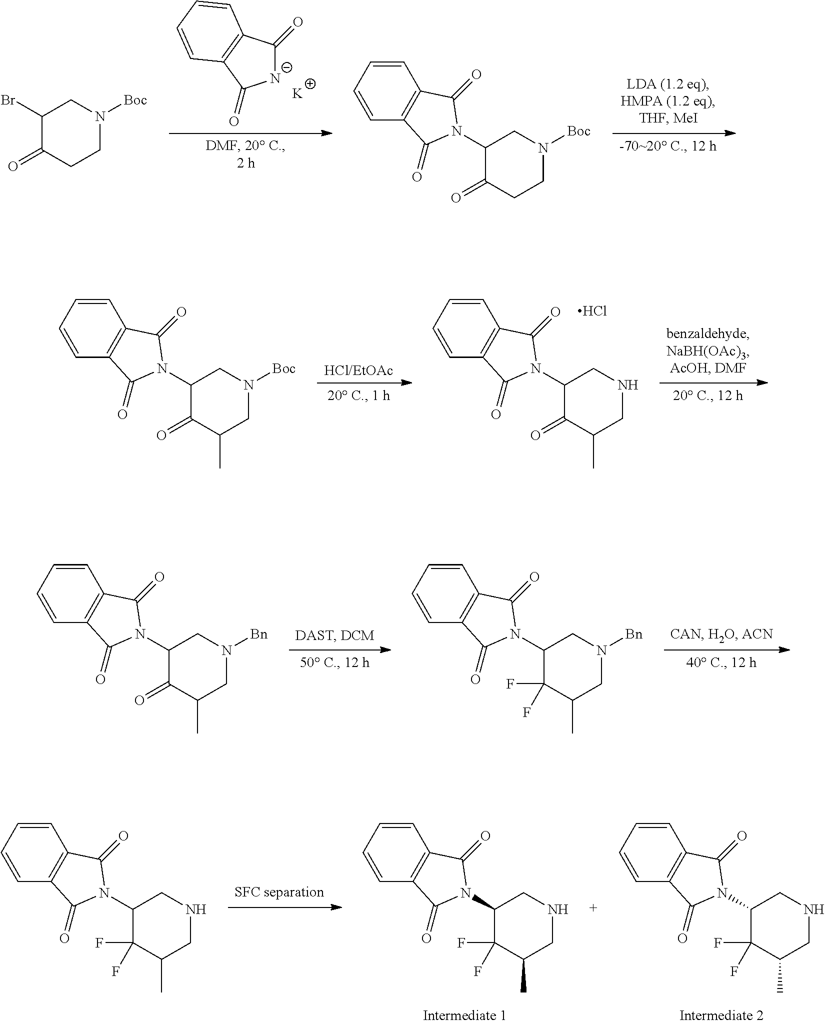

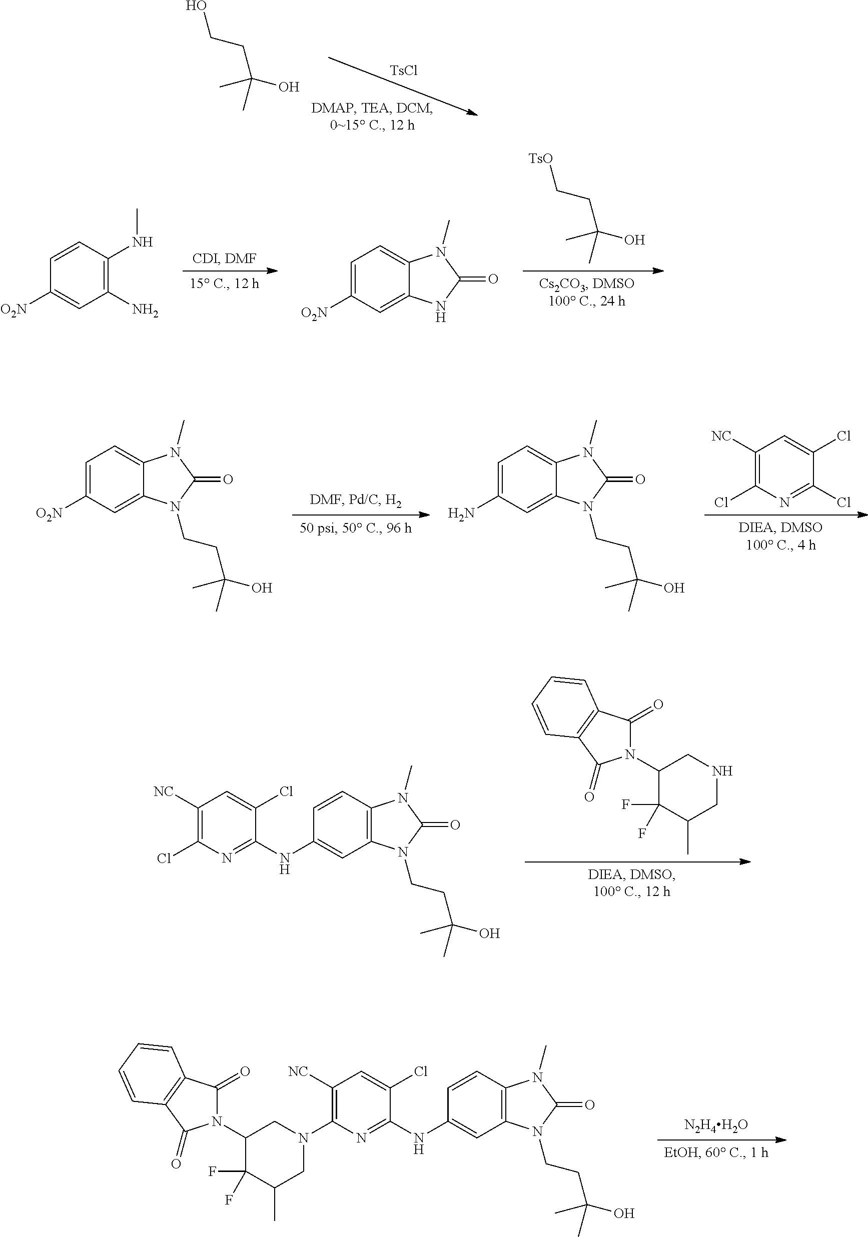

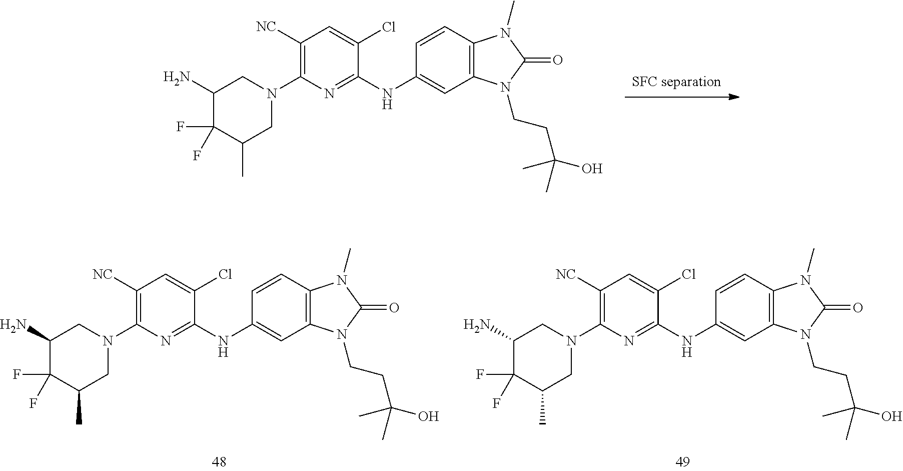 [SMILES: CC1CN(c2nc(Nc3ccc4c(c3)n(CCC(C)(C)O)c(=O)n4C)c(Cl)cc2C#N)CC(N)C1(F)F ; "SFC separation" ; C[C@@H]1CN(c2nc(Nc3ccc4c(c3)n(CCC(C)(C)O)c(=O)n4C)c(Cl)cc2C#N)C[C@H](N)C1(F)F ; C[C@H]1CN(c2nc(Nc3ccc4c(c3)n(CCC(C)(C)O)c(=O)n4C)c(Cl)cc2C#N)C[C@@H](N)C1(F)F]

### tert-Butyl 3-(1,3-dioxoisoindolin-2-yl)-4-oxopiperidine-1-carboxylate

A mixture of tert-butyl 3-bromo-4-oxo-piperidine-1-carboxylate (100 g, 359.53 mmol) and (1,3-dioxoisoindolin-2-yl) potassium (73.25 g, 395.48 mmol) in DMF (800 mL) was stirred at 20° C. for 2 hr. The mixture was added slowly to water (2.5 L), forming a precipitate which was filtered and the filter cake was washed with water (500 mL) and dried in vacuo to give the title compound as a white solid (80 g, 59% yield, 91% purity). ¹H NMR (400 MHz, DMSO-d₆) δ=7.97-7.82 (m, 4H), 4.88 (dd, J=7.2, 11.6 Hz, 1H), 4.41-4.11 (m, 2H), 3.76-3.51 (m, 1H), 3.29-3.16 (m, 1H), 2.85-2.71 (m, 1H), 2.47 (t, J=3.0 Hz, 1H), 1.47-1.43 (s, 9H).

### tert-Butyl 3-(1,3-dioxoisoindolin-2-yl)-5-methyl-4-oxopiperidine-1-carboxylate

To a solution of tert-butyl 3-(1,3-dioxoisoindolin-2-yl)-4-oxo-piperidine-1-carboxylate (25 g, 72.60 mmol) in THE (250 mL) was added LDA (2 M, 43.56 mL) dropwise at −70° C., and the mixture was stirred at −70° C. for 45 min, then HMPA (15.61 g, 87.12 mmol, 15.31 mL) was added dropwise and stirred for another 30 min. Finally, iodomethane (15.46 g, 108.90 mmol, 6.78 mL) was added dropwise at −70° C. and the resulting mixture was stirred at 20° C. for 11 hr. The reaction mixture was quenched by addition of sat. NH₄Cl aq. (400 mL), and then it was extracted with DCM (500 mL×3). The combined organic layers were dried over Na₂SO₄, filtered and concentrated under reduced pressure to give a residue which was purified by flash silica gel chromatography (Silica Flash Column, Eluent of 25˜50% Ethyl acetate/Petroleum ether gradient) to give compound the title compound as a pink solid (4.3 g, 7% yield, 87% purity). ¹H NMR (400 MHz, CDCl₃) δ=7.90-7.83 (m, 2H), 7.77-7.74 (m, 2H), 4.86 (dd, J=7.2, 11.2 Hz, 1H), 4.65-4.29 (m, 2H), 3.97-3.81 (m, 1H), 2.90 (s, 1H), 2.67 (s, 1H), 1.52 (s, 9H), 1.13 (d, J=6.8 Hz, 3H).

### 2-(5-Methyl-4-oxopiperidin-3-yl)isoindoline-1,3-dione

A mixture of tert-butyl 3-(1,3-dioxoisoindolin-2-yl)-5-methyl-4-oxo-piperidine-1-carboxylate (4.3 g, 12.00 mmol) in HCl/EtOAc (4 M, 21.50 mL) was stirred at 20° C. for 1 hr. The reaction mixture was concentrated under reduced pressure to give the title compound as a white solid (3.5 g, crude, HCl).

### 2-(1-Benzyl-5-methyl-4-oxopiperidin-3-yl)isoindoline-1,3-dione

A mixture of 2-(5-methyl-4-oxo-3-piperidyl)isoindoline-1,3-dione (3.5 g, 9.50 mmol, 80% purity, HCl), benzaldehyde (1.21 g, 11.40 mmol, 1.15 mL) and AcOH (285.25 mg, 4.75 mmol, 271.67 μL) in DMF (35 mL) was stirred at 20° C. for 2 hr, then sodium triacetoxyborohydride (4.03 g, 19.00 mmol, 2 eq) was added to the mixture, and the reaction mixture was stirred at 20° C. for 10 hr. The reaction mixture was treated with water (50 mL), forming a precipitate which was filtered and the solid was collected and dried in vacuo. The crude product was triturated with (PE:EtOAc=15:1, 60 mL) at 20° C. for 30 min to give the title compound as a white solid (3 g, 75% yield, 83% purity). ¹H NMR (400 MHz, CDCl₃) δ=7.76-7.70 (m, 2H), 7.65-7.59 (m, 2H), 7.29-7.16 (m, 5H), 5.01-4.92 (m, 1H), 3.69-3.54 (m, 2H), 3.21-3.04 (m, 3H), 2.76-2.64 (m, 1H), 2.34-2.26 (m, 1H), 0.98 (d, J=6.8 Hz, 3H).

### 2-(1-Benzyl-4,4-difluoro-5-methylpiperidin-3-yl)isoindoline-1,3-dione

To a solution of 2-(1-benzyl-5-methyl-4-oxo-3-piperidyl)isoindoline-1,3-dione (3 g, 8.61 mmol) in DCM (30 mL) was added DAST (20.82 g, 129.16 mmol, 17.07 mL) in one portion, and then the reaction mixture was stirred at 50° C. for 12 h. The reaction mixture was diluted with DCM (50 mL), then sat. NaHCO₃ aq. (200 mL) was added to the stirring mixture. The organic phase was separated and the aqueous phase was extracted with EtOAc (100 mL×3). The combined organic phase was washed with brine (150 mL), dried with anhydrous Na₂SO₄, filtered and the filtrate was concentrated in vacuo and purified by flash silica gel chromatography (Silica Flash Column, Eluent of 5˜30% Ethyl acetate/Petroleum ether gradient) to give the title compound as a white solid (2 g, 3.78 mmol, 44% yield, 70% purity). ¹H NMR (400 MHz, CDCl₃) δ=7.81-7.74 (m, 2H), 7.69-7.62 (m, 2H), 7.27-7.15 (m, 5H), 4.71-4.55 (m, 1H), 3.67-3.59 (m, 1H), 3.55-3.46 (m, 2H), 2.85-2.76 (m, 2H), 2.35-2.15 (m, 2H), 0.97 (d, J=6.4 Hz, 3H).

### 2-(4,4-Difluoro-5-methylpiperidin-3-yl)isoindoline-1,3-dione

To a mixture of 2-(1-benzyl-4,4-difluoro-5-methyl-3-piperidyl)isoindoline-1,3-dione (2 g, 5.40 mmol) in H₂O (20 mL) and ACN (80 mL) was added CAN (29.60 g, 54.00 mmol, 26.91 mL), and the reaction mixture was stirred at 40° C. for 12 hr. The reaction mixture was treated with sat.K₂CO₃ aq. (50 mL), forming a precipitate which was filtered, and the filter cake was washed with EtOAc (80 mL). The filtrate was extracted with EtOAc (100 mL×3), and the combined organic phase was washed with brine (150 mL), dried with anhydrous Na₂SO₄, filtered and the filtrate was concentrated in vacuo. The residue was purified by flash silica gel chromatography (Silica Flash Column, Eluent of 80˜100% Ethyl acetate/Petroleum ether gradient). Then it was further purified by prep-HPLC (column: Waters Xbridge Prep OBD C18 150*40 mm*10 μm; mobile phase: [water(NH₃H₂O+NH₄HCO₃)-ACN]; B %: 20%-50%, 8 min) to give the title compound as a yellow solid (550 mg, 1.95 mmol, 36% yield, 99% purity). ¹H NMR (400 MHz, DMSO-d₆) δ=7.94-7.83 (m, 4H), 4.50-4.34 (m, 1H), 3.72 (t, J=12.4 Hz, 1H), 3.15-3.03 (m, 1H), 2.98-2.88 (m, 1H), 2.54 (s, 1H), 2.41-2.31 (m, 1H), 2.22-2.01 (m, 1H), 0.90 (d, J=6.4 Hz, 3H).

### 2-((3S,5R)-4,4-Difluoro-5-methylpiperidin-3-yl)isoindoline-1,3-dione and 2-((3R,5S)-4,4-difluoro-5-methylpiperidin-3-yl)isoindoline-1,3-dione

2-(4,4-Difluoro-5-methyl-3-piperidyl)isoindoline-1,3-dione (550 mg) was separated by SFC (column: DAICEL CHIRALPAK IG (250 mm*50 mm, 10 μm); mobile phase: [0.1% NH₃H₂O ETOH]; B %: 40%-40%, 3.8 min) to give 2-[(3S,5R)-4,4-difluoro-5-methyl-3-piperidyl]isoindoline-1,3-dione as a white solid (200 mg, 520.93 μmol, 27% yield) and 2-[(3R,5S)-4,4-difluoro-5-methyl-3-piperidyl]isoindoline-1,3-dione as a white solid (190 mg, 460.99 μmol, 23% yield).

Intermediate 1: ¹H NMR (400 MHz, DMSO-d₆) δ=7.95-7.81 (m, 4H), 4.52-4.32 (m, 1H), 3.73 (t, J=12.4 Hz, 1H), 3.14-3.03 (m, 1H), 2.96-2.90 (m, 1H), 2.58 (s, 1H), 2.36 (t, J=12.6 Hz, 1H), 2.20-2.03 (m, 1H), 0.90 (d, J=6.8 Hz, 3H).

Intermediate 2: ¹H NMR (400 MHz, DMSO-d₆) δ=7.93-7.82 (m, 4H), 4.55-4.30 (m, 1H), 3.73 (t, J=12.4 Hz, 1H), 3.14-3.04 (m, 1H), 2.96-2.91 (m, 1H), 2.70 (s, 1H), 2.36 (t, J=12.0 Hz, 1H), 2.21-2.02 (m, 1H), 0.91 (d, J=6.8 Hz, 3H).

### 3-Hydroxy-3-methylbutyl 4-methylbenzenesulfonate

To a mixture of 3-methylbutane-1,3-diol (50 g, 480.09 mmol, 51.23 mL) in DCM (1 L) was added DMAP (5.87 g, 48.01 mmol) and TEA (97.16 g, 960.18 mmol, 133.64 mL) at 15° C. under a nitrogen atmosphere. 4-Methylbenzenesulfonyl chloride (91.53 g, 480.09 mmol) was added at 0° C. slowly. The reaction was stirred at 15° C. for 12 hours, then poured into sat. aq. NaHCO₃ (1000 mL). The biphasic layers were separated and the aqueous layer extracted with DCM (2000 mL). The combined organic layers were washed with brine (1500 mL), dried over anhydrous Na₂SO₄, filtered and concentrated in vacuo. The residue was purified by column chromatography (silica gel, eluted with petroleum ether/ethyl acetate=10/1 to 2/1) to give the title compound as a colorless oil (250 g, 953.23 mmol, 99% yield, 98% purity), which was used without further purification. ¹H NMR (400 MHz, CDCl₃) δ ppm 7.75-7.80 (m, 2H), 7.34 (d, J=7.99 Hz, 2H), 4.19 (t, J=6.91 Hz, 2H), 2.44 (s, 3H), 1.84 (t, J=6.85 Hz, 2H), 1.68 (s, 1H), 1.20 (s, 6H).

### 1-Methyl-5-nitro-1H-benzo[d]imidazol-2(3H)-one

A mixture of N-1-methyl-4-nitrobenzene-1,2-diamine (50 g, 299.11 mmol) and di(imidazol-1-yl)methanone (48.50 g, 299.11 mmol) in DMF (500 mL) was stirred at 15° C. for 12 hours. The resulting suspension was filtered and the filter cake was washed by EtOAc (100 mL) and concentrated under reduced pressure to give the crude title compound as a red solid (92 g, 468.15 mmol, 78% yield, 98% purity), which was used without further purification. ¹H NMR (400 MHz, DMSO-d₆) δ 11.38 (br s, 1H), 7.99 (dd, J=8.70, 2.15 Hz, 1H), 7.71 (d, J=2.03 Hz, 1H), 7.26 (d, J=8.70 Hz, 1H), 3.34 (s, 3H).

### 3-(3-Hydroxy-3-methylbutyl)-1-methyl-5-nitro-1H-benzo[d]imidazol-2(3H)-one

To a mixture of 3-hydroxy-3-methylbutyl 4-methylbenzenesulfonate (24.07 g, 93.19 mmol) and 1-methyl-5-nitro-1H-benzo[d]imidazol-2(3H)-one (15 g, 77.66 mmol) in DMSO (400 mL) was added Cs₂CO₃ (50.60 g, 155.31 mmol) in one portion under a nitrogen atmosphere. The mixture was stirred at 100° C. for 12 hours. (3-Hydroxy-3-methyl-butyl) 4-methylbenzenesulfonate (6.02 g, 23.30 mmol) was added. The mixture was stirred at 100° C. for 12 hours. Then water (600 mL) was added and the biphasic mixture was extracted with ethyl acetate (500 mL×2). The combined organic phases was dried with anhydrous Na₂SO₄, filtered and concentrated to give the crude title compound as a black oil (46 g, 154.92 mmol, 99% yield, 94% purity), which was used without further purification. ¹H NMR (400 MHz, DMSO-d₆) δ 8.02 (dd, J=8.70, 2.15 Hz, 1H), 7.96 (d, J=2.15 Hz, 1H), 7.32 (d, J=8.70 Hz, 1H), 4.52 (s, 1H), 3.91-4.00 (m, 2H), 3.38 (s, 3H), 1.67-1.75 (m, 2H), 1.16 (s, 6H). M+H⁺=280.0.

### 5-Amino-3-(3-hydroxy-3-methylbutyl)-1-methyl-1H-benzo[d]imidazol-2(3H)-one

To a solution of 3-(3-hydroxy-3-methylbutyl)-1-methyl-5-nitro-1H-benzo[d]imidazol-2(3H)-one (15 g, 53.71 mmol) in DMF (200 mL) was added Pd/C (10 g, 10%) under an argon atmosphere. The suspension was degassed under vacuum and purged with H₂ several times. The mixture was stirred under H₂ (50 psi) at 50° C. for 96 hours. The reaction mixture was filtered and the filtrate was concentrated under reduced pressure. The residue was purified by column chromatography (silica gel, eluted with DCM:MeOH=20:1 to 5:1) to give the title compound as a pink solid (13 g, 51.44 mmol, 96% yield, 99% purity). ¹H NMR (400 MHz, DMSO-d₆) δ 6.78 (d, J=8.31 Hz, 1H), 6.37 (d, J=1.83 Hz, 1H), 6.30 (dd, J=8.25, 1.90 Hz, 1H), 4.79 (s, 2H), 4.45 (s, 1H), 3.73-3.83 (m, 2H), 3.21 (s, 3H), 1.59-1.72 (m, 2H), 1.16 (s, 6H). M+H⁺=250.1.

### 2,5-Dichloro-6-((3-(3-hydroxy-3-methylbutyl)-1-methyl-2-oxo-2,3-dihydro-1H-benzo[d]imidazol-5-yl)amino)nicotinonitrile

To a mixture of 5-amino-3-(3-hydroxy-3-methyl-butyl)-1-methyl-benzimidazol-2-one (3 g, 12.03 mmol) and 2,5,6-trichloropyridine-3-carbonitrile (2.50 g, 12.03 mmol) in DMSO (30 mL) was added DIEA (3.11 g, 24.07 mmol, 4.19 mL) in one portion under N₂. The mixture was stirred at 100° C. for 12 hours. The mixture was added water (20 mL), the precipitated solid was filtered and washed with H₂O (30 mL) and ethyl acetate (20 mL), then the solid was dried under reduced pressure to give the title compound as a black solid (4 g, 6.53 mmol, 54% yield, 69% purity). ¹H NMR (400 MHz, DMSO-d₆) δ ppm 9.50 (s, 1H), 8.28 (s, 1H), 7.35 (m, 1H), 7.20 (dd, J=8.44, 1.59 Hz, 1H), 7.11 (m, 1H), 4.44 (s, 1H), 3.87 (m, 2H), 2.54 (s, 3H), 1.72 (m, 2H), 1.17 (s, 6H).

### 5-Chloro-2-(3-(1,3-dioxoisoindolin-2-yl)-4,4-difluoro-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile

To a mixture of 2,5-dichloro-6-[[3-(3-hydroxy-3-methyl-butyl)-1-methyl-2-oxo-benzimidazol-5-yl]amino]pyridine-3-carbonitrile (1.5 g, 3.57 mmol) and 2-(4,4-difluoro-5-methyl-3-piperidyl) isoindoline-1,3-dione (1.10 g, 3.93 mmol) in DMSO (15 mL) was added DIEA (922.52 mg, 7.14 mmol, 1.24 mL) in one portion under N₂. The mixture was stirred at 100° C. for 12 hours. The mixture was cooled to 15° C. and poured into water (30 mL). The aqueous phase was extracted with ethyl acetate (20 mL×3). The combined organic phase was washed with brine (50 mL), dried with anhydrous Na₂SO₄, filtered and concentrated in vacuo to give the title compound as a yellow solid (2.25 g, 2.35 mmol, 66% yield, 69% purity). ¹H NMR (400 MHz, DMSO-d₆) δ 9.13 (s, 1H), 8.01 (s, 1H), 7.45-7.95 (m, 4H), 7.11-7.20 (m, 1H), 7.00-7.08 (m, 1H), 6.81 (d, J=8.34 Hz, 1H), 4.12-4.54 (m, 4H), 3.83-3.93 (m, 1H), 3.58-3.77 (m, 1H), 3.07-3.25 (m, 1H), 2.82 (s, 2H), 2.54 (s, 3H), 2.08-2.28 (m, 1H), 1.67-1.76 (m, 1H), 1.05-1.21 (m, 6H), 0.80-1.02 (m, 3H).

### 2-(3-Amino-4,4-difluoro-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile

To a mixture of 5-chloro-2-[3-(1,3-dioxoisoindolin-2-yl)-4,4-difluoro-5-methyl-1-piperidyl]-6-[[3-(3-hydroxy-3-methyl-butyl)-1-methyl-2-oxo-benzimidazol-5-yl]amino]pyridine-3-carbonitrile (2.25 g, 3.39 mmol) in EtOH (25 mL) was added N₂H₄·H₂O (2.99 g, 50.82 mmol, 2.91 mL, 85% purity) in one portion under N₂. The mixture was stirred at 60° C. for 1 hour. The reaction mixture was concentrated under reduced pressure. The residue was purified by prep-HPLC (neutral condition; column: Welch Xtimate C18 250*70 mm #10 μm; mobile phase: [water (NH4HCO3)-ACN]; B %: 25%-60%, 20 min) to give the title compound as a yellow solid (1.5 g, 83% yield, 88% purity). ¹H NMR (400 MHz, DMSO-d₆) δ 9.13 (s, 1H), 8.01 (s, 1H), 7.45-7.95 (m, 4H), 7.11-7.20 (m, 1H), 7.00-7.08 (m, 1H), 6.81 (d, J=8.34 Hz, 1H), 4.12-4.54 (m, 4H), 3.83-3.93 (m, 1H), 3.58-3.77 (m, 1H), 3.07-3.25 (m, 1H), 2.82 (s, 2H), 2.54 (s, 3H), 2.08-2.28 (m, 1H), 1.67-1.76 (m, 1H), 1.05-1.21 (m, 6H), 0.80-1.02 (m, 3H).

### 2-((3R,5S)-3-amino-4,4-difluoro-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile and 2-((3S,5R)-3-amino-4,4-difluoro-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile

The mixture of 2-(3-amino-4,4-difluoro-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile was separated by SFC (condition: column: DAICEL CHIRALCEL OJ (250 mm*50 mm, 10 μm); mobile phase: [0.1% NH₃H₂O MEOH]; B %: 20%-20%, 5.3 min) to give 2-((3R,5S)-3-amino-4,4-difluoro-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile as a white solid and 2-((3S,5R)-3-amino-4,4-difluoro-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile as a white solid.

### 2-((3S,5R)-3-Amino-4,4-difluoro-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile (48)

¹H NMR (400 MHz, DMSO-d₆) δ ppm 9.00 (s, 1H), 7.95 (s, 1H), 7.29 (d, J=1.63 Hz, 1H), 7.22 (dd, J=8.32, 1.81 Hz, 1H), 7.11 (d, J=8.38 Hz, 1H), 4.47 (s, 1H), 4.10-4.25 (m, 1H), 3.93-4.02 (m, 1H), 3.84-3.93 (m, 2H), 2.58-2.94 (m, 3H), 1.92-2.12 (m, 1H), 1.61-1.76 (m, 4H), 1.15 (s, 6H), 0.78 (d, J=6.75 Hz, 3H).

### 2-((3R,5S)-3-Amino-4,4-difluoro-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile (49)

¹H NMR (400 MHz, DMSO-d₆) δ 9.00 (s, 1H), 7.95 (s, 1H), 7.29 (d, J=1.75 Hz, 1H), 7.22 (dd, J=8.38, 1.75 Hz, 1H), 7.11 (d, J=8.38 Hz, 1H), 4.47 (s, 1H), 4.11-4.25 (m, 1H), 3.78-4.04 (m, 3H), 2.75-2.95 (m, 2H), 2.68 (br t, J=12.69 Hz, 1H), 1.88-2.13 (m, 1H), 1.50-1.80 (m, 4H), 1.15 (s, 6H), 0.78 (d, J=6.75 Hz, 3H).

The absolute configurations of compounds 48 & 49 were randomly assigned based on the amino group and methyl group being in cis-conformation.

### Example 7: Synthesis of 5-chloro-2-[(3S,4R,5R)-4-fluoro-3-hydroxy-5-methyl-1-piperidyl]-6-[[3-(3-hydroxy-3-methyl-butyl)-1-methyl-2-oxo-benzimidazol-5-yl]amino]pyridine-3-carbonitrile (50) and 5-chloro-2-[(3R,4S,5S)-4-fluoro-3-hydroxy-5-methyl-1-piperidyl]-6-[[3-(3-hydroxy-3-methyl-butyl)-1-methyl-2-oxo-benzimidazol-5-yl]amino]pyridine-3-carbonitrile (51)

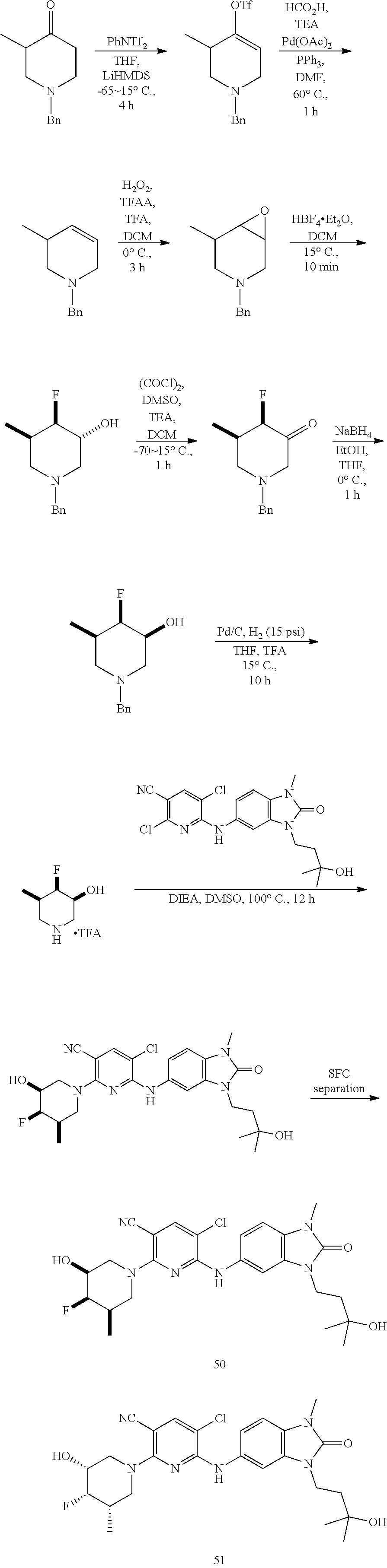

### 1-Benzyl-3-methyl-1,2,3,6-tetrahydropyridin-4-yl trifluoromethanesulfonate

To a solution of 1-benzyl-3-methyl-piperidin-4-one (50 g, 245.97 mmol) in THE (500 mL) was added LiHMDS (1 M, 295.16 mL) (1M in THF) dropwise at −65° C. After addition, the mixture was stirred at −65° C. for 1 hr, then 1,1,1-trifluoro-N-phenyl-N-(trifluoromethylsulfonyl)methanesulfonamide (96.66 g, 270.56 mmol) in THE (400 mL) was added dropwise at −65° C. The resulting mixture was stirred at 15° C. for 3 hr. The mixture (combined with another batch of same scale) was quenched with sat. NH₄Cl (800 mL) slowly and added water (800 mL). The mixture was extracted with ethyl acetate (800 mL×3). The combined organic phase was dried with anhydrous Na₂SO₄, filtered and concentrated to give the residue. The residue was purified by silica gel column chromatography (silica gel, Petroleum ether/Ethyl acetate=1/0, 20/1) to get 180 g crude product. The crude product was dissolved in ethyl acetate (1 L), then water (1 L) was added to the mixture, followed by the addition of 1N HCl until pH=3. A white precipitate formed and the mixture was filtered to get the filter cake. Water (1 L) was added to the filter cake, and then sat. Na₂CO₃ was added until pH=8. The mixture was extracted with ethyl acetate (1 L×3). The combined organic phase was dried with anhydrous Na₂SO₄, filtered and concentrated to give the title compound as a yellow oil (140 g, 93% purity). ¹H NMR (400 MHz, CDCl₃) δ ppm 7.33-7.36 (m, 4H), 7.27-7.32 (m, 1H), 5.69-5.74 (m, 1H), 3.57-3.67 (m, 2H), 3.06-3.18 (m, 2H), 2.79 (dd, J=11.31, 4.95 Hz, 1H), 2.62-2.72 (m, 1H), 2.37 (dd, J=11.37, 5.87 Hz, 1H), 1.16 (d, J=6.85 Hz, 3H).

### 1-Benzyl-3-methyl-1,2,3,6-tetrahydropyridine

A mixture of 1-benzyl-3-methyl-1,2,3,6-tetrahydropyridin-4-yl trifluoromethanesulfonate (30 g, 89.46 mmol), Pd(OAc)₂ (401.69 mg, 1.79 mmol), PPh₃ (938.58 mg, 3.58 mmol) and TEA (27.16 g, 268.38 mmol, 37.36 mL) in DMF (300 mL) was stirred at 60° C. for 10 min under N₂, then formic acid (4.12 g, 89.46 mmol, 3.37 mL) was added to the mixture in one portion. The mixture was stirred at 60° C. for 50 min under N₂ atmosphere. Water (2 L) was added to the mixture (combined with other 3 batches of same scale) followed by extraction with ethyl acetate (3×800 mL). The combined organic phase was washed with brine (1 L), dried with anhydrous Na₂SO₄, filtered and concentrated in vacuo. The residue was purified by column chromatography (silica gel, Petroleum ether/Ethyl acetate=1/0 to 20/1) to give the title compound as a yellow oil (32 g, 148.65 mmol, 42% yield, 87% purity). ¹H NMR (400 MHz, CDCl₃) δ ppm 7.30-7.39 (m, 4H), 7.27 (s, 2H), 7.23-7.26 (m, 1H), 5.59-5.68 (m, 2H), 3.52-3.66 (m, 2H), 3.02-3.14 (m, 1H), 2.74-2.88 (m, 2H), 2.37-2.48 (m, 1H), 1.98 (dd, J=10.94, 8.13 Hz, 1H), 0.96 (d, J=7.09 Hz, 3H).

### 3-Benzyl-5-methyl-7-oxa-3-azabicyclo[4.1.0]heptane

To a solution of H₂O₂ (15.14 g, 133.49 mmol, 12.83 mL, 30% purity) in DCM (150 mL) was added a solution of trifluoroacetic anhydride (TFAA) (84.11 g, 400.47 mmol, 55.70 mL) in DCM (50 mL) dropwise at 0° C., the resulting suspension was stirred for 2 hr at 0° C. Meanwhile TFA (3.04 g, 26.70 mmol, 1.98 mL) was added dropwise into a solution of 1-benzyl-3-methyl-1,2,3,6-tetrahydropyridine (5 g, 26.70 mmol) in DCM (100 ml) at 0° C. and stirred for 2 hr. Cold TFA/piperidine solution was added into the H₂O₂/TFAA solution dropwise and stirred for 1 hr at 0° C. The mixture was washed with saturated sodium sulfite solution (300 mL) slowly under N₂ at 0° C., then sat. NaHCO₃ was used to adjust the system pH=8. The mixture was extracted with DCM (3×200 mL). The wet starch potassium iodide paper was used to detect the peroxide, and the combined organic phase was dried with anhydrous Na₂SO₄, filtered and concentrated in vacuo. The residue was purified by silica gel column chromatography (silica gel, Petroleum ether/Ethyl acetate=1/0, 10/1) to give the title compound a yellow oil (2.9 g, 13.12 mmol, 49% yield, 92% purity). ¹H NMR (400 MHz, CDCl₃) δ ppm 7.30-7.33 (m, 4H), 7.22-7.27 (m, 1H), 3.47 (s, 2H), 3.22 (t, J=3.70 Hz, 1H), 2.97 (d, J=3.93 Hz, 1H), 2.84-2.92 (m, 1H), 2.76-2.83 (m, 1H), 2.46 (dd, J=11.50, 4.71 Hz, 1H), 2.20 (t, J=6.44 Hz, 1H), 1.88 (dd, J=11.44, 6.08 Hz, 1H), 1.10 (d, J=7.15 Hz, 3H).

### (3R,4R,5R)-1-Benzyl-4-fluoro-5-methylpiperidin-3-ol

To a solution of 3-benzyl-5-methyl-7-oxa-3-azabicyclo[4.1.0]heptane (2.9 g, 14.27 mmol) in DCM (45 mL) was added ethoxyethane;trifluoroborane;hydrofluoride (9.24 g, 28.53 mmol, 7.83 mL, 50% purity) in one portion at 15° C. under N₂. The mixture was stirred at 15° C. for 10 min. Sat. NaHCO₃ (20 mL) was added to the mixture. The mixture was extracted with DCM (3×15 mL). The combined organic phase was dried with anhydrous Na₂SO₄, filtered and concentrated to give the residue. The residue was purified by column chromatography (silica gel, Petroleum ether/Ethyl acetate=1/0 to 5/1) to give the title compound as a yellow solid (2 g, 8.51 mmol, 60% yield, 95% purity). ¹H NMR (400 MHz, CDCl₃) δ ppm 7.27-7.38 (m, 5H), 4.35-4.52 (m, 1H), 3.87 (br s, 1H), 3.54 (s, 2H), 2.81-3.01 (m, 1H), 2.74 (br d, J=10.37 Hz, 1H), 2.48-2.59 (m, 2H), 1.99-2.25 (m, 2H), 0.99 (d, J=6.79 Hz, 3H).

### (4R,5R)-1-Benzyl-4-fluoro-5-methylpiperidin-3-one

To a solution of (COCl)₂ (1.71 g, 13.44 mmol, 1.18 mL) in DCM (50 mL) was added DMSO (2.10 g, 26.87 mmol, 2.10 mL) in DCM (5 mL) dropwise at −65° C. After stirring at −65° C. for 15 mins a solution of (3R,4R,5R)-1-benzyl-4-fluoro-5-methyl-piperidin-3-ol (2 g, 8.96 mmol, 1 eq) in DCM (20 mL) was added dropwise, slowly at −65° C. The mixture was stirred at −65° C. for further 15 mins, and TEA (4.53 g, 44.79 mmol, 6.23 mL) was added to the mixture. After addition, the mixture was stirred at 15° C. for 0.5 h. Water (150 mL) was added slowly at 0° C. and the mixture was extracted with DCM (3×100 mL). The combined organic phase was dried with anhydrous Na₂SO₄, filtered and concentrated to give the title compound as a yellow oil (2 g, 5.69 mmol, 64% yield, 63% purity) (in 20 mL THF).

### (3S,4R,5R)-1-Benzyl-4-fluoro-5-methylpiperidin-3-ol

To a solution of (4R,5R)-1-benzyl-4-fluoro-5-methylpiperidin-3-one (1 g, 4.52 mmol) in EtOH (10 mL) and THE (10 mL) was added NaBH₄ (205.17 mg, 5.42 mmol) at 0° C. under N₂. The mixture was stirred at 0° C. for 1 hr. The reaction was quenched with cold water (50 mL) and extracted with ethyl acetate (50 mL×3). The organic layer was collected, washed with water, brine, dried over Na₂SO₄ and concentrated in vacuo. The residue was purified by flash silica gel column chromatography (ISCO®; 20 g SepaFlash® Silica Flash Column, Eluent of 0˜25% Ethyl acetate/Petroleum ether gradient @120 mL/min) to give 500 mg of crude product. The crude product was purified by prep-HPLC (column: Phenomenex C18 75*30 mm*3 μm; mobile phase: [water(NH₄HCO₃)-ACN]; B %: 25%-45%, 8 min) to give the title compound as a white solid (400 mg, 1.68 mmol, 37% yield, 94% purity). ¹H NMR (400 MHz, CDCl₃) δ ppm 7.24-7.36 (m, 5H), 4.50-4.71 (m, 1H), 3.70-3.84 (m, 1H), 3.55 (d, J=1.71 Hz, 2H), 2.90 (br dd, J=10.39, 5.01 Hz, 1H), 2.49-2.59 (m, 1H), 2.07-2.18 (m, 1H), 1.81-2.03 (m, 3H), 1.02 (d, J=6.72 Hz, 3H).

### (3S,4R,5R)-4-Fluoro-5-methylpiperidin-3-ol

To a mixture of Pd/C (0.2 g, 10% purity) in THE (20 mL) was added (3S,4R,5R)-1-benzyl-4-fluoro-5-methylpiperidin-3-ol (400 mg, 1.79 mmol) and TFA (612.79 mg, 5.37 mmol, 397.92 μL) under Ar. The mixture was stirred at 15° C. for 10 hr under H₂ (15 psi). The mixture was filtered and the filtrate was concentrated in vacuo without further purification to give the title compound as a white solid (450 mg, crude, TFA). ¹H NMR (400 MHz, DMSO-d₆) δ ppm 4.52-4.76 (m, 1H), 3.70-3.91 (m, 1H), 3.02-3.21 (m, 2H), 2.70-2.86 (m, 1H), 2.59 (q, J=12.15 Hz, 1H), 1.94-2.11 (m, 1H), 0.95-1.01 (m, 3H).

### 5-Chloro-2-((3S,4R,5R)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile

To a solution of 2,5-dichloro-6-[[3-(3-hydroxy-3-methyl-butyl)-1-methyl-2-oxo-benzimidazol-5-yl]amino]pyridine-3-carbonitrile (100 mg, 237.93 μmol) in DMSO (1 mL) was added DIEA (153.75 mg, 1.19 mmol, 207.22 μL) and 4-fluoro-5-methyl-piperidin-3-ol (60.54 mg, 356.89 μmol, HCl). The mixture was stirred at 100° C. for 12 hr. The solution was filtered and concentrated in vacuo. The residue was purified by prep-HPLC (column: Phenomenex C18 75*30 mm*3 μm; mobile phase: [water(NH₄HCO₃)-ACN]; B %: 25%-55%, 8 min) to give the title compound as a white solid (80 mg, 154.59 μmol, 65% yield, 99% purity). ¹H NMR (400 MHz, DMSO-d₆) δ=8.94 (s, 1H), 7.92 (s, 1H), 7.32 (d, J=1.7 Hz, 1H), 7.27-7.22 (m, 1H), 7.10 (d, J=8.4 Hz, 1H), 5.26 (d, J=6.0 Hz, 1H), 4.65-4.58 (m, 1H), 4.51-4.40 (m, 2H), 4.10 (br dd, J=4.3, 12.9 Hz, 1H), 3.96-3.75 (m, 4H), 3.59-3.43 (m, 1H), 2.94 (t, J=11.9 Hz, 1H), 2.74-2.60 (m, 2H), 1.94-1.76 (m, 1H), 1.74-1.66 (m, 2H), 1.18-1.14 (m, 6H), 0.84-0.78 (m, 3H).

### 5-chloro-2-((3S,4R,5R)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile (50) and 5-chloro-2-((3R,4S,5S)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile (51)

Racemic 5-chloro-2-((3S,4R,5R)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile (80 mg, 154.59 μmol) was purified by SFC (column: ChiralPak IH, 250*30 mm, 10 μm; mobile phase: [Neu-IPA]; B %: 45%-45%, 10 min) to give 5-chloro-2-((3S,4R,5R)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile as a white solid (30 mg, 57.45 μmol, 30% yield, 99% purity) and 5-chloro-2-((3R,4S,5S)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile as a white solid (30 mg, 56.00 μmol, 29% yield, 96% purity).

50: ¹H NMR (400 MHz, DMSO-d₆) δ=8.94 (s, 1H), 7.92 (s, 1H), 7.32 (d, J=1.5 Hz, 1H), 7.28-7.21 (m, 1H), 7.10 (d, J=8.3 Hz, 1H), 5.76 (s, 1H), 5.27 (d, J=5.9 Hz, 1H), 4.64-4.48 (m, 1H), 4.47 (s, 1H), 4.17-4.05 (m, 1H), 3.95-3.80 (m, 3H), 3.59-3.42 (m, 1H), 3.30 (br s, 1H), 2.95 (t, J=12.0 Hz, 1H), 2.74-2.65 (m, 1H), 2.34 (s, 1H), 1.96-1.76 (m, 1H), 1.74-1.61 (m, 2H), 1.17 (s, 6H), 0.82 (d, J=6.9 Hz, 3H).

51: ¹H NMR (400 MHz, DMSO-d₆) δ=8.93 (s, 1H), 7.92 (s, 1H), 7.32 (d, J=1.6 Hz, 1H), 7.24 (dd, J=1.7, 8.3 Hz, 1H), 7.09 (d, J=8.4 Hz, 1H), 5.26 (d, J=6.0 Hz, 1H), 4.65-4.47 (m, 1H), 4.46 (s, 1H), 4.15-4.06 (m, 1H), 3.94-3.79 (m, 3H), 3.60-3.42 (m, 1H), 3.29 (s, 1H), 2.94 (t, J=11.9 Hz, 1H), 2.76-2.62 (m, 2H), 2.36-2.29 (m, 1H), 1.98-1.76 (m, 1H), 1.75-1.64 (m, 2H), 1.16 (s, 6H), 0.81 (d, J=6.9 Hz, 3H).

The absolute configurations of compounds 50 & 51 were randomly assigned based on all substituents of the piperidine ring being in cis-conformation.

### Example 8: Synthesis of 5-fluoro-2-[(3S,4R,5R)-4-fluoro-3-hydroxy-5-methyl-1-piperidyl]-6-[[3-(3-hydroxy-3-methyl-butyl)-1-methyl-2-oxo-benzimidazol-5-yl]amino]pyridine-3-carbonitrile (52) and Synthesis of 5-fluoro-2-[(3R,4S,5S)-4-fluoro-3-hydroxy-5-methyl-1-piperidyl]-6-[[3-(3-hydroxy-3-methyl-butyl)-1-methyl-2-oxo-benzimidazol-5-yl]amino]pyridine-3-carbonitrile (53)

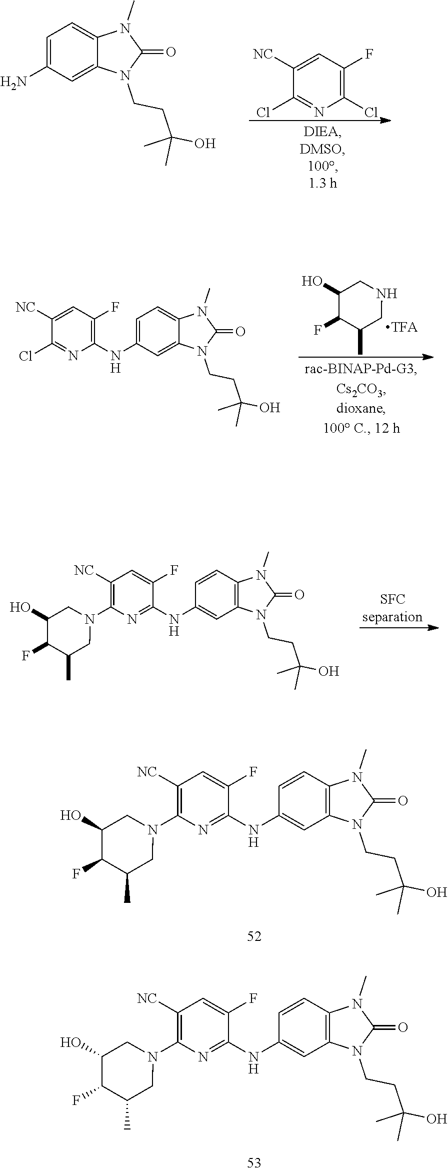

### 2-Chloro-5-fluoro-6-((3-(3-hydroxy-3-methylbutyl)-1-methyl-2-oxo-2,3-dihydro-1H-benzo[d]imidazol-5-yl)amino)nicotinonitrile

To a mixture of 5-amino-3-(3-hydroxy-3-methylbutyl)-1-methyl-1H-benzo[d]imidazol-2(3H)-one (10 g, 40.11 mmol) and 2,6-dichloro-5-fluoro-pyridine-3-carbonitrile (7.66 g, 40.11 mmol) in DMSO (100 mL) was added DIEA (10.37 g, 80.22 mmol, 13.97 mL) in one portion under N₂. The mixture was stirred at 100° C. for 1.3 hours. Water (150 mL) was added to the mixture and the resulting suspension was filtered and the filter cake was dried under reduced pressure to get the crude title compound as a white solid (16 g, crude), which was used without further purification. ¹H NMR (400 MHz, DMSO-d₆) δ ppm 9.95 (s, 1H), 8.02-8.24 (m, 1H), 7.51 (d, J=3.00 Hz, 1H), 7.25-7.35 (m, 1H), 7.13 (dd, J=8.38, 5.13 Hz, 1H), 4.44 (d, J=5.25 Hz, 1H), 3.79-3.96 (m, 2H), 3.24-3.40 (m, 3H), 1.64-1.80 (m, 2H), 1.18 (d, J=5.00 Hz, 5H). M+H⁺=404.1.

### 5-Fluoro-2-((3S,4R,5R)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile

To a suspension of 2-chloro-5-fluoro-6-((3-(3-hydroxy-3-methylbutyl)-1-methyl-2-oxo-2,3-dihydro-1H-benzo[d]imidazol-5-yl)amino)nicotinonitrile (300 mg, 742.87 μmol), 4-fluoro-5-methyl-piperidin-3-ol (367.26 mg, 1.49 mmol, TFA) and Cs₂CO₃ (2.42 g, 7.43 mmol) in dioxane (6 mL) was added rac-BINAP-Pd-G3 (73.72 mg, 74.29 μmol) under a nitrogen atmosphere. The mixture was stirred at 100° C. for 12 hr. The mixture was filtered and the filtrate was concentrated and then purified by prep-HPLC to give the title compound as a white solid (60 mg, 114.01 μmol, 15% yield, 95% purity). ¹H NMR (400 MHz, DMSO-d₆) δ ppm 9.41 (s, 1H), 7.77 (d, J=10.88 Hz, 1H), 7.44 (s, 1H), 7.38 (d, J=8.31 Hz, 1H), 7.08 (d, J=8.44 Hz, 1H), 5.27 (d, J=5.87 Hz, 1H), 4.50-4.66 (m, 1H), 4.47 (s, 1H), 4.02 (dd, J=12.41, 4.71 Hz, 1H), 3.85-3.91 (m, 2H), 3.80 (br dd, J=12.72, 3.30 Hz, 1H), 3.50-3.66 (m, 1H), 2.96 (t, J=11.80 Hz, 1H), 2.74 (t, J=12.47 Hz, 1H), 1.85-2.02 (m, 1H), 1.66-1.73 (m, 2H), 1.16 (s, 6H), 0.91 (d, J=6.85 Hz, 3H).

### 5-Fluoro-2-((3S,4R,5R)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile (52)

5-Fluoro-2-((3S,4R,5R)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile (70 mg, 139.85 μmol) was separated by SFC to give the title compound as a yellow solid (25.7 mg, 50.83 μmol, 36% yield, 99% purity). ¹H NMR (400 MHz, MeCN-d₃) δ ppm 7.76 (s, 1H), 7.55 (d, J=1.83 Hz, 1H), 7.44 (d, J=10.64 Hz, 1H), 7.20 (dd, J=8.44, 1.96 Hz, 1H), 7.00 (d, J=8.44 Hz, 1H), 4.55-4.72 (m, 1H), 4.10 (dd, J=12.10, 3.18 Hz, 1H), 3.90-4.02 (m, 2H), 3.82-3.90 (m, 1H), 3.61-3.77 (m, 2H), 3.33 (s, 3H), 2.97-3.06 (m, 2H), 2.84 (t, J=12.53 Hz, 1H), 1.98-2.11 (m, 1H), 1.80 (t, J=7.95 Hz, 2H), 1.22 (d, J=2.81 Hz, 6H), 0.99 (d, J=6.97 Hz, 3H). M+H⁺=501.3.

### 5-Fluoro-2-((3R,4S,5S)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile (53)

5-Fluoro-2-((3S,4R,5R)-4-fluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile (70 mg, 139.85 μmol) was separated by SFC to give the title compound as a yellow solid (24.8 mg, 49.05 μmol, 35% yield, 99% purity). ¹H NMR (400 MHz, MeCN-d₃) δ ppm 7.76 (s, 1H), 7.55 (d, J=1.83 Hz, 1H), 7.45 (d, J=10.76 Hz, 1H), 7.20 (dd, J=8.38, 1.90 Hz, 1H), 7.00 (d, J=8.31 Hz, 1H), 4.54-4.72 (m, 1H), 4.06-4.15 (m, 1H), 3.90-4.02 (m, 2H), 3.86 (dd, J=13.20, 3.18 Hz, 1H), 3.59-3.78 (m, 2H), 3.33 (s, 3H), 2.99-3.06 (m, 1H), 2.98 (s, 1H), 2.84 (t, J=12.53 Hz, 1H), 1.98-2.12 (m, 1H), 1.77-1.84 (m, 2H), 1.22 (d, J=2.69 Hz, 6H), 0.99 (d, J=6.97 Hz, 3H). M+H⁺=501.3.

The absolute configurations of compounds 52 & 53 were randomly assigned based on all substituents of the piperidine ring being in cis-conformation.

### Example 9: Synthesis of 5-fluoro-2-((3S,4R,5R)-4-fluoro-3,5-dimethylpiperidin-1-yl)-6-((3-((S)-3-hydroxybutyl)-1-methyl-2-oxo-2,3-dihydro-1H-benzo[d]imidazol-5-yl)amino) nicotinonitrile (54) and 5-fluoro-2-((3S,4S,5R)-4-fluoro-3,5-dimethylpiperidin-1-yl)-6-((3-((R)-3-hydroxybutyl)-1-methyl-2-oxo-2,3-dihydro-1H-benzo[d]imidazol-5-yl)amino) nicotinonitrile (55)

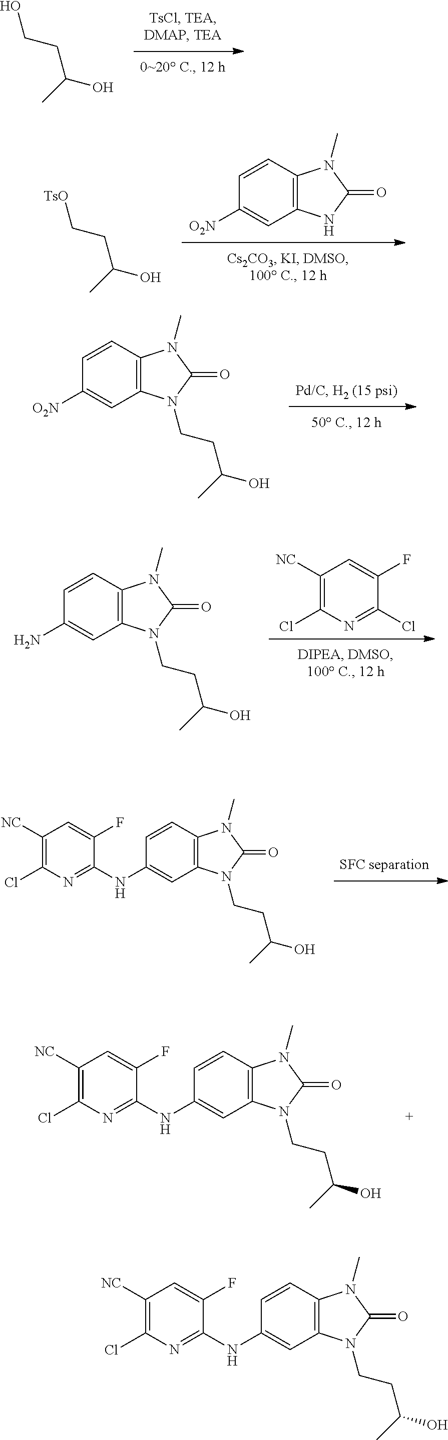

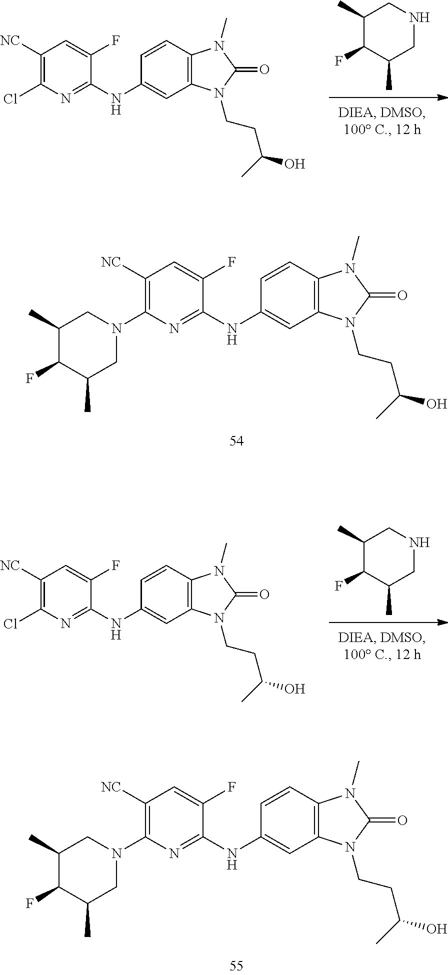

### 3-Hydroxybutyl 4-methylbenzenesulfonate

To a mixture of butane-1,3-diol (25 g, 277.41 mmol, 51.23 mL) in DCM (500 mL) was added DMAP (3.39 g, 27.74 mmol) and TEA (56.14 g, 554.81 mmol, 77.22 mL) in one portion under N₂. Then 4-methylbenzenesulfonyl chloride (52.89 g, 277.41 mmol) was added at 0° C. slowly. The mixture was stirred at 20° C. for 12 hours. The mixture was poured into sat. NaHCO₃ (500 mL), extracted with DCM (500 mL×3). The combined organic layer was washed with brine (1.2 L), dried over anhydrous Na₂SO₄, filtered and concentrated in vacuo. The residue was purified by column chromatography (SiO₂, Petroleum ether/Ethyl acetate=10/1 to 2/1) to give the title compound as a purple oil (58 g, 227.71 mmol, 41% yield, 96% purity). ¹H NMR (400 MHz, CDCl₃) δ 7.73 (br d, J=7.83 Hz, 2H), 7.30 (br d, J=7.82 Hz, 2H), 4.11-4.25 (m, 1H), 4.00-4.11 (m, 1H), 3.78-3.93 (m, 1H), 2.39 (s, 3H), 1.58-2.06 (m, 3H), 1.11 (br d, J=6.11 Hz, 3H).

### 3-Hydroxybutyl 4-methylbenzenesulfonate

To a mixture of butane-1,3-diol (25 g, 277.41 mmol, 51.23 mL) in DCM (500 mL) was added DMAP (3.39 g, 27.74 mmol) and TEA (56.14 g, 554.81 mmol, 77.22 mL) in one portion under N₂. Then 4-methylbenzenesulfonyl chloride (52.89 g, 277.41 mmol) was added at 0° C. slowly. The mixture was stirred at 20° C. for 12 hours. The mixture was poured into sat. NaHCO₃ (500 mL), extracted with DCM (500 mL×3). The combined organic layer was washed with brine (1.2 L), dried over anhydrous Na₂SO₄, filtered and concentrated in vacuo. The residue was purified by column chromatography (SiO₂, Petroleum ether/Ethyl acetate=10/1 to 2/1) to give the title compound as a purple oil (58 g, 227.71 mmol, 41% yield, 96% purity). ¹H NMR (400 MHz, CDCl₃) δ 7.73 (br d, J=7.83 Hz, 2H), 7.30 (br d, J=7.82 Hz, 2H), 4.11-4.25 (m, 1H), 4.00-4.11 (m, 1H), 3.78-3.93 (m, 1H), 2.39 (s, 3H), 1.58-2.06 (m, 3H), 1.11 (br d, J=6.11 Hz, 3H).

### 3-(3-Hydroxybutyl)-1-methyl-5-nitro-1H-benzo[d]imidazol-2(3H),-one

To a solution of 3-methyl-6-nitro-1H-benzimidazol-2-one (3 g, 15.53 mmol) and 3-hydroxybutyl 4-methylbenzenesulfonate (7.59 g, 31.06 mmol) in DMSO (30 mL), Cs₂CO₃ (10.12 g, 31.06 mmol) and KI (1.29 g, 7.77 mmol) was added. The flask was degassed and purged with N₂ for 3 times, and then the mixture was stirred at 100° C. for 12 hr under N₂ atmosphere. Water (50 mL) was added to the mixture. The mixture was extracted with ethyl acetate (50 mL×3). The combined organic phase was dried with anhydrous Na₂SO₄, filtered and concentrated to yield a residue that was purified by column chromatography (silica gel, Petroleum ether/Ethyl acetate=10:1 to 1:1) and then was triturated with Petroleum ether/Ethyl acetate=3:1 (15 mL) at 15° C. for 15 min to give the title compound as a yellow solid (2 g, 6.26 mmol, 40% yield, 83% purity). ¹H NMR (400 MHz, DMSO-d₆) δ ppm 1.09 (d, J=6.25 Hz, 3H), 1.61-1.77 (m, 2H), 3.40 (s, 3H), 3.57-3.67 (m, 1H), 3.93-4.03 (m, 2H), 4.63 (d, J=4.88 Hz, 1H), 7.35 (d, J=8.63 Hz, 1H), 8.04-8.07 (m, 1H), 8.08 (d, J=2.13 Hz, 1H).

### 5-Amino-3-(3-hydroxybutyl)-1-methyl-1H-benzo[d]imidazol-2(3H),-one

To a solution of Pd/C (0.5 g, 10% purity) in DMF (20 mL) was added 3-(3-hydroxybutyl)-1-methyl-5-nitro-1H-benzo[d]imidazol-2(3H)-one (2 g, 7.54 mmol) under Ar. The mixture was stirred at 50° C. for 12 hr under H₂ (15 psi). The mixture was filtered and the filtrate was concentrated in vacuo to give the title compound as a gray solid (1.7 g, 6.86 mmol, 91% yield, 95% purity), which was used without further purification. ¹H NMR (400 MHz, DMSO-d₆) δ ppm 1.09 (d, J=6.11 Hz, 3H), 1.56-1.68 (m, 2H), 3.22 (s, 3H), 3.61 (br s, 1H), 3.68-3.85 (m, 2H), 4.64 (br d, J=3.55 Hz, 1H), 4.78 (br s, 2H), 6.31 (dd, J=8.31, 1.71 Hz, 1H), 6.41 (d, J=1.59 Hz, 1H), 6.79 (d, J=8.19 Hz, 1H).

### 2-Chloro-5-fluoro-6-((3-(3-hydroxybutyl)-1-methyl-2-oxo-2,3-dihydro-1H-benzo[d]imidazol-5-yl)amino)nicotinonitrile

To a solution of 5-amino-3-(3-hydroxybutyl)-1-methyl-benzimidazol-2-one (200 mg, 850.05 μmol) and 2,6-dichloro-5-fluoro-pyridine-3-carbonitrile (194.82 mg, 1.02 mmol) in DMSO (2 mL) was added DIPEA (219.72 mg, 1.70 mmol, 296.13 μL). The mixture was stirred at 100° C. for 12 hr. The mixture was poured into water (5 mL) and stirred for 10 min. The mixture was filtered and the filter cake was washed with ethyl acetate (3 mL). The filter cake was dried in vacuo to give the title compound as a yellow solid (300 mg, 644.26 μmol, 84% yield, 84% purity). ¹H NMR (400 MHz, DMSO-d₆) δ 9.94 (s, 1H), 8.11 (d, J=10.63 Hz, 1H), 7.50 (d, J=1.38 Hz, 1H), 7.29 (dd, J=8.44, 1.56 Hz, 1H), 7.12 (d, J=8.38 Hz, 1H), 4.63 (d, J=4.63 Hz, 1H), 3.74-3.96 (m, 2H), 3.57-3.71 (m, 1H), 3.32 (s, 3H), 1.59-1.80 (m, 2H), 1.10 (d, J=6.13 Hz, 3H).

### (S)-2-Chloro-5-fluoro-6-((3-(3-hydroxybutyl)-1-methyl-2-oxo-2,3-dihydro-1H-benzo[d]imidazol-5-yl)amino)nicotinonitrile and (R)-2-chloro-5-fluoro-6-((3-(3-hydroxybutyl)-1-methyl-2-oxo-2,3-dihydro-1H-benzo[d]imidazol-5-yl)amino)nicotinonitrile

The residue was purified by prep-HPLC (neutral condition; column: Phenomenex C18 80*40 mm*3 μm; mobile phase: [water(NH₄HCO₃)-ACN]; B %: 15%-45%, 8 min). The residue was further separated by SFC (condition: column: REGIS(S,S)WHELK-O1 (250 mm*25 mm, 10 μm); mobile phase: [0.1% NH₃H₂O in IPA]; B %: 45%-45%, 7 min) to give (S)-2-chloro-5-fluoro-6-((3-(3-hydroxybutyl)-1-methyl-2-oxo-2,3-dihydro-1H-benzo[d]imidazol-5-yl)amino)nicotinonitrile as a white solid (40 mg, 97.47 μmol, 15% yield, 95% purity) and (R)-2-chloro-5-fluoro-6-((3-(3-hydroxybutyl)-1-methyl-2-oxo-2,3-dihydro-1H-benzo [d]imidazol-5-yl)amino)nicotinonitrile as a white solid (40 mg, 92.14 μmol, 14% yield, 90% purity). ¹H NMR (400 MHz, MeOD-d₄) δ ppm 7.72-7.80 (m, 1H), 7.68 (s, 1H), 7.35 (br d, J=8.25 Hz, 1H), 7.13 (br d, J=8.25 Hz, 1H), 4.02 (br t, J=7.00 Hz, 2H), 3.78 (br d, J=4.38 Hz, 1H), 3.43 (s, 3H), 1.97-2.10 (m, 1H), 1.74-1.97 (m, 2H), 1.27-1.39 (m, 1H), 1.22 (br d, J=6.00 Hz, 3H).

### 5-Fluoro-2-((3S,4R,5R)-4-fluoro-3,5-dimethylpiperidin-1-yl)-6-((3-((S)-3-hydroxybutyl)-1-methyl-2-oxo-2,3-dihydro-1H-benzo[d]imidazol-5-yl)amino)nicotinonitrile (54)

To a mixture of (S)-2-chloro-5-fluoro-6-((3-(3-hydroxybutyl)-1-methyl-2-oxo-2,3-dihydro-1H-benzo[d]imidazol-5-yl)amino)nicotinonitrile (40 mg, 102.61 μmol) and (3S,4S,5R)-4-fluoro-3,5-dimethyl-piperidine (37.74 mg, 153.92 μmol, TFA) in DMSO (0.5 mL) was added DIEA (26.52 mg, 205.23 μmol, 35.75 μL) under N₂. The mixture was stirred at 100° C. for 12 hr. The residue was purified by prep-HPLC (neutral condition; column: Phenomenex C18 75*30 mm*3 μm; mobile phase: [water(NH₄HCO₃)-ACN]; B %: 25%-55%, 8 min) to give the title compound as a white solid (16 mg, 33.02 μmol, 32% yield, 100% purity). ¹H NMR (400 MHz, DMSO-d₆) δ 9.37 (s, 1H), 7.76 (d, J=11.00 Hz, 1H), 7.50 (d, J=1.71 Hz, 1H), 7.37 (dd, J=8.44, 1.83 Hz, 1H), 7.09 (d, J=8.44 Hz, 1H), 4.54-4.64 (m, 2H), 4.45 (s, 1H), 3.76-3.98 (m, 4H), 3.55-3.68 (m, 1H), 2.80 (t, J=12.59 Hz, 2H), 1.77-1.98 (m, 2H), 1.54-1.77 (m, 2H), 1.08 (d, J=6.11 Hz, 3H), 0.90 (dd, J=6.85, 2.57 Hz, 6H).

### 5-Fluoro-2-((3S,4S,5R)-4-fluoro-3,5-dimethylpiperidin-1-yl)-6-((3-((R)-3-hydroxybutyl)-1-methyl-2-oxo-2,3-dihydro-1H-benzo[d]imidazol-5-yl)amino)nicotinonitrile (55)

To a mixture of (R)-2-chloro-5-fluoro-6-((3-(3-hydroxybutyl)-1-methyl-2-oxo-2,3-dihydro-1H-benzo[d]imidazol-5-yl)amino)nicotinonitrile (40 mg, 102.61 μmol) and (3S,4S,5R)-4-fluoro-3,5-dimethyl-piperidine (37.74 mg, 153.92 μmol, TFA) in DMSO (0.5 mL) was added DIEA (26.52 mg, 205.22 umol, 35.75 μL) under N₂. The mixture was stirred at 100° C. for 12 hr. The residue was purified by prep-HPLC (neutral condition; column: Phenomenex C18 75*30 mm*3 μm; mobile phase: [water(NH₄HCO₃)-ACN]; B %: 25%-55%, 8 min) to give the title compound as a white solid (9.5 mg, 19.61 μmol, 19% yield, 100% purity). ¹H NMR (400 MHz, DMSO-d₆) δ 9.38 (s, 1H), 7.76 (d, J=11.00 Hz, 1H), 7.50 (d, J=1.83 Hz, 1H), 7.37 (dd, J=8.44, 1.83 Hz, 1H), 7.09 (d, J=8.44 Hz, 1H), 4.55-4.68 (m, 2H), 4.45 (s, 1H), 3.76-3.99 (m, 4H), 3.55-3.70 (m, 1H), 2.80 (br t, J=12.59 Hz, 2H), 1.55-2.03 (m, 4H), 1.05-1.11 (m, 3H), 0.90 (dd, J=6.91, 2.51 Hz, 6H).

The absolute configurations of compounds 54 & 55 were randomly assigned based on all substituents of the piperidine ring being in cis-conformation.

### Example 10: Synthesis of 5-fluoro-2-[(3R,4R,5S)-4-fluoro-3-(2-hydroxyethyl)-5-methyl-1-piperidyl]-6-[[3-(3-hydroxybutyl)-1-methyl-2-oxo-benzimidazol-5-yl]amino]pyridine-3-carbonitrile (56) and 5-fluoro-2-[(3S,4S,5R)-4-fluoro-3-(2-hydroxyethyl)-5-methyl-1-piperidyl]-6-[[3-(3-hydroxybutyl)-1-methyl-2-oxo-benzimidazol-5-yl]amino]pyridine-3-carbonitrile (57)

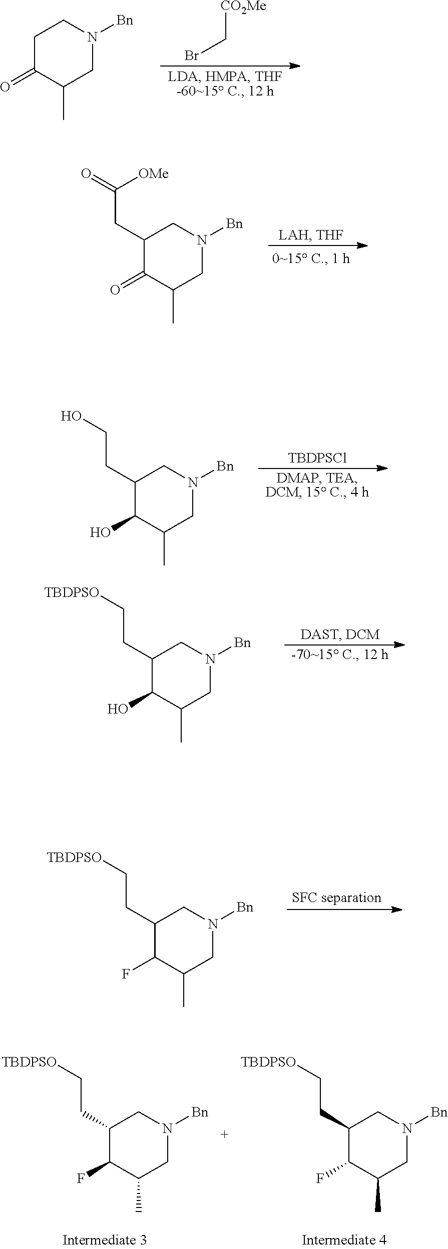

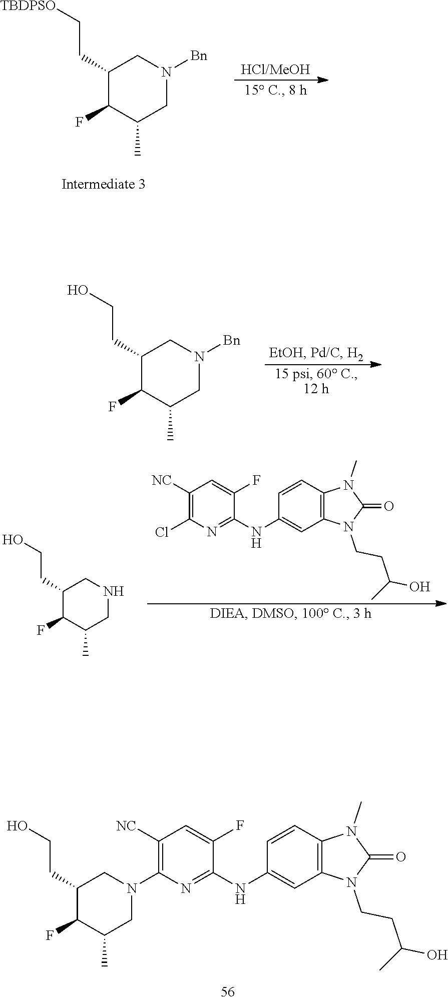

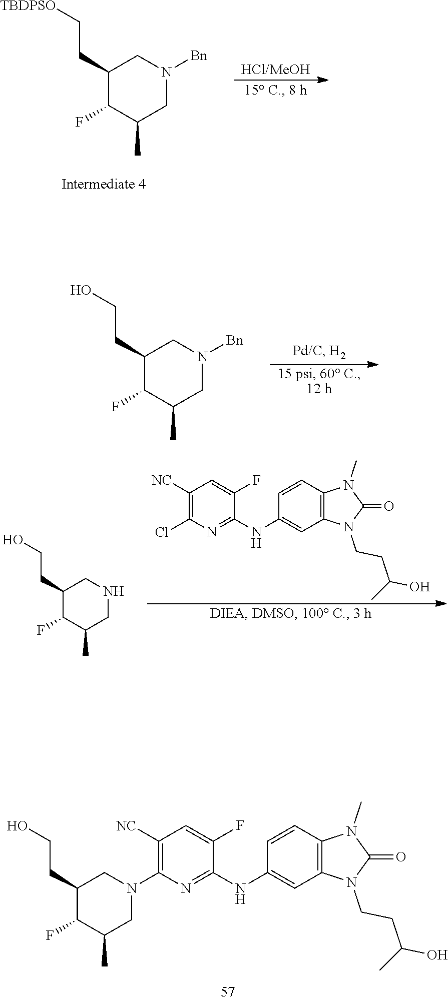

### Methyl 2-(1-benzyl-5-methyl-4-oxopiperidin-3-yl)acetate

To a mixture of 1-benzyl-3-methyl-piperidin-4-one (25 g, 122.98 mmol) in THE (250 mL) was added LDA (2 M, 79.94 mL) dropwise at −65° C. and the mixture was stirred at −65° C. for 30 min, then HMPA (28.65 g, 159.88 mmol, 28.09 mL) was added dropwise and the mixture was stirred at −65° C. for 30 min. Then methyl 2-bromoacetate (24.46 g, 159.88 mmol, 15.10 mL) was added dropwise. The mixture was warmed to 15° C. and stirred for 12 hr. Then water (600 mL) was added to the mixture (combined with another batch of same scale) and extracted with EtOAc (800 mL×3). The combined organic layer was dried over Na₂SO₄, filtered and concentrated under reduced pressure to give the residue. The residue was purified by column chromatography (SiO₂, Petroleum ether/Ethyl acetate=1/0 to 3/1) to give the title compound as a yellow solid (40 g, 116.22 mmol, 47% yield, 80% purity). (¹H NMR (400 MHz, DMSO-d₆) δ 7.37-7.27 (m, 5H), 3.66-3.62 (m, 2H), 3.59-3.54 (m, 3H), 3.20-3.01 (m, 3H), 2.78 (td, J=6.1, 11.8 Hz, 1H), 2.56 (d, J=7.6 Hz, 1H), 2.24-1.99 (m, 3H), 0.83 (d, J=6.6 Hz, 3H).

### 1-Benzyl-3-(2-hydroxyethyl)-5-methylpiperidin-4-ol

To a solution of methyl 2-(1-benzyl-5-methyl-4-oxo-3-piperidyl)acetate (10 g, 36.32 mmol) in THE (100 mL) was added LAH (2.76 g, 72.64 mmol) at 0° C. The mixture was stirred at 20° C. for 1 hr. The mixture was added water (3 mL) and 2M NaOH (3 mL) at 0° C. The mixture was dried with anhydrous Na₂SO₄ and stirred for 20 min at 15° C. Then the mixture was filtered and the filtrate was concentrated under reduced pressure to give the title compound as a yellow oil (9 g, crude). ¹H NMR (400 MHz, DMSO-d₆) δ 7.35-7.20 (m, 7H), 4.62 (d, J=6.5 Hz, 1H), 4.44 (t, J=5.0 Hz, 1H), 3.52-3.42 (m, 2H), 2.85 (br d, J=10.3 Hz, 1H), 2.70-2.64 (m, 1H), 1.87-1.77 (m, 1H), 1.69-1.41 (m, 5H), 1.14-1.07 (m, 1H), 0.83 (d, J=6.4 Hz, 3H).

### 1-Benzyl-3-(2-((tert-butyldiphenylsilyl)oxy)ethyl)-5-methyl-piperidin-4-ol

To a solution of 1-benzyl-3-(2-hydroxyethyl)-5-methyl-piperidin-4-ol (5.00 g, 20.05 mmol) in DCM (60 mL) was added TBDPSCl (6.06 g, 22.06 mmol, 5.66 mL), DMAP (734.93 mg, 6.02 mmol) and TEA (10.15 g, 100.26 mmol, 13.96 mL). The mixture was stirred at 15° C. for 4 hr. After addition of water (100 mL), the solution was extracted with EtOAc (100 mL×3). The combined organic phase was washed with brine (100 mL), dried over anhydrous Na₂SO₄, filtered and concentrated in vacuo to give a residue that was purified by flash silica gel column chromatography (Silica Flash Column, Eluent of 0˜30% Ethyl acetate/Petroleum ether gradient) to give the title compound as a yellow oil (7.5 g, 14.11 mmol, 70% yield, 92% purity). ¹H NMR (400 MHz, DMSO-d₆) δ 7.71-7.54 (m, 4H), 7.46-7.36 (m, 6H), 7.33-7.18 (m, 4H), 4.50 (d, J=6.7 Hz, 1H), 4.03 (q, J=7.1 Hz, 1H), 3.70-3.55 (m, 2H), 3.41-3.29 (m, 3H), 2.90-2.63 (m, 2H), 2.06-1.95 (m, 1H), 1.68-1.48 (m, 3H), 1.29-1.12 (m, 1H), 1.01-0.91 (m, 9H), 0.83 (d, J=6.4 Hz, 3H).

### 1-Benzyl-3-(2-((tert-butyldiphenylsilyl)oxy)ethyl)-4-fluoro-5-methylpiperidine

To a solution of 1-benzyl-3-(2-((tert-butyldiphenylsilyl)oxy)ethyl)-5-methyl-piperidin-4-ol (5.00 g, 10.25 mmol) in DCM (50 mL) was added DAST (3.30 g, 20.50 mmol, 2.71 mL) at −65° C. The mixture was stirred at −70° C. for 0.5 hr and stirred at 15° C. for 11.5 hr. Sat. NaHCO₃ (50 mL) was added to the mixture and extracted with DCM (50 mL×3). The combined organic phase was dried with anhydrous Na₂SO₄, filtered and concentrated under reduced pressure. The residue was purified by flash silica gel column chromatography (Silica Flash Column, Eluent of 0˜30% Ethyl acetate/Petroleum ether gradient) to give the title compound as a yellow oil (3.3 g, 6.71 mmol, 65% yield, 99% purity). ¹H NMR (400 MHz, CDCl₃) δ 7.69-7.62 (m, 4H), 7.44-7.36 (m, 5H), 7.33-7.27 (m, 5H), 3.81-3.59 (m, 3H), 3.56-3.39 (m, 2H), 3.06-2.76 (m, 2H), 2.08-1.88 (m, 3H), 1.80-1.62 (m, 2H), 1.44-1.31 (m, 1H), 1.04 (s, 9H), 0.96 (d, J=6.5 Hz, 3H).

### 2-[(3R,4R,5S)-1-benzyl-4-fluoro-5-methyl-3-piperidyl]ethoxy-tert-butyl-diphenyl-silane (intermediate 3) and 2-[(3S,4S,5R)-1-benzyl-4-fluoro-5-methyl-3-piperidyl]ethoxy-tert-butyl-diphenyl-silane (intermediate 4)

Racemic 1-benzyl-3-(2-((tert-butyldiphenylsilyl)oxy)ethyl)-4-fluoro-5-methylpiperidine was purified by SFC (column: DAICEL CHIRALCEL OD (250 mm*50 mm, 10 μm); mobile phase: [0.1% NH₃H₂O IPA]; B %: 25%-25%, 5 min) to give 2-[(3R,4R,5S)-1-benzyl-4-fluoro-5-methyl-3-piperidyl]ethoxy-tert-butyl-diphenyl-silane as a white solid (1.5 g, 3.03 mmol, 45% yield, 99% purity). ¹H NMR (400 MHz, DMSO-d₆) δ 7.60-7.54 (m, 4H), 7.48-7.37 (m, 6H), 7.33-7.21 (m, 5H), 3.89-3.68 (m, 1H), 3.64 (t, J=6.3 Hz, 2H), 3.48-3.38 (m, 2H), 2.96-2.85 (m, 1H), 2.80-2.70 (m, 1H), 1.98-1.75 (m, 3H), 1.74-1.64 (m, 2H), 1.40-1.29 (m, 1H), 0.95 (s, 9H), 0.88 (d, J=6.2 Hz, 3H) and 2-[(3S,4S,5R)-1-benzyl-4-fluoro-5-methyl-3-piperidyl]ethoxy-tert-butyl-diphenyl-silane as a white solid (1.5 g, 3.03 mmol, 45% yield, 99% purity). ¹H NMR (400 MHz, DMSO-d₆) δ 7.60-7.53 (m, 4H), 7.48-7.37 (m, 6H), 7.33-7.20 (m, 5H), 3.88-3.69 (m, 1H), 3.64 (t, J=6.3 Hz, 2H), 3.43 (d, J=0.7 Hz, 2H), 2.96-2.70 (m, 2H), 2.00-1.75 (m, 3H), 1.74-1.66 (m, 2H), 1.41-1.28 (m, 1H), 0.95 (s, 9H), 0.88 (d, J=6.3 Hz, 3H).

### 2-[(3R,4R,5S)-1-Benzyl-4-fluoro-5-methyl-3-piperidyl]ethanol

A solution of 2-[(3R,4R,5S)-1-benzyl-4-fluoro-5-methyl-3-piperidyl]ethoxy-tert-butyl-diphenyl-silane (1.5 g, 3.06 mmol) in HCl/MeOH (50 mL) was stirred at 15° C. for 8 hr. After addition of sat. aq. NaHCO₃ (30 mL), the solution was extracted with EtOAc (30 mL×3). The combined organic phase was washed with brine (30 mL), dried over anhydrous Na₂SO₄, filtered and concentrated in vacuo to give the title compound as a yellow oil (600 mg, 2.39 mmol, 78% yield, 100% purity). ¹H NMR (400 MHz, DMSO-d₆) δ 7.35-7.21 (m, 5H), 4.40 (t, J=5.1 Hz, 1H), 3.88-3.63 (m, 1H), 3.55-3.46 (m, 1H), 3.44-3.31 (m, 4H), 2.99-2.85 (m, 1H), 2.78-2.65 (m, 1H), 1.81-1.61 (m, 4H), 1.30-1.13 (m, 1H), 0.88 (d, J=6.4 Hz, 3H).

### 2-[(3R,4R,5S)-4-Fluoro-5-methyl-3-piperidyl]ethanol

To a solution of 2-[(3R,4R,5S)-1-benzyl-4-fluoro-5-methyl-3-piperidyl]ethanol (600 mg, 2.39 mmol) in EtOH (40 mL) was added Pd/C (300 mg, 10% purity) under Ar atmosphere. The flask was degassed and purged with H₂ for 3 times. The mixture was stirred under H₂ (15 Psi) at 60° C. for 12 hr. The reaction mixture was filtered through a pad of Celite® and the filtrate was concentrated in vacuo to give the title compound as a grey oil (200 mg, 372.17 μmol, 16% yield, 30% purity). ¹H NMR (400 MHz, DMSO-d₆) δ 4.43 (br dd, J=6.5, 11.8 Hz, 1H), 3.54-3.35 (m, 3H), 3.34-3.21 (m, 1H), 2.82-2.61 (m, 2H), 2.13-1.88 (m, 2H), 1.81 (dtd, J=4.2, 6.7, 13.8 Hz, 1H), 1.39-1.29 (m, 1H), 1.22 (t, J=7.3 Hz, 1H), 1.05 (t, J=7.0 Hz, 1H), 1.01-0.95 (m, 3H).

### 5-Fluoro-2-[(3R,4R,5S)-4-fluoro-3-(2-hydroxyethyl)-5-methyl-1-piperidyl]-6-[[3-(3-hydroxybutyl)-1-methyl-2-oxo-benzimidazol-5-yl]amino]pyridine-3-carbonitrile (50)

To a solution of 2-chloro-5-fluoro-6-[[3-(3-hydroxybutyl)-1-methyl-2-oxo-benzimidazol-5-yl]amino]pyridine-3-carbonitrile (20 mg, 51.31 μmol) in DMSO (1 mL) was added 2-[(3R,4R,5S)-4-fluoro-5-methyl-3-piperidyl]ethanol (8.27 mg, 51.31 μmol) and DIEA (26.52 mg, 205.24 μmol, 35.75 μL). The mixture was stirred at 100° C. for 2 hr. The solution was filtered and concentrated under vacuum. The residue was purified by prep-HPLC (column: Phenomenex C18 75*30 mm*3 μm; mobile phase: [water(NH₄HCO₃)-ACN]; B %: 10%-40%, 8 min) to give the title compound as a yellow solid (3 mg, 5.36 μmol, 10% yield, 92% purity). ¹H NMR (400 MHz, DMSO-d₆) δ 9.39 (br s, 1H), 7.78 (br d, J=10.9 Hz, 1H), 7.52-7.31 (m, 2H), 7.14-7.00 (m, 1H), 4.62 (br d, J=4.0 Hz, 1H), 4.49-4.37 (m, 1H), 4.29-4.14 (m, 1H), 4.08-4.00 (m, 1H), 3.98-3.90 (m, 1H), 3.89-3.78 (m, 2H), 3.62 (br d, J=1.5 Hz, 1H), 3.47-3.36 (m, 3H), 2.78-2.69 (m, 1H), 2.67-2.60 (m, 1H), 1.82-1.62 (m, 6H), 1.37-1.25 (m, 2H), 1.09 (br d, J=5.5 Hz, 3H), 0.92 (br s, 3H).

### 2-[(3S,4S,5R)-1-Benzyl-4-fluoro-5-methyl-3-piperidyl]ethanol

A solution of 2-[(3S,4S,5R)-1-benzyl-4-fluoro-5-methyl-3-piperidyl]ethoxy-tert-butyl-diphenyl-silane (1.5 g, 3.06 mmol) in HCl/MeOH (1.25 M, 50 mL) was stirred at 15° C. for 8 hr. After addition of sat. aq. NaHCO₃ (30 mL), the solution was extracted with EtOAC (30 mL×3). The combined organic phases was washed with brine (30 mL), dried over anhydrous Na₂SO₄, filtered and concentrated in vacuo to give the crude title compound as a yellow oil (400 mg, 1.59 mmol, 52% yield, 100% purity), which was used without further purifications. ¹H NMR (400 MHz, DMSO-d₆) δ 7.39-7.20 (m, 5H), 4.40 (t, J=5.1 Hz, 1H), 3.90-3.65 (m, 1H), 3.56-3.47 (m, 1H), 3.42-3.31 (m, 4H), 2.98-2.67 (m, 2H), 1.81-1.61 (m, 4H), 1.30-1.14 (m, 1H), 0.89 (d, J=6.4 Hz, 3H). M+H⁺=252.1.

### 2-[(3S,4S,5R)-4-Fluoro-5-methyl-3-piperidyl]ethanol

To a solution of 2-[(3S,4S,5R)-1-benzyl-4-fluoro-5-methyl-3-piperidyl]ethanol (400 mg, 1.59 mmol) in EtOH (5 mL) was added Pd/C (50 mg, 10% purity) under an argon atmosphere. The suspension was degassed and purged with H₂ for 3 times. The mixture was stirred under H₂ (15 Psi) at 60° C. for 12 hr. The reaction mixture was filtered through a pad of Celite® and the filtrate was concentrated under reduced pressure to give the crude title compound as a grey oil (200 mg, crude), which was used without further purifications. ¹H NMR (400 MHz, DMSO-d₆) δ 4.48-4.38 (m, 1H), 4.23-4.02 (m, 1H), 3.53-3.38 (m, 3H), 3.33-3.22 (m, 1H), 2.85-2.60 (m, 2H), 2.12-1.90 (m, 2H), 1.81 (dtd, J=4.2, 6.8, 13.8 Hz, 1H), 1.41-1.29 (m, 1H), 1.26-1.19 (m, 1H), 1.06 (t, J=7.0 Hz, 1H), 1.02-0.96 (m, 3H).

### 5-Fluoro-2-[(3S,4S,5R)-4-fluoro-3-(2-hydroxyethyl)-5-methyl-1-piperidyl]-6-[[3-(3-hydroxybutyl)-1-methyl-2-oxo-benzimidazol-5-yl]amino]pyridine-3-carbonitrile (56)

To a solution of 2-chloro-5-fluoro-6-[[3-(3-hydroxybutyl)-1-methyl-2-oxo-benzimidazol-5-yl]amino]pyridine-3-carbonitrile (20 mg, 51.31 μmol) in DMSO (1 mL) was added 2-[(3S,4S,5R)-4-fluoro-5-methyl-3-piperidyl]ethanol (14.12 mg, 87.60 μmol) and DIEA (26.52 mg, 205.24 μmol, 35.75 μL). The mixture was stirred at 100° C. for 2 hrs. The solution was filtered and the filtrate concentrated under vacuum to give a residue that was purified by prep-HPLC to give the title compound as a yellow solid (5 mg, 8.94 μmol, 17% yield, 92% purity). ¹H NMR (400 MHz, DMSO-d₆) δ 9.39 (s, 1H), 7.77 (d, J=10.9 Hz, 1H), 7.49 (dd, J=1.9, 2.9 Hz, 1H), 7.39 (br d, J=8.4 Hz, 1H), 7.09 (d, J=8.5 Hz, 1H), 4.62 (d, J=4.6 Hz, 1H), 4.48-4.40 (m, 1H), 4.28-4.18 (m, 1H), 4.08-4.01 (m, 1H), 3.98-3.91 (m, 1H), 3.90-3.78 (m, 2H), 3.62 (br s, 1H), 3.46-3.37 (m, 3H), 2.73 (br t, J=12.3 Hz, 1H), 2.68-2.60 (m, 1H), 1.84-1.62 (m, 6H), 1.37-1.22 (m, 2H), 1.09 (d, J=6.1 Hz, 3H), 0.93 (dd, J=2.4, 6.4 Hz, 3H). M+H⁺=515.3.

The absolute configurations of compounds 56 & 57 were randomly assigned based on the amino group and methyl group being in cis-conformation.

### Example 11: Synthesis of 2-((6-((3-chloro-5-cyano-6-((3R,5S)-4,4-difluoro-3,5-dimethylpiperidin-1-yl)pyridin-2-yl)amino)-1-(2-(dimethylamino)ethyl)-2-oxo-1,2-dihydroquinolin-3-yl)oxy)-N-methylacetamide (11)

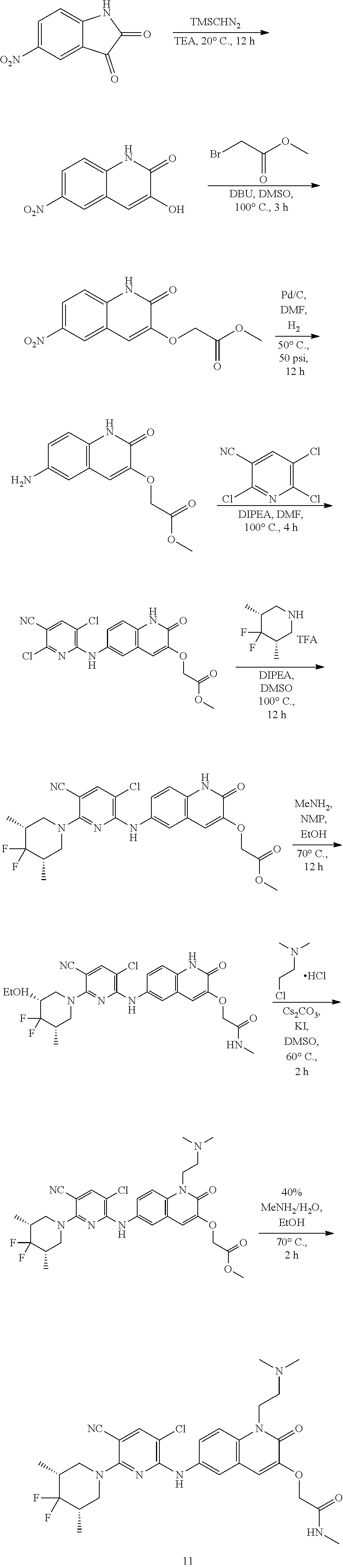

### 3-Hydroxy-6-nitroquinolin-2(1H)-one

To a mixture of 5-nitroindoline-2,3-dione (20 g, 104.10 mmol) and TEA (21.07 g, 208.19 mmol, 28.98 mL) in EtOH (600 mL) was added TMSCHN₂ (2 M, 62.46 mL) dropwise at 20° C. The reaction mixture was stirred at 20° C. for 12 hr and was concentrated in vacuo. Then 1N HCl (600 mL) was added to the concentrated reaction mixture and stirred for 3 hr. The resulting suspension was filtered and the filter cake was collected and dried in vacuo to give the title compound as an orange-red solid (40 g, crude). ¹H NMR (400 MHz, DMSO-d₆) δ 12.54 (s, 1H), 10.38-9.71 (m, 1H), 8.51 (d, J=2.4 Hz, 1H), 8.13 (dd, J=2.4, 9.2 Hz, 1H), 7.39 (d, J=9.2 Hz, 1H), 7.31 (s, 1H).

### Methyl 2-((6-nitro-2-oxo-1,2-dihydroquinolin-3-yl)oxy)acetate

To a solution of 3-hydroxy-6-nitro-1H-quinolin-2-one (20 g, 97.01 mmol) in DMSO (200 mL) was added DBU (17.72 g, 116.42 mmol, 17.55 mL) and then methyl 2-bromoacetate (17.81 g, 116.42 mmol, 10.99 mL), and the resulting mixture was stirred at 100° C. for 3 hr. The reaction mixture was treated with water (600 mL) which generated a precipitate. The suspension was filtered and the filter cake was washed with EtOAc (800 mL), the solid was dried in vacuo to give the title compound as an orange-red solid (26 g, crude). ¹H NMR (400 MHz, DMSO-d₆) δ 12.51 (s, 1H), 8.54 (d, J=2.4 Hz, 1H), 8.20-8.17 (m, 1H), 7.49 (s, 1H), 7.39 (d, J=9.2 Hz, 1H), 4.89 (s, 2H), 3.77-3.71 (s, 3H).

### Methyl 2-((6-amino-2-oxo-1,2-dihydroquinolin-3-yl)oxy)acetate

To a solution of methyl 2-[(6-nitro-2-oxo-1H-quinolin-3-yl)oxy]acetate (16 g, 57.51 mmol) in DMF (200 mL) was added Pd/C (5 g, 10% purity) under Ar atmosphere. The flask was degassed and purged with H₂ for 3 times. The mixture was stirred under H₂ (50 psi) at 50° C. for 12 hr. The reaction mixture was filtered and the filtrate was concentrated in vacuo to give the title compound as a grey-black solid (12 g, crude). ¹H NMR (400 MHz, DMSO-d₆) δ 11.60 (s, 1H), 7.95 (s, 1H), 7.03-6.98 (m, 2H), 6.72 (dd, J=2.4, 8.8 Hz, 1H), 6.68 (d, J=2.0 Hz, 1H), 6.05-5.39 (m, 2H), 4.83 (s, 2H), 3.71 (s, 3H).

### Methyl 2-((6-((3,6-dichloro-5-cyanopyridin-2-yl)amino)-2-oxo-1,2-dihydroquinolin-3-yl)oxy) acetate

To a mixture of methyl 2-[(6-amino-2-oxo-1H-quinolin-3-yl)oxy]acetate (3 g, 12.09 mmol) and 2,5,6-trichloropyridine-3-carbonitrile (2.51 g, 12.09 mmol) in DMF (30 mL) was added DIPEA (3.12 g, 24.17 mmol, 4.21 mL), and the reaction mixture was stirred at 100° C. for 4 hr. The mixture was cooled to 15° C. and water (˜50 mL) was added, forming a precipitate. The mixture was filtered and the filter cake was washed with EtOAc (˜80 mL) and dried in vacuo to give the title compound as a brown solid (15 g, crude). ¹H NMR (400 MHz, DMSO-d₆) δ 12.02 (s, 1H), 9.58 (s, 1H), 8.35 (s, 1H), 7.65 (d, J=2.0 Hz, 1H), 7.48 (dd, J=2.0, 8.8 Hz, 1H), 7.27 (d, J=8.8 Hz, 1H), 7.21 (s, 1H), 4.88 (s, 2H), 3.72 (s, 3H).

### Methyl 2-((6-((3-chloro-5-cyano-6-((3R,5S)-4,4-difluoro-3,5-dimethylpiperidin-1-yl)pyridin-2-yl)amino)-2-oxo-1,2-dihydroquinolin-3-yl)oxy)acetate

To a solution of methyl 2-[[6-[(3,6-dichloro-5-cyano-2-pyridyl)amino]-2-oxo-1H-quinolin-3-yl]oxy]acetate (5 g, 11.93 mmol) and (3S,5R)-4,4-difluoro-3,5-dimethyl-piperidine (4.08 g, 15.51 mmol, TFA) in DMSO (50 mL) was added DIPEA (7.71 g, 59.63 mmol, 10.39 mL) and the reaction mixture was stirred at 100° C. for 12 h. The mixture was cooled to 15° C. and water (˜50 mL) was added, forming a precipitate. The mixture was filtered and the filter cake was washed by EtOAc (˜80 mL) and dried n vacuo. The residue was purified by column chromatography (SiO₂, Petroleum ether/Ethyl acetate=1/1 to 0/1) to give a residue which was then triturated with a mixture of solvent (PE:EtOAc=1:1, 40 mL) at 20° C. for 30 min to give the title compound as a pale brown solid (2.3 g, 3.98 mmol, 33% yield, 92% purity). ¹H NMR (400 MHz, DMSO-d₆) δ 11.96 (s, 1H), 9.08 (s, 1H), 7.97 (s, 1H), 7.66 (s, 1H), 7.49 (dd, J=2.0, 8.8 Hz, 1H), 7.25 (d, J=8.4 Hz, 1H), 7.20 (s, 1H), 4.85 (s, 2H), 4.10 (d, J=13.2 Hz, 2H), 3.72 (s, 3H), 2.77 (t, J=12.4 Hz, 2H), 2.14-1.93 (m, 2H), 0.82 (br d, J=6.4 Hz, 6H).

### 2-((6-((3-Chloro-5-cyano-6-((3R,5S)-4,4-difluoro-3,5-dimethylpiperidin-1-yl)pyridin-2-yl)amino)-2-oxo-1,2-dihydroquinolin-3-yl)oxy)-N-methylacetamide

To a mixture of methyl 2-[[6-[[3-chloro-5-cyano-6-[(3S,5R)-4,4-difluoro-3,5-dimethyl-1-piperidyl]-2-pyridyl]amino]-2-oxo-1H-quinolin-3-yl]oxy]acetate (10 g, 23.85 mmol) in EtOH (90 mL) and NMP (10 mL) was added MeNH₂ (14.82 g, 190.83 mmol, 40% purity), and the reaction mixture was stirred at 70° C. for 12 hr. The mixture was cooled to 20° C., then the reaction mixture was filtered and the filter cake was collected and dried under reduced pressure to give the title compound as a pale brown solid (9.2 g, 21.12 mmol, 89% yield, 96% purity).

### Methyl 2-((6-((3-chloro-5-cyano-6-((3R,5S)-4,4-difluoro-3,5-dimethylpiperidin-1-yl)pyridin-2-yl)amino)-1-(2-(dimethylamino)ethyl)-2-oxo-1,2-dihydroquinolin-3-yl)oxy)acetate

A mixture of methyl 2-[[6-[[3-chloro-5-cyano-6-[(3S,5R)-4,4-difluoro-3,5-dimethyl-1-piperidyl]-2-pyridyl]amino]-2-oxo-1H-quinolin-3-yl]oxy]acetate (350 mg, 657.97 μmol), 2-chloro-N,N-dimethyl-ethanamine (123.21 mg, 855.36 μmol, HCl), Cs₂CO₃ (643.14 mg, 1.97 mmol), KI (54.61 mg, 328.99 μmol) in DMSO (5 mL) was stirred at 60° C. for 2 hr under N₂ atmosphere. Water (5 mL) was added to the mixture, the precipitated solids were filtered and washed with H₂O (20 mL) and petroleum ether/ethyl acetate (1/1, 5 mL), then purified by prep-HPLC (column: Phenomenex C18 80*40 mm*3 μm; mobile phase: [water (NH₄HCO₃)-ACN]; B %: 50%-70%, 8 min) to give the title compound as a yellow solid (50 mg, 78.36 μmol, 12% yield, 95% purity). ¹H NMR (400 MHz, DMSO-d₆) δ 9.14 (s, 1H), 7.99 (s, 1H), 7.75 (d, J=2.0 Hz, 1H), 7.63 (dd, J=2.4, 9.2 Hz, 1H), 7.46 (d, J=9.2 Hz, 1H), 7.22 (s, 1H), 4.86 (s, 2H), 4.38 (t, J=6.8 Hz, 2H), 4.12 (br d, J=12.4 Hz, 2H), 3.72 (s, 3H), 2.78 (br t, J=12.4 Hz, 2H), 2.52 (d, J=1.6 Hz, 2H), 2.23 (s, 6H), 2.10-1.99 (m, 2H), 0.84 (d, J=6.8 Hz, 6H).

### 2-((6-((3-Chloro-5-cyano-6-((3R,5S)-4,4-difluoro-3,5-dimethylpiperidin-1-yl)pyridin-2-yl)amino)-1-(2-(dimethylamino)ethyl)-2-oxo-1,2-dihydroquinolin-3-yl)oxy)-N-methylacetamide (11)

To a mixture of methyl 2-[[6-[[3-chloro-5-cyano-6-[(3S,5R)-4,4-difluoro-3,5-dimethyl-1-piperidyl]-2-pyridyl]amino]-1-[2-(dimethylamino)ethyl]-2-oxo-3-quinolyl]oxy]acetate (50 mg, 82.91 μmol) in EtOH (5 mL) was added methenamine (5 mL, 40% purity in H₂O). The mixture was stirred at 70° C. for 12 hr. The reaction mixture was concentrated under reduced pressure to give a residue which was purified by prep-HPLC (column: Waters Xbridge BEH C18 100*30 mm*10 μm; mobile phase: [water (NH₄HCO₃)-ACN]; B %: 35%-65%, 10 min) to give the title compound as a white solid (6 mg, 9.25 μmol, 11% yield, 93% purity). ¹H NMR (400 MHz, DMSO-d₆) δ 9.14 (s, 1H), 7.99 (s, 1H), 7.74 (s, 1H), 7.66 (d, J=8.8 Hz, 1H), 7.47 (d, J=9.2 Hz, 1H), 7.22 (s, 2H), 4.53 (s, 2H), 4.39 (t, J=6.8 Hz, 2H), 4.13 (d, J=12.4 Hz, 2H), 2.78 (t, J=12.8 Hz, 2H), 2.67 (d, J=4.4 Hz, 3H), 2.63-2.58 (m, 1H), 2.23 (s, 6H), 2.09-2.03 (m, 3H), 0.84 (d, J=6.8 Hz, 6H).

### Example 12: Synthesis of 2-((6-((3-chloro-5-cyano-6-((3R,5S)-4,4-difluoro-3,5-dimethylpiperidin-1-yl)pyridin-2-yl)amino)-1-(oxetan-3-ylmethyl)-2-oxo-1,2-dihydroquinolin-3-yl)oxy)-N-methylacetamide (7) and 2-((6-((3-chloro-5-cyano-6-((3R,5S)-4,4-difluoro-3,5-dimethylpiperidin-1-yl)pyridin-2-yl)amino)-2-(oxetan-3-ylmethoxy)quinolin-3-yl)oxy)-N-methylacetamide (58)

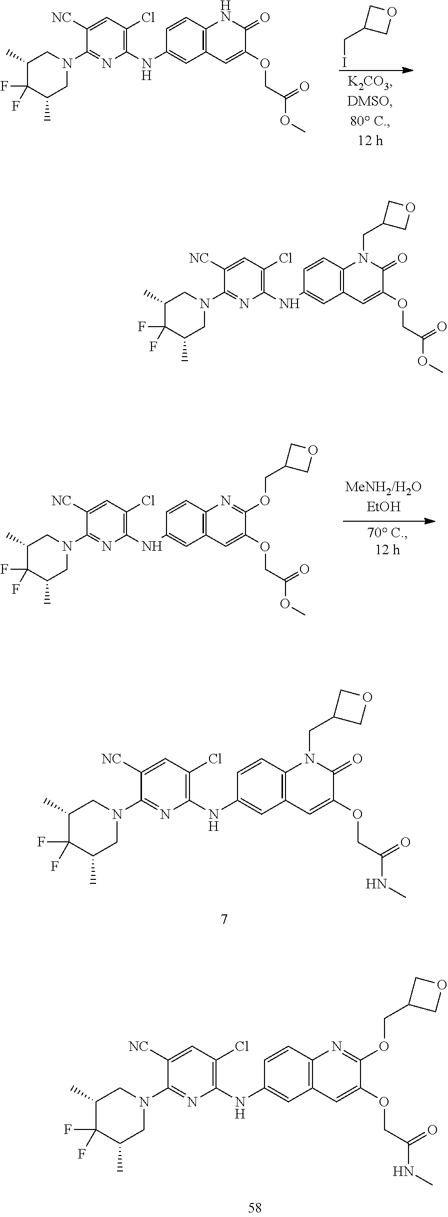 [SMILES: COC(=O)COc1cc2cc(Nc3nc(N4C[C@@H](C)C(F)(F)[C@@H](C)C4)c(C#N)cc3Cl)ccc2[nH]c1=O ; ICC1COC1 ; COC(=O)COc1cc2cc(Nc3nc(N4C[C@@H](C)C(F)(F)[C@@H](C)C4)c(C#N)cc3Cl)ccc2n(CC2COC2)c1=O ; COC(=O)COc1cc2cc(Nc3nc(N4C[C@@H](C)C(F)(F)[C@@H](C)C4)c(C#N)cc3Cl)ccc2nc1OCC1COC1 ; CNC(=O)COc1cc2cc(Nc3nc(N4C[C@@H](C)C(F)(F)[C@@H](C)C4)c(C#N)cc3Cl)ccc2n(CC2COC2)c1=O ; CNC(=O)COc1cc2cc(Nc3nc(N4C[C@@H](C)C(F)(F)[C@@H](C)C4)c(C#N)cc3Cl)ccc2nc1OCC1COC1]

### Methyl 2-[[6-[[3-chloro-5-cyano-6-[(3S,5R)-4,4-difluoro-3,5-dimethyl-1-piperidyl]-2-pyridyl]amino]-1-(oxetan-3-ylmethyl)-2-oxo-3-quinolyl]oxy]acetate and methyl 2-((6-((3-chloro-5-cyano-6-((3R,5S)-4,4-difluoro-3,5-dimethylpiperidin-1-yl)pyridin-2-yl)amino)-2-(oxetan-3-ylmethoxy)quinolin-3-yl)oxy)acetate

To a solution of methyl 2-((6-((3-chloro-5-cyano-6-((3R,5S)-4,4-difluoro-3,5-dimethylpiperidin-1-yl)pyridin-2-yl)amino)-2-oxo-1,2-dihydroquinolin-3-yl)oxy)acetate (500 mg, 939.96 μmol) and K₂CO₃ (259.82 mg, 1.88 mmol) in DMSO (6 mL) was added 3-(iodomethyl)oxetane (223.34 mg, 1.13 mmol), and the reaction mixture was stirred at 80° C. for 12 hr. The reaction mixture was treated with water (9 mL), then a precipitate formed, the suspension was filtered and the filter cake was collected and dried in vacuo to give the mixture of methyl 2-[[6-[[3-chloro-5-cyano-6-[(3S,5R)-4,4-difluoro-3,5-dimethyl-1-piperidyl]-2-pyridyl]amino]-1-(oxetan-3-ylmethyl)-2-oxo-3-quinolyl]oxy]acetate and methyl 2-((6-((3-chloro-5-cyano-6-((3R,5S)-4,4-difluoro-3,5-dimethylpiperidin-1-yl)pyridin-2-yl)amino)-2-(oxetan-3-ylmethoxy)quinolin-3-yl)oxy)acetate as a brown solid (500 mg, crude).

### 2-((6-((3-Chloro-5-cyano-6-((3R,5S)-4,4-difluoro-3,5-dimethylpiperidin-1-yl)pyridin-2-yl)amino)-1-(oxetan-3-ylmethyl)-2-oxo-1,2-dihydroquinolin-3-yl)oxy)-N-methylacetamide and 2-((6-((3-chloro-5-cyano-6-((3R,5S)-4,4-difluoro-3,5-dimethylpiperidin-1-yl)pyridin-2-yl)amino)-2-(oxetan-3-ylmethoxy)quinolin-3-yl)oxy)-N-methylacetamide

A mixture of methyl 2-[[6-[[3-chloro-5-cyano-6-[(3S,5R)-4,4-difluoro-3,5-dimethyl-1-piperidyl]-2-pyridyl]amino]-1-(oxetan-3-ylmethyl)-2-oxo-3-quinolyl]oxy]acetate and methyl 2-((6-((3-chloro-5-cyano-6-((3R,5S)-4,4-difluoro-3,5-dimethylpiperidin-1-yl)pyridin-2-yl)amino)-2-(oxetan-3-ylmethoxy)quinolin-3-yl)oxy)acetate (200 mg, 332.21 μmol) and MeNH₂ (25.79 mg, 332.21 μmol, 10 mL, 40% purity in H₂O) in EtOH (10 mL) was stirred at 70° C. for 12 hr. The reaction mixture was concentrated in vacuo and then dissolved with DMSO (1.5 mL) and purified by prep-HPLC (column: Waters Xbridge Prep OBD C18 150*40 mm*10 μm; mobile phase: [water(NH4HCO3)-ACN]; B %: 35%-65%, 8 min) to give the two title compounds.

### 2-((6-((3-chloro-5-cyano-6-((3R,5S)-4,4-difluoro-3,5-dimethylpiperidin-1-yl)pyridin-2-yl)amino)-1-(oxetan-3-ylmethyl)-2-oxo-1,2-dihydroquinolin-3-yl)oxy)-N-methylacetamide (7)

The title compound was isolated as a white solid (45 mg, 74.79 μmol, 23% yield, 99% purity). ¹H NMR (400 MHz, DMSO-d₆) δ 9.13 (s, 1H), 7.99 (s, 1H), 7.98-7.92 (d, J=4.4 Hz, 1H), 7.80-7.74 (d, J=2.4 Hz, 1H), 7.64-7.58 (m, 1H), 7.57-7.51 (m, 1H), 7.22 (m, 1H), 4.66 (d, J=7.0 Hz, 2H), 4.62-4.56 (m, 2H), 4.55-4.49 (m, 4H), 4.17-4.08 (m, 2H), 3.43 (m, 1H), 2.86-2.72 (m, 2H), 2.67 (d, J=4.8 Hz, 3H), 2.15-1.97 (m, 2H), 0.84 (d, J=6.4 Hz, 6H).

### 2-((6-((3-chloro-5-cyano-6-((3R,5S)-4,4-difluoro-3,5-dimethylpiperidin-1-yl)pyridin-2-yl)amino)-2-(oxetan-3-ylmethoxy)quinolin-3-yl)oxy)-N-methylacetamide (58)

The title compound was isolated as a white solid (45 mg, 74.79 μmol, 23% yield, 99% purity). ¹H NMR (400 MHz, DMSO-d₆) δ 9.16 (m, 1H), 8.01 (m, 1H), 7.91-7.85 (m, 2H), 7.74-7.64 (m, 2H), 7.49 (s, 1H), 4.78-4.72 (m, 2H), 4.67 (d, J=6.8 Hz, 2H), 4.63 (s, 2H), 4.51 (t, J=6.0 Hz, 2H), 4.20-4.12 (d, J=12.4 Hz, 2H), 3.51-3.42 (m, 1H), 2.80 (t, J=12.4 Hz, 2H), 2.66 (d, J=4.8 Hz, 3H), 2.15-1.99 (m, 2H), 0.84 (d, J=6.8 Hz, 6H).

### Example 13: Synthesis of 2-((3S,5R)-3-amino-4,4-difluoro-5-methylpiperidin-1-yl)-5-chloro-6-((3-(2-(1-hydroxycyclopropyl)ethyl)-1-methyl-2-oxo-2,3-dihydro-1H-benzo[d]imidazol-5-yl)amino)nicotinonitrile (59)

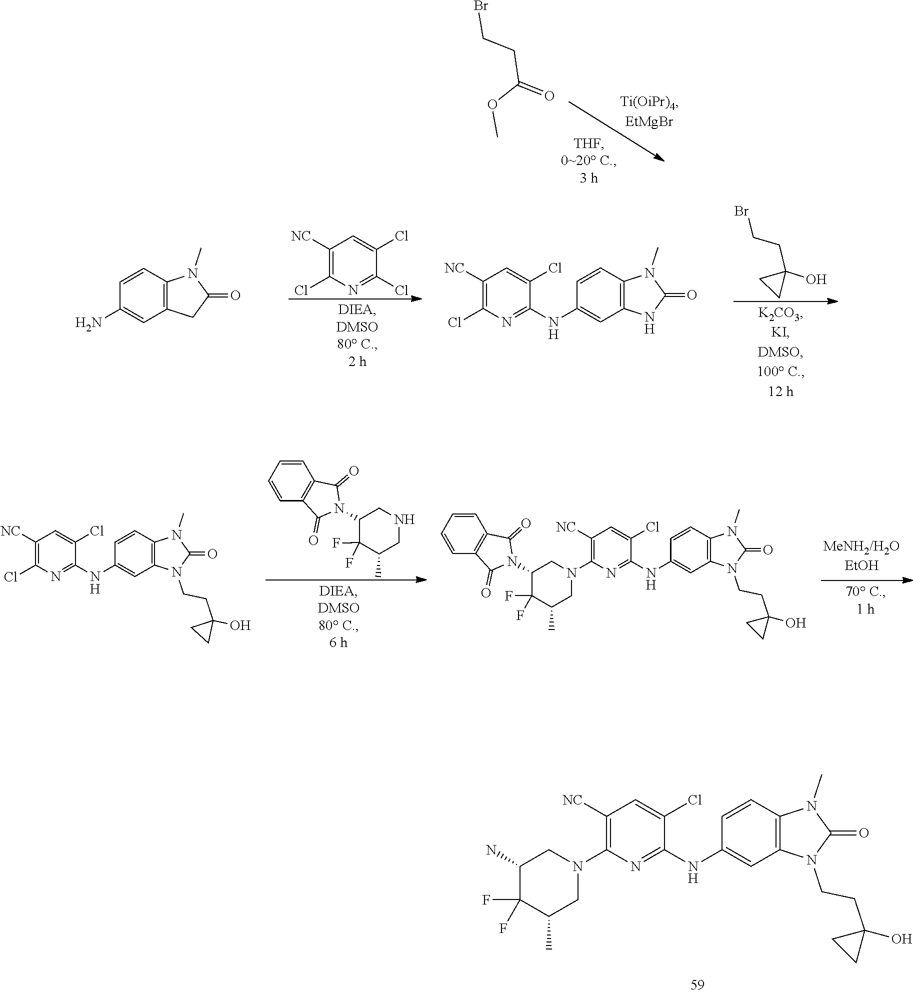

### 2,5-Dichloro-6-((I-methyl-2-oxo-2,3-dihydro-1H-benzo[d]imidazol-5-yl)amino) nicotinonitrile

A flask with mixture of 5-amino-1-methyl-1H-benzo[d]imidazol-2(3H)-one (2 g, 12.26 mmol), 2,5,6-trichloropyridine-3-carbonitrile (2.54 g, 12.26 mmol) and DIEA (3.17 g, 24.51 mmol, 4.27 mL) in DMSO (20 mL) was degassed and purged with N₂ for 3 times, and then the mixture was stirred at 80° C. for 2 hr under N₂ atmosphere. Water (50 mL) was added to the mixture, forming a precipitate which was filtered, the filter cake was dried under reduced pressure to give the title compound as a brown solid (4 g, 88% purity). ¹H NMR (400 MHz, DMSO-d₆) δ 3.28 (s, 3H), 7.06-7.09 (m, 1H), 7.13-7.17 (m, 1H), 7.20 (d, J=1.71 Hz, 1H), 8.32 (s, 1H), 9.43 (s, 1H), 10.90 (s, 1H).

### 1-(2-Bromoethyl)cyclopropanol

To a solution of methyl 3-bromopropanoate (10 g, 59.88 mmol, 6.54 mL) and tetraisopropoxytitanium (17.02 g, 59.88 mmol, 17.67 mL) in THF (400 mL) was added EtMgBr (3 M, 43.91 mL) (3 M solution in diethyl ether) dropwise at 0° C. under N₂. The mixture was stirred at 20° C. for 3 hr. The reaction mixture was quenched with saturated ammonium chloride solution (600 mL). Then the mixture was added to Celite® and filtered to give the filtrate. The filtrate was extracted with EtOAc (3×500 mL). The combined organic phase was dried with anhydrous Na₂SO₄, filtered and concentrated in vacuo. The residue was purified by column chromatography (SiO₂, Petroleum ether/Ethyl acetate=0/1 to 10/1) to give the title compound as a yellow oil (7 g, 42.42 mmol, 35% yield). ¹H NMR (400 MHz, DMSO-d₆) δ 5.15 (s, 1H), 3.61 (t, J=7.6 Hz, 2H), 1.99 (t, J=7.6 Hz, 2H), 0.56 (dd, J=6.8, 4.8 Hz, 1H), 0.43 (dd, J=6.4, 4.4 Hz, 1H).

### 2,5-Dichloro-6-((3-(2-(1-hydroxycyclopropyl)ethyl)-1-methyl-2-oxo-2,3-dihydro-1H-benzo[d]imidazol-5-yl)amino)nicotinonitrile

A flask with solution of 2,5-dichloro-6-((1-methyl-2-oxo-2,3-dihydro-1H-benzo[d]imidazol-5-yl)amino)nicotinonitrile (1.5 g, 4.49 mmol), 1-(2-bromoethyl)cyclopropanol (1.48 g, 8.98 mmol), K₂CO₃ (1.24 g, 8.98 mmol) and KI (372.58 mg, 2.24 mmol) in DMSO (15 mL) was degassed and purged with N₂ for 3 times, then the mixture was stirred at 100° C. for 12 hr under N₂ atmosphere. The mixture was concentrated in vacuo without further work-up. The residue was purified by prep-HPLC (column: Xtimate C18 10 μm 250 mm*80 mm; mobile phase: [water(NH₄HCO₃)-ACN]; B %: 35%-65%, 20 min) to give the title compound as a yellow solid (600 mg, 1.42 mmol, 32% yield, 99% purity). ¹H NMR (400 MHz, DMSO-d₆) δ 0.27-0.33 (m, 2H), 0.51-0.57 (m, 2H), 1.76-1.82 (m, 2H), 3.34 (s, 3H), 3.95-4.03 (m, 2H), 5.22 (s, 1H), 7.14-7.17 (m, 1H), 7.18-7.21 (m, 1H), 7.36 (d, J=1.63 Hz, 1H), 8.35 (s, 1H), 9.53 (s, 1H).

### 5-Chloro-2-((3S,5R)-3-(1,3-dioxoisoindolin-2-yl)-4,4-difluoro-5-methylpiperidin-1-yl)-6-((3-(2-(I-hydroxycyclopropyl)ethyl)-1-methyl-2-oxo-2,3-dihydro-1H-benzo[d]imidazol-5-yl)amino)nicotinonitrile

A flask with solution of 2,5-dichloro-6-((3-(2-(1-hydroxycyclopropyl)ethyl)-1-methyl-2-oxo-2,3-dihydro-1H-benzo[d]imidazol-5-yl)amino)nicotinonitrile (150 mg, 358.61 μmol), 2-[(3R,5S)-4,4-difluoro-5-methyl-3-piperidyl]isoindoline-1,3-dione (150.76 mg, 537.92 μmol) and DIEA (231.74 mg, 1.79 mmol, 312.31 μL) in DMSO (1.5 mL) was degassed and purged with N₂ for 3 times, and then the mixture was stirred at 80° C. for 6 h under N₂ atmosphere. The mixture was concentrated in vacuo without further work-up. The residue was purified by prep-HPLC (column: Waters Xbridge BEH C18 100*30 mm*10 μm; mobile phase: [water(NH₄HCO₃)-ACN]; B %: 40%-70%, 8 min) to give the title compound as a yellow solid (90 mg, 28% yield, 99% purity). ¹H NMR (400 MHz, DMSO-d₆) δ 0.21-0.29 (m, 2H), 0.49 (br d, J=5.13 Hz, 2H), 0.84-0.89 (m, 3H), 1.17 (t, J=7.09 Hz, 2H), 1.62-1.76 (m, 2H), 2.13-2.28 (m, 1H), 2.80-2.86 (m, 3H), 3.11-3.20 (m, 1H), 3.77-3.84 (m, 2H), 4.14-4.38 (m, 4H), 5.19 (s, 1H), 6.82 (d, J=8.34 Hz, 1H), 7.05 (dd, J=8.34, 1.55 Hz, 1H), 7.19 (d, J=1.79 Hz, 1H), 7.70-7.83 (m, 2H), 7.85-7.91 (m, 2H), 8.02 (s, 1H), 9.11 (s, 1H).

### 2-((3S,5R)-3-Amino-4,4-difluoro-5-methylpiperidin-1-yl)-5-chloro-6-((3-(2-(1-hydroxycyclopropyl)ethyl)-1-methyl-2-oxo-2,3-dihydro-1H-benzo[d]imidazol-5-yl)amino)nicotinonitrile (59)

To a solution of 5-chloro-2-((3R,5S)-3-(1,3-dioxoisoindolin-2-yl)-4,4-difluoro-5-methylpiperidin-1-yl)-6-((3-(2-(1-hydroxycyclopropyl)ethyl)-1-methyl-2-oxo-2,3-dihydro-1H-benzo[d]imidazol-5-yl)amino)nicotinonitrile (90 mg, 135.93 μmol) in EtOH (4 mL) was added MeNH₂/H₂O (4 mL, 40% in water). The mixture was stirred at 70° C. for 1 hr. The mixture was concentrated in vacuo without further work-up. The residue was purified by prep-HPLC (column: Waters Xbridge BEH C18 100*30 mm*10 μm; mobile phase: [water(NH₄HCO₃)-ACN]; B %: 30%-60%, 8 min) to give the title compound as a white solid (25 mg, 46.52 μmol, 34% yield, 99% purity). ¹H NMR (400 MHz, DMSO-d₆) δ 0.22-0.31 (m, 2H), 0.46-0.56 (m, 2H), 0.79 (d, J=6.72 Hz, 3H), 1.60-1.71 (m, 2H), 1.73-1.80 (m, 2H), 1.94-2.13 (m, 1H), 2.76-2.92 (m, 2H), 3.33 (s, 3H), 4.00 (br t, J=7.64 Hz, 2H), 4.14-4.24 (m, 1H), 5.27 (s, 1H), 7.13 (d, J=8.44 Hz, 1H), 7.23 (dd, J=8.44, 1.83 Hz, 1H), 7.35 (d, J=1.83 Hz, 1H), 7.96 (s, 1H), 8.97 (s, 1H).

The absolute configuration of compound 59 was randomly assigned based on the amino group and methyl group being in cis-conformation.

### Example 14: Synthesis of 2-[(3R,5S)-3-amino-4,4-difluoro-5-methyl-1-piperidyl]-5-fluoro-6-[[1-methyl-2-oxo-3-[[(R)-2-oxooxazolidin-4-yl]methyl]benzimidazol-5-yl]amino]pyridine-3-carbonitrile (60) and 2-((3R,5S)-3-amino-4,4-difluoro-5-methylpiperidin-1-yl)-5-fluoro-6-((1-methyl-2-oxo-3-(((S)-2-oxooxazolidin-4-yl)methyl)-2,3-dihydro-1H-benzo[d]imidazol-5-yl)amino)nicotinonitrile (61)

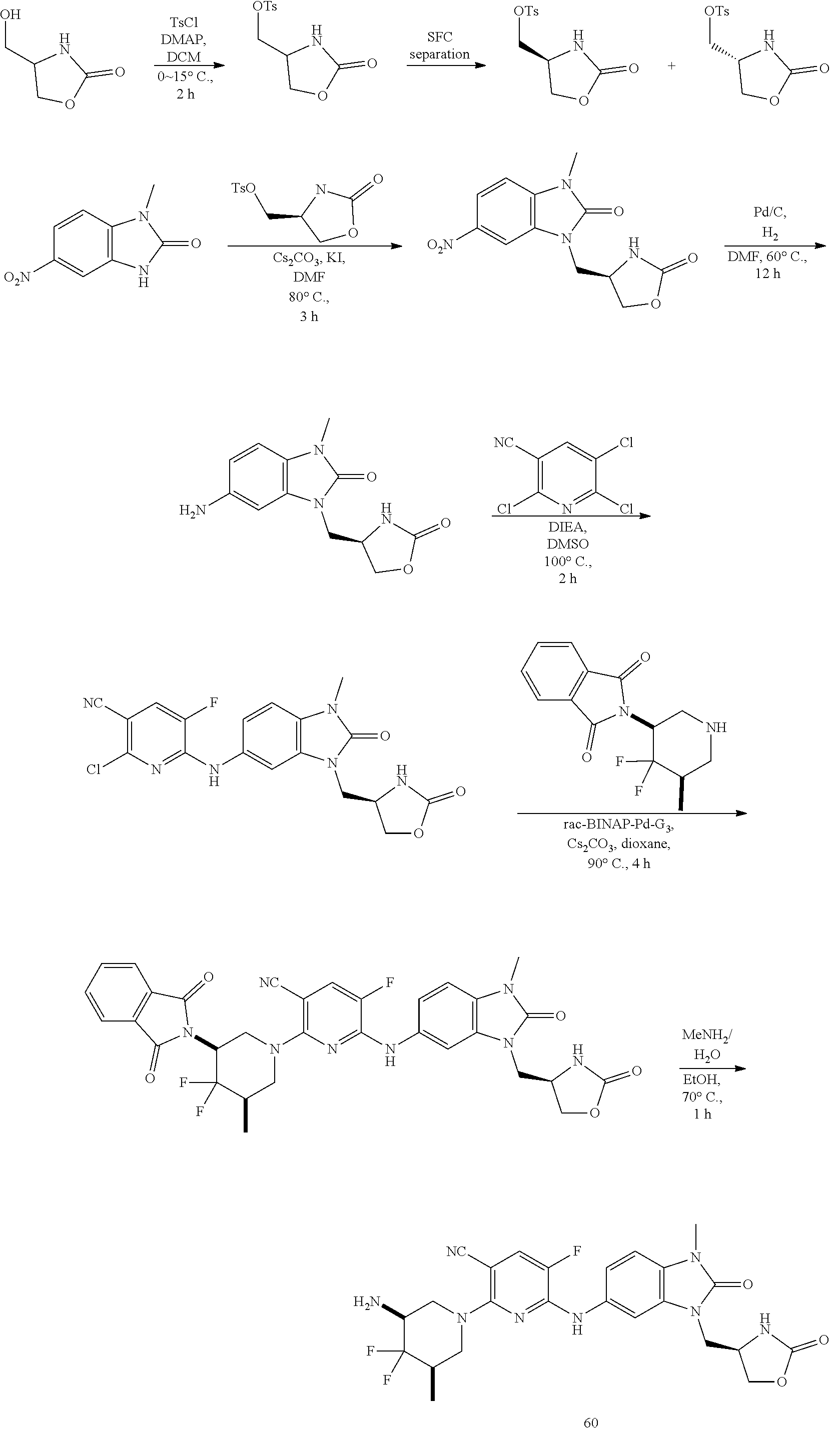

### (2-Oxooxazolidin-4-yl)methyl 4-methylbenzenesulfonate

To a solution of 4-(hydroxymethyl)oxazolidin-2-one (12.5 g, 106.74 mmol) and DMAP (26.08 g, 213.49 mmol) in DCM (500 mL) was added 4-methylbenzenesulfonyl chloride (24.42 g, 128.09 mmol) at 0° C. The mixture was stirred at 0° C. for 1 hr and then warmed up to 15° C. for 1 hr. The reaction mixture was washed with 1N HCl (1 L), H₂O (1 L), sat. NaHCO₃ (1 L), sat. NaCl (1 L), dried over anhydrous Na₂SO₄ and concentrated in vacuo to give a residue which was purified by column chromatography (SiO₂, Petroleum ether/Ethyl acetate=5/1 to 0/1) to give the title compound as a white solid (45 g, 159.24 mmol, 75% yield, 96% purity). ¹H NMR (400 MHz, MeCN-d₃) δ 7.79 (d, J=8 Hz, 2H), 7.45 (d, J=8 Hz, 2H), 5.99 (br s, 1H), 4.32-4.40 (m, 1H), 3.93-4.07 (m, 4H), 2.45 (s, 3H).

### (S)-(2-Oxooxazolidin-4-yl)methyl 4-methylbenzenesulfonate and (R)-(2-oxooxazolidin-4-yl)methyl 4-methylbenzenesulfonate

Racemic (2-oxooxazolidin-4-yl)methyl 4-methylbenzenesulfonate (10 g, 36.86 mmol) was separated with SFC (column: Phenomenex-Cellulose-2 (250 mm*50 mm, 10 μm); mobile phase: [0.1% NH₃H₂O EtOH]; B %: 60%-60%, 7.7 min) to give two enantiopure compounds. (S)-(2-oxooxazolidin-4-yl)methyl 4-methylbenzenesulfonate as a yellow solid (4.9 g, 17.70 mmol, 48% yield, 98% purity). ¹H NMR (400 MHz, MeCN-d₃) δ 7.80 (d, J=8 Hz, 2H), 7.46 (d, J=8 Hz, 2H), 5.95 (br s, 1H), 4.32-4.40 (m, 1H), 3.93-4.07 (m, 4H), 2.45 (s, 3H). (R)-(2-oxooxazolidin-4-yl)methyl 4-methylbenzenesulfonate as a yellow solid (4.9 g, 17.70 mmol, 48% yield, 98% purity). ¹H NMR (400 MHz, MeCN-d₃) δ 7.79 (d, J=8.34 Hz, 2H), 7.45 (d, J=8.11 Hz, 2H), 5.98 (br s, 1H), 4.31-4.41 (m, 1H), 3.92-4.08 (m, 4H), 2.45 (s, 3H).

### (R)-4-[(3-Methyl-6-nitro-2-oxo-benzimidazol-1-yl)methyl]oxazolidin-2-one

A flask with mixture of 3-methyl-6-nitro-1H-benzimidazol-2-one (500 mg, 2.59 mmol), [(S)-2-oxooxazolidin-4-yl]methyl 4-methylbenzenesulfonate (842.69 mg, 3.11 mmol), K₂CO₃ (715.52 mg, 5.18 mmol) and KI (214.85 mg, 1.29 mmol) in DMSO (10 mL) was degassed and purged with N₂ for 3 times, and then the mixture was stirred at 80° C. for 3 hr under N₂ atmosphere. The reaction mixture was treated with water (20 mL), forming a precipitate that was filtered and the filter cake was washed with EtOAc (10 mL) and dried under reduced pressure to give the title compound a black solid (600 mg, 2.03 mmol, 79% yield, 99% purity).

### (R)-4-[(6-Amino-3-methyl-2-oxo-benzimidazol-1-yl)methyl]oxazolidin-2-one

To a solution of (R)-4-[(3-methyl-6-nitro-2-oxo-benzimidazol-1-yl)methyl]oxazolidin-2-one (600 mg, 2.05 mmol) in DMF (2 mL) was added Pd/C (200 mg, 10% purity) under Ar. The suspension was degassed under vacuum and purged with H₂ for 3 times. The mixture was stirred under H₂ (50 psi) at 60° C. for 12 hours. The reaction mixture was filtered, and the filtrate was concentrated under reduced pressure to get a residue which was purified by column chromatography (SiO₂, Ethyl acetate/Ethanol=1/0 to 0/1) to give the title compound as a yellow solid (500 mg, 1.91 mmol, 93% yield). ¹H NMR (400 MHz, DMSO-d₆) δ 7.84 (s, 1H), 6.89 (d, J=8 Hz, 1H), 6.61 (d, J=1.8 Hz, 1H), 6.48 (dd, J=8, 2 Hz, 1H), 6.15 (br s, 2H), 4.31-4.40 (m, 1H), 4.09-4.19 (m, 2H), 3.77-3.85 (m, 2H), 3.25 (s, 3H).

### 2-Chloro-5-fluoro-6-[[1-methyl-2-oxo-3-[[(R)-2-oxooxazolidin-4-yl]methyl]benzimidazol-5-yl]amino]pyridine-3-carbonitrile

To a solution of (R)-4-[(6-amino-3-methyl-2-oxo-benzimidazol-1-yl)methyl]oxazolidin-2-one (500 mg, 1.91 mmol) and 2,6-dichloro-5-fluoro-pyridine-3-carbonitrile (364.12 mg, 1.91 mmol) in DMSO (10 mL) was added DIPEA (492.79 mg, 3.81 mmol, 664.13 μL). The mixture was stirred at 100° C. for 2 hr. The mixture was treated with water (20 mL), forming a precipitate that was filtered and dried under reduce pressure to give the title compound as a brown solid (550 mg, 1.17 mmol, 62% yield, 89% purity). ¹H NMR (400 MHz, DMSO-d₆) δ 9.99 (s, 1H), 8.14 (d, J=10.4 Hz, 1H), 7.83 (s, 1H), 7.49 (d, J=1.2 Hz, 1H), 7.25 (br dd, J=8, 1.6 Hz, 1H), 7.15 (d, J=8 Hz, 1H), 4.32-4.44 (m, 1H), 4.14-4.24 (m, 2H), 3.80-3.97 (m, 2H), 3.33 (br s, 3H).

### 2-((3R,5S)-3-(1,3-dioxoisoindolin-2-yl)-4,4-difluoro-5-methylpiperidin-1-yl)-5-fluoro-6-((I-methyl-2-oxo-3-(((R)-2-oxooxazolidin-4-yl)methyl)-2,3-dihydro-1H-benzo[d]imidazol-5-yl)amino)nicotinonitrile

To a solution of 2-chloro-5-fluoro-6-[[1-methyl-2-oxo-3-[[(R)-2-oxooxazolidin-4-yl]methyl]benzimidazol-5-yl]amino]pyridine-3-carbonitrile (500 mg, 1.20 mmol), 2-[(3R,5S)-4,4-difluoro-5-methyl-3-piperidyl]isoindoline-1,3-dione (336.22 mg, 1.20 mmol) and Cs₂CO₃ (781.73 mg, 2.40 mmol) in dioxane (5 mL) was added rac-BINAP-Pd-G3 (119.05 mg, 119.96 μmol). The mixture was stirred at 90° C. for 4 hr. The reaction mixture was filtered through a pad of Celite® and the filtrate concentrated under reduced pressure to give a residue which was purified by prep-HPLC (column: Waters Xbridge BEH C18 100*30 mm*10 μm; mobile phase: [water(NH₄HCO₃)-ACN]; B %: 30%-60%, 8 min) to give the title compound (150 mg, 19% yield, 98% purity). ¹H NMR (400 MHz, MeCN-d₃) δ 7.87 (br s, 1H), 7.83 (dd, J=5.46, 3.07 Hz, 2H), 7.67-7.76 (m, 2H), 7.51 (d, J=10.54 Hz, 1H), 7.09-7.22 (m, 2H), 6.75 (d, J=8.28 Hz, 1H), 6.08 (s, 1H), 4.48-4.57 (m, 1H), 4.33-4.47 (m, 2H), 4.24-4.32 (m, 3H), 4.17-4.23 (m, 2H), 3.69-3.87 (m, 2H), 3.21 (dd, J=14.12, 12.11 Hz, 1H), 2.84 (s, 3H), 0.97 (d, J=6.78 Hz, 3H).

### 2-[(3R,5S)-3-Amino-4,4-difluoro-5-methyl-1-piperidyl]-5-fluoro-6-[[1-methyl-2-oxo-3-[[(R)-2-oxooxazolidin-4-yl]methyl]benzimidazol-5-yl]amino]pyridine-3-carbonitrile (60)

To a solution of 2-[(3R,5S)-3-(1,3-dioxoisoindolin-2-yl)-4,4-difluoro-5-methyl-1-piperidyl]-5-fluoro-6-[[1-methyl-2-oxo-3-[[(R)-2-oxooxazolidin-4-yl]methyl]benzimidazol-5-yl]amino]pyridine-3-carbonitrile (100 mg, 151.38 μmol) in EtOH (3.5 mL) was added MeNH₂ (3.5 mL, 40% purity). The mixture was stirred at 70° C. for 1 hr. The reaction mixture was concentrated under reduced pressure to give a residue which was purified by prep-HPLC (column: Waters Xbridge BEH C18 100*30 mm*10 μm; mobile phase: [water(NH₄HCO₃)-ACN]; B %: 25%-45%, 8 min) to the title compound as a white solid (10 mg, 18.66 μmol, 12% yield, 99% purity). ¹H NMR (400 MHz, MeCN-d₃) δ 7.81 (br d, J=1.13 Hz, 1H), 7.55 (d, J=1.88 Hz, 1H), 7.46-7.50 (m, 1H), 7.22 (dd, J=8.44, 1.94 Hz, 1H), 7.04 (d, J=8.50 Hz, 1H), 6.36 (br s, 1H), 4.36-4.46 (m, 1H), 4.22-4.32 (m, 2H), 4.14-4.21 (m, 1H), 4.01-4.08 (m, 1H), 3.94-4.00 (m, 2H), 3.36 (s, 3H), 3.00-3.16 (m, 1H), 2.78-2.90 (m, 2H), 2.18-2.25 (m, 1H), 0.98 (d, J=6.88 Hz, 3H).

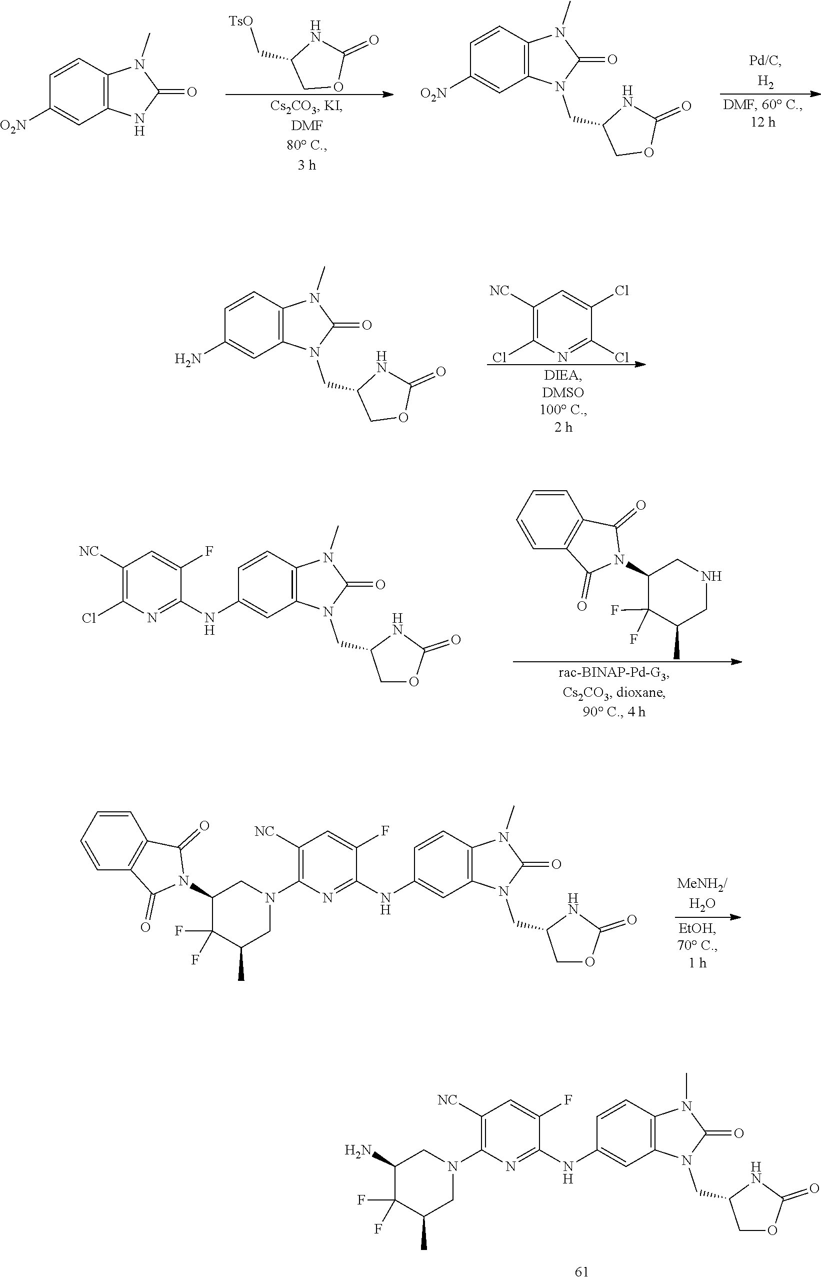

### (S)-4-((3-Methyl-6-nitro-2-oxo-2,3-dihydro-1H-benzo[d]imidazol-1-yl)methyl)oxazolidin-2-one

A flask with mixture of 3-methyl-6-nitro-1H-benzimidazol-2-one (500 mg, 2.59 mmol), [(4R)-2-oxooxazolidin-4-yl]methyl 4-methylbenzenesulfonate (842.69 mg, 3.11 mmol), K₂CO₃ (715.52 mg, 5.18 mmol) and KI (214.85 mg, 1.29 mmol) in DMSO (10 mL) was degassed and purged with N₂ for 3 times, and then the mixture was stirred at 80° C. for 3 hr under N₂ atmosphere. The reaction mixture was treated with water (20 mL), forming a precipitate, which was filtered and the filter cake was washed with EtOAc (10 mL) then was dried under reduced pressure to give the title compound as a black solid (600 mg, 2.03 mmol, 79% yield, 99% purity).

### (S)-4-((6-Amino-3-methyl-2-oxo-2,3-dihydro-1H-benzo[d]imidazol-1-yl)methyl)oxazolidin-2-one

To a solution of (4R)-4-[(3-methyl-6-nitro-2-oxo-benzimidazol-1-yl)methyl]oxazolidin-2-one (600 mg, 2.05 mmol) in DMF (2 mL) was added Pd/C (200 mg, 10% purity) under Ar. The flask was degassed and purged with H₂ for 3 times. The mixture was stirred under H₂ (50 psi) at 60° C. for 10 hours. The reaction mixture was filtered, and the filtrate was concentrated under reduced pressure to give a residue which was purified by column chromatography (SiO₂, Ethyl acetate/Ethanol=1/0 to 0/1) to give the title compound as a yellow solid (500 mg, 1.70 mmol, 83% yield, 89% purity). ¹H NMR (400 MHz, DMSO-d₆) δ 7.85 (s, 1H), 6.80 (d, J=8.23 Hz, 1H), 6.47 (d, J=1.67 Hz, 1H), 6.34 (dd, J=8.23, 1.79 Hz, 1H), 4.76 (br s, 2H), 4.30-4.39 (m, 1H), 4.07-4.20 (m, 2H), 3.78 (br d, J=5.72 Hz, 2H), 3.23 (s, 3H).

### (S)-2-Chloro-5-fluoro-6-((I-methyl-2-oxo-3-((2-oxooxazolidin-4-yl)methyl)-2,3-dihydro-1H-benzo[d]imidazol-5-yl)amino)nicotinonitrile

To a solution of (4S)-4-[(6-amino-3-methyl-2-oxo-benzimidazol-1-yl)methyl]oxazolidin-2-one (500 mg, 1.91 mmol) and 2,6-dichloro-5-fluoro-pyridine-3-carbonitrile (364.12 mg, 1.91 mmol) in DMSO (10 mL) was added DIEA (492.79 mg, 3.81 mmol, 664.13 μL). The mixture was stirred at 100° C. for 2 hr. The mixture was treated with water (20 mL), forming a precipitate which was filtered and dried under reduced pressure to give the title compound as a brown solid (560 mg, 1.25 mmol, 66% yield, 93% purity). ¹H NMR (400 MHz, DMSO-d₆) δ 9.98 (br s, 1H), 8.13 (d, J=10.61 Hz, 1H), 7.83 (s, 1H), 7.49 (s, 1H), 7.10-7.33 (m, 2H), 4.31-4.46 (m, 1H), 4.19 (br d, J=5.25 Hz, 2H), 3.88 (br dd, J=12.22, 5.30 Hz, 2H), 3.33 (s, 3H).

### 2-((3R,5S)-3-(1,3-dioxoisoindolin-2-yl)-4,4-difluoro-5-methylpiperidin-1-yl)-5-fluoro-6-((I-methyl-2-oxo-3-(((S)-2-oxooxazolidin-4-yl)methyl)-2,3-dihydro-1H-benzo[d]imidazol-5-yl)amino)nicotinonitrile

To a solution of 2-chloro-5-fluoro-6-[[1-methyl-2-oxo-3-[[(4S)-2-oxooxazolidin-4-yl]methyl]benzimidazol-5-yl]amino]pyridine-3-carbonitrile (500 mg, 1.20 mmol), 2-[(3R,5S)-4,4-difluoro-5-methyl-3-piperidyl]isoindoline-1,3-dione (336.22 mg, 1.20 mmol) and Cs₂CO₃ (781.73 mg, 2.40 mmol) in dioxane (5 mL) was added rac-BINAP-Pd-G3 (119.05 mg, 119.96 μmol). The mixture was stirred at 90° C. for 4 hr. The reaction mixture was filtered and the filtrate was concentrated under reduced pressure to give a residue which was purified by prep-HPLC (column: Waters Xbridge BEH C18 100*30 mm*10 μm; mobile phase: [water(NH₄HCO₃)-ACN]; B %: 30%-60%, 8 min) to give the title compound as a yellow solid (120 mg, 174.39 μmol, 15% yield, 96% purity). ¹H NMR (400 MHz, MeCN-d₃) δ 7.90 (br d, J=1.00 Hz, 1H), 7.83 (dd, J=5.46, 3.07 Hz, 2H), 7.74 (br s, 2H), 7.51 (d, J=10.54 Hz, 1H), 7.21 (d, J=1.88 Hz, 1H), 7.10 (dd, J=8.28, 1.76 Hz, 1H), 6.81 (d, J=8.41 Hz, 1H), 5.94 (s, 1H), 4.45-4.57 (m, 1H), 4.32-4.44 (m, 2H), 4.22-4.32 (m, 3H), 4.11-4.22 (m, 2H), 3.75 (dd, J=14.56, 6.15 Hz, 1H), 3.60-3.68 (m, 1H), 3.15-3.25 (m, 1H), 2.98 (s, 3H), 0.97 (d, J=6.78 Hz, 3H).

### 2-((3R,5S)-3-amino-4,4-difluoro-5-methylpiperidin-1-yl)-5-fluoro-6-((I-methyl-2-oxo-3-(((S)-2-oxooxazolidin-4-yl)methyl)-2,3-dihydro-1H-benzo[d]imidazol-5-yl)amino)nicotinonitrile (61)

To a solution of 2-[(3R,5S)-3-(1,3-dioxoisoindolin-2-yl)-4,4-difluoro-5-methyl-1-piperidyl]-5-fluoro-6-[[1-methyl-2-oxo-3-[[(4S)-2-oxooxazolidin-4-yl]methyl]benzimidazol-5-yl]amino]pyridine-3-carbonitrile (100.00 mg, 151.38 μmol) in EtOH (5 mL) was added MeNH₂ (5 mL, 40% purity). The mixture was stirred at 70° C. for 1 hr. The reaction mixture was concentrated under reduced pressure to give a residue which was purified by prep-HPLC (column: Phenomenex C18 75*30 mm*3 μm; mobile phase: [water(NH₄HCO₃)-ACN]; B %: 25%-45%, 8 min) to give the title compound as a white solid (10 mg, 18.66 μmol, 12% yield, 99% purity). ¹H NMR (400 MHz, DMSO-d₆) δ 9.45 (br s, 1H), 7.78-7.90 (m, 2H), 7.55 (s, 1H), 7.26-7.33 (m, 1H), 7.12 (d, J=8.34 Hz, 1H), 4.33-4.41 (m, 1H), 4.16-4.25 (m, 2H), 4.06-4.15 (m, 1H), 3.94-3.99 (m, 1H), 3.91 (br s, 2H), 3.33 (br s, 3H), 2.89-3.04 (m, 1H), 2.70-2.84 (m, 2H), 2.08-2.20 (m, 1H), 1.65-1.79 (m, 2H), 0.88 (br d, J=6.68 Hz, 3H).

The absolute configurations of compounds 60 & 61 were randomly assigned based on the amino group and methyl group being in cis-conformation.

### Example 15: Synthesis of 5-chloro-2-((3R,5S)-4,4-difluoro-3,5-dimethylpiperidin-1-yl)-6-((1-methyl-3-((3-methyloxetan-3-yl)methoxy)-2-oxo-1,2-dihydroquinolin-6-yl)amino)nicotinonitrile (6)

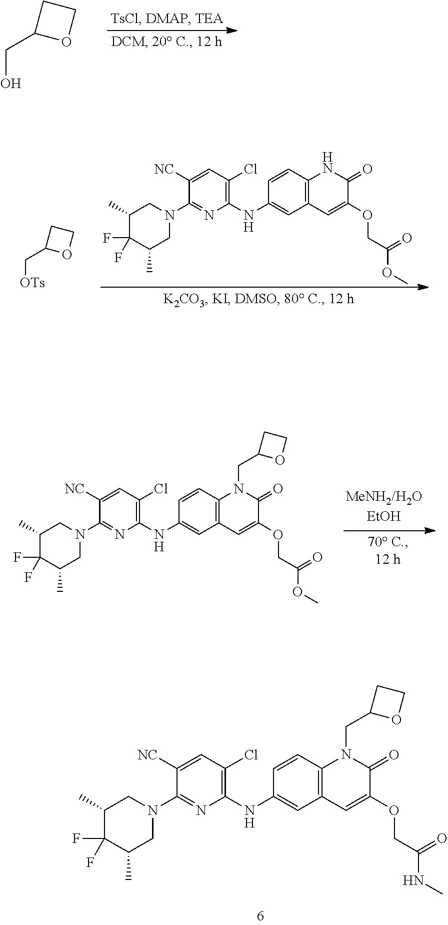 [SMILES: OCC1CCO1 ; Cc1ccc(S(=O)(=O)OCC2CCO2)cc1 ; COC(=O)COc1cc2cc(Nc3nc(N4C[C@@H](C)C(F)(F)[C@@H](C)C4)c(C#N)cc3Cl)ccc2[nH]c1=O ; COC(=O)COc1cc2cc(Nc3nc(N4C[C@@H](C)C(F)(F)[C@@H](C)C4)c(C#N)cc3Cl)ccc2n(CC2CCO2)c1=O ; CNC(=O)COc1cc2cc(Nc3nc(N4C[C@@H](C)C(F)(F)[C@@H](C)C4)c(C#N)cc3Cl)ccc2n(CC2CCO2)c1=O]

### Oxetan-2-ylmethyl 4-methylbenzenesulfonate

To a solution of oxetan-2-ylmethanol (200 mg, 2.27 mmol) and 4-methylbenzenesulfonyl chloride (519.33 mg, 2.72 mmol) in DCM (3 mL) was added DMAP (27.73 mg, 227.00 μmol) and TEA (459.40 mg, 4.54 mmol, 631.92 μL). The mixture was stirred at 20° C. for 12 hr. The mixture was concentrated under reduced pressure to give a residue which was purified by prep-TLC (SiO₂, Petroleum ether:Ethyl acetate=2:1) to give the title compound as a white solid (440 mg, 1.82 mmol, 80% yield). ¹H NMR (400 MHz, CDCl₃) δ 7.83 (d, J=8.4 Hz, 2H), 7.36 (d, J=8.0 Hz, 2H), 4.96-4.90 (m, 1H), 4.64-4.58 (m, 1H), 4.54-4.89 (m, 1H), 4.16 (d, J=4.0 Hz, 2H), 2.76-2.67 (m, 1H), 2.62-2.53 (m, 1H), 2.46 (s, 3H).

### Methyl 2-((6-((3-chloro-5-cyano-6-((3R,5S)-4,4-difluoro-3,5-dimethylpiperidin-1-yl)pyridin-2-yl)amino)-1-(oxetan-2-ylmethyl)-2-oxo-1,2-dihydroquinolin-3-yl)oxy)acetate

A mixture of methyl 2-[[6-[[3-chloro-5-cyano-6-[(3S,5R)-4,4-difluoro-3,5-dimethyl-1-piperidyl]-2-pyridyl]amino]-2-oxo-1H-quinolin-3-yl]oxy]acetate (100 mg, 187.99 μmol), oxetan-2-ylmethyl 4-methylbenzenesulfonate (136.65 mg, 563.98 μmol), K₂CO₃ (77.94 mg, 563.98 μmol), KI (15.60 mg, 94.00 μmol) in DMSO (1 mL was stirred at 80° C. for 12 hr under N₂ atmosphere. Water (3 mL) was added, forming a precipitate which was filtered and washed with H₂O (3 mL) and ethyl acetate (5 mL) and the solid was dried under reduced pressure to give the title compound as a brown solid (100 mg, crude).

### 2-((6-((3-Chloro-5-cyano-6-((3R,5S)-4,4-difluoro-3,5-dimethylpiperidin-1-yl)pyridin-2-yl)amino)-1-(oxetan-2-ylmethyl)-2-oxo-1,2-dihydroquinolin-3-yl)oxy)-N-methylacetamide (6)

To a mixture of methyl 2-[[6-[[3-chloro-5-cyano-6-[(3S,5R)-4,4-difluoro-3,5-dimethyl-1-piperidyl]-2-pyridyl]amino]-1-(oxetan-2-ylmethyl)-2-oxo-3-quinolyl]oxy]acetate (100 mg, 166.11 μmol) in EtOH (5 mL) was added methenamine (5 mL, 40% purity in H₂O). The mixture was stirred at 70° C. for 12 hr. The reaction mixture was concentrated under reduced pressure to give a residue which was purified by prep-HPLC (column: Waters Xbridge BEH C18 100*25 mm*5 μm; mobile phase: [water(NH₄HCO₃)-ACN]; B %: 45%-75%, 10 min) to give the title compound as a white solid (5 mg, 7.51 μmol, 5% yield, 90% purity). ¹H NMR (400 MHz, DMSO-d₆) δ 9.12 (S, 1H), 7.99 (S, 1H), 7.94 (d, J=4.4 Hz, 1H), 7.74 (d, J=2.4 Hz, 1H), 7.69 (d, J=9.2 Hz, 1H), 7.61 (dd, J=2.4, 9.2 Hz, 1H), 7.24 (s, 1H), 5.05-5.01 (m, 1H), 4.68-4.50 (m, 2H), 4.54 (s, 2H), 4.50-4.40 (m, 2H), 4.14 (d, J=12.8 Hz, 2H), 2.82-2.74 (m, 2H), 2.67-2.66 (m, 4H), 2.12-2.00 (m, 2H), 1.23 (s, 1H), 0.84 (d, J=6.8 Hz, 6H).

### Example 16: Synthesis of 2-((6-((3-chloro-5-cyano-6-((3R,5S)-4,4-difluoro-3,5-dimethylpiperidin-1-yl)pyridin-2-yl)amino)-1-(3-hydroxypropyl)-2-oxo-1,2-dihydroquinolin-3-yl)oxy)-N-methylacetamide (18)

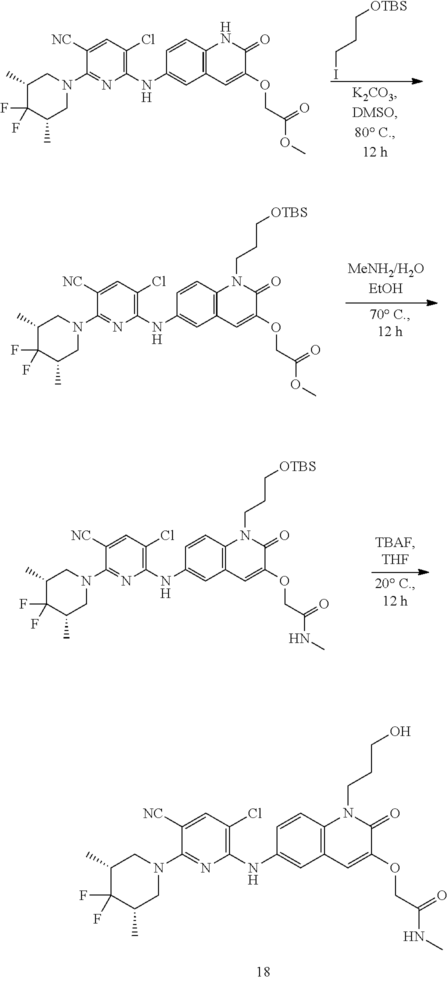 [SMILES: COC(=O)COc1cc2cc(Nc3nc(N4C[C@@H](C)C(F)(F)[C@@H](C)C4)c(C#N)cc3Cl)ccc2[nH]c1=O ; CC(C)(C)[Si](C)(C)OCCCI ; COC(=O)COc1cc2cc(Nc3nc(N4C[C@@H](C)C(F)(F)[C@@H](C)C4)c(C#N)cc3Cl)ccc2n(CCCO[Si](C)(C)C(C)(C)C)c1=O ; CNC(=O)COc1cc2cc(Nc3nc(N4C[C@@H](C)C(F)(F)[C@@H](C)C4)c(C#N)cc3Cl)ccc2n(CCCO[Si](C)(C)C(C)(C)C)c1=O ; CNC(=O)COc1cc2cc(Nc3nc(N4C[C@@H](C)C(F)(F)[C@@H](C)C4)c(C#N)cc3Cl)ccc2n(CCCO)c1=O]

### Methyl 2-((1-(3-((tert-butyldimethylsilyl)oxy)propyl)-6-((3-chloro-5-cyano-6-((3R,5S)-4,4-difluoro-3,5-dimethylpiperidin-1-yl)pyridin-2-yl)amino)-2-oxo-1,2-dihydroquinolin-3-yl)oxy)acetate

A mixture of methyl 2-[[6-[[3-chloro-5-cyano-6-[(3S,5R)-4,4-difluoro-3,5-dimethyl-1-piperidyl]-2-pyridyl]amino]-2-oxo-1H-quinolin-3-yl]oxy]acetate (100 mg, 187.99 μmol), tert-butyl-(3-iodopropoxy)-dimethyl-silane (56.44 mg, 187.99 μmol, 5.41 μL), K₂CO₃ (51.96 mg, 375.98 μmol), in DMSO (2 mL) was stirred at 80° C. for 12 hr. The mixture was cooled to 20° C. and water (1 mL) was added, forming a precipitate which was filtered and the filter cake was washed with H₂O (10 mL×2), EtOAc (10 mL×2), then the filter cake was concentrated to give the title compound as a yellow solid (150 mg, crude), which was used without purification.

### 2-((1-(3-((tert-Butyldimethylsilyl)oxy)propyl)-6-((3-chloro-5-cyano-6-((3R,5S)-4,4-difluoro-3,5-dimethylpiperidin-1-yl)pyridin-2-yl)amino)-2-oxo-1,2-dihydroquinolin-3-yl)oxy)-N-methylacetamide

A mixture of methyl 2-[[1-[3-[tert-butyl(dimethyl)silyl]oxypropyl]-6-[[3-chloro-5-cyano-6-[(3S,5R)-4,4-difluoro-3,5-dimethyl-1-piperidyl]-2-pyridyl]amino]-2-oxo-3-quinolyl]oxy]acetate (150 mg, 85.19 μmol, 40% purity), MeNH₂ (370.59 μmol, 9 mL, 40% purity in H₂O) in EtOH (10 mL) was stirred at 70° C. for 12 hr. The mixture was concentrated to give the title compound as a yellow solid (100 mg, 56.88 μmol, 67% yield, 40% purity), which was used without purification.

### 2-((6-((3-Chloro-5-cyano-6-((3R,5S)-4,4-difluoro-3,5-dimethylpiperidin-1-yl)pyridin-2-yl)amino)-1-(3-hydroxypropyl)-2-oxo-1,2-dihydroquinolin-3-yl)oxy)-N-methylacetamide (18)

To a solution of 2-[[1-[3-[tert-butyl(dimethyl)silyl]oxypropyl]-6-[[3-chloro-5-cyano-6-[(3S,5R)-4,4-difluoro-3,5-dimethyl-1-piperidyl]-2-pyridyl]amino]-2-oxo-3-quinolyl]oxy]-N-methyl-acetamide (100 mg, 142.19 μmol) in THE (2 mL) was added TBAF (1 M, 213.28 μL) and then the mixture was stirred at 20° C. for 12 hr. The mixture was concentrated and the residue was purified directly with prep-HPLC (column: Phenomenex Luna 80*30 mm*3 μm; mobile phase: [water(HCl)-ACN]; B %: 30%-60%, 8 min) to give the title compound as a white solid (6 mg, 9.50 μmol, 7% yield, 99% purity, HCl). ¹H NMR (400 MHz, DMSO-d₆) δ ppm 9.08-9.18 (m, 1H), 7.95-8.04 (m, 2H), 7.78 (d, J=2.38 Hz, 1H), 7.61-7.68 (m, 1H), 7.50-7.57 (m, 1H), 7.22-7.28 (m, 1H), 4.63-4.78 (m, 1H), 4.50-4.60 (m, 2H), 4.27-4.40 (m, 2H), 4.13 (br d, J=12.51 Hz, 2H), 3.54 (br t, J=5.69 Hz, 2H), 2.79 (br t, J=12.76 Hz, 2H), 2.67 (d, J=4.50 Hz, 3H), 1.96-2.15 (m, 2H), 1.75-1.86 (m, 2H), 0.82-0.86 (m, 6H).

### Example 17: Synthesis of 2-((6-((3-chloro-5-cyano-6-((3R,5S)-4,4-difluoro-3-(2-hydroxyethyl)-5-methylpiperidin-1-yl)pyridin-2-yl)amino)-1-methyl-2-oxo-1,2-dihydroquinolin-3-yl) oxy)-N-methylacetamide (62) and 2-((6-((3-chloro-5-cyano-6-((3S,5R)-4,4-difluoro-3-(2-hydroxyethyl)-5-methylpiperidin-1-yl)pyridin-2-yl)amino)-1-methyl-2-oxo-1,2-dihydroquinolin-3-yl) oxy)-N-methylacetamide (63)

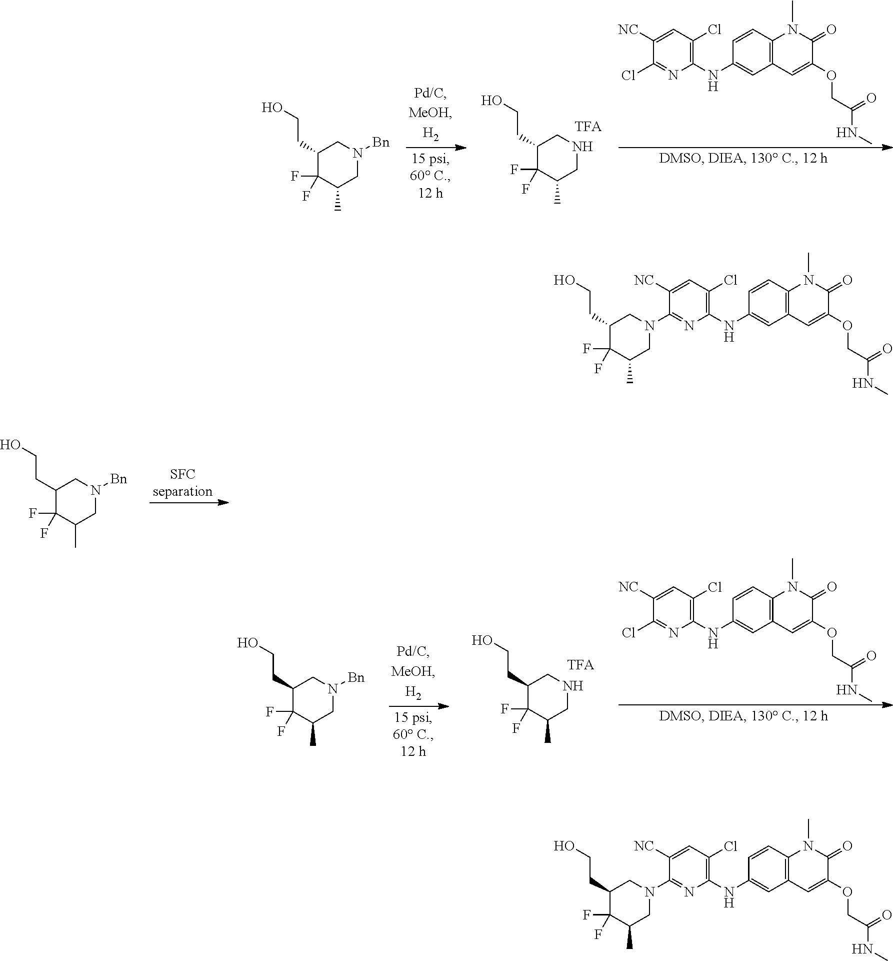

### 2-((3R,5S)-1-Benzyl-4,4-difluoro-5-methylpiperidin-3-yl)ethanol and 2-((3S,5R)-1-benzyl-4,4-difluoro-5-methylpiperidin-3-yl)ethanol

2-(1-Benzyl-4,4-difluoro-5-methyl-3-piperidyl)ethanol (1.2 g, 4.46 mmol) was separated by SFC (column: Phenomenex-Cellulose-2 (250 mm*30 mm, 5 μm); mobile phase: [n-Heptane-IPA (0.1% NH₃·H₂O)]; B %: 10%-10%, 12 min) to give 2-[(3R,5S)-1-benzyl-4,4-difluoro-5-methyl-3-piperidyl]ethanol as a colorless oil (540 mg, 1.98 mmol, 44% yield, 99% purity); ¹H NMR (400 MHz, DMSO-d₆) δ=7.33-7.25 (m, 5H), 4.50 (t, J=5.2 Hz, 1H), 3.58-3.50 (m, 1H), 3.47-3.33 (m, 3H), 2.95 (d, J=10 Hz, 2H), 2.72 (d, J=10 Hz, 2H), 2.20-2.04 (m, 2H), 1.91-1.81 (m, 3H), 1.25-1.19 (m, 1H), 0.87 (d, J=6.8 Hz, 3H); and 2-[(3S,5R)-1-benzyl-4,4-difluoro-5-methyl-3-piperidyl]ethanol as a colorless oil (510 mg, 1.79 mmol, 40% yield, 94% purity); ¹H NMR (400 MHz, DMSO-d₆) δ 7.35-7.25 (m, 5H), 4.50 (t, J=5.2 Hz, 1H), 3.58-3.50 (m, 1H), 3.47-3.33 (m, 3H), 2.95 (d, J=10 Hz, 2H), 2.72 (d, J=10 Hz, 2H), 2.20-2.04 (m, 2H), 1.91-1.81 (m, 3H), 1.25-1.19 (m, 1H), 0.87 (d, J=6.8 Hz, 3H).

### 2-((3R,5S)-4,4-Difluoro-5-methylpiperidin-3-yl)ethanol

To a solution of 2-[(3R, 5S)-1-benzyl-4,4-difluoro-5-methyl-3-piperidyl]ethanol (200 mg, 742.59 μmol) in MeOH (2 mL) was added TFA (338.69 mg, 2.97 mmol, 219.93 μL) and Pd/C (10%, 50 mg) under Ar. The mixture was stirred under H₂ (15 psi) at 60° C. for 12 hours. The reaction mixture was filtered and the filtrate was concentrated to give the title compound as a yellow oil (140 mg, crude, TFA). ¹H NMR (400 MHz, MeOD-d₄) δ 3.66-3.63 (m, 2H), 3.62-3.60 (m, 1H), 3.41 (d, J=12 Hz, 1H), 2.99-2.86 (m, 2H), 2.50-2.33 (m, 2H), 2.09-2.00 (m, 1H), 1.52-1.43 (m, 1H), 1.09 (d, J=6.8 Hz, 3H).

### 2-((6-((3-chloro-5-cyano-6-((3R, 5S)-4,4-difluoro-3-(2-hydroxyethyl)-5-methylpiperidin-1-yl) pyridin-2-yl) amino)-1-methyl-2-oxo-1, 2-dihydroquinolin-3-yl) oxy)-N-methylacetamide (62)

To a solution of 2-[[6-[(3,6-dichloro-5-cyano-2-pyridyl)amino]-1-methyl-2-oxo-3-quinolyl]oxy]-N-methyl-acetamide (40 mg, 92.54 μmol) and 2-[(3R,5S)-4,4-difluoro-5-methyl-3-piperidyl]ethanol (54.27 mg, 185.07 μmol, TFA) in DMSO (0.5 mL) was added DIPEA (47.84 mg, 370.15 μmol, 64.47 μL). The mixture was stirred at 100° C. for 3 hr. The mixture was concentrated under reduced pressure. The residue was purified by prep-HPLC (column: Phenomenex C18 80*40 mm*3 μm; mobile phase: [water(NH₄HCO₃)-ACN]; B %: 30%-60%, 8 min) to give the title compound as a white solid (8 mg, 13.63 μmol, 15% yield, 98% purity). ¹H NMR (400 MHz, DMSO-d₆) δ 9.19 (s, 1H), 8.06 (s, 1H), 8.00 (d, J=4.4 Hz, 1H), 7.83 (d, J=2.4 Hz, 1H), 7.71 (dd, J=2.4, 8.8 Hz, 1H), 7.39 (d, J=9.2 Hz, 1H), 7.30 (s, 1H), 4.61 (s, 2H), 4.60-4.57 (m, 1H), 4.42 (d, J=13.2 Hz, 1H), 4.17 (d, J=12.4 Hz, 1H), 3.74 (s, 3H), 2.90-2.76 (m, 2H), 2.73 (d, J=4.8 Hz, 3H), 2.23-2.01 (m, 3H), 1.89-1.80 (m, 1H), 1.36-1.27 (m, 2H), 0.89 (d, J=6.4 Hz, 3H).

### 2-((3S,5R)-4,4-Difluoro-5-methylpiperidin-3-yl)ethanol

To a solution of 2-[(3S,5R)-1-benzyl-4,4-difluoro-5-methyl-3-piperidyl]ethanol (200 mg, 742.59 μmol) in MeOH (2 mL) was added TFA (338.69 mg, 2.97 mmol, 219.93 μL) and Pd/C (10%, 50 mg) under Ar. The mixture was stirred under H₂ (15 psi) at 60° C. for 12 hours. The reaction mixture was filtered and the filtrate was concentrated to give the title compound as a yellow oil (120 mg, crude, TFA). ¹H NMR (400 MHz, MeOD-d₄) δ 4.43-4.34 (m, 2H), 3.71-3.68 (m, 1H), 3.45-3.42 (m, 1H), 3.08-2.93 (m, 2H), 2.57-2.43 (m, 2H), 2.08-2.00 (m, 1H), 1.51-1.42 (m, 1H), 1.07 (d, J=6.8 Hz, 3H).

### 2-((6-((3-chloro-5-cyano-6-((3S,5R)-4,4-difluoro-3-(2-hydroxyethyl)-5-methylpiperidin-1-yl)pyridin-2-yl)amino)-1-methyl-2-oxo-1,2-dihydroquinolin-3-yl)oxy)-N-methylacetamide (63)

To a solution of 2-[[6-[(3,6-dichloro-5-cyano-2-pyridyl)amino]-1-methyl-2-oxo-3-quinolyl]oxy]-N-methyl-acetamide (40 mg, 92.54 μmol) and 2-[(3S,5R)-4,4-difluoro-5-methyl-3-piperidyl]ethanol (54.27 mg, 185.07 μmol, TFA) in DMSO (0.5 mL) was added DIPEA (35.88 mg, 277.61 μmol, 48.35 μL). The mixture was stirred at 100° C. for 3 hr. The mixture was purified by prep-HPLC (column: Waters Xbridge BEH C18 100*30 mm*10 μm; mobile phase: [water(NH₄HCO₃)-ACN]; B %: 25%-55%, 8 min) to give the title compound as a white solid (11 mg, 18.78 μmol, 20% yield, 98% purity). ¹H NMR (400 MHz, DMSO-d₆) δ 9.13 (s, 1H), 8.00 (s, 1H), 7.92 (d, J=4.4 Hz, 1H), 7.77 (d, J=2.4 Hz, 1H), 7.64 (dd, J=2.4, 9.2 Hz, 1H), 7.48 (d, J=9.2 Hz, 1H), 7.24 (s, 1H), 4.55 (s, 2H), 4.52 (t, J=5.2 Hz, 1H), 4.36 (d, J=14 Hz, 1H), 4.11 (d, J=11.2 Hz, 1H), 3.68 (s, 3H), 2.83-2.72 (m, 2H), 2.66 (d, J=4.8 Hz, 3H), 2.16-1.94 (m, 3H), 1.82-1.74 (m, 1H), 1.28-1.23 (m, 2H), 0.82 (d, J=6.8 Hz, 3H).

The absolute configurations of compounds 62 & 63 were randomly assigned based on the aliphatic alcohol group and methyl group being in cis-conformation.

### Example 18: Synthesis of 2-((6-((3-chloro-5-cyano-6-((3R,4r,5S)-4-fluoro-3,5-dimethylpiperidin-1-yl)pyridin-2-yl)amino)-1-methyl-2-oxo-1,2-dihydroquinolin-3-yl)oxy)-N-methylacetamide (64)

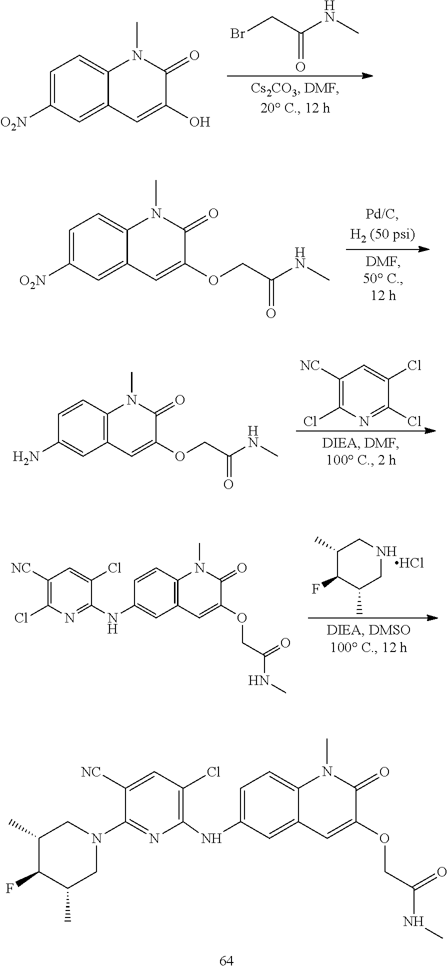

### N-Methyl-2-((I-methyl-6-nitro-2-oxo-1,2-dihydroquinolin-3-yl)oxy)acetamide

A mixture of 3-hydroxy-1-methyl-6-nitroquinolin-2(1H)-one (5 g, 22.71 mmol), 2-bromo-N-methyl-acetamide (4.14 g, 27.25 mmol), Cs₂CO₃ (14.80 g, 45.42 mmol) in DMF (100 mL) was stirred at 20° C. for 12 hr. The mixture was then poured into water (150 mL), the precipitated solid was filtered and washed with H₂O (200 mL) and MTBE (200 mL), then the solid was dried under reduced pressure to give the title compound as a yellow solid (3.3 g, 10.51 mmol, 46% yield, 93% purity).

### 2-((6-Amino-1-methyl-2-oxo-1,2-dihydroquinolin-3-yl)oxy)-N-methylacetamide

To a solution of N-methyl-2-((1-methyl-6-nitro-2-oxo-1,2-dihydroquinolin-3-yl)oxy)acetamide (3.3 g, 11.33 mmol) in DMF (60 mL) was added Pd/C (1 g, 11.33 mmol, 10% purity) under argon atmosphere. The mixture was stirred under H₂ (50 Psi) at 50° C. for 12 hr. The reaction mixture was filtered and the filtrate was concentrated under reduced pressure to give the title compound as a yellow solid (1.8 g, 6.13 mmol, 54% yield, 89% purity).

### 2-((6-((3,6-Dichloro-5-cyanopyridin-2-yl)amino)-1-methyl-2-oxo-1,2-dihydroquinolin-3-yl)oxy)-N-methylacetamide

A mixture of 2-((6-amino-1-methyl-2-oxo-1,2-dihydroquinolin-3-yl)oxy)-N-methylacetamide (1 g, 3.83 mmol), 2,5,6-trichloropyridine-3-carbonitrile (793.97 mg, 3.83 mmol), DIEA (989.32 mg, 7.65 mmol, 1.33 mL) in DMF (20 mL) was degassed and purged with N₂ for 3 times, and then the mixture was stirred at 100° C. for 12 hr under N₂ atmosphere. The mixture was cooled to 20° C. Water (10 mL) was added, forming a precipitate which was filtered and the filter cake was washed by EtOAc (20 mL) and dried in vacuo to give the title compound as a white solid (1.1 g, 1.35 mmol, 35% yield, 53% purity). ¹H NMR (400 MHz, DMSO-d₆) δ 9.64 (s, 1H), 8.38 (s, 1H), 7.94 (dd, J=6.60, 1.59 Hz, 1H), 7.74 (s, 1H), 7.66 (d, J=8.93 Hz, 1H), 7.53 (d, J=9.29 Hz, 1H), 7.22 (s, 1H), 4.58 (s, 2H), 3.69 (s, 3H), 2.66 (s, 3H).

### 2-((6-((3-Chloro-5-cyano-6-((3R,4r, 5S)-4-fluoro-3,5-dimethylpiperidin-1-yl)pyridin-2-yl)amino)-1-methyl-2-oxo-1,2-dihydroquinolin-3-yl)oxy)-N-methylacetamide (64)

To a solution of 2-((6-((3,6-dichloro-5-cyanopyridin-2-yl)amino)-1-methyl-2-oxo-1,2-dihydroquinolin-3-yl)oxy)-N-methylacetamide (50 mg, 115.67 μmol) and (3S,4R,5R)-4-fluoro-3,5-dimethyl-piperidine (38.78 mg, 231.34 μmol, HCl) in DMSO (1 mL) was added DIEA (74.75 mg, 578.36 μmol, 100.74 μL) under N₂. The mixture was stirred at 100° C. for 12 hr. The mixture was purified by prep-HPLC (column: Phenomenex C18 80*40 mm*3 μm; mobile phase: [water(NH₄HCO₃)-ACN]; B %: 35%-65%, 8 min) to give the title compound as a white solid (12 mg, 22.07 μmol, 19% yield, 97% purity). ¹H NMR (400 MHz, DMSO-d₆) δ 9.09 (s, 1H), 7.92-7.97 (m, 2H), 7.81 (d, J=2.20 Hz, 1H), 7.64 (dd, J=8.99, 2.38 Hz, 1H), 7.46 (d, J=9.05 Hz, 1H), 7.23 (s, 1H), 4.53 (s, 2H), 4.09-4.18 (m, 2H), 3.80-3.99 (m, 1H), 3.67 (s, 3H), 2.64-2.71 (m, 5H), 1.73 (dd, J=9.29, 4.65 Hz, 2H), 0.87 (d, J=6.48 Hz, 6H).

### Example 19: Synthesis of 2-((6-((3-chloro-5-cyano-6-((3R,4r,5S)-4-hydroxy-3,5-dimethylpiperidin-1-yl) pyridin-2-yl) amino)-1-methyl-2-oxo-1,2-dihydroquinolin-3-yl) oxy)-N-methyl acetamide (47) and 2-((6-((3-chloro-5-cyano-6-((3R,4s,5S)-4-hydroxy-3,5-dimethylpiperidin-1-yl) pyridin-2-yl) amino)-1-methyl-2-oxo-1, 2-dihydroquinolin-3-yl) oxy)-N methylacetamide (46)

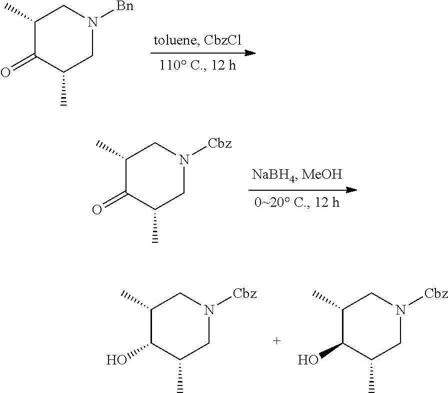 [SMILES: C[C@@H]1CN(Cc2ccccc2)C[C@H](C)C1=O ; C[C@@H]1CN(C(=O)OCc2ccccc2)C[C@H](C)C1=O ; C[C@@H]1CN(C(=O)OCc2ccccc2)C[C@H](C)[C@@H]1O ; C[C@@H]1CN(C(=O)OCc2ccccc2)C[C@H](C)[C@H]1O]

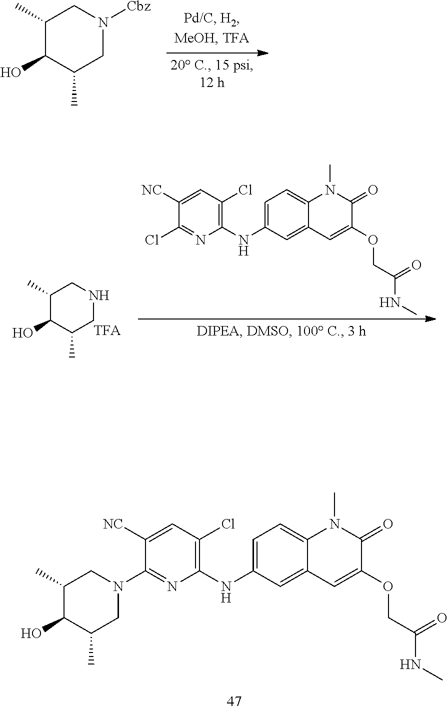

### (3R,5S)-Benzyl 3,5-dimethyl-4-oxopiperidine-1-carboxylate

To a solution of (3S,5R)-1-benzyl-3,5-dimethyl-piperidin-4-one (400 mg, 1.84 mmol) in toluene (4 mL) was added benzyl carbonochloridate (480.44 mg, 2.82 mmol, 400.37 μL). The mixture was stirred at 110° C. for 12 hr. The residue was purified by flash silica gel chromatography (Silica Flash Column, Eluent of 0˜30% Ethylacetate/Petroleum) to give the title compound as a white solid (300 mg, 1.04 mmol, 57% yield, 91% purity). ¹H NMR (400 MHz, CDCl₃) δ 7.41-7.32 (m, 5H), 5.20 (d, J=4.4 Hz, 2H), 4.50-4.41 (m, 2H), 2.73-2.58 (m, 4H), 1.03 (d, J=6.4 Hz, 6H).

### Benzyl (3S,4R,5R)-4-hydroxy-3,5-dimethyl-piperidine-1-carboxylate

To a solution of benzyl (3S,5R)-3,5-dimethyl-4-oxo-piperidine-1-carboxylate (4.1 g, 15.69 mmol) in MeOH (45 mL) was added NaBH₄ (712.30 mg, 18.83 mmol) at 0° C. The mixture was stirred at 20° C. for 12 hr. The reaction mixture was quenched by addition 1N HCl (10 mL) at 0° C., then the mixture was concentrated under reduced pressure. The residue was dissolved in ethyl acetate (20 mL) and washed with water (10 mL×3), the combined organic phase was dried with anhydrous Na₂SO₄, filtered and concentrated under reduced pressure. The residue was purified by prep-HPLC (column: Agela DuraShell C18 250*80 mm*10 μm; mobile phase: [water (NH₄HCO₃)-ACN]; B %: 25%-50%, 20 min) to give benzyl (3S,4S,5R)-4-hydroxy-3,5-dimethyl-piperidine-1-carboxylate as a colorless oil (1.4 g, 5.26 mmol, 34% yield, 99% purity) as a colorless oil; ¹H NMR (400 MHz, MeOD-d₄) δ 7.36-7.31 (m, 5H), 5.11 (s, 2H), 4.09-4.04 (m, 2H), 2.74 (t, J=9.6 Hz, 1H), 2.52-2.46 (m, 2H), 1.50-1.39 (m, 2H), 0.99 (d, J=6.0 Hz, 6H); and benzyl (3S,4R,5R)-4-hydroxy-3,5-dimethyl-piperidine-1-carboxylate as a colorless oil (0.9 g, 3.35 mmol, 21% yield, 98% purity); ¹H NMR (400 MHz, MeOD-d₄) δ=7.36-7.29 (m, 5H), 5.10 (s, 2H), 3.77 (dd, J=4.4, 13.2 Hz, 2H), 3.54 (s, 1H), 2.77-2.72 (m, 2H), 1.70-1.60 (m, 2H), 0.94 (d, J=6.0 Hz, 6H).

### (3R,4R,5S)-3,5-Dimethylpiperidin-4-ol

To a solution of benzyl (3S,5R)-4-hydroxy-3,5-dimethyl-piperidine-1-carboxylate (200 mg, 759.50 μmol) in MeOH (2 mL) was added TFA (346.39 mg, 3.04 mmol, 224.93 μL) and Pd/C (50 mg, 10% purity) under Ar atmosphere. The mixture was stirred under H₂ (15 Psi) at 20° C. for 12 hr. The mixture was filtered and the filtrate was concentrated under reduced pressure to give the title compound as a yellow oil (150 mg, crude, TFA). ¹H NMR (400 MHz, DMSO-d₆) δ 4.33 (s, 2H), 3.18-3.16 (m, 2H), 2.76-2.71 (m, 1H), 2.62-2.53 (m, 2H), 1.68-1.57 (m, 2H), 0.92 (d, J=6.8 Hz, 6H).

### 2-((6-((3-Chloro-5-cyano-6-((3R,4r,5S)-4-hydroxy-3,5-dimethylpiperidin-1-yl)pyridin-2-yl)amino)-1-methyl-2-oxo-1,2-dihydroquinolin-3-yl)oxy)-N-methylacetamide (47)

To a solution of 2-[[6-[(3,6-dichloro-5-cyano-2-pyridyl)amino]-1-methyl-2-oxo-3-quinolyl]oxy]-N-methyl-acetamide (50 mg, 115.67 μmol) and (3S,4R,5R)-3,5-dimethylpiperidin-4-ol (45.00 mg, 185.02 μmol, TFA) in DMSO (1 mL) was added DIPEA (74.75 mg, 578.36 μmol, 100.74 μL). The mixture was stirred at 100° C. for 3 hr. Water (2 mL) was added to the reaction mixture, forming a precipitate which was filtered and the filter cake was washed with water (5 mL×2), EtOAc (5 mL×2), the filter cake was dried under reduced pressure to give the title compound as a white solid (30 mg, 56.21 μmol, 49% yield, 98% purity). ¹H NMR (400 MHz, DMSO-d₆) δ 9.04 (s, 1H), 7.95-7.92 (m, 2H), 7.83 (s, 1H), 7.66-7.64 (m, 1H), 7.46-7.44 (m, 1H), 7.21 (s, 1H), 4.61 (d, J=4.8 Hz, 1H), 4.53 (s, 2H), 4.14 (d, J=11.2 Hz, 2H), 3.67 (s, 3H), 2.66-2.61 (m, 6H), 1.42 (s, 2H), 0.83 (s, 6H).

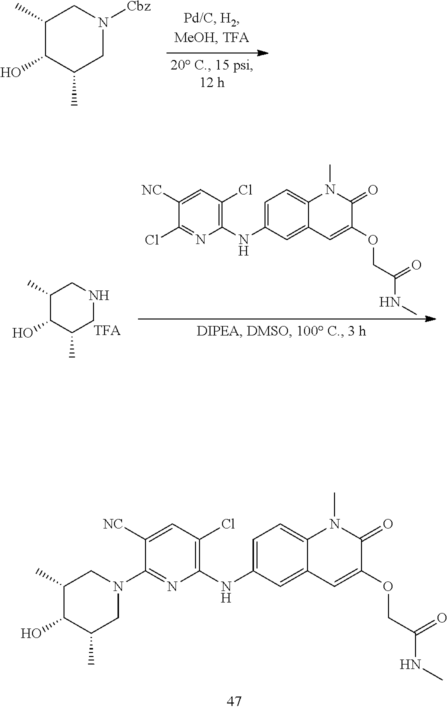

### (3R,4S,5S)-3,5-dimethylpiperidin-4-ol

To a solution of benzyl (3S,4S,5R)-4-hydroxy-3,5-dimethyl-piperidine-1-carboxylate (200 mg, 759.50 μmol) in MeOH (2 mL) was added TFA (346.39 mg, 3.04 mmol, 224.93 μL) and Pd/C (50 mg, 10% purity) under Ar atmosphere. The mixture was stirred under H₂ (15 Psi) at 20° C. for 12 hr. The mixture was filtered and the filtrate was concentrated under reduced pressure to give the title compound as a yellow oil (150 mg, crude, TFA). ¹H NMR (400 MHz, MeOD-d₄) δ 3.05 (s, 1H), 2.74-2.70 (m, 2H), 2.61-2.55 (m, 2H), 1.69-1.65 (m, 2H), 0.74 (d, J=6.8 Hz, 6H).

### 2-((6-((3-Chloro-5-cyano-6-((3R,4s,5S)-4-hydroxy-3,5-dimethylpiperidin-1-yl)pyridin-2-yl)amino)-1-methyl-2-oxo-1,2-dihydroquinolin-3-yl)oxy)-N-methylacetamide (46)

To a solution of 2-[[6-[(3,6-dichloro-5-cyano-2-pyridyl)amino]-1-methyl-2-oxo-3 quinolyl]oxy]-N-methyl-acetamide (25 mg, 57.84 μmol) and (3S,4S,5R)-3,5-dimethylpiperidin-4-ol (21.10 mg, 86.75 μmol, TFA) in DMSO (0.5 mL) was added DIPEA (37.37 mg, 289.18 μmol, 50.37 μL). The mixture was stirred at 100° C. for 3 hr. Water (2 mL) was added to the reaction mixture, forming a precipitate which was filtered and the filter cake was washed with water (5 mL×2), EtOAc (5 mL×2), the filter cake was dried under reduced pressure to give the title compound as a white solid (9.4 mg, 17.52 μmol, 30% yield, 98% purity). ¹H NMR (400 MHz, DMSO-d₆) δ 9.00 (s, 1H), 7.95 (s, 1H), 7.89 (s, 1H), 7.83 (s, 1H), 7.67-7.64 (m, 1H), 7.46-7.44 (m, 1H), 7.20 (s, 1H), 4.57-4.53 (m, 3H), 3.87 (d, J=11.2 Hz, 2H), 3.67 (s, 3H), 3.43 (s, 1H), 2.83 (t, J=12.3 Hz, 2H), 2.67 (m, 3H), 1.66 (s, 2H), 0.78 (d, J=6.4 Hz, 6H).

### Example 20: Synthesis of 2-((6-((3-chloro-5-cyano-6-((3R,4S,5S)-4-fluoro-3, 5-dimethylpiperidin-1-yl) pyridin-2-yl)amino)-1-methyl-2-oxo-1,2-dihydroquinolin-3-yl)oxy)-N-methylacetamide (45)

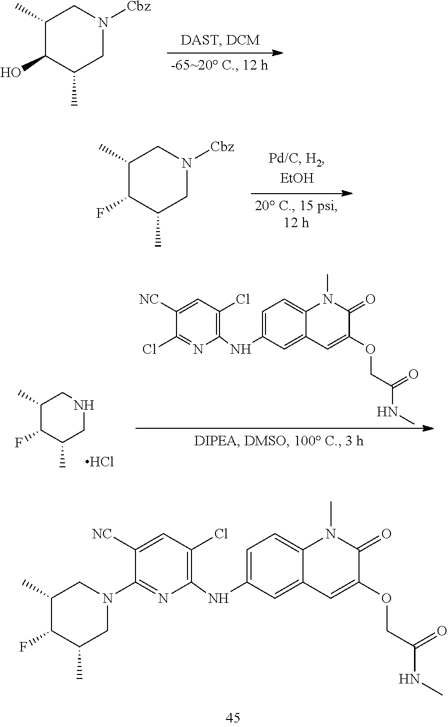

### (3R,4S,5S)-Benzyl 4-fluoro-3,5-dimethylpiperidine-1-carboxylate

To a solution of benzyl (3S,4R,5R)-4-hydroxy-3,5-dimethyl-piperidine-1-carboxylate (300.00 mg, 1.14 mmol) in DCM (3 mL) was added DAST (367.27 mg, 2.28 mmol, 301.04 μL) at −65° C. Then the mixture was stirred at 20° C. for 12 hr. Sat. NaHCO₃ was added to the mixture until pH 8-9 was reached and then extracted with ethyl acetate (10 mL×2). The combined organic phase was dried with anhydrous Na₂SO₄, filtered and concentrated under reduced pressure. The residue was purified by flash silica gel chromatography (Silica Flash Column, Eluent of 0˜10% Ethylacetate/Petroleum) to give a semi-purified product. The product was further purified by p-TLC (Eluent of 25% Ethylacetate/Petroleum) to give the title compound as a yellow oil (50 mg, 150.76 μmol, 13% yield, 80% purity). ¹H NMR (400 MHz, MeOD-d₄) δ 7.40-7.28 (m, 5H), 5.11 (s, 2H), 4.44 (d, J=10 Hz, 1H), 4.10-4.06 (m, 1H), 3.90 (dd, J=4.4, 13.2 Hz, 1H), 2.71-2.53 (m, 2H), 1.82-1.68 (m, 2H), 1.00-0.98 (m, 6H).

### (3R,4S,5S)-4-Fluoro-3,5-dimethylpiperidine

To a solution of benzyl (3S,4S,5R)-4-fluoro-3,5-dimethyl-piperidine-1-carboxylate (40 mg, 150.76 μmol) in EtOH (1 mL) was added Pd/C (10%, 8 mg) under Ar. The mixture was stirred under H₂ (15 psi) at 20° C. for 12 hours. The reaction mixture was filtered and the filtrate was added HCl/dioxane (2 mL) and stirred at 20° C. for 0.5 hr. The solution was concentrated under reduced pressure to give the title compound a white oil (20 mg, 57.26 μmol, 38% yield, 48% purity, HCl). ¹H NMR (400 MHz, MeOD-d₄) δ 4.61-4.49 (m, 1H), 3.37-3.33 (m, 2H), 3.24-3.16 (m, 2H), 2.32-2.13 (m, 2H), 1.11-1.10 (m, 6H).

### 2-((6-((3-Chloro-5-cyano-6-((3R,4S,5S)-4-fluoro-3,5-dimethylpiperidin-1-yl)pyridin-2-yl)amino)-1-methyl-2-oxo-1,2-dihydroquinolin-3-yl)oxy)-N-methylacetamide (45)

To a solution of 2-[[6-[(3,6-dichloro-5-cyano-2-pyridyl)amino]-1-methyl-2-oxo-3-quinolyl]oxy]-N-methyl-acetamide (30 mg, 69.40 μmol) and (3S,4S,5R)-4-fluoro-3,5-dimethyl-piperidine (18.62 mg, 111.04 μmol, HCl) in DMF (0.5 mL) was added DIPEA (26.91 mg, 208.20 μmol, 36.27 μL). The mixture was stirred at 100° C. for 3 hr. The mixture was purified by prep-HPLC (column: Phenomenex C18 75*30 mm*3 μm; mobile phase: [water(NH₄HCO₃)-ACN]; B %: 35%-65%, 8 min) to give the title compound as a white solid (4.5 mg, 8.38 μmol, 12% yield, 98% purity). ¹H NMR (400 MHz, DMSO-d₆) δ 9.07 (s, 1H), 7.95 (s, 2H), 7.82 (d, J=2.0 Hz, 1H), 7.64 (dd, J=2.0, 9.2 Hz, 1H), 7.46 (d, J=9.2 Hz, 1H), 7.22 (s, 1H), 4.56-4.43 (m, 3H), 3.98 (br dd, J=3.6, 13.2 Hz, 2H), 3.67 (s, 3H), 2.78 (t, J=12.4 Hz, 2H), 2.66 (d, J=4.4 Hz, 3H), 1.89-1.76 (m, 2H), 0.84 (d, J=6.8 Hz, 6H).

### Example 21: Synthesis of 2-((6-((6-((3S,5R)-3-amino-4,4-difluoro-5-methylpiperidin-1-yl)-3-chloro-5-cyanopyridin-2-yl)amino)-1-methyl-2-oxo-1,2-dihydroquinolin-3-yl)oxy)-N-methylacetamide (43) and 2-((6-((6-((3R,5S)-3-amino-4,4-difluoro-5-methylpiperidin-1-yl)-3-chloro-5-cyanopyridin-2-yl)amino)-1-methyl-2-oxo-1,2-dihydroquinolin-3-yl)oxy)-N-methylacetamide (44)

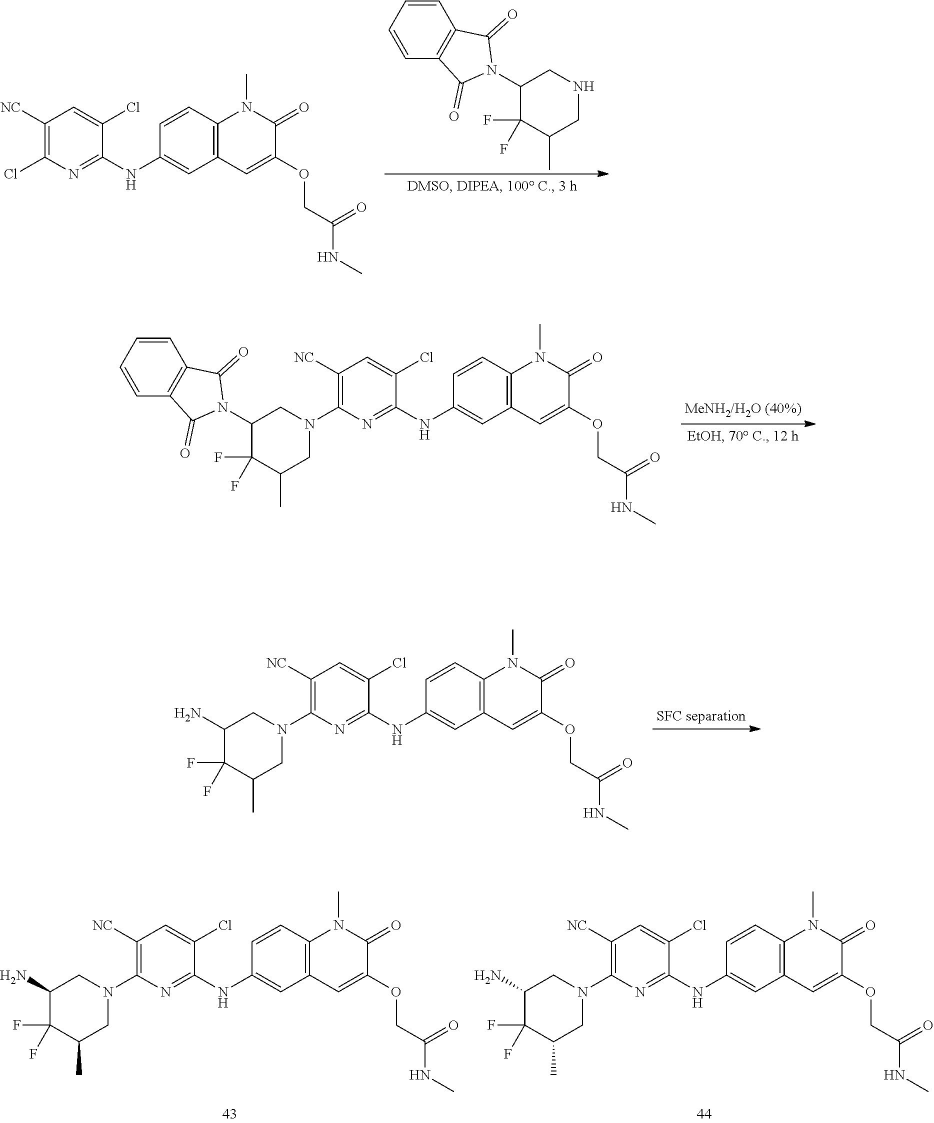

### 2-((6-((3-Chloro-5-cyano-6-(3-(1,3-dioxoisoindolin-2-yl)-4,4-difluoro-5-methylpiperidin-1-yl)pyridin-2-yl)amino)-1-methyl-2-oxo-1,2-dihydroquinolin-3-yl)oxy)-N-methylacetamide

To a solution of 2-(4,4-difluoro-5-methyl-3-piperidyl)isoindoline-1,3-dione (250.00 mg, 892.00 μmol) and 2-[[6-[(3,6-dichloro-5-cyano-2-pyridyl)amino]-1-methyl-2-oxo-3-quinolyl]oxy]-N-methyl-acetamide (296.60 mg, 686.15 μmol) in DMSO (3 mL) was added DIPEA (177.36 mg, 1.37 mmol, 239.03 μL), and the reaction mixture was stirred at 100° C. for 3 hr. The reaction mixture was treated with water (10 mL), forming a precipitate which was filtered and the filter cake was washed, collected and dried in vacuo to give the title compound as a yellow solid (450 mg, crude).

### 2-((6-((6-(3-Amino-4,4-difluoro-5-methylpiperidin-1-yl)-3-chloro-5-cyanopyridin-2-yl)amino)-1-methyl-2-oxo-1,2-dihydroquinolin-3-yl)oxy)-N-methylacetamide

To a solution of 2-[[6-[[3-chloro-5-cyano-6-[3-(1,3-dioxoisoindolin-2-yl)-4,4-difluoro-5-methyl-1-piperidyl]-2-pyridyl]amino]-1-methyl-2-oxo-3-quinolyl]oxy]-N-methyl-acetamide (400 mg, 591.66 μmol) in EtOH (20 mL) was added MeNH₂ (86.13 mg, 1.11 mmol, 20 mL, 40% purity in H₂O), and the reaction mixture was stirred at 70° C. for 12 hr. The reaction mixture (combined with another batch at 50 mg scale) was concentrated in vacuo and purified by prep-HPLC (column: Phenomenex C18 80*40 mm*3 μm; mobile phase: [water(NH4HCO3)-ACN]; B %: 25%-45%, 8 min) to give the title compound as a white solid (68 mg, 98.90% purity).

### 2-((6-((6-((3S,5R)-3-Amino-4,4-difluoro-5-methylpiperidin-1-yl)-3-chloro-5-cyanopyridin-2-yl)amino)-1-methyl-2-oxo-1,2-dihydroquinolin-3-yl)oxy)-N-methylacetamide (43) and 2-((6-((6-((3R,5S)-3-amino-4,4-difluoro-5-methylpiperidin-1-yl)-3-chloro-5-cyanopyridin-2-yl)amino)-1-methyl-2-oxo-1,2-dihydroquinolin-3-yl)oxy)-N-methylacetamide (44)

Racemate (68 mg) was separated by SFC (column: DAICEL CHIRALPAK AD (250 mm*30 mm, 10 μm); mobile phase: [0.1% NH3H2O ETOH]; B %: 50%-50%, 15 min) and further purified by prep-HPLC (column: C18-1 150*30 mm*5 μm; mobile phase: [water(NH₄HCO₃)-ACN]; B %: 20%-60%, 20 min) to give 2-[[6-[[6-[(3R,5S)-3-amino-4,4-difluoro-5-methyl-1-piperidyl]-3-chloro-5-cyano-2-pyridyl]amino]-1-methyl-2-oxo-3-quinolyl]oxy]-N-methyl-acetamide as a white solid (8.5 mg, 15.38 μmol, 12% yield, 99% purity); ¹H NMR (400 MHz, DMSO-d₆) δ 9.08 (s, 1H), 8.00 (s, 1H), 7.94 (m, 2H), 7.65 (dd, J=2.4, 9.2 Hz, 1H), 7.48 (d, J=9.2 Hz, 1H), 7.36 (s, 1H), 4.55 (s, 2H), 4.31-4.22 (d, J=9.2 Hz, 1H), 4.16-4.06 (d, J=11.6 Hz, 1H), 3.67 (s, 3H), 3.05-2.91 (m, 1H), 2.87-2.74 (m, 2H), 2.66 (d, J=4.4 Hz, 3H), 2.16-2.01 (m, 1H), 1.94-1.61 (m, 2H), 0.85 (d, J=6.4 Hz, 3H); and 2-[[6-[[6-[(3S,5R)-3-amino-4,4-difluoro-5-methyl-1-piperidyl]-3-chloro-5-cyano-2-pyridyl]amino]-1-methyl-2-oxo-3-quinolyl]oxy]-N-methyl-acetamide as a white solid (13 mg, 23.25 μmol, 19% yield, 98% purity); ¹H NMR (400 MHz, DMSO-d₆) δ 9.08 (s, 1H), 8.00 (s, 1H), 7.94 (m, 2H), 7.65 (dd, J=2.4, 9.2 Hz, 1H), 7.48 (d, J=9.2 Hz, 1H), 7.36 (s, 1H), 4.55 (s, 2H), 4.26 (d, J=12.8 Hz, 1H), 4.11 (dd, J=2.4, 14.0 Hz, 1H), 3.67 (s, 3H), 3.05-2.91 (m, 1H), 2.86-2.74 (m, 2H), 2.66 (d, J=4.4 Hz, 3H), 2.16-2.00 (m, 1H), 1.76 (s, 2H), 0.85 (d, J=6.8 Hz, 3H).

The absolute configurations of compounds 43 & 44 were randomly assigned based on the amino group and methyl group being in cis-conformation.

### Example 22: Synthesis of 2-((1-(azetidin-3-ylmethyl)-6-((3-chloro-5-cyano-6-((3R,5S)-4,4-difluoro-3,5-dimethylpiperidin-1-yl)pyridin-2-yl)amino)-2-oxo-1,2-dihydroquinolin-3-yl)oxy)-N-methylacetamide (65)

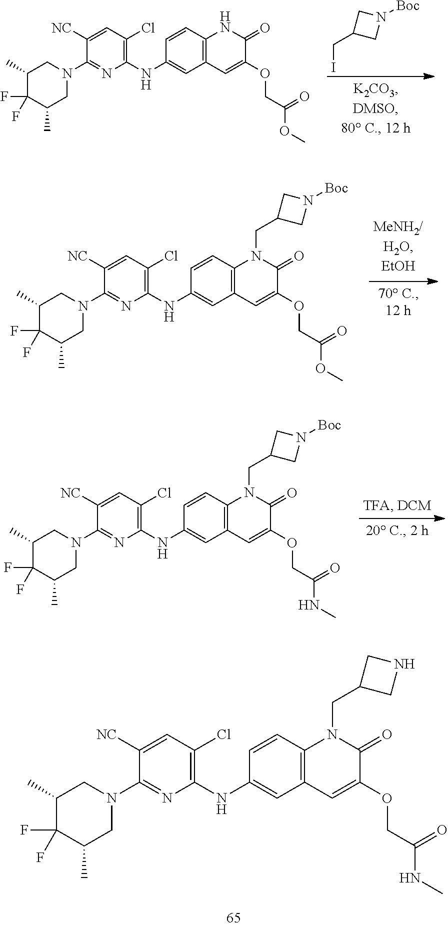 [SMILES: COC(=O)COc1cc2cc(Nc3nc(N4C[C@@H](C)C(F)(F)[C@@H](C)C4)c(C#N)cc3Cl)ccc2[nH]c1=O ; CC(C)(C)OC(=O)N1CC(CI)C1 ; COC(=O)COc1cc2cc(Nc3nc(N4C[C@@H](C)C(F)(F)[C@@H](C)C4)c(C#N)cc3Cl)ccc2n(CC2CN(C(=O)OC(C)(C)C)C2)c1=O ; CNC(=O)COc1cc2cc(Nc3nc(N4C[C@@H](C)C(F)(F)[C@@H](C)C4)c(C#N)cc3Cl)ccc2n(CC2CN(C(=O)OC(C)(C)C)C2)c1=O ; CNC(=O)COc1cc2cc(Nc3nc(N4C[C@@H](C)C(F)(F)[C@@H](C)C4)c(C#N)cc3Cl)ccc2n(CC2CNC2)c1=O]

### tert-Butyl 3-((6-((3-chloro-5-cyano-6-((3R,5S)-4,4-difluoro-3,5-dimethylpiperidin-1-yl)pyridin-2-yl)amino)-3-(2-methoxy-2-oxoethoxy)-2-oxoquinolin-1(2H)-yl)methyl)azetidine-1-carboxylate

A mixture of methyl 2-[[6-[[3-chloro-5-cyano-6-[(3S,5R)-4,4-difluoro-3,5-dimethyl-1-piperidyl]-2-pyridyl]amino]-2-oxo-1H-quinolin-3-yl]oxy]acetate (100 mg, 187.99 μmol), tert-butyl 3-(iodomethyl)azetidine-1-carboxylate (55.86 mg, 187.99 μmol, 5.41 μL), K₂CO₃ (51.96 mg, 375.98 μmol), in DMSO (1.5 mL) was stirred at 80° C. for 12 hr. The mixture (combined with another batch at same scale) was cooled to 20° C. and water (2 mL) was added, forming a precipitate which was filtered and washed with H₂O (50 mL) and ethyl acetate (20 mL), then the solid was dried under reduced pressure to give the title compound as a brown solid (200 mg, crude). LCMS: [M+H]⁺=701.3.

### tert-Butyl 3-((6-((3-chloro-5-cyano-6-((3R,5S)-4,4-difluoro-3,5-dimethylpiperidin-1-yl)pyridin-2-yl)amino)-3-(2-(methylamino)-2-oxoethoxy)-2-oxoquinolin-1(2H)-yl)methyl)azetidine-1-carboxylate

A mixture of tert-butyl 3-[[6-[[3-chloro-5-cyano-6-[(3S,5R)-4,4-difluoro-3,5-dimethyl-1-piperidyl]-2-pyridyl]amino]-3-(2-methoxy-2-oxo-ethoxy)-2-oxo-1-quinolyl]methyl]azetidine-1-carboxylate (200 mg, 285.24 μmol), MeNH₂/H₂O (10 mL, 40% purity) in EtOH (10 mL) was stirred at 70° C. for 12 hr. The mixture was concentrated to give the title compound as a yellow solid (250 mg, crude), which was used without further purification. LCMS: [M+H]⁺=700.2.

### 2-((1-(Azetidin-3-ylmethyl)-6-((3-chloro-5-cyano-6-((3R,5S)-4,4-difluoro-3,5-dimethylpiperidin-1-yl)pyridin-2-yl)amino)-2-oxo-1,2-dihydroquinolin-3-yl)oxy)-N-methylacetamide (65)

A mixture of tert-butyl 3-[[6-[[3-chloro-5-cyano-6-[(3S,5R)-4,4-difluoro-3,5-dimethyl-1-piperidyl]-2-pyridyl]amino]-3-[2-(methylamino)-2-oxo-ethoxy]-2-oxo-1-quinolyl]methyl]azetidine-1-carboxylate (250 mg, 357.05 μmol) in DCM (2 mL) and TFA (1 mL) was stirred at 20° C. for 1 hr. The mixture was concentrated in vacuo and purified by p-HPLC (column: Waters Xbridge BEH C18 100*25 mm*5 μm; mobile phase: [water(NH₄HCO₃)-ACN]; B %: 30%-60%, 10 min) to give the title compound as a white solid (25 mg, 37.34 μmol, 10% yield, 90% purity). ¹H NMR (400 MHz, DMSO-d₆) δ 9.01-9.21 (m, 1H), 8.00 (s, 1H), 7.95 (br d, J=4.63 Hz, 1H), 7.75 (d, J=2.13 Hz, 1H), 7.61 (dd, J=8.94, 1.81 Hz, 1H), 7.51 (br d, J=9.01 Hz, 1H), 7.22 (s, 1H), 4.49-4.61 (m, 4H), 4.12 (br d, J=12.51 Hz, 2H), 3.41 (br d, J=5.00 Hz, 4H), 2.99-3.10 (m, 1H), 2.78 (br t, J=13.01 Hz, 2H), 2.67 (d, J=4.50 Hz, 4H), 1.96-2.17 (m, 2H), 0.83 (d, J=6.63 Hz, 6H).

### Example 23: Synthesis of 2-((3R,5S)-3-amino-5-methylpiperidin-1-yl)-5-chloro-6-(((S)-2-cyclopropyl-3,3-difluoro-7-methyl-6-oxo-1,2,3,4,6,7-hexahydro-[1,4]oxazepino[2,3-c]quinolin-10-yl)amino)nicotinonitrile (24) and 2-((3S,5R)-3-amino-5-methylpiperidin-1-yl)-5-chloro-6-(((S)-2-cyclopropyl-3,3-difluoro-7-methyl-6-oxo-1,2,3,4,6,7-hexahydro-[1,4]oxazepino[2,3-c]quinolin-10-yl)amino)nicotinonitrile (25)

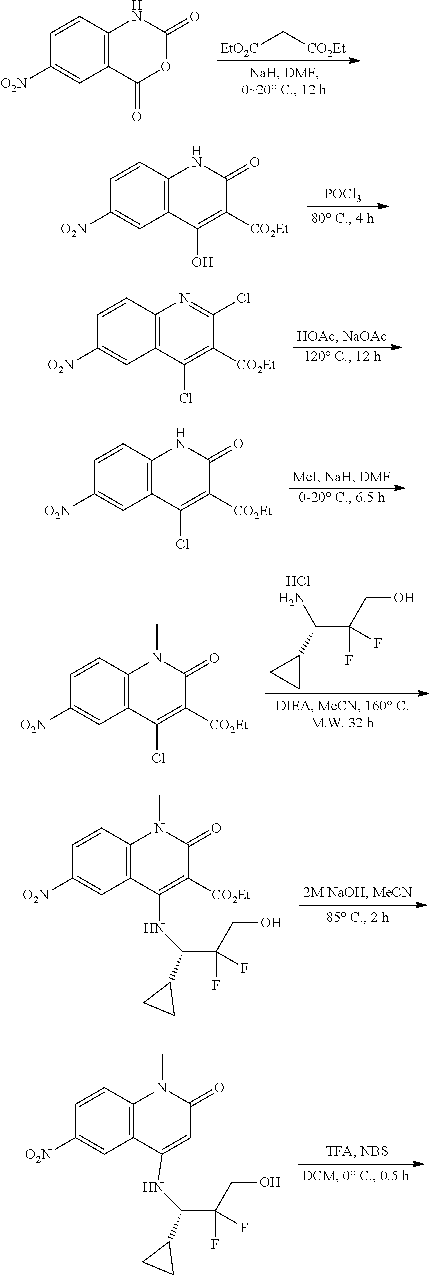

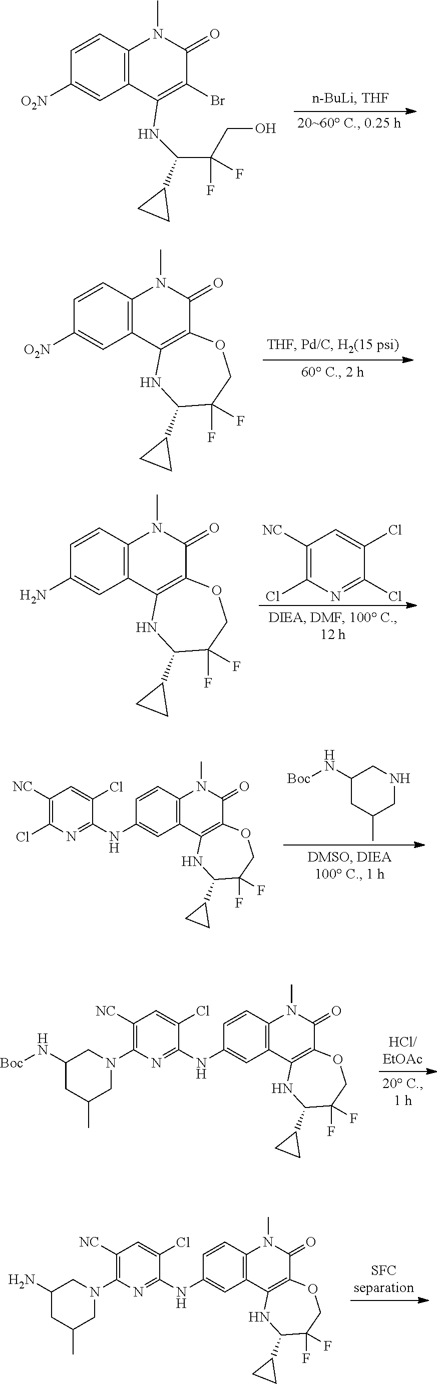

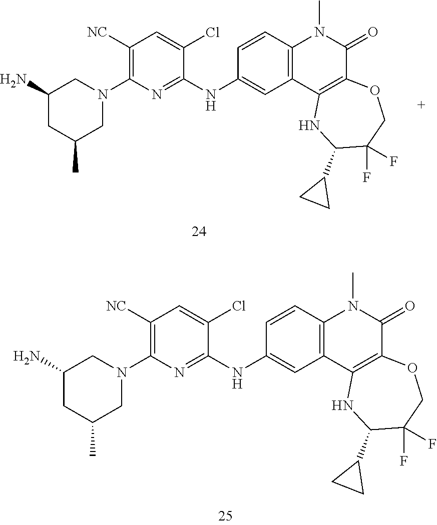 [SMILES: C[C@H]1C[C@@H](N)CN(c2nc(Nc3ccc4c(c3)c3c(c(=O)n4C)OCC(F)(F)[C@H](C4CC4)N3)c(Cl)cc2C#N)C1 ; C[C@@H]1C[C@H](N)CN(c2nc(Nc3ccc4c(c3)c3c(c(=O)n4C)OCC(F)(F)[C@H](C4CC4)N3)c(Cl)cc2C#N)C1]

### Ethyl 4-hydroxy-6-nitro-2-oxo-1,2-dihydroquinoline-3-carboxylate

To a solution of 6-nitro-1H-benzo[d][1,3]oxazine-2,4-dione (20 g, 96.09 mmol) and diethyl malonate (23.09 g, 144.14 mmol, 21.78 mL) in DMF (500 mL) was added NaH (7.69 g, 192.19 mmol, 60% purity) at 0° C. The reaction mixture was allowed to warm to 20° C. and stirred for 12 hr. The reaction mixture was cooled to 0° C. and water (1000 mL) was added. The aqueous mixture was neutralized to pH=7 with 1N HCl aq and the resulting mixture was stirred at 20° C. for 30 min. The resulting yellow precipitate was filtered and washed with water (1 L). The yellow precipitate was dried under reduced pressure to give the title compound as a yellow solid (46 g, 160.49 mmol, 84% yield, 97% purity). ¹H NMR (400 MHz, DMSO-d₆) δ 12.08 (s, 1H), 8.73 (d, J=1.9 Hz, 1H), 8.47-8.36 (m, 1H), 7.95 (s, 1H), 7.41 (d, J=9.0 Hz, 1H), 4.32 (q, J=7.1 Hz, 2H), 2.89 (s, 2H), 2.73 (s, 3H), 1.30 (t, J=7.0 Hz, 3H).

### Ethyl 2,4-dichloro-6-nitroquinoline-3-carboxylate

A mixture of ethyl 4-hydroxy-6-nitro-2-oxo-1,2-dihydroquinoline-3-carboxylate (46 g, 165.34 mmol) in POCl₃ (450 mL), then the mixture was stirred at 80° C. for 4 hr under N₂ atmosphere. The reaction mixture was concentrated under reduced pressure to give a residue which was triturated with MTBE (100 mL) at 25° C. for 30 min to give the title compound as a yellow solid (37 g, 114.07 mmol, 69% yield, 97% purity). ¹H NMR (400 MHz, DMSO-d₆) δ 8.99 (d, J=2.5 Hz, 1H), 8.67 (dd, J=2.5, 9.2 Hz, 1H), 8.31 (d, J=9.2 Hz, 1H), 4.53 (q, J=7.0 Hz, 2H), 1.39 (t, J=7.1 Hz, 3H).

### Ethyl 4-chloro-6-nitro-2-oxo-1,2-dihydroquinoline-3-carboxylate

A mixture of ethyl 2,4-dichloro-6-nitroquinoline-3-carboxylate (35 g, 111.07 mmol), NaOAc (10.02 g, 122.18 mmol) in AcOH (350 mL), then the mixture was stirred at 120° C. for 12 hr under N₂ atmosphere. Water (500 mL) was added to the reaction mixture (combined with other batch at 2 g scale), forming a precipitate which was filtered and washed with H₂O (1 L), then the solid was dried under reduced pressure to give the title compound as a yellow solid (25 g, 92% purity). ¹H NMR (400 MHz, DMSO-d₆) δ 12.99 (s, 1H), 8.66 (d, J=2.5 Hz, 1H), 8.49 (dd, J=2.5, 9.0 Hz, 1H), 7.55 (d, J=9.0 Hz, 1H), 4.38 (q, J=7.0 Hz, 2H), 1.32 (t, J=7.1 Hz, 3H).

### Ethyl 4-chloro-1-methyl-6-nitro-2-oxo-1,2-dihydroquinoline-3-carboxylate

To a mixture of ethyl 4-chloro-6-nitro-2-oxo-1,2-dihydroquinoline-3-carboxylate (15 g, 50.56 mmol) in DMF (150 mL) was added NaH (2.83 g, 70.79 mmol, 60% purity) at 0° C. and the mixture was stirred at 0° C. for 0.5 hr, then iodomethane (35.88 g, 252.81 mmol, 15.74 mL) was added to the mixture and the mixture was stirred at 20° C. for 6 hr. Water (50 mL) was added to the mixture, forming a precipitate which was filtered and washed with H₂O (100 mL), then the solid was dried under reduced pressure. The residue was purified by column chromatography (SiO₂, Petroleum ether/Ethyl acetate=30/1 to 2/1) to give a semi-purified product which was further purified by reversed-phase MPLC (neutral condition: Column: 330 g Agela C18; Mobile phase: [water-ACN]; Gradient B %: 25-50% 20 min; 50-50% 20 min) to give the title compound as a yellow solid (9.3 g, 29.68 mmol, 59% yield, 99% purity). ¹H NMR (400 MHz, DMSO-d₆) δ 8.71 (d, J=2.6 Hz, 1H), 8.54 (dd, J=2.6, 9.3 Hz, 1H), 7.87 (d, J=9.4 Hz, 1H), 4.39 (q, J=7.1 Hz, 2H), 3.69 (s, 3H), 1.32 (t, J=7.1 Hz, 3H).

### (S)-Ethyl 4-((1-cyclopropyl-2,2-difluoro-3-hydroxypropyl)amino)-1-methyl-6-nitro-2-oxo-1,2-dihydroquinoline-3-carboxylate

To a solution of ethyl 4-chloro-1-methyl-6-nitro-2-oxo-1,2-dihydroquinoline-3-carboxylate (500 mg, 1.61 mmol) and (S)-3-amino-3-cyclopropyl-2,2-difluoropropan-1-ol hydrochloride (413.65 mg, 2.20 mmol) in MeCN (5 mL) was added DIEA (519.40 mg, 4.02 mmol, 700.00 μL) under N₂. The mixture was stirred at 160° C. for 32 hr under microwave. The mixture was concentrated in vacuo to give the title compound as a brown solid (680 mg, 799.28 μmol, 50% yield, 50% purity). LCMS: [M+H]⁺=426.1.

### (S)-4-((1-Cyclopropyl-2,2-difluoro-3-hydroxypropyl)amino)-1-methyl-6-nitroquinolin-2(1H)-one

To a solution of (S)-ethyl 4-((1-cyclopropyl-2,2-difluoro-3-hydroxypropyl)amino)-1-methyl-6-nitro-2-oxo-1,2-dihydroquinoline-3-carboxylate (680 mg, 1.60 mmol) in MeCN (5 mL) was added NaOH (2 M, 25.00 mL). The mixture was stirred at 85° C. for 2 hr. Water (20 mL) was then added, followed by the addition of 3N HCl until pH=6. A precipitate formed which was filtered and filter cake was dried with reduce pressure to give the title compound as a yellow solid (400 mg, 520.77 μmol, 33% yield, 46% purity). ¹H NMR (400 MHz, DMSO-d₆) δ 9.30 (d, J=2.50 Hz, 1H), 8.39 (dd, J=9.36, 2.44 Hz, 1H), 7.62 (d, J=9.42 Hz, 1H), 7.47 (d, J=8.70 Hz, 1H), 5.72 (s, 1H), 5.59 (t, J=6.14 Hz, 1H), 3.72-3.89 (m, 2H), 3.55 (s, 3H), 3.46-3.53 (m, 1H), 1.29-1.38 (m, 1H), 0.64-0.72 (m, 1H), 0.60 (dq, J=9.40, 4.77 Hz, 1H), 0.46-0.54 (m, 1H), 0.24 (dq, J=9.55, 4.84 Hz, 1H).

### (S)-3-Bromo-4-((1-cyclopropyl-2,2-difluoro-3-hydroxypropyl)amino)-1-methyl-6-nitroquinolin-2(1H)-one

To a solution of (S)-4-((1-cyclopropyl-2,2-difluoro-3-hydroxypropyl)amino)-1-methyl-6-nitroquinolin-2(1H)-one (400 mg, 1.13 mmol) and NBS (201.49 mg, 1.13 mmol) in DCM (8 mL) was added TFA (645.42 mg, 5.66 mmol, 419.10 μL) dropwise at 0° C. under N₂. The mixture was stirred at 0° C. for 0.5 hr. Sat. NaHCO₃ (50 mL) was added to the mixture and was extracted with DCM (20 mL×3). The combined organic phase was washed with brine (20 mL), dried with anhydrous Na₂SO₄, filtered and concentrated in vacuo. The residue was purified by flash silica gel chromatography (ISCO®; 20 g SepaFlash® Silica Flash Column, Eluent of 0˜30% Ethyl acetate/Petroleum ether gradient @100 mL/min) to give the title compound as a white solid (400 mg, 832.92 μmol, 74% yield, 90% purity). ¹H NMR (400 MHz, DMSO-d₆) δ 8.95 (d, J=2.45 Hz, 1H), 8.43 (dd, J=9.35, 2.51 Hz, 1H), 7.75 (d, J=9.41 Hz, 1H), 5.85 (d, J=11.13 Hz, 1H), 5.62 (t, J=5.14 Hz, 1H), 3.96-4.06 (m, 1H), 3.77-3.90 (m, 2H), 3.71 (s, 3H), 1.20-1.33 (m, 1H), 0.52-0.70 (m, 3H), 0.41-0.51 (m, 1H).

### (S)-2-Cyclopropyl-3,3-difluoro-7-methyl-10-nitro-1,2,3,4-tetrahydro-[1,4]oxazepino[2,3-c]quinolin-6(7H)-one

To a solution of (S)-3-bromo-4-((1-cyclopropyl-2,2-difluoro-3-hydroxypropyl)amino)-1-methyl-6-nitroquinolin-2(1H)-one (400 mg, 925.46 μmol) in THE (10 mL) was added t-BuLi (1.3 M, 1.14 mL) (1.3 M in pentane) at 20° C. under N₂. The mixture was stirred at 60° C. for 0.25 hr. Water (40 mL) was added and the aqueous mixture was extracted with CH₂Cl₂ (20 mL×2). The organic extracts were combined, washed with brine (50 mL), dried with Na₂SO₄, filtered and concentrated in vacuo. The residue was purified by flash silica gel chromatography (ISCO®; 20 g SepaFlash® Silica Flash Column, Eluent of 0˜30% Ethyl acetate/Petroleum ether gradient @100 mL/min) to give the title compound as a yellow solid (250 mg, 711.63 μmol, 77% yield). ¹H NMR (400 MHz, DMSO-d₆) δ 9.11 (d, J=2.41 Hz, 1H), 8.34 (dd, J=9.32, 2.52 Hz, 1H), 7.65 (d, J=9.43 Hz, 1H), 7.01 (d, J=3.73 Hz, 1H), 4.34-4.56 (m, 2H), 3.61 (s, 3H), 3.22-3.30 (m, 1H), 1.29-1.40 (m, 1H), 0.68-0.77 (m, 1H), 0.49-0.59 (m, 2H), 0.30-0.38 (m, 1H).

### (S)-10-Amino-2-cyclopropyl-3,3-difluoro-7-methyl-1,2,3,4-tetrahydro-[1,4]oxazepino[2,3-c]quinolin-6(7H)-one

To a mixture of Pd/C (0.05 g, 768.56 μmol, 10% purity) in THE (10 mL) was added (S)-2-cyclopropyl-3,3-difluoro-7-methyl-10-nitro-1,2,3,4-tetrahydro-[1,4]oxazepino[2,3-c]quinolin-6(7H)-one (250 mg, 711.63 μmol) under Ar. The suspension was degassed under vacuum and purged with H₂ several times. The mixture was stirred under H₂ (15 psi) at 60° C. for 2 hours. The mixture was filtered and the filtrate was concentrated in vacuo to give the title compound as a yellow solid (150 mg, 261.42 μmol, 37% yield, 56% purity). LCMS: [M+H]⁺=322.1.

### (S)-2,5-Dichloro-6-((2-cyclopropyl-3,3-difluoro-7-methyl-6-oxo-1,2,3,4,6,7-hexahydro-[1,4]oxazepino[2,3-c]quinolin-10-yl)amino)nicotinonitrile

To a solution of (S)-10-amino-2-cyclopropyl-3,3-difluoro-7-methyl-1,2,3,4-tetrahydro-[1,4]oxazepino[2,3-c]quinolin-6(7H)-one (150 mg, 466.82 μmol) and 2,5,6-trichloropyridine-3-carbonitrile (96.84 mg, 466.82 μmol) in DMF (5 mL) was added DIEA (120.67 mg, 933.65 μmol, 162.62 μL). The mixture was stirred at 100° C. for 12 hr. The mixture was cooled to 20° C. and water (10 mL) was added, forming a precipitate which was filtered and the filter cake was concentrated in vacuo to give the title compound as a yellow solid (150 mg, 152.34 μmol, 33% yield, 50% purity). LCMS: [M+H]⁺=492.0.

### tert-Butyl (1-(5-chloro-3-cyano-6-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-5-methylpiperidin-3-yl)carbamate

To a solution of (S)-2,5-dichloro-6-((2-cyclopropyl-3,3-difluoro-7-methyl-6-oxo-1,2,3,4,6,7-hexahydro-[1,4]oxazepino[2,3-c]quinolin-10-yl)amino)nicotinonitrile (150 mg, 304.69 μmol) and tert-butyl N-(5-methyl-3-piperidyl)carbamate (78.36 mg, 365.63 μmol) in DMSO (2 mL) was added DIEA (78.76 mg, 609.38 μmol, 106.14 μL). The mixture was stirred at 100° C. for 1 hr. The mixture was cooled to 20° C. and water (5 mL) was added, forming a precipitate which was filtered and the filter cake was concentrated in vacuo to give the title compound as a yellow solid (150 mg, 111.92 μmol, 37% yield, 50% purity). LCMS: [M+H]⁺=670.2.

### 2-(3-Amino-5-methylpiperidin-1-yl)-5-chloro-6-(((S)-2-cyclopropyl-3,3-difluoro-7-methyl-6-oxo-1,2,3,4,6,7-hexahydro-[1,4]oxazepino[2,3-c]quinolin-10-yl)amino)nicotinonitrile

To a solution of tert-butyl (1-(5-chloro-3-cyano-6-(((S)-2-cyclopropyl-3,3-difluoro-7-methyl-6-oxo-1,2,3,4,6,7-hexahydro-[1,4]oxazepino[2,3-c]quinolin-10-yl)amino)pyridin-2-yl)-5-methylpiperidin-3-yl)carbamate (150 mg, 223.83 μmol) in HCl/EtOAc (4 M, 7.50 mL). The mixture was stirred at 20° C. for 1 hr. The mixture was concentrated in vacuo and used without further work up. The residue was purified by prep-HPLC (column: Waters Xbridge BEH C18 100*30 mm*10 μm; mobile phase: [water(NH₄HCO₃)-ACN]; B %: 30%-60%, 10 min) to give the title compound as a yellow solid (40 mg, 70.07 μmol, 31% yield, 99% purity). ¹H NMR (400 MHz, DMSO-d₆) δ 9.02 (s, 1H), 8.09-8.13 (m, 1H), 7.90 (s, 1H), 7.64 (dd, J=8.99, 1.65 Hz, 1H), 7.44 (dd, J=9.05, 1.10 Hz, 1H), 6.26-6.33 (m, 1H), 4.26-4.51 (m, 3H), 4.14 (d, J=11.74 Hz, 1H), 3.98-4.06 (m, 1H), 3.56 (s, 3H), 2.37-2.45 (m, 2H), 2.23-2.35 (m, 2H), 1.76-1.85 (m, 2H), 1.52 (s, 1H), 1.28-1.37 (m, 1H), 0.72-0.82 (m, 2H), 0.68 (d, J=7.09 Hz, 3H), 0.52 (d, J=5.14 Hz, 2H), 0.28-0.36 (m, 1H).

### 2-((3R,5S)-3-Amino-5-methylpiperidin-1-yl)-5-chloro-6-(((S)-2-cyclopropyl-3,3-difluoro-7-methyl-6-oxo-1,2,3,4,6,7-hexahydro-[1,4]oxazepino[2,3-c]quinolin-10-yl)amino)nicotinonitrile (24)

2-(3-Amino-5-methylpiperidin-1-yl)-5-chloro-6-(((S)-2-cyclopropyl-3,3-difluoro-7-methyl-6-oxo-1,2,3,4,6,7-hexahydro-[1,4]oxazepino[2,3-c]quinolin-10-yl)amino)nicotinonitrile (70.00 mg, 122.80 μmol) was separated by SFC (column: DAICEL CHIRALPAK IC (250 mm*30 mm, 10 μm); mobile phase: [ACN/IPA(0.1% NH₃H₂O)]; B %: 65%-65%, 30 min) to give the title compound as a yellow solid (8 mg, 13.31 μmol, 11% yield, 95% purity). ¹H NMR (400 MHz, DMSO-d₆) δ 9.16 (s, 1H), 8.09 (d, J=1.67 Hz, 1H), 8.02 (s, 1H), 7.92 (s, 2H), 7.68 (dd, J=8.94, 1.79 Hz, 1H), 7.45 (d, J=9.06 Hz, 1H), 6.29 (s, 1H), 4.24-4.48 (m, 3H), 3.94 (d, J=12.64 Hz, 1H), 3.57 (s, 3H), 3.18-3.26 (m, 1H), 3.06 (t, J=11.32 Hz, 1H), 2.82 (t, J=11.98 Hz, 1H), 2.28-2.36 (m, 1H), 1.95 (d, J=12.16 Hz, 1H), 1.59-1.71 (m, 1H), 1.30-1.37 (m, 1H), 1.23 (s, 1H), 1.05 (q, J=11.96 Hz, 1H), 0.68 (d, J=6.44 Hz, 3H), 0.53 (t, J=5.66 Hz, 2H), 0.28-0.35 (m, 1H).

### 2-((3S,5R)-3-Amino-5-methylpiperidin-1-yl)-5-chloro-6-(((S)-2-cyclopropyl-3,3-difluoro-7-methyl-6-oxo-1,2,3,4,6,7-hexahydro-[1,4]oxazepino[2,3-c]quinolin-10-yl)amino)nicotinonitrile (25)

2-(3-Amino-5-methylpiperidin-1-yl)-5-chloro-6-(((S)-2-cyclopropyl-3,3-difluoro-7-methyl-6-oxo-1,2,3,4,6,7-hexahydro-[1,4]oxazepino[2,3-c]quinolin-10-yl)amino)nicotinonitrile (70.00 mg, 122.80 μmol) was separated by SFC (column: DAICEL CHIRALPAK IC (250 mm*30 mm, 10 μm); mobile phase: [ACN/IPA(0.1% NH₃H₂O)]; B %: 65%-65%, 30 min) to give the title compound as a yellow solid (8.2 mg, 14.18 μmol, 12% yield, 99% purity). ¹H NMR (400 MHz, DMSO-d₆) δ 9.22 (s, 1H), 8.14 (s, 1H), 8.09 (s, 1H), 7.94 (d, J=2.38 Hz, 2H), 7.73-7.78 (m, 1H), 7.51 (d, J=9.06 Hz, 1H), 6.33 (s, 1H), 4.29-4.53 (m, 3H), 3.99 (d, J=12.52 Hz, 1H), 3.63 (s, 3H), 3.24-3.32 (m, 1H), 3.07-3.16 (m, 1H), 2.86 (t, J=11.86 Hz, 1H), 2.39 (t, J=12.16 Hz, 1H), 2.02 (d, J=11.21 Hz, 1H), 1.70 (dt, J=5.27, 2.67 Hz, 1H), 1.35-1.42 (m, 1H), 1.29 (s, 1H), 1.12 (q, J=12.20 Hz, 1H), 0.76 (d, J=6.44 Hz, 3H), 0.59 (d, J=5.01 Hz, 2H), 0.38 (d, J=4.41 Hz, 1H).

The absolute configurations of compounds 24 & 25 were randomly assigned based on the amino group and methyl group being in cis-conformation.

### Example 24: Synthesis of 2-((6-((6-((3R,5S)-3-amino-5-methylpiperidin-1-yl)-3-chloro-5-cyanopyridin-2-yl)amino)-1-methyl-2-oxo-1,2-dihydroquinolin-3-yl)oxy)-N-methylacetamide (39) and 2-((6-((6-((3S,5R)-3-amino-5-methylpiperidin-1-yl)-3-chloro-5-cyanopyridin-2-yl)amino)-1-methyl-2-oxo-1,2-dihydroquinolin-3-yl)oxy)-N-methylacetamide (40)

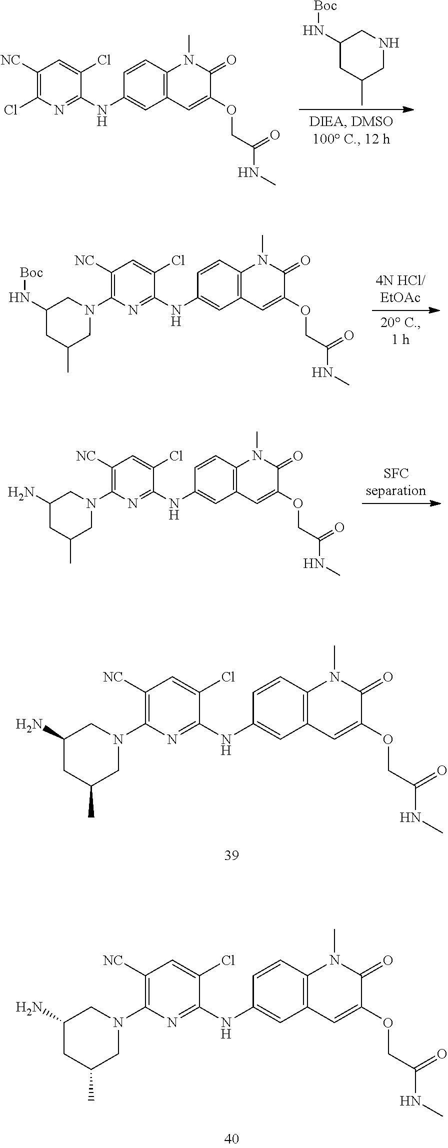 [SMILES: CNC(=O)COc1cc2cc(Nc3nc(Cl)c(C#N)cc3Cl)ccc2n(C)c1=O ; CC1CNCC(NC(=O)OC(C)(C)C)C1 ; CNC(=O)COc1cc2cc(Nc3nc(N4CC(C)CC(NC(=O)OC(C)(C)C)C4)c(C#N)cc3Cl)ccc2n(C)c1=O ; CNC(=O)COc1cc2cc(Nc3nc(N4CC(C)CC(N)C4)c(C#N)cc3Cl)ccc2n(C)c1=O ; CNC(=O)COc1cc2cc(Nc3nc(N4C[C@@H](C)C[C@@H](N)C4)c(C#N)cc3Cl)ccc2n(C)c1=O ; CNC(=O)COc1cc2cc(Nc3nc(N4C[C@H](C)C[C@H](N)C4)c(C#N)cc3Cl)ccc2n(C)c1=O]

### tert-Butyl (1-(5-chloro-3-cyano-6-((1-methyl-3-(2-(methylamino)-2-oxoethoxy)-2-oxo-1,2-dihydroquinolin-6-yl)amino)pyridin-2-yl)-5-methylpiperidin-3-yl)carbamate

A flask with mixture of 2-((6-((3,6-dichloro-5-cyanopyridin-2-yl)amino)-1-methyl-2-oxo-1,2-dihydroquinolin-3-yl)oxy)-N-methylacetamide (300 mg, 694.03 μmol), tert-butyl N-(5-methyl-3-piperidyl)carbamate (178.48 mg, 832.83 μmol) and DIEA (179.40 mg, 1.39 mmol, 241.77 μL) in DMSO (6 mL) was degassed and purged with N₂ for 3 times, and then the mixture was stirred at 100° C. for 12 hr under N₂ atmosphere. The mixture was cooled to 20° C. and water (10 mL) was added, forming a precipitate which was filtered and the filter cake was washed by EtOAc (20 mL) and concentrated in vacuo to give the title compound as a grey solid (300 mg, 418.95 μmol, 60% yield, 85% purity) as gray solid. LCMS: [M+H]⁺=610.2.

### 2-((6-((6-(3-Amino-5-methylpiperidin-1-yl)-3-chloro-5-cyanopyridin-2-yl)amino)-1-methyl-2-oxo-1,2-dihydroquinolin-3-yl)oxy)-N-methylacetamide

A mixture of tert-butyl (1-(5-chloro-3-cyano-6-((1-methyl-3-(2-(methylamino)-2-oxoethoxy)-2-oxo-1,2-dihydroquinolin-6-yl)amino)pyridin-2-yl)-5-methylpiperidin-3-yl)carbamate (300 mg, 491.72 μmol) and HCl/EtOAc (4 N HCl in EtOAc, 20 mL) was stirred at 20° C. for 1 hr. The mixture was concentrated in vacuo without further work up and the residue was purified by prep-HPLC (column: Phenomenex Luna C18 80*40 mm*3 μm; mobile phase: [water(HCl)-ACN]; B %: 20%-50%, 7 min) to give the title compound as a yellow solid (150 mg, HCl, 99% purity). ¹H NMR (400 MHz, DMSO-d₆, 26° C.) δ 9.12 (s, 1H), 8.26 (s, 2H), 8.00-8.08 (m, 2H), 7.86 (d, J=2.20 Hz, 1H), 7.71 (dd, J=9.05, 2.20 Hz, 1H), 7.54 (d, J=9.17 Hz, 1H), 7.31 (s, 1H), 4.56-4.62 (m, 2H), 4.32 (d, J=9.17 Hz, 1H), 4.01 (d, J=11.49 Hz, 1H), 3.69 (s, 3H), 3.10-3.21 (m, 1H), 2.84 (t, J=11.74 Hz, 1H), 2.68 (d, J=4.52 Hz, 3H), 2.39-2.46 (m, 1H), 2.03-2.11 (m, 1H), 1.72-1.86 (m, 1H), 1.16 (q, J=11.86 Hz, 1H), 0.84 (d, J=6.48 Hz, 3H).

### 2-((6-((6-((3R,5S)-3-Amino-5-methylpiperidin-1-yl)-3-chloro-5-cyanopyridin-2-yl)amino)-1-methyl-2-oxo-1,2-dihydroquinolin-3-yl)oxy)-N-methylacetamide (39)

2-((6-((6-(3-Amino-5-methylpiperidin-1-yl)-3-chloro-5-cyanopyridin-2-yl)amino)-1-methyl-2-oxo-1,2-dihydroquinolin-3-yl)oxy)-N-methylacetamide (150 mg, 274.50 μmol, HCl) was separated by SFC (column: DAICEL CHIRALPAK AD (250 mm*30 mm, 10 μm); mobile phase: 0.1% NH₃H₂O EtOH; B %: 47%-47%, 7 min) to give the title compound as a white solid (10 mg, 18.50 μmol, 7% yield, 94% purity). ¹H NMR (400 MHz, DMSO-d₆, 24° C.) δ 8.98 (s, 1H), 7.98 (s, 2H), 7.92 (s, 1H), 7.67 (dd, J=8.80, 1.83 Hz, 1H), 7.47 (d, J=8.93 Hz, 1H), 7.29 (s, 1H), 4.54 (s, 2H), 4.24 (d, J=11.62 Hz, 1H), 4.13 (d, J=12.72 Hz, 1H), 3.67 (s, 3H), 2.66 (d, J=4.52 Hz, 4H), 2.32-2.45 (m, 1H), 1.88 (d, J=12.23 Hz, 1H), 1.68-1.75 (m, 1H), 1.64 (d, J=9.29 Hz, 1H), 1.18-1.30 (m, 1H), 0.79 (d, J=6.48 Hz, 3H).

### 2-((6-((6-((3S,5R)-3-Amino-5-methylpiperidin-1-yl)-3-chloro-5-cyanopyridin-2-yl)amino)-1-methyl-2-oxo-1,2-dihydroquinolin-3-yl)oxy)-N-methylacetamide (40)

2-((6-((6-(3-amino-5-methylpiperidin-1-yl)-3-chloro-5-cyanopyridin-2-yl)amino)-1-methyl-2-oxo-1,2-dihydroquinolin-3-yl)oxy)-N-methylacetamide (150 mg, 274.50 μmol, HCl) was separated by SFC (column: DAICEL CHIRALPAK AD (250 mm*30 mm, 10 μm); mobile phase: 0.1% NH₃H₂O EtOH; B %: 47%-47%, 7 min) to give the title compound as a white solid (10 mg, 18.50 μmol, 7% yield, 94% purity). ¹H NMR (400 MHz, DMSO-d₆, 24° C.) δ 9.13 (s, 1H), 8.25 (s, 2H), 8.01-8.11 (m, 2H), 7.86 (s, 1H), 7.70 (d, J=8.93 Hz, 1H), 7.54 (d, J=9.17 Hz, 1H), 7.31 (s, 1H), 4.59 (s, 2H), 4.31 (d, J=10.88 Hz, 1H), 4.00 (d, J=11.25 Hz, 1H), 3.68 (s, 3H), 3.15 (t, J=10.58 Hz, 1H), 2.83 (t, J=11.80 Hz, 1H), 2.67 (d, J=4.40 Hz, 3H), 2.37-2.45 (m, 1H), 2.02-2.10 (m, 1H), 1.78 (d, J=3.55 Hz, 1H), 1.09-1.29 (m, 2H), 0.83 (d, J=6.48 Hz, 3H).

The absolute configurations of compounds 39 & 40 were randomly assigned based on the amino group and methyl group being in cis-conformation.

### Example 25: Synthesis of 2-((3R,5S)-3-amino-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitril (35) and 2-((3S,5R)-3-amino-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile (36)

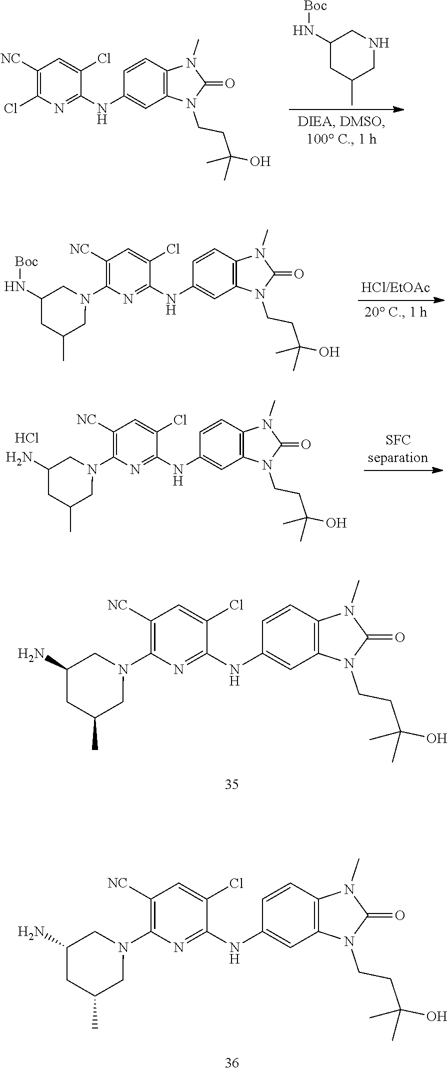

### tert-Butyl (1-(5-chloro-3-cyano-6-((3-(3-hydroxy-3-methylbutyl)-1-methyl-2-oxo-2,3-dihydro-1H-benzo[d]imidazol-5-yl)amino)pyridin-2-yl)-5-methylpiperidin-3-yl)carbamate

A flask with mixture of 2,5-dichloro-6-((3-(3-hydroxy-3-methylbutyl)-1-methyl-2-oxo-2,3-dihydro-1H-benzo[d]imidazol-5-yl)amino)nicotinonitrile (100 mg, 237.93 μmol), tert-butyl (5-methylpiperidin-3-yl)carbamate (61.19 mg, 285.52 μmol) and DIEA (61.50 mg, 475.86 μmol, 82.89 μL) in DMSO (1 mL) was degassed and purged with N₂ for 3 times, and then the mixture was stirred at 100° C. for 1 hr under N₂ atmosphere. Water (5 mL) was added, forming a precipitate which was filtered and the filter cake was dried under reduced pressure to give the title compound as a yellow solid (150 mg, 213.51 μmol, 90% yield, 85% purity). LCMS: [M-Boc+H]⁺=498.2.

### 2-(3-Amino-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile

A mixture of tert-butyl (1-(5-chloro-3-cyano-6-((3-(3-hydroxy-3-methylbutyl)-1-methyl-2-oxo-2,3-dihydro-1H-benzo[d]imidazol-5-yl)amino)pyridin-2-yl)-5-methylpiperidin-3-yl)carbamate (130 mg, 217.34 μmol) and HCl/EtOAc (4 M, 13 mL) was stirred at 20° C. for 1 hr under N₂ atmosphere. The mixture was concentrated in vacuo to give the title compound as a yellow solid (25 mg, 46.43 μmol, 21% yield, 99% purity, HCl). ¹H NMR (400 MHz, DMSO-d₆) δ 0.78 (d, J=6.60 Hz, 3H), 1.17 (s, 7H), 1.67-1.75 (m, 3H), 2.02 (br d, J=11.98 Hz, 1H), 2.34-2.42 (m, 1H), 2.83 (t, J=11.92 Hz, 1H), 3.07-3.17 (m, 1H), 3.32 (s, 2H), 3.85-3.90 (m, 2H), 3.93-3.99 (m, 1H), 4.26-4.32 (m, 1H), 7.12 (d, J=8.31 Hz, 1H), 7.25-7.33 (m, 2H), 7.98 (s, 1H), 8.09 (br s, 3H), 8.99 (s, 1H).

### 2-((3R,5S)-3-Amino-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile (35) and 2-((3S,5R)-3-amino-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile (36)

Racemic 2-(3-amino-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile (50 mg, HCl) was separated by SFC (column: DAICEL CHIRALPAK AD (250 mm*30 mm, 10 μm); mobile phase: [0.1% NH₃H₂O IPA]; B %: 30%-30%, 10 min) to give 2-((3S,5R)-3-amino-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile as a white solid (11.6 mg, 22.90 μmol, 24% yield, 98% purity); ¹H NMR (400 MHz, DMSO-d₆) δ 0.78 (d, J=6.60 Hz, 3H), 1.08-1.16 (m, 2H), 1.17 (s, 6H), 1.67-1.74 (m, 3H), 2.04 (br d, J=11.49 Hz, 1H), 2.40 (br d, J=11.86 Hz, 1H), 2.85 (br t, J=11.86 Hz, 1H), 3.10 (br t, J=10.82 Hz, 1H), 3.32 (br s, 2H), 3.84-3.91 (m, 2H), 3.96 (br d, J=11.49 Hz, 1H), 4.28 (br d, J=9.66 Hz, 1H), 7.14 (d, J=8.44 Hz, 1H), 7.28-7.31 (m, 1H), 7.32 (s, 1H), 7.98 (s, 1H), 8.22 (br s, 3H), 8.99 (s, 1H); and 2-((3R,5S)-3-amino-5-methylpiperidin-1-yl)-5-chloro-6-((3-(3-hydroxy-3-methylbutyl)-1-methyl-2-oxo-2,3-dihydro-1H-benzo[d]imidazol-5-yl)amino)nicotinonitrile as a white solid (12.2 mg, 24.27 μmol, 26% yield, 99 purity). ¹H NMR (400 MHz, DMSO-d₆) δ 0.76 (d, J=6.60 Hz, 3H), 0.79-0.87 (m, 1H), 1.16 (s, 6H), 1.57-1.65 (m, 1H), 1.67-1.73 (m, 1H), 1.86 (br d, J=12.23 Hz, 1H), 2.33 (br t, J=12.10 Hz, 2H), 2.60-2.69 (m, 2H), 3.32 (br s, 2H), 3.86-3.92 (m, 2H), 4.04 (br d, J=11.74 Hz, 1H), 4.16-4.22 (m, 1H), 7.10 (d, J=8.44 Hz, 1H), 7.25 (dd, J=8.44, 1.59 Hz, 1H), 7.38 (d, J=1.47 Hz, 1H), 7.89 (s, 1H), 8.87 (s, 1H).

The absolute configurations of compounds 35 & 36 were randomly assigned based on the amino group and methyl group being in cis-conformation.

### Example 26: Synthesis of chloro-5-cyanopyridin-2-yl)amino)-1-(oxetan-3-ylmethyl)-2-oxo-1,2-dihydroquinolin-3-yl)oxy)-N-methylacetamide (41) and 2-((6-((6-((3R,5S)-3-amino-4,4-difluoro-5-methylpiperidin-1-yl)-3-chloro-5-cyanopyridin-2-yl)amino)-1-(oxetan-3-ylmethyl)-2-oxo-1,2-dihydroquinolin-3-yl)oxy)-N-methylacetamide (42)

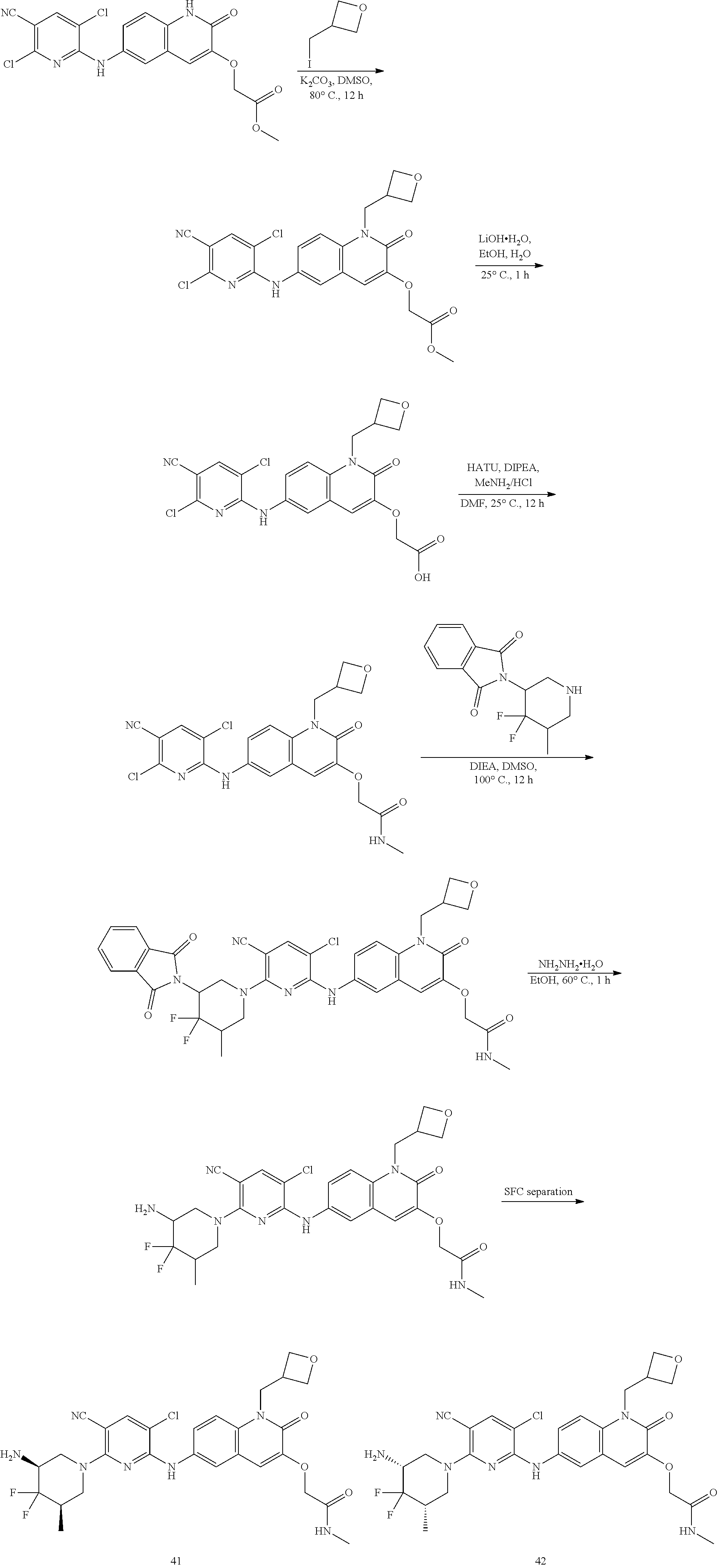

### Methyl 2-((6-((3,6-dichloro-5-cyanopyridin-2-yl)amino)-1-(oxetan-3-ylmethyl)-2-oxo-1,2-dihydroquinolin-3-yl)oxy)acetate

To a solution of methyl 2-[[6-[(3,6-dichloro-5-cyano-2-pyridyl)amino]-2-oxo-1H-quinolin-3-yl]oxy]acetate (3.5 g, 8.35 mmol) in DMSO (35 mL) was added K₂CO₃ (2.31 g, 16.70 mmol) and 3-(iodomethyl)oxetane (1.65 g, 8.35 mmol). The mixture was stirred at 80° C. for 12 hr. The reaction mixture was cooled to 15° C. and water (20 mL) was added, forming a precipitate which was filtered and the filter cake was dried in vacuo to give the title compound as a yellow solid (4 g, crude). LCMS: [M+H]⁺=489.0.

### 2-((6-((3,6-Dichloro-5-cyanopyridin-2-yl)amino)-1-(oxetan-3-ylmethyl)-2-oxo-1,2-dihydroquinolin-3-yl)oxy)acetic acid

To a solution of methyl 2-[[6-[(3,6-dichloro-5-cyano-2-pyridyl)amino]-1-(oxetan-3-ylmethyl)-2-oxo-3-quinolyl]oxy]acetate (4 g, 8.17 mmol) in EtOH (40 mL) and H₂O (40 mL) was added LiOH H₂O (1.72 g, 40.87 mmol). The mixture was stirred at 25° C. for 1 hr. The mixture was concentrated in vacuo to give the title compound as a yellow solid (4 g, crude). LCMS: [M+H]⁺=474.9.

### 2-((6-((3,6-Dichloro-5-cyanopyridin-2-yl)amino)-1-(oxetan-3-ylmethyl)-2-oxo-1,2-dihydroquinolin-3-yl)oxy)-N-methylacetamide

To a solution of 2-[[6-[(3,6-dichloro-5-cyano-2-pyridyl)amino]-1-(oxetan-3-ylmethyl)-2-oxo-3-quinolyl]oxy]acetic acid (4 g, 8.42 mmol) and MeNH₂ (1.14 g, 16.83 mmol, HCl) in DMF (30 mL) was added HATU (6.40 g, 16.83 mmol) and DIPEA (4.35 g, 33.66 mmol, 5.86 mL). The mixture was stirred at 25° C. for 12 hr. Water (60 mL) was added and then the mixture was extracted with ethyl acetate (50 mL×2). The combined organic phase was washed with brine (100 mL), dried with anhydrous Na₂SO₄, filtered and concentrated in vacuo. The crude product was triturated with EtOAc (20 mL) at 25° C. for 10 min to give the title compound as a yellow solid (1.2 g, 1.72 mmol, 20% yield, 70% purity). LCMS: [M+H]⁺=488.2.

### 2-((6-((3-Chloro-5-cyano-6-(3-(1,3-dioxoisoindolin-2-yl)-4,4-difluoro-5-methylpiperidin-1-yl)pyridin-2-yl)amino)-1-(oxetan-3-ylmethyl)-2-oxo-1,2-dihydroquinolin-3-yl)oxy)-N-methylacetamide

To a solution of 2-[[6-[(3,6-dichloro-5-cyano-2-pyridyl)amino]-1-(oxetan-3-ylmethyl)-2-oxo-3-quinolyl]oxy]-N-methylacetamide (200 mg, 409.57 μmol) and 2-(4,4-difluoro-5-methyl-3-piperidyl)isoindoline-1,3-dione (114.79 mg, 409.57 μmol) in DMSO (2 mL) was added DIPEA (105.87 mg, 819.13 μmol, 142.68 μL). The mixture was stirred at 100° C. for 12 hr. The mixture was cooled to 20° C. and water (3 mL) was added. The mixture was filtered and the filter cake was concentrated in vacuo to get a residue which was purified by prep-HPLC (column: Waters Xbridge BEH C18 100*30 mm*10 μm; mobile phase: [water(NH₄HCO₃)-ACN]; B %: 40%-65%, 8 min) to give the title compound as a yellow solid (100 mg, 136.59 μmol, 25% yield).

### 2-((6-((6-(3-amino-4,4-difluoro-5-methylpiperidin-1-yl)-3-chloro-5-cyanopyridin-2-yl)amino)-1-(oxetan-3-ylmethyl)-2-oxo-1,2-dihydroquinolin-3-yl)oxy)-N-methylacetamide (41)

To a solution of 2-[[6-[[3-chloro-5-cyano-6-[3-(1,3-dioxoisoindolin-2-yl)-4,4-difluoro-5-methyl-1-piperidyl]-2-pyridyl]amino]-1-(oxetan-3-ylmethyl)-2-oxo-3 quinolyl]oxy]-N-methyl-acetamide (100 mg, 136.59 μmol) in EtOH (4 mL) was added NH₂NH₂·H₂O (102.56 mg, 2.05 mmol, 99.58 μL). The mixture was stirred at 60° C. for 1 hr. The mixture was cooled to 20° C. and concentrated in vacuo. The residue was purified by prep-HPLC (column: Phenomenex C18 80*40 mm*3 μm; mobile phase: [water(NH₄HCO₃)-ACN]; B %: 15%-45%, 8 min) to give the title compound as a white solid (40 mg, 61.58 μmol, 45% yield, 93% purity). ¹H NMR (400 MHz, DMSO-d₆) δ 9.08 (s, 1H), 8.00 (s, 1H), 7.91-7.97 (m, 2H), 7.60-7.66 (m, 1H), 7.52-7.57 (m, 1H), 7.36 (s, 1H), 4.66 (br d, J=7.00 Hz, 2H), 4.57-4.63 (m, 2H), 4.48-4.56 (m, 4H), 4.21-4.31 (m, 1H), 4.11 (br d, J=12.01 Hz, 1H), 3.43 (dt, J=14.07, 7.10 Hz, 1H), 2.89-3.06 (m, 1H), 2.74-2.87 (m, 2H), 2.67 (d, J=4.63 Hz, 3H), 1.98-2.18 (m, 1H), 1.77 (br d, J=1.63 Hz, 2H), 0.86 (d, J=6.63 Hz, 3H).

### 2-((6-((6-((3S,5R)-3-Amino-4,4-difluoro-5-methylpiperidin-1-yl)-3-chloro-5-cyanopyridin-2-yl)amino)-1-(oxetan-3-ylmethyl)-2-oxo-1,2-dihydroquinolin-3-yl)oxy)-N-methylacetamide (41) and 2-((6-((6-((3R,5S)-3-amino-4,4-difluoro-5-methylpiperidin-1-yl)-3-chloro-5-cyanopyridin-2-yl)amino)-1-(oxetan-3-ylmethyl)-2-oxo-1,2-dihydroquinolin-3-yl)oxy)-N-methylacetamide (42)

2-[[6-[[6-(3-amino-4,4-difluoro-5-methyl-1-piperidyl)-3-chloro-5-cyano-2-pyridyl]amino]-1-(oxetan-3-ylmethyl)-2-oxo-3-quinolyl]oxy]-N-methyl-acetamide (40 mg, 66.44 μmol) was separated by SFC (column: DAICEL CHIRALCEL OD (250 mm*30 mm, 10 μm); mobile phase: [0.1% NH₃H₂O EtOH]; B %: 55%-55%, 10 min) to give 2-[[6-[[6-[(3S,5R)-3-amino-4,4-difluoro-5-methyl-1-piperidyl]-3-chloro-5-cyano-2-pyridyl]amino]-1-(oxetan-3-ylmethyl)-2-oxo-3-quinolyl]oxy]-N-methyl-acetamide as a white solid (12 mg, 19.73 μmol, 30% yield, 99% purity); ¹H NMR (400 MHz, DMSO-d₆) δ 9.07 (s, 1H), 8.00 (s, 1H), 7.93 (br d, J=1.79 Hz, 2H), 7.61-7.65 (m, 1H), 7.52-7.56 (m, 1H), 7.36 (s, 1H), 4.66 (br d, J=6.91 Hz, 2H), 4.57-4.62 (m, 2H), 4.47-4.56 (m, 4H), 4.08-4.30 (m, 2H), 3.43 (dt, J=14.04, 6.99 Hz, 1H), 3.30 (br s, 1H), 2.92-3.05 (m, 1H), 2.80 (td, J=12.58, 7.27 Hz, 2H), 2.67 (d, J=4.53 Hz, 3H), 1.99-2.17 (m, 1H), 1.77 (br s, 1H), 0.86 (d, J=6.68 Hz, 3H); and 2-[[6-[[6-[(3R,5S)-3-amino-4,4-difluoro-5-methyl-1-piperidyl]-3-chloro-5-cyano-2-pyridyl]amino]-1-(oxetan-3-ylmethyl)-2-oxo-3-quinolyl]oxy]-N-methyl-acetamide as a white solid; ¹H NMR (400 MHz, DMSO-d₆) δ 9.07 (s, 1H), 8.00 (s, 1H), 7.91-7.97 (m, 2H), 7.60-7.66 (m, 1H), 7.52-7.57 (m, 1H), 7.36 (s, 1H), 4.66 (br d, J=7.03 Hz, 2H), 4.57-4.63 (m, 2H), 4.49-4.56 (m, 4H), 4.07-4.30 (m, 2H), 3.39-3.48 (m, 1H), 3.30 (br s, 1H), 2.92-3.05 (m, 1H), 2.80 (td, J=12.55, 7.21 Hz, 2H), 2.67 (d, J=4.53 Hz, 3H), 1.99-2.15 (m, 1H), 1.72-1.90 (m, 1H), 0.86 (d, J=6.68 Hz, 3H).

The absolute configurations of compounds 41 & 42 were randomly assigned based on the amino group and methyl group being in cis-conformation.

### Example 27: Synthesis of 2-((3S,5R)-3-amino-4,4-difluoro-5-methylpiperidin-1-yl)-5-chloro-6-((3-(3-hydroxy-3-methylbutyl)-1-(oxetan-3-ylmethyl)-2-oxo-2,3-dihydro-1H-benzo[d]imidazol-5-yl)amino)nicotinonitrile (37) and 2-((3R,5S)-3-amino-4,4-difluoro-5-methylpiperidin-1-yl)-5-chloro-6-((3-(3-hydroxy-3-methylbutyl)-1-(oxetan-3-ylmethyl)-2-oxo-2,3-dihydro-1H-benzo[d]imidazol-5-yl)amino)nicotinonitrile (38)

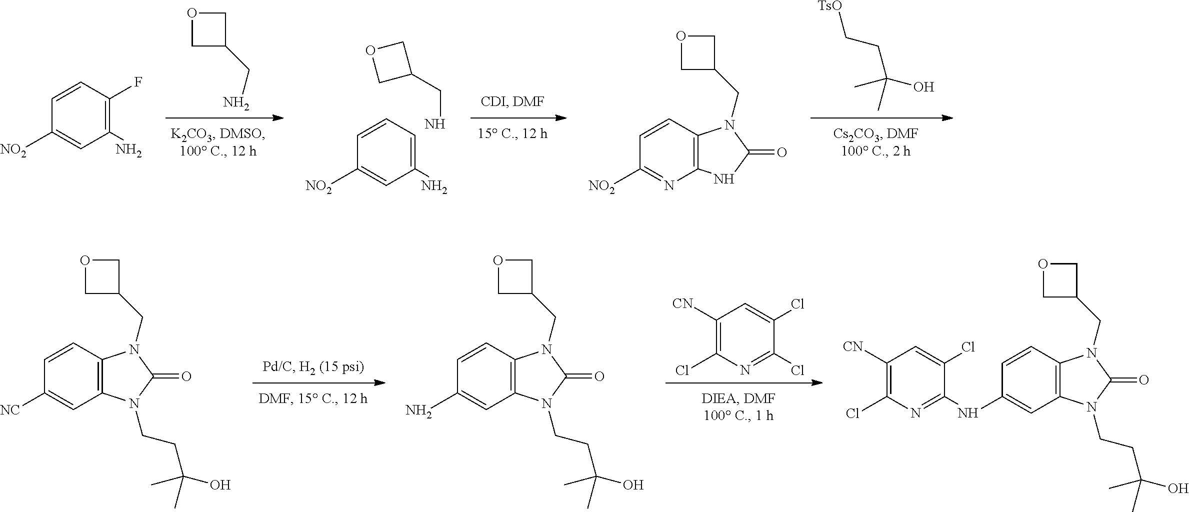

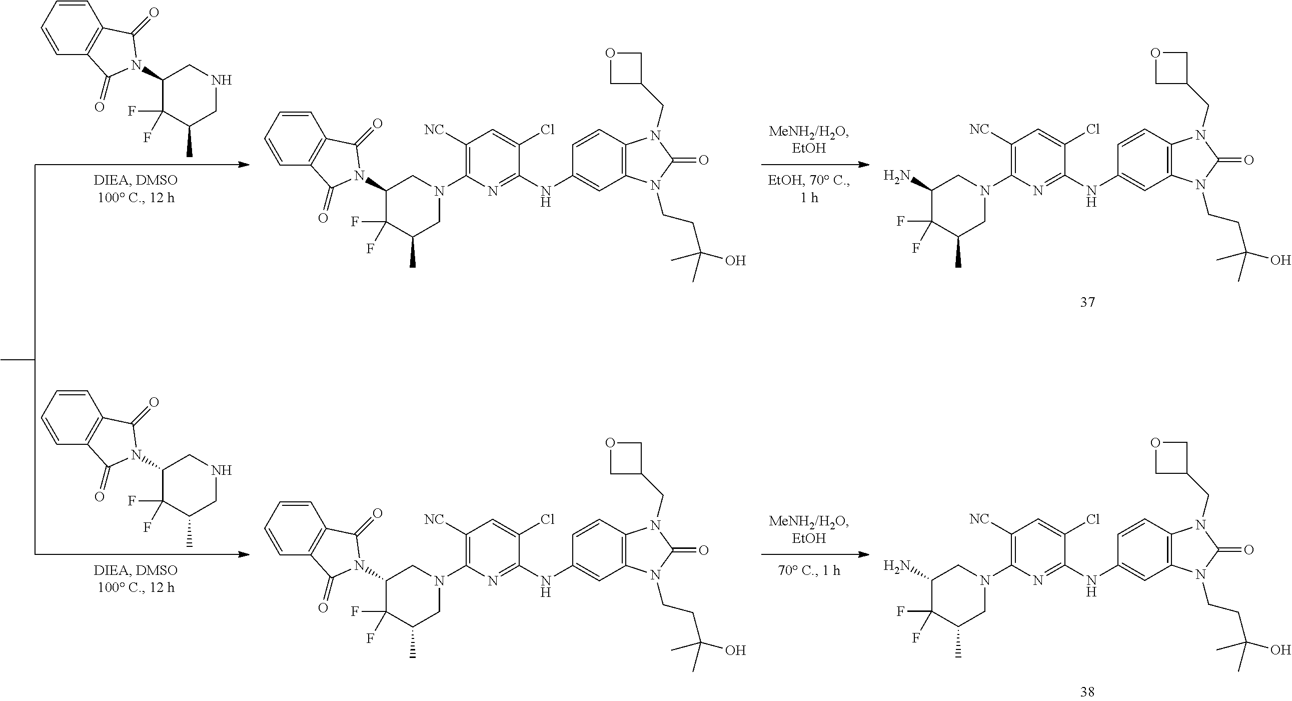 [SMILES: C[C@@H]1CNC[C@H](N2C(=O)c3ccccc3C2=O)C1(F)F ; C[C@@H]1CN(c2nc(Nc3ccc4c(c3)n(CCC(C)(C)O)c(=O)n4CC3COC3)c(Cl)cc2C#N)C[C@H](N2C(=O)c3ccccc3C2=O)C1(F)F ; C[C@@H]1CN(c2nc(Nc3ccc4c(c3)n(CCC(C)(C)O)c(=O)n4CC3COC3)c(Cl)cc2C#N)C[C@H](N)C1(F)F ; C[C@H]1CNC[C@@H](N2C(=O)c3ccccc3C2=O)C1(F)F ; C[C@H]1CN(c2nc(Nc3ccc4c(c3)n(CCC(C)(C)O)c(=O)n4CC3COC3)c(Cl)cc2C#N)C[C@@H](N2C(=O)c3ccccc3C2=O)C1(F)F ; C[C@H]1CN(c2nc(Nc3ccc4c(c3)n(CCC(C)(C)O)c(=O)n4CC3COC3)c(Cl)cc2C#N)C[C@@H](N)C1(F)F]

### 4-Nitro-N1-(oxetan-3-ylmethyl)benzene-1,2-diamine

To a solution of 2-fluoro-5-nitro-aniline (2 g, 12.81 mmol) and oxetan-3-ylmethanamine (1.34 g, 15.37 mmol) in DMSO (20 mL) was added K₂CO₃ (2.66 g, 19.22 mmol). The mixture was stirred at 100° C. for 12 hr. Water (200 mL) was added and then the mixture was extracted with DCM (300 mL×4). The combined organic phase was dried with anhydrous Na₂SO₄, filtered and concentrated to give a residue which was purified by column chromatography (silica gel, Petroleum ether/Ethyl acetate=5/1 to 0/1) to give the title compound as a white solid (2.5 g, 10.53 mmol, 82% yield, 94% purity). ¹H NMR (400 MHz, DMSO-d₆, 25° C.) δ 7.51 (dd, J=8.82, 2.62 Hz, 1H), 7.40 (d, J=2.62 Hz, 1H), 6.52 (d, J=8.82 Hz, 1H), 5.93 (t, J=5.01 Hz, 1H), 5.15 (s, 2H), 4.69 (dd, J=7.51, 6.08 Hz, 2H), 4.31 (t, J=5.90 Hz, 2H), 3.50 (dd, J=7.27, 5.36 Hz, 2H), 3.20-3.28 (m, 1H).

### 6-Nitro-3-(oxetan-3-ylmethyl)-1H-benzimidazol-2-one

A flask with mixture of 4-nitro-N1-(oxetan-3-ylmethyl)benzene-1,2-diamine (1 g, 4.48 mmol) and CDI (1.09 g, 6.72 mmol) in DMF (10 mL) was degassed and purged with N₂ for 3 times, and the mixture was stirred at 15° C. for 12 hr under N₂ atmosphere. Then water (80 mL) was added and the mixture was extracted with ethyl acetate (45 mL×3). The combined organic phase was dried with anhydrous Na₂SO₄, filtered and concentrated to give a residue which was purified by column chromatography (silica gel, Petroleum ether/Ethyl acetate=4/1 to 0/1) to give the title compound as a yellow solid (1.1 g, 4.10 mmol, 92% yield, 93% purity). ¹H NMR (400 MHz, DMSO-d₆, 25° C.) δ 8.00 (dd, J=8.70, 2.26 Hz, 1H), 7.74 (d, J=2.15 Hz, 1H), 7.41 (d, J=8.70 Hz, 1H), 4.61 (dd, J=7.63, 6.20 Hz, 2H), 4.41 (t, J=6.08 Hz, 2H), 4.18 (d, J=7.15 Hz, 2H), 3.33-3.44 (m, 1H).

### 3-(3-Hydroxy-3-methyl-butyl)-5-nitro-1-(oxetan-3-ylmethyl)benzimidazol-2-one

A flask with mixture of 6-nitro-3-(oxetan-3-ylmethyl)-1H-benzimidazol-2-one (1.3 g, 5.22 mmol), 3-hydroxy-3-methylbutyl 4-methylbenzenesulfonate (2.02 g, 7.83 mmol) and Cs₂CO₃ (5.10 g, 15.66 mmol) in DMF (10 mL) was degassed and purged with N₂ for 3 times, and the mixture was stirred at 100° C. for 2 hr under N₂ atmosphere. Then water (60 mL) was added and the mixture was extracted with ethyl acetate (50 mL×3). The combined organic phase was dried with anhydrous Na₂SO₄, filtered and concentrated to give a residue which was purified by column chromatography (silica gel, Petroleum ether/Ethyl acetate=3/1 to 0/1) to give the title compound as a yellow solid (1 g, 92% purity). ¹H NMR (400 MHz, DMSO-d₆, 25° C.) δ 8.05 (dd, J=8.69, 2.06 Hz, 1H), 8.00 (d, J=2.00 Hz, 1H), 7.48 (d, J=8.63 Hz, 1H), 4.61 (dd, J=7.63, 6.25 Hz, 2H), 4.51 (s, 1H), 4.40 (t, J=6.07 Hz, 2H), 4.23 (d, J=7.13 Hz, 2H), 3.96-4.02 (m, 2H), 3.39 (dt, J=13.91, 6.86 Hz, 1H), 1.70-1.76 (m, 2H), 1.16 (s, 6H).

### 5-Amino-3-(3-hydroxy-3-methyl-butyl)-1-(oxetan-3-ylmethyl)benzimidazol-2-one

To a mixture of Pd/C (200 mg, 10% purity) in DMF (10 mL) was added 3-(3-hydroxy-3-methyl-butyl)-5-nitro-1-(oxetan-3-ylmethyl)benzimidazol-2-one (1 g, 2.98 mmol). The mixture was stirred at 15° C. for 12 hr under H₂ (15 psi). The mixture was added Celite® and filtered to give a filtrate which was concentrated in vacuo to give the title compound as a red solid (900 mg, 2.71 mmol, 91% yield, 92% purity). ¹H NMR (400 MHz, DMSO-d₆, 25° C.) δ 6.85 (d, J=8.25 Hz, 1H), 6.38 (d, J=1.88 Hz, 1H), 6.30 (dd, J=8.25, 2.00 Hz, 1H), 4.81 (br s, 2H), 4.58 (dd, J=7.75, 6.13 Hz, 2H), 4.47 (s, 1H), 4.38 (t, J=6.07 Hz, 2H), 4.01 (d, J=7.00 Hz, 2H), 3.74-3.86 (m, 2H), 3.27-3.35 (m, 1H), 1.58-1.73 (m, 2H), 1.16 (s, 6H).

### 2,5-Dichloro-6-[[3-(3-hydroxy-3-methyl-butyl)-1-(oxetan-3-ylmethyl)-2-oxo-benzimidazol-5-yl]amino]pyridine-3-carbonitrile

A flask with mixture of 5-amino-3-(3-hydroxy-3-methyl-butyl)-1-(oxetan-3-ylmethyl) benzimidazol-2-one (800 mg, 2.62 mmol), 2,5,6-trichloropyridine-3-carbonitrile (543.46 mg, 2.62 mmol) and DIEA (677.17 mg, 5.24 mmol, 912.63 μL) in DMF (9 mL) was degassed and purged with N₂ for 3 times, then the mixture was stirred at 100° C. for 1 hr under N₂ atmosphere. The mixture was concentrated in vacuo without further work-up. The residue was purified by prep-HPLC (column: Welch Xtimate C18 250*70 mm #10 μm; mobile phase: [water(NH₄HCO₃)-ACN]; B %: 30%-60%, 20 min) to give the title compound as a white solid (700 mg, 1.40 mmol, 53% yield, 95% purity). ¹H NMR (400 MHz, DMSO-d₆, 25° C.) δ 9.54 (s, 1H), 8.35 (s, 1H), 7.35 (d, J=1.67 Hz, 1H), 7.22-7.26 (m, 1H), 7.16-7.19 (m, 1H), 4.62 (dd, J=7.69, 6.14 Hz, 2H), 4.42-4.44 (m, 2H), 4.40 (s, 1H), 4.14 (d, J=7.03 Hz, 2H), 3.85-3.92 (m, 2H), 3.35-3.45 (m, 1H), 1.65-1.77 (m, 2H), 1.17 (s, 6H).

### 5-Chloro-2-((3S,5R)-3-(1,3-dioxoisoindolin-2-yl)-4,4-difluoro-5-methylpiperidin-1-yl)-6-((3-(3-hydroxy-3-methylbutyl)-1-(oxetan-3-ylmethyl)-2-oxo-2,3-dihydro-1H-benzo[d]imidazol-5-yl)amino)nicotinonitrile

A flask with mixture of 2,5-dichloro-6-[[3-(3-hydroxy-3-methyl-butyl)-1-(oxetan-3-ylmethyl)-2-oxo-benzimidazol-5-yl]amino]pyridine-3-carbonitrile (150 mg, 314.89 μmol), 2-((3S,5R)-4,4-difluoro-5-methylpiperidin-3-yl)isoindoline-1,3-dione (88.25 mg, 314.89 μmol) and DIEA (203.49 mg, 1.57 mmol, 274.24 μL) in DMSO (1.5 mL) was degassed and purged with N₂ for 3 times, and then the mixture was stirred at 100° C. for 12 hr under N₂ atmosphere. The mixture was concentrated in vacuo without further work-up. The residue was purified by prep-HPLC (column: Waters Xbridge BEH C18 100*30 mm*10 μm; mobile phase: [water(NH₄HCO₃)-ACN]; B %: 40%-60%, 8 min) to give the title compound as a yellow solid (170 mg, 228.98 μmol, 73% yield, 97% purity). LCMS: [M+H]⁺=720.0.

### 2-((3S,5R)-3-Amino-4,4-difluoro-5-methylpiperidin-1-yl)-5-chloro-6-((3-(3-hydroxy-3-methylbutyl)-1-(oxetan-3-ylmethyl)-2-oxo-2,3-dihydro-1H-benzo[d]imidazol-5-yl)amino)nicotinonitrile (37)

To a solution of 5-chloro-2-((3S,5R)-3-(1,3-dioxoisoindolin-2-yl)-4,4-difluoro-5-methylpiperidin-1-yl)-6-((3-(3-hydroxy-3-methylbutyl)-1-(oxetan-3-ylmethyl)-2-oxo-2,3-dihydro-1H-benzo[d]imidazol-5-yl)amino)nicotinonitrile (170 mg, 236.06 μmol) in EtOH (10 mL) was added MeNH₂/H₂O (10 mL, 40% purity). The mixture was stirred at 70° C. for 1 hr. The mixture was concentrated in vacuo and purified directly with prep-HPLC (column: Phenomenex C18 80*40 mm*3 μm; mobile phase: [water(NH₄HCO₃)-ACN]; B %: 20%-50%, 8 min) to give the title compound as a white solid (35 mg, 55.76 μmol, 24% yield, 94% purity). ¹H NMR (400 MHz, DMSO-d₆, 22° C.) δ 7.97 (s, 1H), 7.29 (s, 1H), 7.23 (s, 2H), 4.61 (s, 2H), 4.50 (s, 1H), 4.42 (s, 2H), 4.15 (s, 3H), 3.97 (br s, 1H), 3.89 (br s, 2H), 3.36-3.46 (m, 2H), 2.77-2.91 (m, 2H), 2.64-2.71 (m, 1H), 1.92-2.09 (m, 1H), 1.68-1.73 (m, 2H), 1.66 (br s, 1H), 1.16 (d, J=0.73 Hz, 6H), 0.76 (d, J=6.72 Hz, 3H).

### 5-Chloro-2-((3R,5S)-3-(1,3-dioxoisoindolin-2-yl)-4,4-difluoro-5-methylpiperidin-1-yl)-6-((3-(3-hydroxy-3-methylbutyl)-1-(oxetan-3-ylmethyl)-2-oxo-2,3-dihydro-1H-benzo[d]imidazol-5-yl)amino)nicotinonitrile

A flask with mixture of 2,5-dichloro-6-[[3-(3-hydroxy-3-methyl-butyl)-1-(oxetan-3-ylmethyl)-2-oxo-benzimidazol-5-yl]amino]pyridine-3-carbonitrile (150 mg, 314.89 μmol), 2-((3R,5S)-4,4-difluoro-5-methylpiperidin-3-yl)isoindoline-1,3-dione (88.25 mg, 314.89 μmol) and DIEA (203.49 mg, 1.57 mmol, 274.24 μL) in DMSO (1.5 mL) was degassed and purged with N₂ for 3 times, and then the mixture was stirred at 100° C. for 12 hr under N₂ atmosphere. The mixture was concentrated in vacuo without further work-up. The residue was purified by prep-HPLC (column: Waters Xbridge BEH C18 100*30 mm*10 μm; mobile phase: [water(NH₄HCO₃)-ACN]; B %: 40%-60%, 8 min) to give the title compound as a white solid (150 mg, 202.04 μmol, 64% yield, 97% purity). LCMS: [M+H]⁺=720.0.

### 2-((3R,5S)-3-Amino-4,4-difluoro-5-methylpiperidin-1-yl)-5-chloro-6-((3-(3-hydroxy-3-methylbutyl)-1-(oxetan-3-ylmethyl)-2-oxo-2,3-dihydro-1H-benzo[d]imidazol-5-yl)amino)nicotinonitrile (38)

To a solution of 5-chloro-2-((3R,5S)-3-(1,3-dioxoisoindolin-2-yl)-4,4-difluoro-5-methylpiperidin-1-yl)-6-((3-(3-hydroxy-3-methylbutyl)-1-(oxetan-3-ylmethyl)-2-oxo-2,3-dihydro-1H-benzo[d]imidazol-5-yl)amino)nicotinonitrile (150 mg, 208.29 μmol) in EtOH (10 mL) was added MeNH₂/H₂O (10 mL, 40% purity). The mixture was stirred at 70° C. for 1 hr. The mixture was concentrated in vacuo and purified directly with prep-HPLC (column: Phenomenex C18 80*40 mm*3 μm; mobile phase: [water(NH₄HCO₃)-ACN]; B %: 20%-50%, 8 min) to give the title compound as a white solid (30 mg, 50.33 μmol, 24% yield, 99% purity). ¹H NMR (400 MHz, DMSO-d₆, 22° C.) δ 9.02 (s, 1H), 7.96 (s, 1H), 7.28 (s, 1H), 7.22 (s, 2H), 4.60 (dd, J=7.76, 6.05 Hz, 2H), 4.49 (s, 1H), 4.41 (t, J=6.05 Hz, 2H), 4.11-4.19 (m, 3H), 3.94 (br dd, J=13.51, 2.14 Hz, 1H), 3.85-3.91 (m, 2H), 3.35-3.43 (m, 2H), 2.78-2.91 (m, 2H), 2.63-2.70 (m, 1H), 1.94-2.09 (m, 1H), 1.67-1.72 (m, 2H), 1.65 (br s, 1H), 1.15 (s, 6H), 0.75 (d, J=6.72 Hz, 3H).

The absolute configurations of compounds 37 & 38 were randomly assigned based on the amino group and methyl group being in cis-conformation.

### Example 28: Synthesis of 2-((1-(2-aminoethyl)-6-((3-chloro-5-cyano-6-((3S,4S,5R)-4-fluoro-3,5-dimethylpiperidin-1-yl)pyridin-2-yl)amino)-2-oxo-1,2-dihydroquinolin-3-yl)oxy)-N-methylacetamide (66)

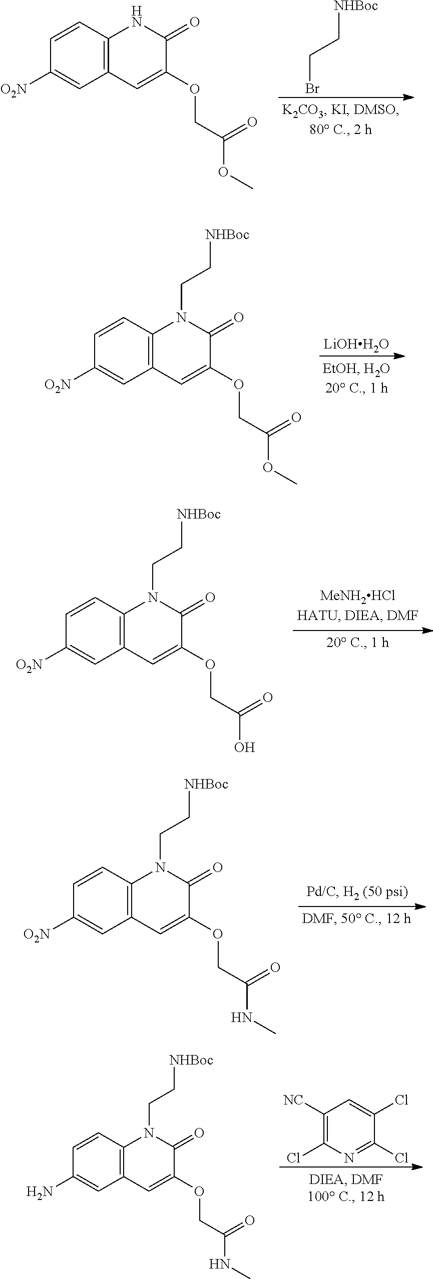

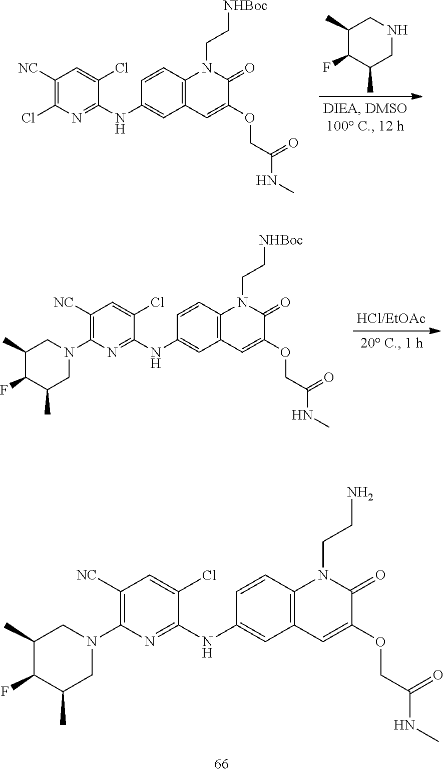

### Methyl 2-((1-(2-((tert-butoxycarbonyl)amino)ethyl)-6-nitro-2-oxo-1,2-dihydroquinolin-3-yl)oxy)acetate

A flask with mixture of methyl 2-[(6-nitro-2-oxo-1H-quinolin-3-yl)oxy]acetate (500 mg, 1.80 mmol), tert-butyl N-(2-bromoethyl)carbamate (2.01 g, 8.99 mmol), K₂CO₃ (496.77 mg, 3.59 mmol) and KI (149.16 mg, 898.58 μmol, 0.5 eq) in DMSO (10 mL) was degassed and purged with N₂ for 3 times, and the mixture was stirred at 80° C. for 2 hr under N₂ atmosphere. Then water (60 mL) was added and the mixture was extracted with ethyl acetate (50 mL×3). The combined organic phase was dried with anhydrous Na₂SO₄, filtered and concentrated to give the title compound as a brown oil (1 g, crude). LCMS: [M+H]⁺=422.2.

### 2-((1-(2-((tert-Butoxycarbonyl)amino)ethyl)-6-nitro-2-oxo-1,2-dihydroquinolin-3-yl)oxy)acetic acid

To a solution of methyl 2-((1-(2-((tert-butoxycarbonyl)amino)ethyl)-6-nitro-2-oxo-1,2-dihydroquinolin-3-yl)oxy)acetate (800 mg, 1.90 mmol) in EtOH (4 mL) and H₂O (4 mL) was added LiOH H₂O (159.33 mg, 3.80 mmol) and then the mixture was stirred at 20° C. for 1 hr. The mixture was concentrated in vacuo to give the title compound as a yellow solid (800 mg, crude). LCMS: [M+H]⁺=408.0.

### tert-Butyl (2-(3-(2-(methylamino)-2-oxoethoxy)-6-nitro-2-oxoquinolin-1(2H)-yl) ethyl)carbamate

To a solution of 2-((1-(2-((tert-butoxycarbonyl)amino)ethyl)-6-nitro-2-oxo-1,2-dihydroquinolin-3-yl)oxy)acetic acid (800 mg, 1.96 mmol), methenamine (530.37 mg, 7.86 mmol, HCl), HATU (1.49 g, 3.93 mmol) and DIEA (1.02 g, 7.86 mmol, 1.37 mL) in DMF (6 mL) and the mixture was stirred at 20° C. for 1 hr. Then water (40 mL) was added and the mixture was extracted with ethyl acetate (30 mL×2). The combined organic phase was dried with anhydrous Na₂SO₄, filtered and concentrated to give the title compound as a yellow oil (800 mg, 1.09 mmol, 56% yield, 57% purity). LCMS: [M+H]⁺=421.2.

### tert-Butyl (2-(6-amino-3-(2-(methylamino)-2-oxoethoxy)-2-oxoquinolin-1(2H)-yl) ethyl)carbamate

To a solution of Pd/C (0.1 g, 10% purity) in DMF (15 mL) was added tert-butyl (2-(3-(2-(methylamino)-2-oxoethoxy)-6-nitro-2-oxoquinolin-1(2H)-yl)ethyl)carbamate (600 mg, 1.43 mmol) under Ar. The mixture was stirred at 50° C. for 12 hr under H₂ (50 psi). The mixture was filtered and the filtrate was concentrated in vacuo without further work-up. The residue was purified by prep-HPLC (column: Waters Xbridge BEH C18 250*50 mm*10 μm; mobile phase: [water(NH₄HCO₃)-ACN]; B %: 15%-45%, 10 min) to give the title compound as a white solid (90 mg, 187.91 μmol, 13% yield, 82% purity). ¹H NMR (400 MHz, DMSO-d₆) δ 1.37 (s, 9H), 2.67-2.72 (m, 3H), 3.37 (br d, J=5.87 Hz, 2H), 4.31 (br t, J=5.44 Hz, 2H), 4.56 (s, 2H), 5.22 (br s, 2H), 6.70 (d, J=2.45 Hz, 1H), 6.88 (dd, J=8.80, 2.45 Hz, 1H), 7.01 (br t, J=5.44 Hz, 1H), 7.25 (s, 1H), 7.38 (d, J=8.80 Hz, 1H), 7.84 (br d, J=4.03 Hz, 1H).

### tert-Butyl (2-(6-amino-3-(2-(methylamino)-2-oxoethoxy)-2-oxoquinolin-1(2H)-yl)ethyl)carbamate

A flask with mixture of tert-butyl (2-(6-amino-3-(2-(methylamino)-2-oxoethoxy)-2-oxoquinolin-1(2H)-yl)ethyl)carbamate (60 mg, 153.68 μmol), 2,5,6-trichloropyridine-3-carbonitrile (31.88 mg, 153.68 μmol) and DIEA (39.72 mg, 307.35 μmol, 53.54 μL) in DMF (1 mL) was degassed and purged with N₂ for 3 times, and the mixture was stirred at 100° C. for 12 hr under N₂ atmosphere. Then water (15 mL) was added and the mixture was filtered to give a filter cake which was dried under vacuum to give the title compound as a grey solid (60 mg, 56.98 μmol, 37% yield, 53% purity). LCMS: [M+H]⁺=561.2.

### tert-Butyl (2-(6-((3-chloro-5-cyano-6-((3S,4S,5R)-4-fluoro-3,5-dimethylpiperidin-1-yl)pyridin-2-yl)amino)-3-(2-(methylamino)-2-oxoethoxy)-2-oxoquinolin-1(2H)-yl)ethyl)carbamate

A flask with mixture of tert-butyl (2-(6-amino-3-(2-(methylamino)-2-oxoethoxy)-2-oxoquinolin-1(2H)-yl)ethyl)carbamate (60.00 mg, 106.87 μmol), (3S,4S,5R)-4-fluoro-3,5-dimethyl-piperidine (53.75 mg, 320.62 μmol, HCl) and DIEA (138.13 mg, 1.07 mmol, 186.15 μL) in DMSO (1 mL) was degassed and purged with N₂ for 3 times, and the mixture was stirred at 100° C. for 2 hr under N₂ atmosphere. Then water (20 mL) was added and the mixture was filtered to give a filter cake which was dried under vacuum to give the title compound as a grey solid (60 mg, 62.78 μmol, 59% yield, 69% purity). LCMS: [M−56]+=600.2, [M−100]+=556.3.

### 2-((1-(2-Aminoethyl)-6-((3-chloro-5-cyano-6-((3S,4S,5R)-4-fluoro-3,5-dimethylpiperidin-1-yl)pyridin-2-yl)amino)-2-oxo-1,2-dihydroquinolin-3-yl)oxy)-N-methylacetamide (66)

A solution of tert-butyl (2-(6-((3-chloro-5-cyano-6-((3S,4S,5R)-4-fluoro-3,5-dimethylpiperidin-1-yl)pyridin-2-yl)amino)-3-(2-(methylamino)-2-oxoethoxy)-2-oxoquinolin-1(2H)-yl)ethyl)carbamate (60 mg, 91.44 μmol) in HCl/EtOAc (4 M, 6 mL) was stirred at 20° C. for 1 hr. The mixture was concentrated in vacuo without further work-up. The residue was purified by prep-HPLC (column: Phenomenex Luna 80*30 mm*3 μm; mobile phase: [water(HCl)-ACN]; B %: 20%-50%, 8 min) to give the title compound as a yellow solid (45 mg, 74.12 μmol, 11% yield, 98% purity, HCl). ¹H NMR (400 MHz, DMSO-d₆) δ 0.86 (d, J=6.85 Hz, 6H), 1.74-1.95 (m, 2H), 2.67 (d, J=4.65 Hz, 3H), 2.79 (t, J=12.65 Hz, 2H), 3.08 (d, J=5.87 Hz, 2H), 3.99 (dd, J=12.59, 3.79 Hz, 2H), 4.53-4.58 (m, 4H), 7.25-7.27 (m, 1H), 7.63-7.66 (m, 2H), 7.85-7.88 (m, 1H), 7.95-8.01 (m, 2H), 8.12 (s, 3H), 9.07-9.12 (m, 1H).

### Example 29: Synthesis of 2-((6-((3-chloro-5-cyano-6-((3S,4S,5R)-4-fluoro-3,5-dimethylpiperidin-1yl)pyridine-2-yl)amino)-1-(2-hydroxy-3-(methylamino)propyl)-2-oxo-1,2-dihydroquinolin-3-yl)oxy)-N-methylacetamide (67)

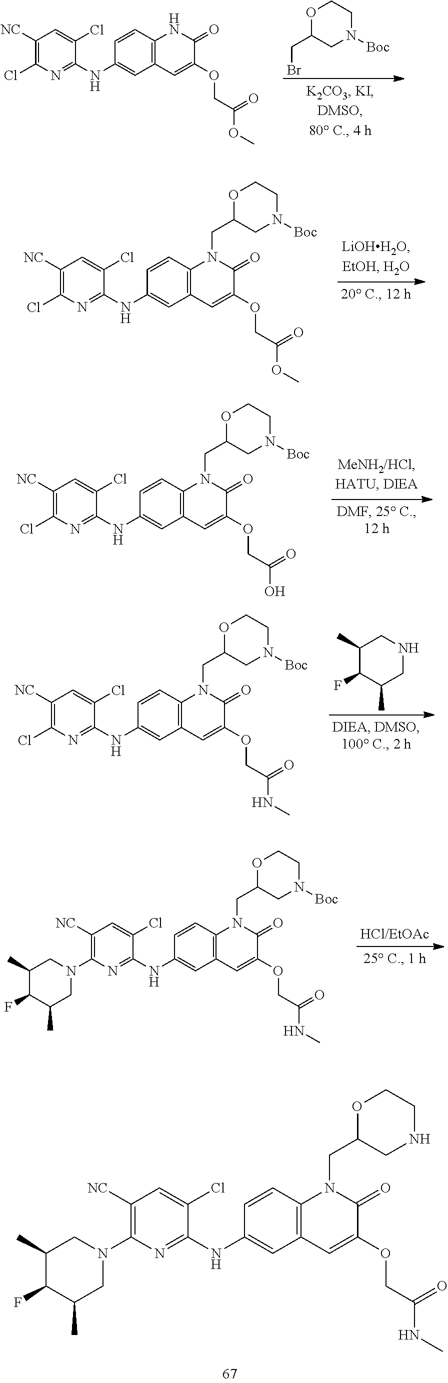

### tert-Butyl 2-((6-((3,6-dichloro-5-cyanopyridin-2-yl)amino)-3-(2-methoxy-2-oxoethoxy)-2-oxoquinolin-1(2H)-yl)methyl)morpholine-4-carboxylate

To a solution of methyl 2-[[6-[(3,6-dichloro-5-cyano-2-pyridyl)amino]-2-oxo-1H-quinolin-3-yl]oxy]acetate (2 g, 4.77 mmol) and tert-butyl 2-(bromomethyl)morpholine-4-carboxylate (1.47 g, 5.25 mmol) in DMSO (25 mL) was added K₂CO₃ (1.32 g, 9.54 mmol) and KI (395.98 mg, 2.39 mmol). The mixture was stirred at 80° C. for 4 hr. The mixture was added water (50 mL) and extracted with ethyl acetate (50 mL×3). The combined organic phase was dried with anhydrous Na₂SO₄, filtered and concentrated under reduced pressure to give the title compound as a white solid (2.9 g, crude). LCMS: [M+H]⁺=618.1.

### 2-((1-((4-(tert-Butoxycarbonyl)morpholin-2-yl)methyl)-6-((3,6-dichloro-5-cyanopyridin-2-yl)amino)-2-oxo-1,2-dihydroquinolin-3-yl)oxy)acetic acid

To a solution of tert-butyl 2-[[6-[(3,6-dichloro-5-cyano-2-pyridyl)amino]-3-(2-methoxy-2-oxo-ethoxy)-2-oxo-1-quinolyl]methyl]morpholine-4-carboxylate (2.9 g, 4.69 mmol) in EtOH (20 mL) and H₂O (20 mL) was added LiOH·H₂O (255.80 mg, 6.10 mmol). The mixture was stirred at 20° C. for 12 hr. The mixture was purified by prep-HPLC (column: Agela DuraShell C₁₈ 250*80 mm*10 μm; mobile phase: [water(NH₄HCO₃)-ACN]; B %: 30%-60%, 20 min) to give the title compound as a white solid. ¹H NMR (400 MHz, DMSO-d₆)=9.67 (s, 1H), 8.38 (s, 1H), 7.73 (s, 1H), 7.63 (s, 2H), 7.16 (s, 1H), 4.52-4.14 (m, 4H), 3.84-3.56 (m, 4H), 3.17-2.67 (m, 4H), 1.38 (s, 9H).

### tert-Butyl 2-((6-((3,6-dichloro-5-cyanopyridin-2-yl)amino)-3-(2-(methylamino)-2-oxoethoxy)-2-oxoquinolin-1(2H)-yl)methyl)morpholine-4-carboxylate

To a solution of 2-[[1-[(4-tert-butoxycarbonylmorpholin-2-yl)methyl]-6-[(3,6-dichloro-5-cyano-2-pyridyl)amino]-2-oxo-3-quinolyl]oxy]acetic acid (1 g, 1.65 mmol) and methenamine (223.41 mg, 3.31 mmol, HCl) in DMF (10 mL) was added HATU (1.26 g, 3.31 mmol) and DIPEA (855.29 mg, 6.62 mmol, 1.15 mL). The mixture was stirred at 25° C. for 12 hr. Water (10 mL) was added to the mixture, forming a precipitate which was filtered and the filter cake was washed with water (20 mL×2), PE (20 mL×2), the filter cake was dried under reduced pressure to give the title compound as a white solid. LCMS: [M+H]⁺=617.2.

### tert-Butyl (3-(6-((3-chloro-5-cyano-6-((3S,4S,5R)-4-fluoro-3,5-dimethylpiperidin-1-yl)pyridin-2-yl)amino)-3-(2-(methylamino)-2-oxoethoxy)-2-oxoquinolin-1(2H)-yl)-2-methylpropyl)carbamate

To a solution of tert-butyl 2-[[6-[(3,6-dichloro-5-cyano-2-pyridyl)amino]-3-[2-(methylamino)-2-oxo-ethoxy]-2-oxo-1-quinolyl]methyl]morpholine-4-carboxylate (100 mg, 161.95 μmol) and (3S,4S,5R)-4-fluoro-3,5-dimethyl-piperidine (32.58 mg, 194.34 μmol, HCl) in DMSO (1 mL) was added DIPEA (62.79 mg, 485.85 μmol, 84.63 μL). The mixture was stirred at 100° C. for 2 hr. Water (5 mL) was added to the mixture, forming a precipitate which was filtered and the filter cake was washed with water (10 mL×2) and EA (10 mL×2). The filter cake was dried under reduced pressure to give the title compound as a white solid (90 mg, 126.37 μmol, 78% yield). LCMS: [M+H]⁺=712.3.

### 2-((6-((3-Chloro-5-cyano-6-((3S,4S,5R)-4-fluoro-3,5-dimethylpiperidin-1-yl)pyridin-2-yl)amino)-1-(2-hydroxy-3-(methylamino)propyl)-2-oxo-1,2-dihydroquinolin-3-yl)oxy)-N-methylacetamide (67)

A solution of tert-butyl 2-[[6-[[3-chloro-5-cyano-6-[(3S,4S,5R)-4-fluoro-3,5-dimethyl-1-piperidyl]-2-pyridyl]amino]-3-[2-(methylamino)-2-oxo-ethoxy]-2-oxo-1-quinolyl]methyl]morpholine-4-carboxylate (330 mg, 463.35 μmol) in HCl/EtOAc (4 mL, 4 M) was stirred at 25° C. for 1 hr. The reaction mixture was concentrated under reduced pressure and the residue was purified by prep-HPLC (column: Phenomenex Luna 80*30 mm*3 μm; mobile phase: [water(HCl)-ACN]; B %: 20%-50%, 8 min) to give the title compound as a white solid (30 mg, 45.74 μmol, 10% yield, 99% purity, HCl). ¹H NMR (400 MHz, DMSO-d₆) δ 9.61-9.59 (m, 1H), 9.52-9.50 (m, 1H), 9.08-9.05 (m, 1H), 8.03 (d, J=4.4 Hz, 1H), 7.95-7.94 (m, 1H), 7.82-7.81 (m, 1H), 7.65-7.62 (m, 1H), 7.58-7.56 (m, 1H), 7.25-7.24 (m, 1H), 4.55 (s, 2H), 4.43-4.41 (m, 1H), 4.18-4.15 (m, 2H), 3.99 (d, J=13.2 Hz, 2H), 3.88 (d, J=12.8 Hz, 1H), 3.68-3.65 (m, 1H), 3.34 (d, J=12.4 Hz, 1H), 3.13 (br d, J=12.0 Hz, 1H), 3.03-2.93 (m, 2H), 2.83-2.72 (m, 2H), 2.67 (d, J=4.8 Hz, 3H), 1.90-1.73 (m, 2H), 0.89-0.85 (m, 6H).

### Example 30: Synthesis of 2-((6-((3-chloro-5-cyano-6-((3S,4S,5R)-4-fluoro-3,5-dimethylpiperidin-1-yl)pyridin-2-yl)amino)-1-(2-morpholinoethyl)-2-oxo-1,2-dihydroquinolin-3-yl)oxy)-N-methylacetamide (68)

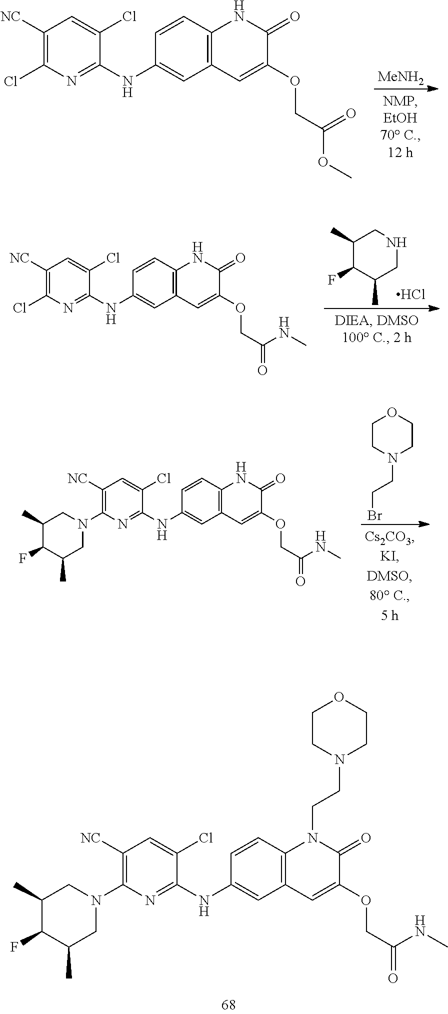

### 2-(4,4-Difluoro-3-hydroxy-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile

To a solution of methyl 2-[[6-[(3,6-dichloro-5-cyano-2-pyridyl)amino]-2-oxo-1H-quinolin-3-yl]oxy]acetate (3 g, 7.16 mmol) in NMP (3 mL) and EtOH (27 mL) was added MeNH₂ (4.44 g, 57.25 mmol, 40% purity). The mixture was stirred at 70° C. for 12 hr. The reaction mixture was cooled to 20° C., and EtOH (90 mL) was added. The suspension was filtered and the filter cake was dried under reduced pressure to give the title compound as a yellow solid (2.6 g, 5.41 mmol, 76% yield, 87% purity). LCMS: [M+H]⁺=418.0.

### 2-((6-((3-chloro-5-cyano-6-(4-fluoro-3,5-dimethylpiperidin-1-yl)pyridin-2-yl)amino)-2-oxo-1,2-dihydroquinolin-3-yl)oxy)-N-methylacetamide

To a solution of 2-[[6-[(3,6-dichloro-5-cyano-2-pyridyl)amino]-2-oxo-1H-quinolin-3-yl]oxy]-N-methyl-acetamide (500 mg, 1.20 mmol) and (3R,5S)-4-fluoro-3,5-dimethyl-piperidine (300.64 mg, 1.79 mmol, HCl) in DMSO (1 mL) was added DIEA (618.04 mg, 4.78 mmol, 832.94 μL). The mixture was stirred at 100° C. for 2 hr. The reaction mixture was treated with water (20 mL), forming a precipitate which was filtered. The filter cake was washed with EtOAc (30 mL), then the filter cake was dried under reduce pressure to give the title compound as a brown solid (600 mg, 818.77 μmol, 68% yield, 70% purity). LCMS: [M+H]⁺=513.3.

### 2-[[6-[[3-Chloro-5-cyano-6-[(3R,5S)-4-fluoro-3,5-dimethyl-1-piperidyl]-2-pyridyl]amino]-1-(2-morpholinoethyl)-2-oxo-3-quinolyl]oxy]-N-methyl-acetamide (68)

To a solution of 2-[[6-[[3-chloro-5-cyano-6-[(3R,5S)-4-fluoro-3,5-dimethyl-1-piperidyl]-2-pyridyl]amino]-2-oxo-1H-quinolin-3-yl]oxy]-N-methyl-acetamide (200 mg, 389.89 μmol) and 4-(2-bromoethyl)morpholine (189.17 mg, 974.73 μmol) in DMSO (2 mL) was added Cs₂CO₃ (254.07 mg, 779.78 μmol) and KI (32.36 mg, 194.95 μmol). The mixture was stirred at 80° C. for 5 hr. The reaction mixture was treated with water (30 mL), forming a precipitate which was filtered. The filter cake was washed with EtOAc (30 mL), collected and dried under reduced pressure to give a residue which was purified by prep-HPLC (column: Phenomenex Luna 80*30 mm*3 μm; mobile phase: [water(HCl)-ACN]; B %: 25%-55%, 8 min) and further separated by SFC (column: DAICEL CHIRALPAK IG (250 mm*30 mm, 10 μm); mobile phase: [0.1% NH₃H₂O EtOH]; B %: 50%-50%, 10 min) to give the title compound as a white solid (18 mg, 23.29 μmol, 29% yield, 81% purity). ¹H NMR (400 MHz, DMSO-d₆) δ 9.07 (s, 1H), 7.88-8.01 (m, 2H), 7.78 (br s, 1H), 7.66 (dd, J=9.13, 2.38 Hz, 1H), 7.49 (br d, J=9.13 Hz, 1H), 7.22 (s, 1H), 4.52-4.58 (m, 2H), 4.43 (br s, 2H), 3.98 (br dd, J=12.94, 3.69 Hz, 2H), 3.58 (br s, 4H), 3.30 (br s, 1H), 2.79 (br t, J=12.69 Hz, 2H), 2.67 (d, J=4.63 Hz, 3H), 2.52-2.53 (m, 4H), 1.75-1.93 (m, 2H), 1.23 (s, 2H), 0.85 (d, J=6.88 Hz, 6H).

### Example 31: Synthesis of 2-((1-(3-aminopropyl)-6-((3-chloro-5-cyano-6-((3S,4S,5R)-4-fluoro-3,5-dimethylpiperidin-1-yl)pyridin-2-yl)amino)-2-oxo-1,2-dihydroquinolin-3-yl)oxy)-N-methylacetamide (69)

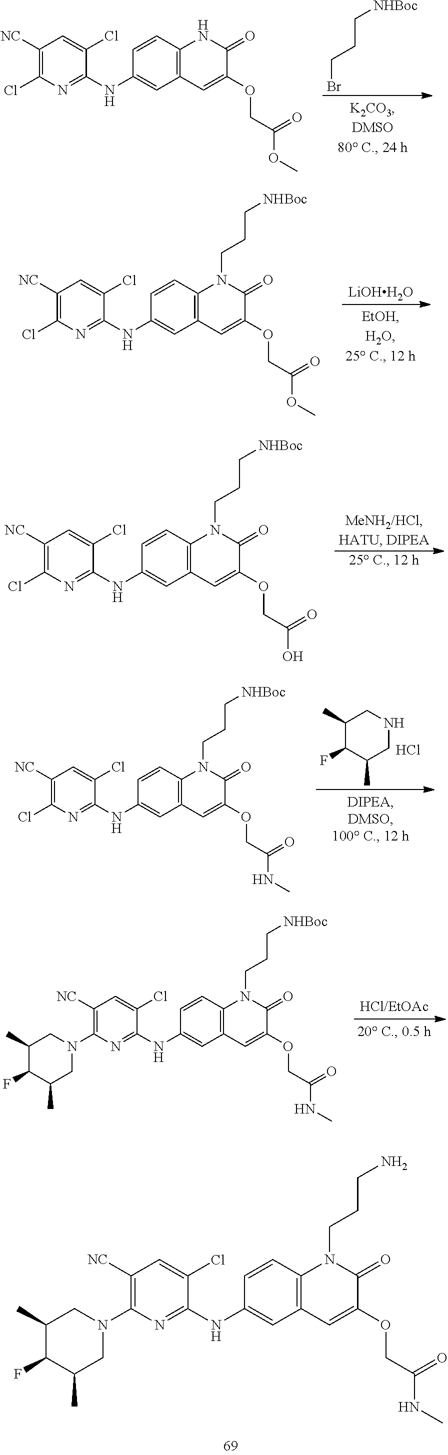

### Methyl 2-((1-(3-((tert-butoxycarbonyl)amino)propyl)-6-((3,6-dichloro-5-cyanopyridin-2-yl)amino)-2-oxo-1,2-dihydroquinolin-3-yl)oxy)acetate

To a solution of methyl 2-[[6-[(3,6-dichloro-5-cyano-2-pyridyl)amino]-2-oxo-1H-quinolin-3-yl]oxy]acetate (1 g, 2.39 mmol) and tert-butyl N-(3-bromopropyl)carbamate (852.02 mg, 3.58 mmol) in DMSO (10 mL) was added K₂CO₃ (659.35 mg, 4.77 mmol, 359.55 μL). The mixture was stirred at 80° C. for 12 hr. Another batch of tert-butyl N-(3-bromopropyl)carbamate (568.01 mg, 2.39 mmol) was added and stirred at 80° C. for 12 hr. The mixture was treated with water (10 mL), forming a precipitate which was filtered and the filter cake was collected and dried in vacuo to give the title compound as a yellow solid (1.4 g, crude). LCMS: [M+H]⁺=576.0.

### 2-((1-(3-((tert-Butoxycarbonyl)amino)propyl)-6-((3,6-dichloro-5-cyanopyridin-2-yl)amino)-2-oxo-1,2-dihydroquinolin-3-yl)oxy)acetic acid

To a solution of methyl 2-[[1-[3-(tert-butoxycarbonylamino)propyl]-6-[(3,6-dichloro-5-cyano-2-pyridyl)amino]-2-oxo-3-quinolyl]oxy]acetate (1.4 g, 2.43 mmol) in EtOH (16 mL) and H₂O (16 mL) was added LiOH H₂O (203.84 mg, 4.86 mmol). The mixture was stirred at 25° C. for 12 hr. The reaction mixture was concentrated under reduced pressure to give the title compound as a yellow solid (1.5 g, crude). LCMS: [M+H]⁺=562.1.

### tert-Butyl(3-(6-((3,6-dichloro-5-cyanopyridin-2-yl)amino)-3-(2-(methylamino)-2-oxoethoxy)-2-oxoquinolin-1(2H)-yl)propyl)carbamate

To a solution of 2-[[1-[3-(tert-butoxycarbonylamino)propyl]-6-[(3,6-dichloro-5-cyano-2-pyridyl)amino]-2-oxo-3-quinolyl]oxy]acetic acid (2.2 g, 3.91 mmol) and methenamine (396.17 mg, 5.87 mmol, HCl) in DMF (22 mL) was added HATU (2.97 g, 7.82 mmol) and DIPEA (2.02 g, 15.65 mmol, 2.73 mL). The mixture was stirred at 25° C. for 12 hr. The reaction mixture was treated with water (30 mL) and extracted with EtOAc (30 mL×3). The combined organic layers were washed with brine, dried over anhydrous Na₂SO₄, filtered and concentrated under reduced pressure to give a residue which was purified by flash silica gel column chromatography (Silica Flash Column, Eluent of 0˜100% DMF/Ethyl acetate ether gradient) and further purified by prep-HPLC (column: Welch Xtimate C18 250*70 mm*10 μm; mobile phase: [water(NH₄HCO₃)-ACN]; B %: 30%-60%, 20 min) to give the title compound as a yellow solid (180 mg, 303.42 μmol, 8% yield, 97% purity).

### tert-Butyl(3-(6-((3-chloro-5-cyano-6-((3S,4S,5R)-4-fluoro-3,5-dimethylpiperidin-1-yl)pyridin-2-yl)amino)-3-(2-(methylamino)-2-oxoethoxy)-2-oxoquinolin-1(2H)-yl)propyl)carbamate

To a solution of tert-butyl N-[3-[6-[(3,6-dichloro-5-cyano-2-pyridyl)amino]-3-[2-(methylamino)-2-oxo-ethoxy]-2-oxo-1-quinolyl]propyl]carbamate (130 mg, 225.91 μmol) and (3S,4S,5R)-4-fluoro-3,5-dimethyl-piperidine (75.75 mg, 451.83 μmol, HCl) in DMSO (2 mL) was added DIPEA (116.79 mg, 903.65 μmol, 157.40 μL). The mixture was stirred at 100° C. for 12 hr. The reactant mixture was treated with water (2 mL), forming a precipitate which was filtered and dried under reduced pressure to give the title compound as an orange-red oil (130 mg). ¹H NMR (400 MHz, DMSO-d₆)=9.07 (s, 1H), 7.94 (s, 1H), 7.80 (br s, 1H), 7.63 (br dd, J=9.00, 2.09 Hz, 1H), 7.48 (br d, J=9.18 Hz, 1H), 7.21 (s, 1H), 6.93 (br t, J=5.19 Hz, 1H), 4.53 (s, 2H), 4.28 (br t, J=7.27 Hz, 2H), 3.97 (br dd, J=13.17, 3.76 Hz, 2H), 3.04 (q, J=6.20 Hz, 2H), 2.78 (br t, J=12.58 Hz, 2H), 2.66 (d, J=4.53 Hz, 3H), 1.87 (br s, 1H), 1.72-1.82 (m, 3H), 1.39 (s, 9H), 0.92-1.03 (m, 1H), 0.80-0.89 (m, 6H).

### 2-((1-(3-Aminopropyl)-6-((3-chloro-5-cyano-6-((3S,4S,5R)-4-fluoro-3,5-dimethylpiperidin-1-yl)pyridin-2-yl)amino)-2-oxo-1,2-dihydroquinolin-3-yl)oxy)-N-methylacetamide (69)

To a solution of tert-butyl N-[3-[6-[[3-chloro-5-cyano-6-[(3S,4S,5R)-4-fluoro-3,5-dimethyl-1-piperidyl]-2-pyridyl]amino]-3-[2-(methylamino)-2-oxo-ethoxy]-2-oxo-1-quinolyl]propyl]carbamate (100 mg, 149.22 μmol) in HCl/EtOAc (10 mL). The mixture was stirred at 20° C. for 0.5 hr. The reaction mixture was concentrated under reduced pressure to give a residue which was purified by prep-HPLC (column: Phenomenex C18 80*40 mm*3 μm; mobile phase: [water(NH₄HCO₃)-ACN]; B %: 28%-58%, 8 min) to give the title compound as a yellow solid (26 mg, 43.33 μmol, 29% yield, 95% purity). ¹H NMR (400 MHz, DMSO-d₆) δ 8.94-9.19 (m, 1H), 7.92-8.00 (m, 2H), 7.80 (br s, 1H), 7.64 (br d, J=8.23 Hz, 1H), 7.55 (br d, J=8.34 Hz, 1H), 7.22 (br s, 1H), 4.53 (br s, 2H), 4.34 (br s, 2H), 3.97 (br d, J=11.68 Hz, 2H), 2.78 (br t, J=12.52 Hz, 2H), 2.60-2.68 (m, 5H), 1.66-1.93 (m, 5H), 0.84 (br d, J=6.44 Hz, 6H).

### Example 32: Synthesis of 5-chloro-2-((3S,4S,5R)-4-fluoro-3,5-dimethylpiperidin-1-yl)-6-((1-methyl-3-((S)-morpholin-2-ylmethoxy)-2-oxo-1,2-dihydroquinolin-6-yl)amino)nicotinonitrile (70) and 5-chloro-2-((3S,4S,5R)-4-fluoro-3,5-dimethylpiperidin-1-yl)-6-((1-methyl-3-((R)-morpholin-2-ylmethoxy)-2-oxo-1,2-dihydroquinolin-6-yl)amino)nicotinonitrile (71)

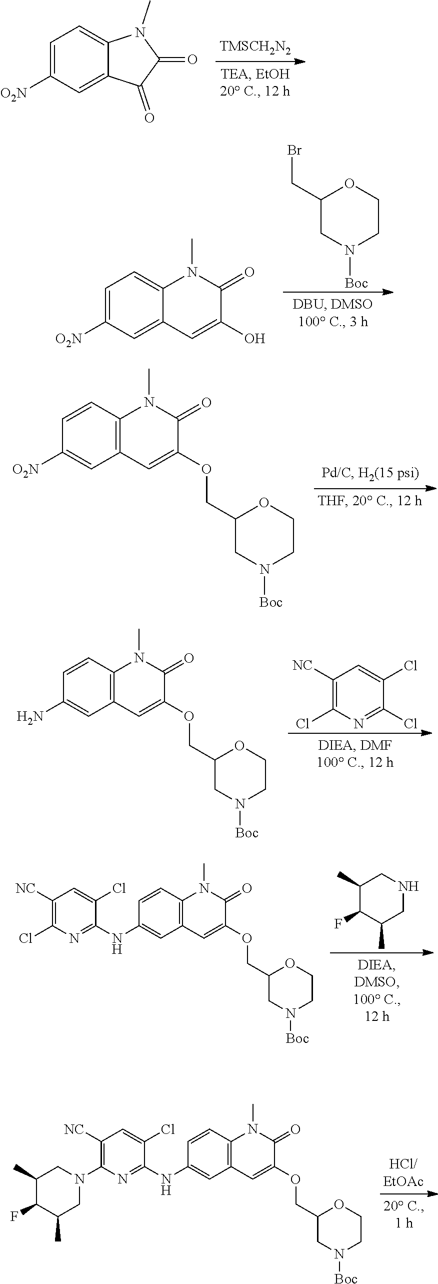

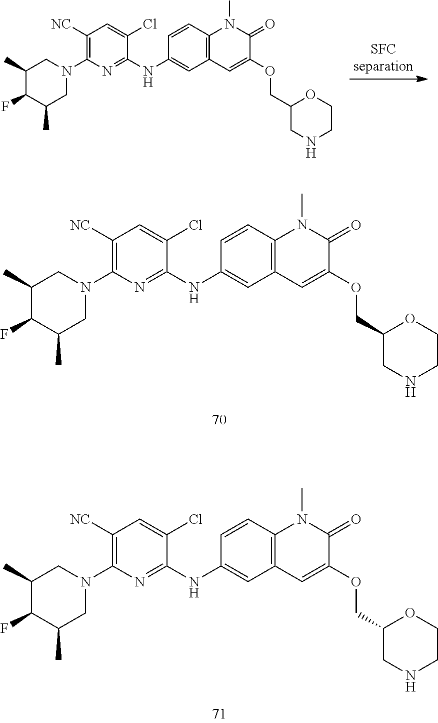 [SMILES: C[C@@H]1CN(c2nc(Nc3ccc4c(c3)cc(OCC3CNCCO3)c(=O)n4C)c(Cl)cc2C#N)C[C@H](C)[C@@H]1F ; C[C@@H]1CN(c2nc(Nc3ccc4c(c3)cc(OC[C@@H]3CNCCO3)c(=O)n4C)c(Cl)cc2C#N)C[C@H](C)[C@@H]1F ; C[C@@H]1CN(c2nc(Nc3ccc4c(c3)cc(OC[C@H]3CNCCO3)c(=O)n4C)c(Cl)cc2C#N)C[C@H](C)[C@@H]1F]

### 3-Hydroxy-1-methyl-6-nitroquinolin-2(1H)-one

To a solution of 1-methyl-5-nitroindoline-2,3-dione (25 g, 121.27 mmol) and TEA (24.54 g, 242.54 mmol, 33.76 mL) in EtOH (750 mL) was added diazomethyl(trimethyl)silane (2 M, 72.76 mL) dropwise under N₂. The mixture was stirred at 20° C. for 12 hr. The mixture was concentrated under reduced pressure and 1N HCl aq (300 mL) was added. Then the mixture was stirred at 20° C. for 2 hr, filtered and washed with DMF/ethyl acetate (10/1, 150 mL), the filter cake was dried under reduced pressure to give the title compound as a brown solid (9.2 g, 35.18 mmol, 15% yield, 84% purity). ¹H NMR (400 MHz, DMSO-d₆) δ 10.08 (s, 1H), 8.55 (d, J=2.69 Hz, 1H), 8.19 (dd, J=9.29, 2.69 Hz, 1H), 7.65 (d, J=9.41 Hz, 1H), 7.34 (s, 1H), 3.74 (s, 3H).

### tert-Butyl 2-(((J-methyl-6-nitro-2-oxo-1,2-dihydroquinolin-3-yl)oxy)methyl)morpholine-4-carboxylate

To a solution of 3-hydroxy-1-methyl-6-nitroquinolin-2(1H)-one (1 g, 4.54 mmol) and tert-butyl 2-(bromomethyl)morpholine-4-carboxylate (1.53 g, 5.45 mmol) in DMSO (20 mL) was added DBU (829.70 mg, 5.45 mmol, 821.49 μL) under N₂. The mixture was stirred at 100° C. for 3 hr. The mixture (combined with another batch of 200 mg scale) was cooled to 20° C. and water (˜30 mL) was added, forming a precipitate which was filtered and the filter cake was washed with EtOAc (20 mL) and concentrated in vacuo to give the title compound as a yellow solid (1.2 g, 2.00 mmol, 44% yield, 70% purity). LCMS: [M−100]+=320.1.

### tert-Butyl 2-(((6-amino-1-methyl-2-oxo-1,2-dihydroquinolin-3-yl)oxy)methyl)morpholine-4-carboxylate

To a mixture of Pd/C (0.1 g, 10% purity) in THE (20 mL) was added tert-butyl 2-(((1-methyl-6-nitro-2-oxo-1,2-dihydroquinolin-3-yl)oxy)methyl)morpholine-4-carboxylate (0.6 g, 1.43 mmol) under Ar. The flask was degassed under vacuum and purged with H₂ several times. The mixture was stirred under H₂ (15 psi) at 20° C. for 12 hours. The mixture was filtered and the filtrate was concentrated in vacuo to give the title compound as a yellow solid (600 mg, 1.39 mmol, 97% yield, 90% purity). ¹H NMR (400 MHz, DMSO-d₆) δ 7.17 (d, J=8.82 Hz, 1H), 7.05 (s, 1H), 6.77 (dd, J=8.88, 2.56 Hz, 1H), 6.71 (d, J=2.50 Hz, 1H), 5.03 (s, 2H), 4.00 (d, J=5.01 Hz, 2H), 3.94 (d, J=13.35 Hz, 1H), 3.86 (d, J=10.25 Hz, 1H), 3.69-3.77 (m, 2H), 3.56 (s, 3H), 3.46 (td, J=11.56, 2.62 Hz, 1H), 2.89 (d, J=3.22 Hz, 2H), 1.41 (s, 9H).

### tert-Butyl 2-(((6-((3,6-dichloro-5-cyanopyridin-2-yl)amino)-1-methyl-2-oxo-1,2-dihydroquinolin-3-yl)oxy)methyl)morpholine-4-carboxylate

A flask with mixture of tert-butyl 2-(((6-amino-1-methyl-2-oxo-1,2-dihydroquinolin-3-yl)oxy)methyl)morpholine-4-carboxylate (1 g, 2.57 mmol), 2,5,6-trichloronicotinonitrile (532.67 mg, 2.57 mmol) and DIEA (663.73 mg, 5.14 mmol, 894.52 μL) in DMF (20 mL) was degassed and purged with N₂ for 3 times, and then the mixture was stirred at 100° C. for 12 hr under N₂ atmosphere. The mixture was cooled to 20° C. and water (20 mL) was added, forming a precipitate which was filtered and the filter cake was dried in vacuo to give the title compound as a yellow solid (1 g, 1.48 mmol, 58% yield, 83% purity). LCMS: [M+H]⁺=560.2.

### tert-Butyl 2-(((6-((3-chloro-5-cyano-6-((3S,4S,5R)-4-fluoro-3,5-dimethylpiperidin-1-yl)pyridin-2-yl)amino)-1-methyl-2-oxo-1,2-dihydroquinolin-3-yl)oxy)methyl)morpholine-4-carboxylate

To a solution of tert-butyl 2-(((6-((3,6-dichloro-5-cyanopyridin-2-yl)amino)-1-methyl-2-oxo-1,2-dihydroquinolin-3-yl)oxy)methyl)morpholine-4-carboxylate (200 mg, 356.87 μmol) and (3S,4S,5R)-4-fluoro-3,5-dimethylpiperidine hydrochloride (119.66 mg, 713.74 μmol, HCl) in DMSO (4 mL) was added DIEA (230.61 mg, 1.78 mmol, 310.80 μL). The mixture was stirred at 100° C. for 12 hr. The mixture (combined with another batch of 100 mg scale) was cooled to 20° C. and water (10 mL) was added, forming a precipitate which was filtered and the filter cake was concentrated in vacuo to give the title compound as a white solid (300 mg, 70% purity). LCMS: [M+H]⁺=655.3.

### 5-Chloro-2-((3S,4S,5R)-4-fluoro-3,5-dimethylpiperidin-1-yl)-6-((1-methyl-3-(morpholin-2-ylmethoxy)-2-oxo-1,2-dihydroquinolin-6-yl)amino)nicotinonitrile

A mixture of tert-butyl 2-(((6-((3-chloro-5-cyano-6-((3S,4S,5R)-4-fluoro-3,5-dimethylpiperidin-1-yl)pyridin-2-yl)amino)-1-methyl-2-oxo-1,2-dihydroquinolin-3-yl)oxy)methyl)morpholine-4-carboxylate (250 mg, 381.59 μmol) and HCl/EtOAc (4 M, 10.00 mL) (4 M HCl in EtOAc) was stirred at 20° C. for 1 hr under N₂ atmosphere. The mixture was concentrated in vacuo without further work-up. The residue was purified by prep-HPLC (column: Phenomenex Luna C18 80*40 mm*3 μm; mobile phase: [water(HCl)-ACN]; B %: 30%-60%, 7 min) to give the title compound as a yellow solid (110 mg, 184.11 μmol, 48% yield, 99% purity, HCl). ¹H NMR (400 MHz, DMSO-d₆) δ 9.27-9.41 (m, 2H), 9.09 (s, 1H), 7.96 (s, 1H), 7.86 (d, J=2.32 Hz, 1H), 7.63 (dd, J=9.05, 2.45 Hz, 1H), 7.47 (d, J=9.05 Hz, 1H), 7.25 (s, 1H), 4.11-4.20 (m, 2H), 4.08 (d, J=4.65 Hz, 2H), 3.96-4.05 (m, 3H), 3.78-3.88 (m, 2H), 3.67 (s, 3H), 2.94-3.04 (m, 2H), 2.79 (t, J=12.59 Hz, 2H), 1.77-1.94 (m, 2H), 0.86 (d, J=6.85 Hz, 6H).

### 5-Chloro-2-((3S,4S,5R)-4-fluoro-3,5-dimethylpiperidin-1-yl)-6-((1-methyl-3-((S)-morpholin-2-ylmethoxy)-2-oxo-1,2-dihydroquinolin-6-yl)amino)nicotinonitrile (70)

Racemic 5-chloro-2-((3S,4S,5R)-4-fluoro-3,5-dimethylpiperidin-1-yl)-6-((1-methyl-3-(morpholin-2-ylmethoxy)-2-oxo-1,2-dihydroquinolin-6-yl)amino)nicotinonitrile (110 mg, 185.97 μmol, HCl) was separated with SFC (column: REGIS(S,S)WHELK-O1 (250 mm*25 mm, 10 μm); mobile phase: [0.1% NH₃H₂O MeOH]; B %: 60%-60%, 40 min) to give the title compound as a yellow solid (40 mg, 71.92 μmol, 39% yield, 99% purity). ¹H NMR (400 MHz, DMSO-d₆) δ 9.04 (s, 1H), 7.93-7.95 (m, 1H), 7.88 (d, J=2.32 Hz, 1H), 7.59 (dd, J=9.05, 2.32 Hz, 1H), 7.43 (d, J=9.05 Hz, 1H), 7.20 (s, 1H), 4.41-4.59 (m, 1H), 3.85-4.04 (m, 5H), 3.74 (s, 2H), 3.65 (s, 3H), 3.48 (td, J=10.64, 3.42 Hz, 1H), 2.89 (dd, J=12.17, 1.41 Hz, 1H), 2.78 (t, J=12.65 Hz, 2H), 2.64-2.72 (m, 3H), 1.76-1.96 (m, 2H), 0.86 (d, J=6.97 Hz, 6H).

### 5-Chloro-2-((3S,4S,5R)-4-fluoro-3,5-dimethylpiperidin-1-yl)-6-((1-methyl-3-((R)-morpholin-2-ylmethoxy)-2-oxo-1,2-dihydroquinolin-6-yl)amino)nicotinonitrile (71)

Racemic 5-chloro-2-((3S,4S,5R)-4-fluoro-3,5-dimethylpiperidin-1-yl)-6-((1-methyl-3-(morpholin-2-ylmethoxy)-2-oxo-1,2-dihydroquinolin-6-yl)amino)nicotinonitrile (110 mg, 185.97 μmol, HCl) was separated with SFC (column: REGIS(S,S)WHELK-O1 (250 mm*25 mm, 10 μm); mobile phase: [0.1% NH₃H₂O MEOH]; B %: 60%-60%, 40 min) to give the title compound as a yellow solid (25 mg, 44.56 μmol, 24% yield, 99% purity). ¹H NMR (400 MHz, DMSO-d₆) δ 9.05 (s, 1H), 7.94 (s, 1H), 7.87 (d, J=2.20 Hz, 1H), 7.58 (dd, J=8.99, 2.26 Hz, 1H), 7.43 (d, J=9.05 Hz, 1H), 7.20 (s, 1H), 4.42-4.59 (m, 1H), 3.87-4.03 (m, 5H), 3.75 (d, J=10.51 Hz, 2H), 3.65 (s, 3H), 3.48 (td, J=10.64, 3.30 Hz, 1H), 2.89 (d, J=10.76 Hz, 1H), 2.78 (t, J=12.65 Hz, 2H), 2.61-2.72 (m, 2H), 1.76-1.93 (m, 2H), 0.86 (d, J=6.85 Hz, 6H).

### Example 33: Synthesis of 2-((6-((3-chloro-5-cyano-6-((3S,4S,5R)-4-fluoro-3,5-dimethylpiperidin-1-yl)pyridin-2-yl)amino)-1-(2-(dimethylamino)ethyl)-2-oxo-1,2-dihydroquinolin-3-yl)oxy)-N-methylacetamide (72)

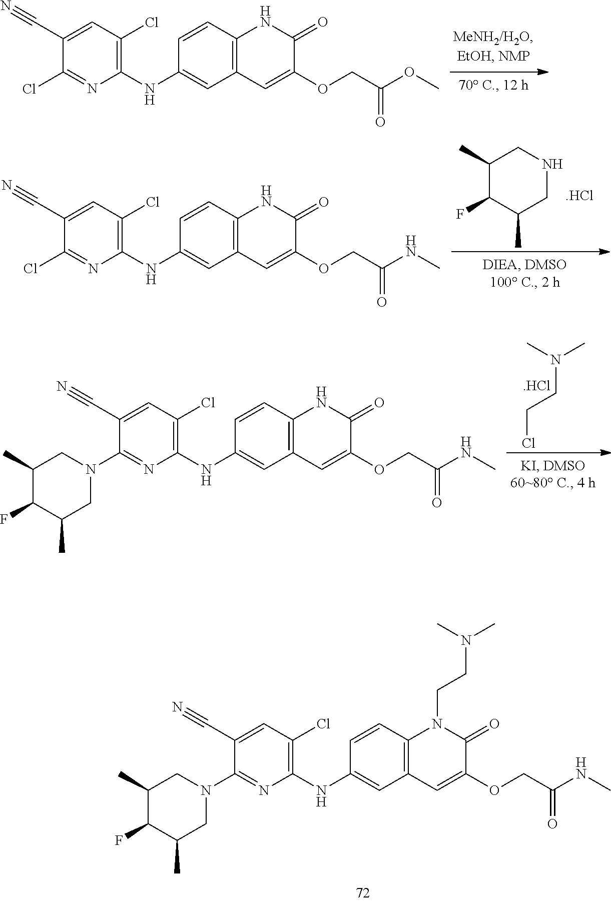

### 2-((6-((3,6-Dichloro-5-cyanopyridin-2-yl)amino)-2-oxo-1,2-dihydroquinolin-3-yl)oxy)-N-methylacetamide

To a solution of methyl 2-[[6-[(3,6-dichloro-5-cyano-2-pyridyl)amino]-2-oxo-1H-quinolin-3-yl]oxy]acetate (3 g, 7.16 mmol) in NMP (3 mL) and EtOH (27 mL) was added MeNH₂ (4.44 g, 57.25 mmol, 40% purity). The mixture was stirred at 70° C. for 12 hr. The reaction mixture was cooled to 20° C. and EtOH (90 mL) was added and the suspension was filtered. The filter cake was dried under reduced pressure to give the title compound as a yellow solid (2.6 g, 5.41 mmol, 76% yield, 87% purity).

### 2-((6-((3-Chloro-5-cyano-6-((3S,4S,5R)-4-fluoro-3,5-dimethylpiperidin-1-yl)pyridin-2-yl)amino)-2-oxo-1,2-dihydroquinolin-3-yl)oxy)-N-methylacetamide

To a solution of 2-[[6-[(3,6-dichloro-5-cyano-2-pyridyl)amino]-2-oxo-1H-quinolin-3-yl]oxy]-N-methyl-acetamide (500 mg, 1.20 mmol) and (3S,4S,5R)-4-fluoro-3,5-dimethyl-piperidine (300.64 mg, 1.79 mmol, HCl) in DMSO (1 mL) was added DIEA (618.04 mg, 4.78 mmol, 832.94 μL). The mixture was stirred at 100° C. for 2 hr, then cooled to room temperature and treated with water (1 mL), forming a precipitate which was filtered. The filter cake was washed with EtOAc (2 mL) and then dried under reduce pressure to give the title compound as a brown solid (600 mg, 818.77 μmol, 68% yield, 70% purity).

### 2-((6-((3-Chloro-5-cyano-6-((3S,4S,5R)-4-fluoro-3,5-dimethylpiperidin-1-yl)pyridin-2-yl)amino)-1-(2-(dimethylamino)ethyl)-2-oxo-1,2-dihydroquinolin-3-yl)oxy)-N-methylacetamide (72)

To a solution of 2-[[6-[[3-chloro-5-cyano-6-[(3S,4S,5R)-4-fluoro-3,5-dimethyl-1-piperidyl]-2-pyridyl]amino]-2-oxo-1H-quinolin-3-yl]oxy]-N-methyl-acetamide (100 mg, 194.95 μmol) and 2-chloro-N,N-dimethyl-ethanamine (42.12 mg, 292.42 μmol, HCl) in DMSO (1 mL) was added KI (16.18 mg, 97.47 μmol), Cs₂CO₃ (190.55 mg, 584.84 μmol). The mixture was stirred at 60° C. and then was heated to 80° C. for 2 hr. The reaction mixture was treated with water (3 mL), forming a precipitate which was filtered and the filter cake was washed with EtOAc (6 mL). The filter cake was collected and dried in vacuo to give the title compound as an orange solid (2.6 mg, 4.23 μmol, 2% yield, 95% purity). ¹H NMR (400 MHz, MeOD-d₄) δ 8.02 (d, J=2.38 Hz, 1H), 7.76-7.80 (m, 1H), 7.75 (s, 1H), 7.58 (d, J=9.18 Hz, 1H), 7.39-7.43 (m, 1H), 4.83-4.84 (m, 2H), 4.61 (s, 2H), 4.41-4.58 (m, 1H), 4.11 (dd, J=12.87, 3.93 Hz, 2H), 3.61 (t, J=6.02 Hz, 2H), 3.09 (s, 6H), 2.85-2.93 (m, 5H), 1.80-2.00 (m, 2H), 0.98 (d, J=6.91 Hz, 6H).

### Example 34: Synthesis of 2-((6-((3-chloro-5-cyano-6-((3S,4S,5R)-4-hydroxy-3,5-dimethylpiperidin-1-yl)pyridin-2-yl)amino)-1-(2-(dimethylamino)ethyl)-2-oxo-1,2-dihydroquinolin-3-yl)oxy)-N-methylacetamide (73)

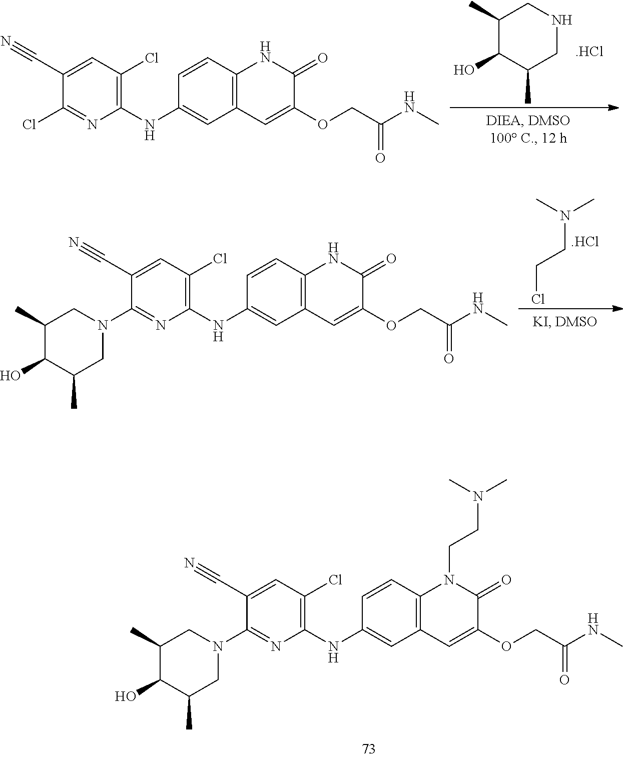

### 2-((6-((3-Chloro-5-cyano-6-((3S,4S,5R)-4-hydroxy-3,5-dimethylpiperidin-1-yl)pyridin-2-yl)amino)-2-oxo-1,2-dihydroquinolin-3-yl)oxy)-N-methylacetamide

To a solution of 2-[[6-[(3,6-dichloro-5-cyano-2-pyridyl)amino]-2-oxo-1H-quinolin-3-yl]oxy]-N-methyl-acetamide (300 mg, 717.30 μmol) and (3R,5S)-3,5-dimethylpiperidin-4-ol (178.24 mg, 1.08 mmol, HCl) in DMSO (1 mL) was added DIPEA (370.82 mg, 2.87 mmol, 499.75 μL). The mixture was stirred at 100° C. for 12 hr. Then water (5 mL) was added, forming a precipitate which was filtered and the filter cake was washed with water (10 mL) and EtOAc (20 mL), then the filter cake was dried in vacuo to give the title compound as a brown solid (250 mg, 457.95 μmol, 64% yield, 94% purity). ¹H NMR (400 MHz, DMSO-d₆) δ 12.04 (s, 1H), 8.99 (s, 1H), 7.99 (m, 1H), 7.92 (s, 1H), 7.76 (d, J=2 Hz, 1H), 7.61 (dd, J=8.8, 2.4 Hz, 1H), 7.30 (d, J=8.8 Hz, 1H), 7.25 (s, 1H), 4.61-4.58 (m, 3H), 3.92 (dd, J=13.2, 3.2 Hz, 2H), 3.49 (d, J=4.4 Hz, 1H), 2.91-2.83 (m, 2H), 2.74 (d, J=4.8 Hz, 3H), 1.72-1.69 (m, 2H), 0.84 (d, J=6.8 Hz, 6H).

### 2-((6-((3-chloro-5-cyano-6-((3S,4S,5R)-4-hydroxy-3,5-dimethylpiperidin-1-yl)pyridin-2-yl)amino)-1-(2-(dimethylamino)ethyl)-2-oxo-1,2-dihydroquinolin-3-yl)oxy)-N-methylacetamide (73)

To a solution of 2-[[6-[[3-chloro-5-cyano-6-[(3R,5S)-4-hydroxy-3,5-dimethyl-1-piperidyl]-2-pyridyl]amino]-2-oxo-1H-quinolin-3-yl]oxy]-N-methyl-acetamide (150 mg, 293.56 μmol) and 2-chloro-N,N-dimethyl-ethanamine (63.43 mg, 440.34 μmol, HCl) in DMSO (3 mL) was added Cs₂CO₃ (286.94 mg, 880.68 μmol) and KI (24.37 mg, 146.78 μmol). The mixture was stirred at 60° C. for 2 hr. Water (2 mL) was added to the mixture (combined with another batch of 70 mg scale) and filtered. The filter cake was dried under vacuum to give a residue which was purified by prep-HPLC (column: Waters Xbridge BEH C18 100*30 mm*10 μm; mobile phase: [water(NH₄HCO₃)-ACN]; B %: 35%-65%, 8 min) to give the title compound as a white solid (96% purity). ¹H NMR (400 MHz, DMSO-d₆) δ 9.00 (br s, 1H), 7.96 (br d, J=4.41 Hz, 1H), 7.88 (s, 1H), 7.77 (d, J=2.15 Hz, 1H), 7.69 (dd, J=9.06, 2.15 Hz, 1H), 7.45 (d, J=9.18 Hz, 1H), 7.19 (s, 1H), 4.52 (s, 3H), 4.39 (br t, J=7.09 Hz, 2H), 3.87 (br dd, J=12.64, 3.10 Hz, 2H), 3.43 (br s, 1H), 3.21-3.29 (m, 2H), 2.83 (br t, J=12.46 Hz, 2H), 2.67 (d, J=4.53 Hz, 3H), 2.23 (s, 6H), 1.59-1.73 (m, 2H), 0.79 (d, J=6.79 Hz, 6H).

### Example 35: Synthesis of 5-chloro-2-((3S,5R)-4,4-difluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile (74) and 5-chloro-2-((3R,5S)-4,4-difluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile (75)

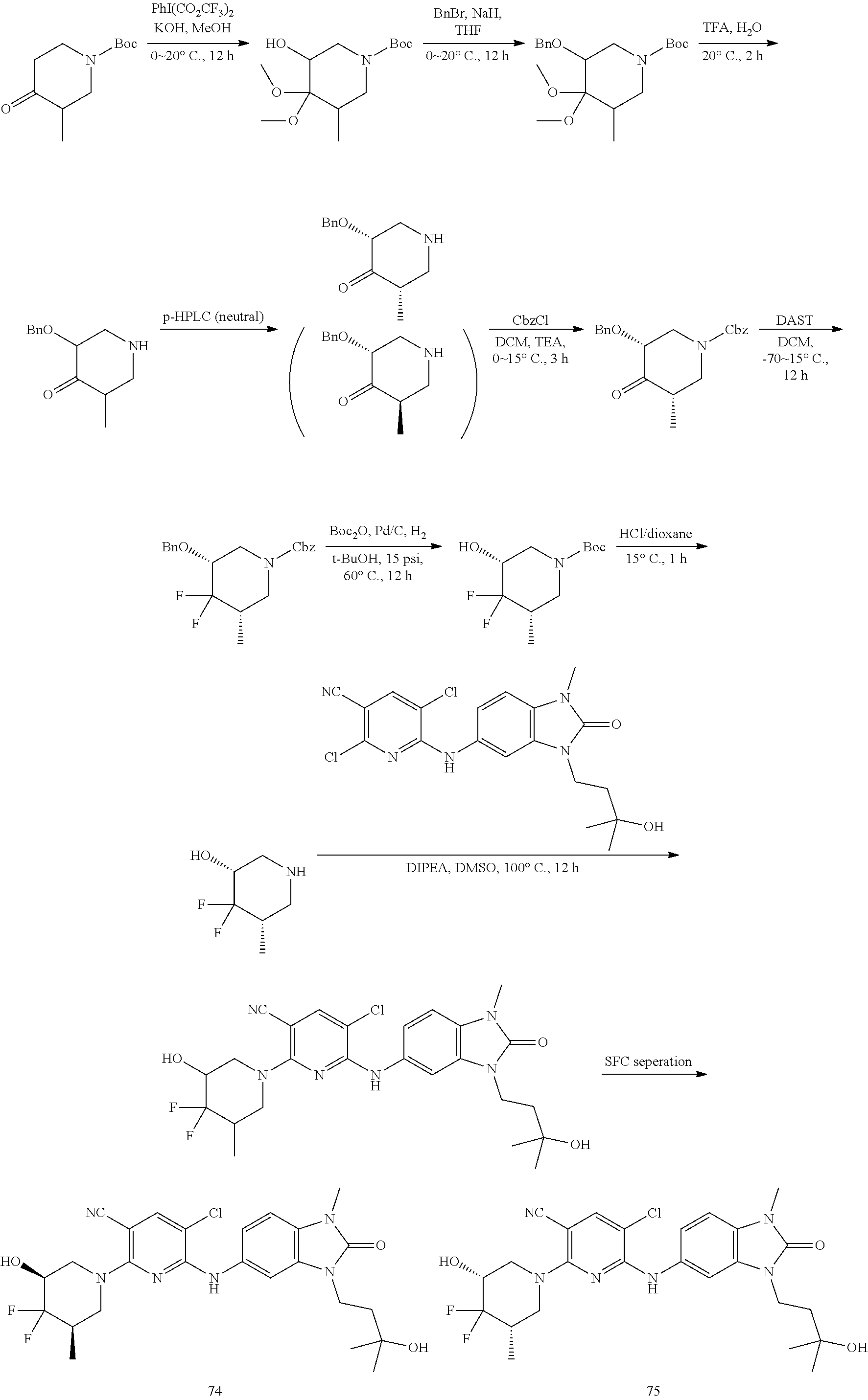

### tert-Butyl 3-hydroxy-4,4-dimethoxy-5-methylpiperidine-1-carboxylate

To a solution of KOH (133.48 g, 2.38 mol) in MeOH (590 mL) was added tert-butyl 3-methyl-4-oxo-piperidine-1-carboxylate (118 g, 553.28 mmol) portion-wise at 0° C., then the mixture was stirred at 0° C. for 20 min. Then [phenyl-(2,2,2-trifluoroacetyl)oxy-λ3-iodanyl]2,2,2-trifluoroacetate (356.90 g, 829.92 mmol, 1.5 eq) was added portion-wise at 0° C. The resulting mixture was stirred at 20° C. for 12 hr. The mixture (combined with other 4 batches of same scale) was concentrated in vacuo. Then water (2 L) was added and extracted with ethyl acetate (1 L×3). The combined organic phase was washed with brine (2 L), dried with anhydrous Na₂SO₄, filtered and concentrated in vacuo. The residue was purified by flash silica gel column chromatography (Silica Flash Column, Eluent of 0˜50% Ethyl acetate/Petroleum ether gradient) to give the title compound as a yellow oil (750 g). ¹H NMR (400 MHz, CDCl₃) δ 4.04-4.29 (m, 1H), 3.72-3.93 (m, 2H), 3.26 (s, 3H), 3.21 (s, 3H), 2.93-3.13 (m, 2H), 1.95-2.10 (m, 2H), 1.44-1.47 (m, 9H), 1.13 (d, J=7.34 Hz, 3H).

### tert-Butyl 3-(benzyloxy)-4,4-dimethoxy-5-methylpiperidine-1-carboxylate

To a solution of NaH (29.85 g, 746.23 mmol, 60% purity) in THE (1 L) was added tert-butyl 3-hydroxy-4,4-dimethoxy-piperidine-1-carboxylate (150 g, 574.02 mmol) in THE (500 mL) dropwise at 0° C. After addition, the mixture was stirred at 0° C. for 30 min, then bromomethylbenzene (108.00 g, 631.42 mmol, 75.00 mL) was added dropwise at 0° C. The resulting mixture was stirred at 20° C. for 11.5 hr (4 batches). The reaction mixture (combined with other 3 batches of same scale) was quenched with sat. NH₄Cl (1 L). The mixture was concentrated and extracted with ethyl acetate (500 mL×3). The combined organic phase was dried with anhydrous Na₂SO₄, filtered and concentrated under reduced pressure. The residue was purified by flash silica gel column chromatography (Silica Flash Column, Eluent of 0˜50% Ethyl acetate/Petroleum ether gradient) to give the title compound as a yellow oil (80 g). ¹H NMR (400 MHz, CDCl₃) δ 7.20-7.44 (m, 5H), 4.74-4.89 (m, 1H), 4.26-4.60 (m, 2H), 3.71-4.01 (m, 1H), 3.43-3.57 (m, 1H), 3.14-3.25 (m, 6H), 2.86-3.12 (m, 2H), 1.98-2.16 (m, 1H), 1.36-1.51 (m, 9H), 1.15 (br dd, J=12.04, 7.27 Hz, 3H).

### 3-(Benzyloxy)-5-methylpiperidin-4-one

A mixture of tert-butyl 3-benzyloxy-4,4-dimethoxy-5-methyl-piperidine-1-carboxylate (84.5 g, 231.21 mmol) in H₂O (300 mL) and TFA (600 mL) was stirred at 20° C. for 2 hours. The reaction mixture was concentrated under reduced pressure to give the title compound as a yellow oil (75 g, 225.02 mmol, 97% yield, TFA). LCMS: [M+H]⁺=220.2.

### (3R,5S)-3-(Benzyloxy)-5-methylpiperidin-4-one

3-Benzyloxy-5-methyl-piperidin-4-one (60.00 g, 180.02 mmol, TFA salt) was mixed with sat. aq. NaHCO₃ (500 mL) and extracted with EtOAc (300 mL×2). The combined organic phase was washed with brine (500 mL), dried over anhydrous Na₂SO₄, filtered and concentrated in vacuo. The residue was purified by flash silica gel column chromatography (Silica Flash Column, Eluent of 30˜100% Ethyl acetate/Petroleum ether gradient) to give 40 g of a mixture of products with two close spots on TLC. The 40 g of the mixture product was purified by prep-HPLC (column: Waters Xbridge BEH C18 100*30 mm*10 μm; mobile phase: [water(NH₃H₂O+NH₄HCO₃)-ACN]; B %: 15%-50%, 8 min) to give the title compound as a colorless oil (16 g, 66.40 mmol, 37% yield, 91% purity). ¹H NMR (400 MHz, CDCl₃) δ 7.28-7.43 (m, 5H), 4.88 (d, J=11.92 Hz, 1H), 4.51 (d, J=11.80 Hz, 1H), 3.95 (dd, J=10.67, 6.74 Hz, 1H), 3.56 (ddd, J=12.28, 6.79, 2.03 Hz, 1H), 3.25-3.38 (m, 1H), 2.79 (dd, J=12.10, 10.91 Hz, 1H), 2.43-2.55 (m, 2H), 0.99-1.11 (m, 3H).

### (3R,5S)-Benzyl 3-(benzyloxy)-5-methyl-4-oxopiperidine-1-carboxylate

To a mixture of (3R,5S)-3-benzyloxy-5-methyl-piperidin-4-one (10.00 g, 45.60 mmol) in DCM (100 mL) was added DIEA (17.68 g, 136.81 mmol, 23.83 mL) in one portion under N₂. Then CbzCl (8.56 g, 50.16 mmol, 7.13 mL) was added dropwise at 0° C. under N₂. The mixture was stirred at 15° C. for 3 hours. The mixture was washed with 10% citric acid (100 mL) and extracted with ethyl acetate (120 mL×2). The combined organic phase was washed with brine (200 mL), dried with anhydrous Na₂SO₄, filtered and concentrated in vacuo. The residue was purified by flash silica gel column chromatography (ISCO®; 40 g SepaFlash® Silica Flash Column, Eluent of 0˜10% Ethyl acetate/Petroleum ether gradient @100 mL/min) to give the title compound as a yellow oil (10.5 g, 27.80 mmol, 61% yield, 94% purity). ¹H NMR (400 MHz, CDCl₃) δ 7.28-7.47 (m, 10H), 5.18 (br s, 2H), 4.87 (d, J=11.63 Hz, 1H), 4.28-4.81 (m, 3H), 3.97 (br s, 1H), 3.00 (br s, 1H), 2.47-2.82 (m, 2H), 1.08 (d, J=6.38 Hz, 3H).

### (3R,5S)-Benzyl 3-(benzyloxy)-4,4-difluoro-5-methylpiperidine-1-carboxylate

To a solution of benzyl (3R,5S)-3-benzyloxy-5-methyl-4-oxo-piperidine-1-carboxylate (3 g, 8.49 mmol) in DCM (40 mL) was added DAST (2.74 g, 16.98 mmol, 2.24 mL) at −70° C. The mixture was stirred at 15° C. for 12 hr. Then the mixture was quenched with sat. NaHCO₃ (˜60 mL) to pH=8 and the mixture was extracted with DCM (40 mL×2). The combined organic phase was washed with brine (100 mL), dried over anhydrous Na₂SO₄, filtered and concentrated in vacuo. The residue was purified by flash silica gel column chromatography (Silica Flash Column, Eluent of 0˜30% Ethyl acetate/Petroleum ether gradient @100 mL/min) to give the title compound as a colorless oil (2 g, 5.33 mmol, 63% yield). ¹H NMR (400 MHz, CDCl₃) δ 7.27-7.46 (m, 10H), 5.11 (br s, 2H), 4.59-4.89 (m, 2H), 3.84-4.39 (m, 2H), 3.54 (br s, 1H), 3.07 (br t, J=11.44 Hz, 1H), 2.83 (br d, J=7.51 Hz, 1H), 1.90-2.07 (m, 1H), 1.09 (br d, J=6.68 Hz, 3H).

### (3R,5S)-tert-Butyl 4,4-difluoro-3-hydroxy-5-methylpiperidine-1-carboxylate

To a solution of benzyl (3R,5S)-3-benzyloxy-4,4-difluoro-5-methyl-piperidine-1-carboxylate (1 g, 2.66 mmol) in t-BuOH (5 mL) was added Pd/C (0.3 g, 10% purity) and Boc₂O (1.16 g, 5.33 mmol, 1.22 mL) under Ar. The suspension was degassed under vacuum and purged with H₂ several times. The mixture was stirred under H₂ (15 psi) at 60° C. for 12 hours. The mixture was filtered through Celite® and the filtrate was concentrated in vacuo to give the title compound as a colorless oil (1.5 g, crude). ¹H NMR (400 MHz, MeOD-d₄) δ 4.04-4.17 (m, 1H), 3.94 (br d, J=10.01 Hz, 1H), 3.61-3.75 (m, 1H), 2.57-2.95 (m, 2H), 1.89-2.10 (m, 1H), 1.47 (s, 9H), 1.04 (d, J=6.88 Hz, 3H).

### (3R,5S)-4,4-Difluoro-5-methylpiperidin-3-ol hydrochloride

A solution of tert-butyl (3R,5S)-4,4-difluoro-3-hydroxy-5-methyl-piperidine-1-carboxylate (1.5 g, 5.97 mmol) in 4N HCl/EtOAc (20 mL) was stirred at 15° C. for 1 hours. The mixture was concentrated in vacuo to give the title compound as a white solid (1.1 g, 5.86 mmol, 98% yield, HCl). ¹H NMR (400 MHz, MeOD-d₄) δ 4.03-4.18 (m, 1H), 3.47 (br d, J=12.38 Hz, 1H), 3.39 (br d, J=12.63 Hz, 1H), 3.04 (br t, J=11.63 Hz, 1H), 2.94 (br t, J=12.26 Hz, 1H), 2.30-2.52 (m, 1H), 1.15 (d, J=6.75 Hz, 3H).

### 5-Chloro-2-(4,4-difluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino) nicotinonitrile

To a solution of 2,5-dichloro-6-[[3-(3-hydroxy-3-methyl-butyl)-1-methyl-2-oxo-benzimidazol-5-yl]amino]pyridine-3-carbonitrile (100 mg, 237.93 μmol) 4,4-difluoro-5-methyl-piperidin-3-ol (66.96 mg, 356.89 μmol, HCl) in DMSO (1 mL) was added DIPEA (123.00 mg, 951.72 μmol, 165.77 μL). The mixture was stirred at 100° C. for 12 hr. The mixture was cooled to 15° C. and purified directly with prep-HPLC (column: Waters Xbridge Prep OBD C18 150*40 mm*10 μm; mobile phase: [water(NH₄HCO₃)-ACN]; B %: 25%-55%, 8 min) to give the title compound as a white solid (50 mg, 91.22 μmol, 38% yield, 98% purity). ¹H NMR (400 MHz, DMSO-d₆) δ 9.01 (s, 1H), 7.95 (s, 1H), 7.29 (s, 1H), 7.22 (br d, J=8.13 Hz, 1H), 7.11 (br d, J=8.25 Hz, 1H), 5.72 (br d, J=5.50 Hz, 1H), 4.46 (s, 1H), 4.23 (br d, J=12.26 Hz, 1H), 3.96 (br d, J=12.38 Hz, 1H), 3.83-3.91 (m, 2H), 3.57-3.73 (m, 1H), 3.32 (br s, 3H), 2.93 (br t, J=11.94 Hz, 1H), 2.71 (br t, J=12.63 Hz, 1H), 1.93-2.14 (m, 1H), 1.63-1.75 (m, 2H), 1.16 (s, 6H), 0.79 (br d, J=6.50 Hz, 3H).

### 5-chloro-2-((3S,5R)-4,4-difluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino) nicotinonitrile (74) and 5-chloro-2-((3R,5S)-4,4-difluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino) nicotinonitrile (75)

Racemic 5-chloro-2-(4,4-difluoro-3-hydroxy-5-methyl-1-piperidyl)-6-[[3-(3-hydroxy-3-methyl-butyl)-1-methyl-2-oxo-benzimidazol-5-yl]amino]pyridine-3-carbonitrile (60 mg, 112.15 μmol) was separated with SFC (column: DAICEL CHIRALPAK IG (250 mm*30 mm, 10 μm); mobile phase: [0.1% NH₃H₂O IPA]; B %: 40%-40%, 20 min) to give 5-chloro-2-((3S,5R)-4,4-difluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl) amino)nicotinonitrile as a white solid (18 mg, 99% purity, 100% ee); ¹H NMR (400 MHz, MeCN-d₃) δ 7.72 (s, 2H), 7.34 (d, J=1.88 Hz, 1H), 7.16 (dd, J=8.38, 1.88 Hz, 1H), 7.02 (d, J=8.38 Hz, 1H), 4.27-4.36 (m, 1H), 4.04-4.12 (m, 1H), 3.91-3.98 (m, 2H), 3.70-3.88 (m, 2H), 3.34 (s, 3H), 2.94-3.03 (m, 1H), 2.80-2.92 (m, 2H), 2.02-2.12 (m, 1H), 1.77-1.83 (m, 2H), 1.22 (s, 6H), 0.93 (d, J=6.75 Hz, 3H); and 5-chloro-2-((3R,5S)-4,4-difluoro-3-hydroxy-5-methylpiperidin-1-yl)-6-((3-(3-hydroxy-3-methylbutyl)-1-methyl-2-oxo-2,3-dihydro-1H-benzo[d]imidazol-5-yl)amino)nicotinonitrile as a white solid (17 mg, 99% purity, 99% ee) as white solid; ¹H NMR (400 MHz, MeCN-d₃) δ 7.72 (s, 2H), 7.34 (d, J=1.75 Hz, 1H), 7.16 (dd, J=8.32, 1.94 Hz, 1H), 7.02 (d, J=8.38 Hz, 1H), 4.24-4.37 (m, 1H), 4.03-4.12 (m, 1H), 3.95 (dd, J=8.82, 7.19 Hz, 2H), 3.70-3.88 (m, 2H), 3.34 (s, 3H), 2.94-3.03 (m, 1H), 2.90 (s, 1H), 2.80-2.89 (m, 1H), 2.01-2.12 (m, 1H), 1.77-1.84 (m, 2H), 1.22 (s, 6H), 0.93 (d, J=6.88 Hz, 3H).

The absolute configurations of compounds 74 & 75 were randomly assigned based on the hydroxyl group and methyl group being in cis-conformation.

### Example 36: Synthesis of 2-((6-((3-chloro-5-cyano-6-(3-hydroxy-5-methylpiperidin-1-yl)pyridin-2-yl)amino)-1-(2-(dimethylamino)ethyl)-2-oxo-1,2-dihydroquinolin-3-yl)oxy)-N-methylacetamide (76)

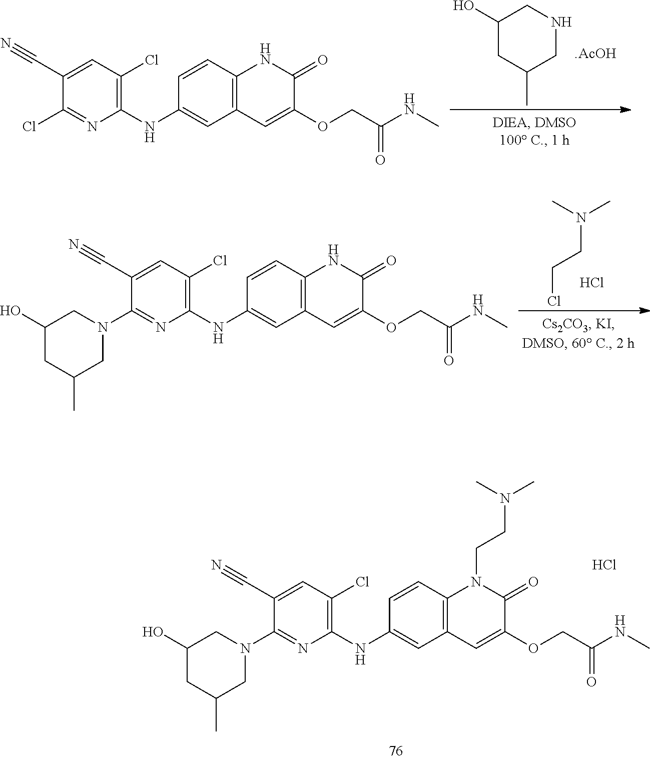

### 2-((6-((3-Chloro-5-cyano-6-(3-hydroxy-5-methylpiperidin-1-yl)pyridin-2-yl)amino)-2-oxo-1,2-dihydroquinolin-3-yl)oxy)-N-methylacetamide

To a solution of 5-methylpiperidin-3-ol (315.40 mg, 1.80 mmol, HOAc) and 2-[[6-[(3,6-dichloro-5-cyano-2-pyridyl)amino]-2-oxo-1H-quinolin-3-yl]oxy]-N-methyl-acetamide (500 mg, 1.20 mmol) in DMSO (5 mL) was added DIEA (620.35 mg, 4.80 mmol, 836.05 μL). The mixture was stirred at 100° C. for 1 hr. The reaction mixture was treated with water (20 mL), forming a precipitate which was filtered and the filter cake was washed with EtOAc (30 mL). The filter cake was collected and dried under reduced pressure to give the title compound as a yellow solid (600 mg, 567.47 μmol, 47% yield, 47% purity). LCMS: [M+H]⁺=497.1.

### 2-((6-((3-Chloro-5-cyano-6-(3-hydroxy-5-methylpiperidin-1-yl)pyridin-2-yl)amino)-1-(2-(dimethylamino)ethyl)-2-oxo-1,2-dihydroquinolin-3-yl)oxy)-N-methylacetamide (76)

To a solution of 2-[[6-[[3-chloro-5-cyano-6-(3-hydroxy-5-methyl-1-piperidyl)-2-pyridyl]amino]-2-oxo-1H-quinolin-3-yl]oxy]-N-methyl-acetamide (500 mg, 1.01 mmol) and 2-chloro-N,N-dimethyl-ethanamine (217.39 mg, 1.51 mmol, HCl) in DMSO (5 mL) was added Cs₂CO₃ (983.47 mg, 3.02 mmol) and KI (83.51 mg, 503.07 μmol). The mixture was stirred at 60° C. for 2 hr. The reaction mixture was treated with water (30 mL), forming a precipitate which was filtered and the filter cake was washed with EtOAc (30 mL). The filter cake was collected and dried under reduced pressure to give a residue which was purified by prep-HPLC (column: Phenomenex Luna 80*30 mm*3 μm; mobile phase: [water(HCl)-ACN]; B %: 15%-35%, 8 min) to give the title compound as a yellow solid (8 mg, 12.53 μmol, 1% yield, 95% purity, HCl). ¹H NMR (400 MHz, DMSO-d₆) δ 10.07 (br s, 1H), 9.03 (s, 1H), 8.04 (d, J=2.03 Hz, 1H), 7.92-7.99 (m, 2H), 7.67-7.71 (m, 1H), 7.61-7.65 (m, 1H), 7.32 (s, 1H), 4.66 (br t, J=6.44 Hz, 2H), 4.54 (s, 2H), 4.33 (br dd, J=12.10, 3.99 Hz, 1H), 4.12 (br d, J=12.76 Hz, 1H), 3.51 (br dd, J=10.55, 4.47 Hz, 2H), 2.91 (d, J=4.65 Hz, 6H), 2.68 (d, J=4.65 Hz, 3H), 2.57 (br d, J=12.28 Hz, 1H), 2.54 (s, 1H), 2.41-2.45 (m, 1H), 1.98 (br d, J=12.04 Hz, 1H), 1.59-1.72 (m, 1H), 1.00 (q, J=11.88 Hz, 1H), 0.82 (d, J=6.56 Hz, 3H).

### Example 37: Synthesis of 2-((6-((3-chloro-5-cyano-6-((3R,4R,5S)-4-fluoro-3,5-dimethylpiperidin-1-yl)pyridin-2-yl)amino)-1-(2-(dimethylamino)ethyl)-2-oxo-1,2-dihydroquinolin-3-yl)oxy)-N-methylacetamide (77)

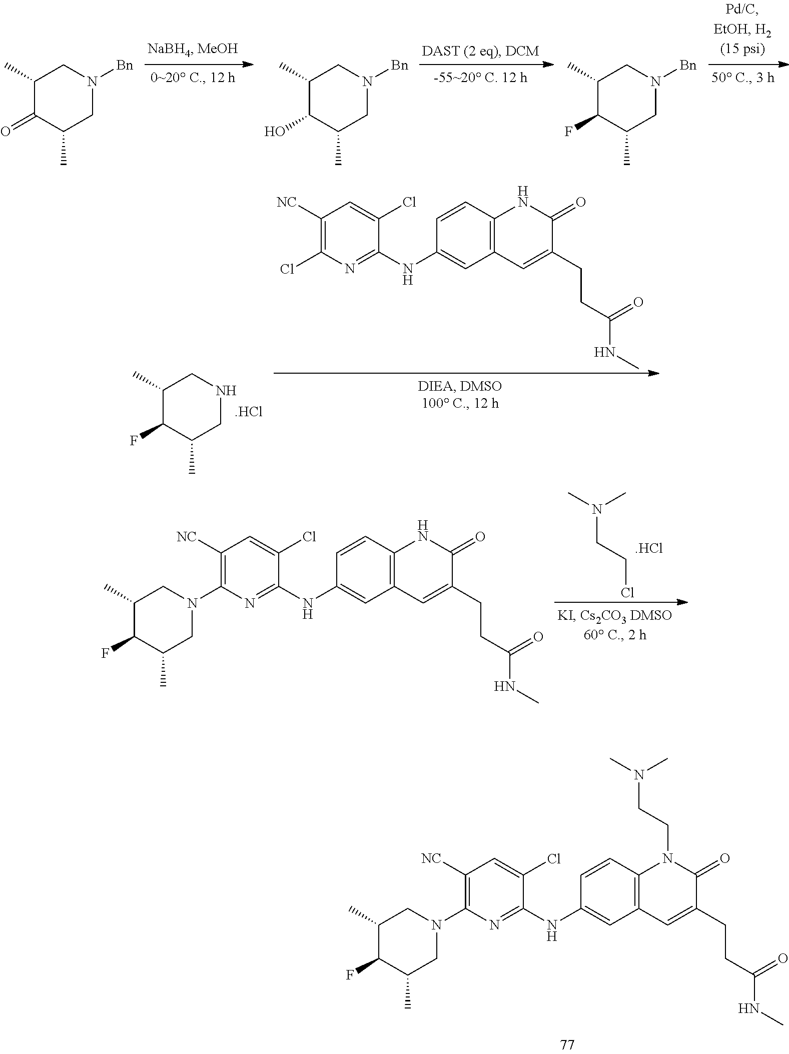

### (3R,4S,5S)-1-Benzyl-3,5-dimethylpiperidin-4-ol

To a solution of (3R,5S)-1-benzyl-3,5-dimethylpiperidin-4-one (4 g, 18.41 mmol) in MeOH (45 mL) was added NaBH₄ (835.67 mg, 22.09 mmol) at 0° C. for 10 min. The mixture was stirred at 20° C. for 12 hr. The reaction mixture was quenched by adding 1N HCl (40 mL) at 20° C., then the mixture was concentrated under reduced pressure. The residue was dissolved in ethyl acetate (40 mL) and washed with water (40 mL×3), the combined organic phase was dried with anhydrous Na₂SO₄, filtered and concentrated under reduced pressure. The residue was purified by flash silica gel column chromatography (Silica Flash Column, Eluent of 0˜20% Ethyl acetate/Petroleum) to give the title compound as a white solid (1 g, 3.65 mmol, 20% yield, 80% purity). LCMS: [M+H]⁺=220.2.

### (3R,4R,5S)-1-Benzyl-4-fluoro-3,5-dimethylpiperidine

To a solution of (3R,4S,5S)-1-benzyl-3,5-dimethylpiperidin-4-ol (650 mg, 2.96 mmol) in DCM (7 mL) was added DAST (955.43 mg, 5.93 mmol, 783.14 μL) at −55° C. under N₂. The mixture was stirred at 20° C. for 12 hr. The mixture was added sat. NaHCO₃ (30 mL), then the mixture was extracted with DCM (3×20 mL). The combined organic phase was dried with anhydrous Na₂SO₄, filtered and concentrated in vacuo. The residue was purified by silica gel column chromatography (silica gel, Petroleum ether/Ethyl acetate=1/0 to 10/1) to give the title compound as a yellow oil (500 mg, 2.09 mmol, 70% yield, 92% purity). ¹H NMR (400 MHz, DMSO-d₆) δ 7.23-7.35 (m, 5H), 3.59-3.78 (m, 1H), 3.45 (s, 2H), 2.72-2.80 (m, 2H), 1.75-1.84 (m, 2H), 1.67-1.74 (m, 2H), 0.90 (d, J=6.36 Hz, 6H).

### (3R,4R,5S)-4-Fluoro-3,5-dimethylpiperidine

To a mixture of Pd/C (0.1 g, 10% purity) in EtOH (5 mL) was added (3R,4R,5S)-1-benzyl-4-fluoro-3,5-dimethylpiperidine (500 mg, 2.26 mmol) under Ar. The flask was degassed under vacuum and purged with H₂ several times. The mixture was stirred under H₂ (15 psi) at 50° C. for 3 hours. The mixture was filtered through a pad of Celite® and washed with EtOH (50 mL). HCl/EtOAc (4M in EtOAc, 3 mL) was added to the filtrate. After stirring at 20° C. for 12 h, the mixture was concentrated in vacuo to give the title compound as a white solid (360 mg, 2.15 mmol, 95% yield, HCl). ¹H NMR (400 MHz, DMSO-d₆) δ 3.91-4.13 (m, 1H), 3.17-3.29 (m, 2H), 2.60-2.69 (m, 2H), 1.98-2.14 (m, 2H), 0.97-1.00 (m, 6H).

### 2-((6-((3-Chloro-5-cyano-6-((3R,4R,5S)-4-fluoro-3,5-dimethylpiperidin-1-yl)pyridin-2-yl)amino)-2-oxo-1,2-dihydroquinolin-3-yl)oxy)-N-methylacetamide

A flask with mixture of 2-[[6-[(3,6-dichloro-5-cyano-2-pyridyl)amino]-2-oxo-1H-quinolin-3-yl]oxy]-N-methyl-acetamide (200 mg, 478.20 μmol), (3R,4R,5S)-4-fluoro-3,5-dimethylpiperidine (120.26 mg, 717.30 μmol, HCl) and DIEA (432.63 mg, 3.35 mmol, 583.06 μL) in DMSO (2 mL) was degassed and purged with N₂ for 3 times, and then the mixture was stirred at 100° C. for 12 hr under N₂ atmosphere. Water (10 mL) was added to the mixture, forming a precipitate which was filtered. The filter cake was dry under reduced pressure and the residue was triturated with Petroleum ether/Ethyl acetate=3:1 (5 mL) at 15° C. for 10 min to give the title compound as a grey solid (280 mg, 436.68 μmol, 91% yield, 80% purity). LCMS: [M+H]⁺=513.2.

### 2-((6-((3-Chloro-5-cyano-6-((3R,4R,5S)-4-fluoro-3,5-dimethylpiperidin-1-yl)pyridin-2-yl)amino)-1-(2-(dimethylamino)ethyl)-2-oxo-1,2-dihydroquinolin-3-yl)oxy)-N-methylacetamide (77)

A flask with mixture of 2-[[6-[[3-chloro-5-cyano-6-[(3S,5R)-4-fluoro-3,5-dimethyl-1-piperidyl]-2-pyridyl]amino]-2-oxo-1H-quinolin-3-yl]oxy]-N-methyl-acetamide (100 mg, 194.95 μmol), 2-chloro-N,N-dimethyl-ethanamine (30.89 mg, 214.44 μmol, HCl), Cs₂CO₃ (190.55 mg, 584.84 μmol) and KI (16.18 mg, 97.47 μmol) in DMSO (1 mL) was degassed and purged with N₂ for 3 times, and then the mixture was stirred at 60° C. for 2 hr under N₂ atmosphere. The mixture was concentrated in vacuo without further work-up. The residue was purified by prep-HPLC (column: Phenomenex C18 80*40 mm*3 μm; mobile phase: [water(NH₄HCO₃)-ACN]; B %: 25%-60%, 8 min) to give the title compound as a white solid (25 mg, 92.27% purity). ¹H NMR (400 MHz, DMSO-d₆) δ 0.87 (d, J=6.48 Hz, 6H), 1.66-1.78 (m, 2H), 2.24 (s, 6H), 2.66-2.68 (m, 5H), 3.30 (br s, 2H), 3.79-3.99 (m, 1H), 4.09-4.19 (m, 2H), 4.39 (br t, J=7.15 Hz, 2H), 4.53 (s, 2H), 7.22 (s, 1H), 7.46 (d, J=9.17 Hz, 1H), 7.66 (dd, J=9.05, 2.32 Hz, 1H), 7.75 (d, J=2.32 Hz, 1H), 7.92-7.97 (m, 2H), 9.09 (s, 1H).

### Example 38: Synthesis of (3R,5S)-1-(5-chloro-3-cyano-6-((1-methyl-3-(2-(methylamino)-2-oxoethoxy)-2-oxo-1,2-dihydroquinolin-6-yl)amino)pyridin-2-yl)-5-methylpiperidine-3-carboxylic acid (78) and (3S,5R)-1-(5-chloro-3-cyano-6-((1-methyl-3-(2-(methylamino)-2-oxoethoxy)-2-oxo-1,2-dihydroquinolin-6-yl)amino)pyridin-2-yl)-5-methylpiperidine-3-carboxylic acid (79)

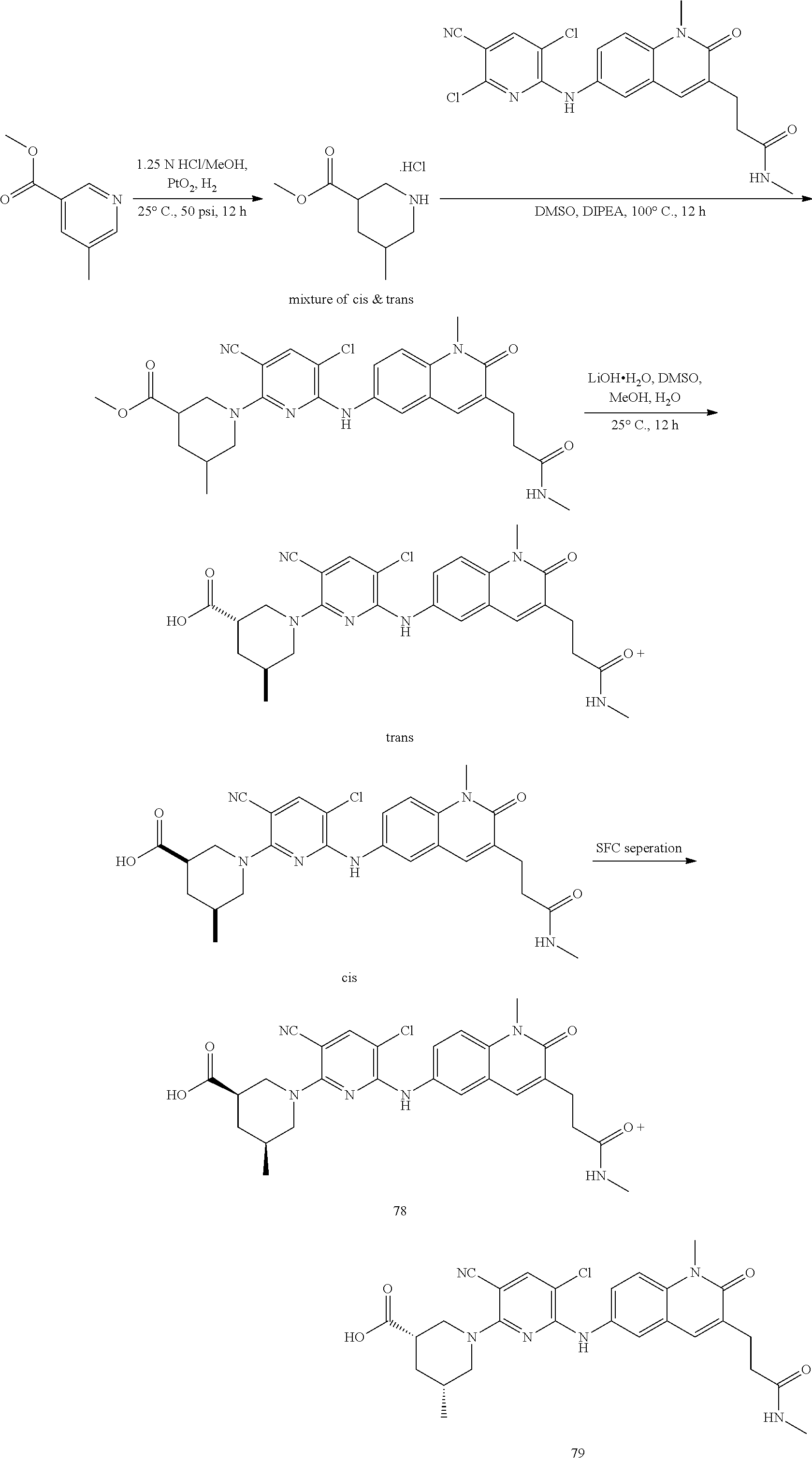

### Methyl 5-methylpiperidine-3-carboxylate hydrochloride

To a solution of methyl 5-methylpyridine-3-carboxylate (20 g, 132.31 mmol) in 1.25 N HCl/MeOH (150 mL) was added PtO₂ (3.00 g, 13.23 mmol) under Ar. The flask was degassed and purged with H₂ several times. The mixture was stirred under H₂ (50 psi) at 25° C. for 12 hours. The reaction mixture was filtered through a pad of Celite® and washed with MeOH (500 mL). The filtrate was concentrated under reduced pressure to give the title compound as a colorless oil (21 g, crude, HCl). LCMS: [M+H]⁺=158.3.

### Methyl 1-(5-chloro-3-cyano-6-((1-methyl-3-(2-(methylamino)-2-oxoethoxy)-2-oxo-1,2-dihydroquinolin-6-yl)amino)pyridin-2-yl)-5-methylpiperidine-3-carboxylate

To a solution of 2-[[6-[(3,6-dichloro-5-cyano-2-pyridyl)amino]-1-methyl-2-oxo-3-quinolyl]oxy]-N-methyl-acetamide (300 mg, 694.03 μmol) and methyl 5-methylpiperidine-3-carboxylate (201.62 mg, 1.04 mmol, HCl) in DMSO (5 mL) was added DIPEA (358.78 mg, 2.78 mmol, 483.54 μL). The mixture was stirred at 100° C. for 12 hr. The mixture was cooled to 20° C. and water (3 mL) was added, forming a precipitate which was filtered and the filter cake was concentrated to give the title compound as a yellow solid (300 mg, 542.49 μmol, 78% yield). LCMS: [M+H]⁺=553.2.

### 1-(5-Chloro-3-cyano-6-((I-methyl-3-(2-(methylamino)-2-oxoethoxy)-2-oxo-1,2-dihydroquinolin-6-yl)amino)pyridin-2-yl)-5-methylpiperidine-3-carboxylic acid (cis)

To a solution of methyl 1-[5-chloro-3-cyano-6-[[1-methyl-3-[2-(methylamino)-2-oxo-ethoxy]-2-oxo-6-quinolyl]amino]-2-pyridyl]-5-methyl-piperidine-3-carboxylate (250 mg, 452.07 μmol) in H₂O (2 mL), MeOH (2 mL) and DMSO (2 mL) was added LiOH H₂O (189.71 mg, 4.52 mmol). The mixture was stirred at 25° C. for 12 hr. Then 1N HCl (10 mL) was added to the mixture (combined with another batch of 50 mg scale) at which point pH=4, and the mixture was extracted with ethyl acetate (10 mL×2). The combined organic phase was washed with brine (20 mL), dried with anhydrous Na₂SO₄, filtered and concentrated in vacuo. The residue was purified by prep-HPLC (column: Phenomenex Luna 80*30 mm*3 μm; mobile phase: [water(TFA)-ACN]; B %: 30%-60%, 8 min) to give the title compound as a white solid (60 mg, 95% purity, cis). ¹H NMR (400 MHz, DMSO-d₆) δ 9.08-9.18 (m, 1H), 7.95-8.04 (m, 2H), 7.78 (d, J=2.38 Hz, 1H), 7.61-7.68 (m, 1H), 7.50-7.57 (m, 1H), 7.22-7.28 (m, 1H), 4.63-4.78 (m, 1H), 4.50-4.60 (m, 2H), 4.27-4.40 (m, 2H), 4.13 (br d, J=12.51 Hz, 2H), 3.54 (br t, J=5.69 Hz, 2H), 2.79 (br t, J=12.76 Hz, 2H), 2.67 (d, J=4.50 Hz, 3H), 1.96-2.15 (m, 2H), 1.75-1.86 (m, 2H), 0.82-0.86 (m, 6H).

### (3R,5S)-1-(5-chloro-3-cyano-6-((1-methyl-3-(2-(methylamino)-2-oxoethoxy)-2-oxo-1,2-dihydroquinolin-6-yl)amino)pyridin-2-yl)-5-methylpiperidine-3-carboxylic acid (78) and (3S,5R)-1-(5-chloro-3-cyano-6-((1-methyl-3-(2-(methylamino)-2-oxoethoxy)-2-oxo-1,2-dihydroquinolin-6-yl)amino)pyridin-2-yl)-5-methylpiperidine-3-carboxylic acid (79)

Racemic 1-[5-chloro-3-cyano-6-[[1-methyl-3-[2-(methylamino)-2-oxo-ethoxy]-2-oxo-6-quinolyl]amino]-2-pyridyl]-5-methyl-piperidine-3-carboxylic acid (60 mg, 111.32 μmol) was separated by SFC (column: DAICEL CHIRALCEL OJ (250 mm*30 mm, 10 μm); mobile phase: [0.1% NH₃H₂O MeOH]; B %: 36%-36%, 10 min) to give (3R,5S)-1-(5-chloro-3-cyano-6-((1-methyl-3-(2-(methylamino)-2-oxoethoxy)-2-oxo-1,2-dihydroquinolin-6-yl)amino)pyridin-2-yl)-5-methylpiperidine-3-carboxylic acid as a white solid (10.7 mg, 19.14 μmol, 17% yield, 96% purity); ¹H NMR (400 MHz, DMSO-d₆) δ 9.00 (s, 1H), 8.15 (br s, 1H), 7.89-7.96 (m, 2H), 7.68 (br d, J=9.01 Hz, 1H), 7.41 (br d, J=9.13 Hz, 1H), 7.21 (s, 1H), 4.59 (s, 2H), 4.46 (br d, J=12.13 Hz, 1H), 4.16 (br d, J=12.38 Hz, 1H), 3.66 (s, 3H), 2.81 (br t, J=12.38 Hz, 1H), 2.66 (br d, J=4.38 Hz, 3H), 2.44 (br s, 1H), 2.02 (br d, J=12.38 Hz, 1H), 1.54-1.73 (m, 1H), 0.82 (br d, J=6.38 Hz, 3H); and (3S,5R)-1-(5-chloro-3-cyano-6-((1-methyl-3-(2-(methylamino)-2-oxoethoxy)-2-oxo-1,2-dihydroquinolin-6-yl)amino)pyridin-2-yl)-5-methylpiperidine-3-carboxylic acid as a white solid (10.5 mg, 18.84 μmol, 17% yield, 97% purity); ¹H NMR (400 MHz, DMSO-d₆) δ 9.02 (s, 1H), 8.01 (br s, 1H), 7.93 (s, 1H), 7.87 (d, J=1.88 Hz, 1H), 7.69 (dd, J=9.07, 2.19 Hz, 1H), 7.41 (d, J=9.13 Hz, 1H), 7.20 (s, 1H), 4.56 (s, 2H), 4.44 (br d, J=12.76 Hz, 1H), 4.16 (br d, J=12.51 Hz, 1H), 3.66 (s, 3H), 2.82 (br t, J=12.38 Hz, 1H), 2.67 (d, J=4.50 Hz, 3H), 2.43 (br s, 1H), 2.02 (br d, J=12.38 Hz, 1H), 1.56-1.71 (m, 1H), 0.82 (d, J=6.50 Hz, 3H).

The absolute configurations of compounds 78 & 79 were randomly assigned based on the carboxy group and methyl group being in cis-conformation.

### Example 39: Synthesis of 2-((3S,5R)-3-amino-4,4-difluoro-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile hydrochloride (80) and 2-((3R,5S)-3-amino-4,4-difluoro-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile hydrochloride (81)

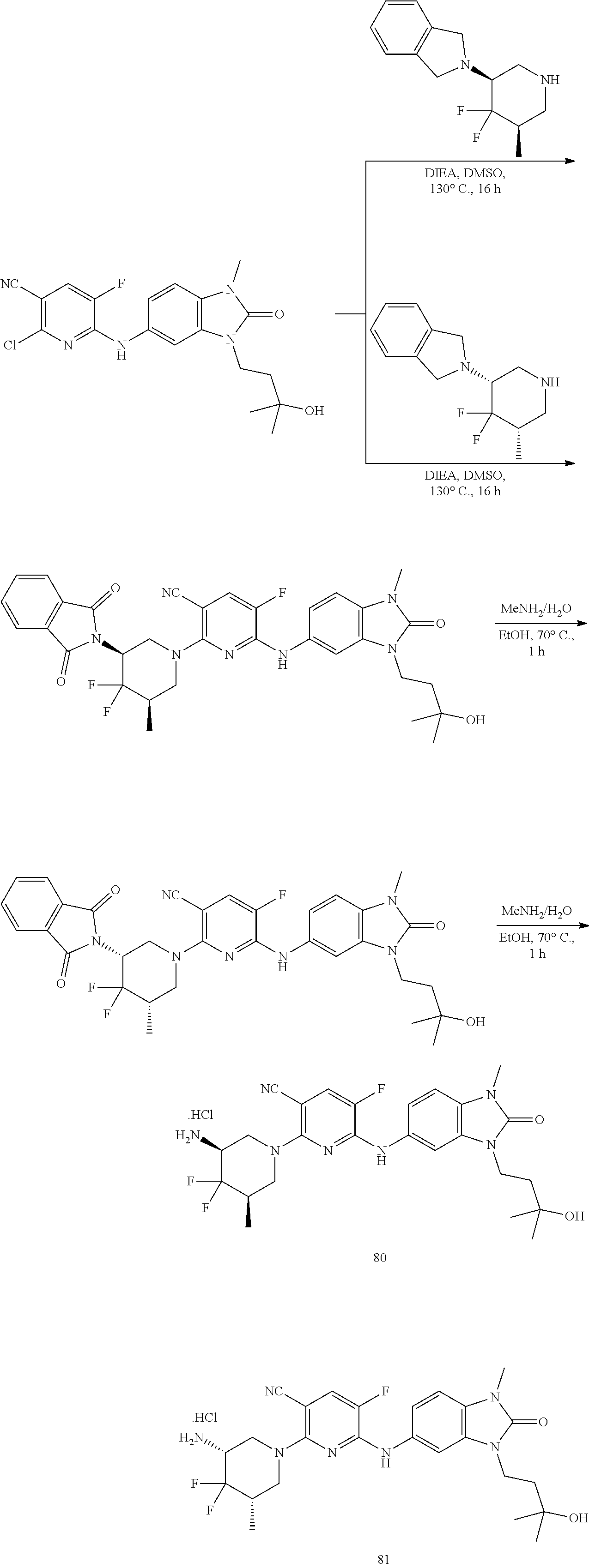

### 2-((3S,5R)-3-(1,3-Dioxoisoindolin-2-yl)-4,4-difluoro-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile

A flask with mixture of 2-chloro-5-fluoro-6-[[3-(3-hydroxy-3-methyl-butyl)-1-methyl-2-oxo-benzimidazol-5-yl]amino]pyridine-3-carbonitrile (200 mg, 495.25 μmol), 2-[(3S,5R)-4,4-difluoro-5-methyl-3-piperidyl]isoindoline-1,3-dione (138.80 mg, 495.25 μmol) and DIEA (320.04 mg, 2.48 mmol, 431.32 μL) in DMSO (3 mL) was degassed and purged with N₂ for 3 times, then the mixture was stirred at 130° C. for 16 hr under N₂ atmosphere. Water (15 mL) was added to the mixture, forming a precipitate which was filtered and washed with H₂O (3 mL), and then the solid was dried under reduced pressure to give the title compound as a red solid (260 mg, 148.54 μmol, 30% yield, 37% purity). LCMS: [M+H]⁺=648.1.

### 2-((3S,5R)-3-Amino-4,4-difluoro-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile hydrochloride (80)

To a solution of 2-((3S,5R)-3-(1,3-dioxoisoindolin-2-yl)-4,4-difluoro-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile (260 mg, 401.45 μmol) in EtOH (16 mL) was added MeNH₂/H₂O (16 mL, 40% purity). The mixture was stirred at 70° C. for 1 hr. The mixture was concentrated in vacuo and purified by prep-HPLC (column: Phenomenex Luna 80*30 mm*3 μm; mobile phase: [water(HCl)-ACN]; B %: 15%-45%, 8 min) to give the title compound as a yellow solid (50 mg, 84.84 μmol, 21% yield, 94% purity, HCl). ¹H NMR (400 MHz, MeOD-d₄, 25° C.) δ 7.60 (d, J=10.49 Hz, 1H), 7.44 (d, J=1.67 Hz, 1H), 7.37 (dd, J=8.40, 1.85 Hz, 1H), 7.15 (d, J=8.46 Hz, 1H), 4.28-4.45 (m, 1H), 3.97-4.08 (m, 3H), 3.74-3.89 (m, 1H), 3.43 (s, 3H), 3.23 (t, J=12.28 Hz, 1H), 2.89 (t, J=12.70 Hz, 1H), 2.33-2.56 (m, 1H), 1.86 (dd, J=8.88, 7.57 Hz, 2H), 1.29 (d, J=2.50 Hz, 6H), 1.06 (d, J=6.68 Hz, 3H).

### 2-((3R,5S)-3-(1,3-Dioxoisoindolin-2-yl)-4,4-difluoro-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)

A flask with mixture of 2-chloro-5-fluoro-6-[[3-(3-hydroxy-3-methyl-butyl)-1-methyl-2-oxo-benzimidazol-5-yl]amino]pyridine-3-carbonitrile (200 mg, 495.25 μmol), 2-[(3R,5S)-4,4-difluoro-5-methyl-3-piperidyl]isoindoline-1,3-dione (138.80 mg, 495.25 μmol) and DIEA (320.04 mg, 2.48 mmol, 431.32 μL) in DMSO (3 mL) was degassed and purged with N₂ for 3 times, and then the mixture was stirred at 130° C. for 16 hr under N₂ atmosphere. Water (15 mL) was added to the mixture, forming a precipitate which filtered and washed with H₂O (3 mL), and then the solid was dried under reduced pressure to give the title compound as a red solid (270 mg, 154.25 μmol, 31% yield, 37% purity). LCMS: [M+H]⁺=648.1.

### 2-((3R,5S)-3-Amino-4,4-difluoro-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile hydrochloride (81)

To a solution of 2-((3R,5S)-3-(1,3-dioxoisoindolin-2-yl)-4,4-difluoro-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile (270 mg, 416.89 μmol) in EtOH (16 mL) was added MeNH₂/H₂O (16 mL, 40% purity). The mixture was stirred at 70° C. for 1 hr. The mixture was concentrated in vacuo and purified directly with prep-HPLC (column: Phenomenex Luna 80*30 mm*3 μm; mobile phase: [water(HCl)-ACN]; B %: 15%-45%, 8 min) to give the title compound as a yellow solid (40 mg, 67.87 μmol, 16% yield, 94% purity, HCl). ¹H NMR (400 MHz, MeOD-d₄, 25° C.) δ 7.60 (d, J=10.49 Hz, 1H), 7.44 (s, 1H), 7.37 (d, J=8.34 Hz, 1H), 7.15 (d, J=8.46 Hz, 1H), 4.32-4.44 (m, 1H), 3.94-4.12 (m, 3H), 3.73-3.89 (m, 1H), 3.43 (s, 3H), 3.23 (t, J=12.22 Hz, 1H), 2.89 (t, J=12.64 Hz, 1H), 2.35-2.54 (m, 1H), 1.86 (t, J=8.11 Hz, 2H), 1.29 (d, J=2.03 Hz, 6H), 1.05 (d, J=6.68 Hz, 3H).

The absolute configurations of compounds 80 & 81 were randomly assigned based on the amino group and methyl group being in cis-conformation.

### Example 40: Synthesis of 2-((3S,5R)-4,4-difluoro-3-hydroxy-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile (82) and 2-((3R,5S)-4,4-difluoro-3-hydroxy-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile (83)

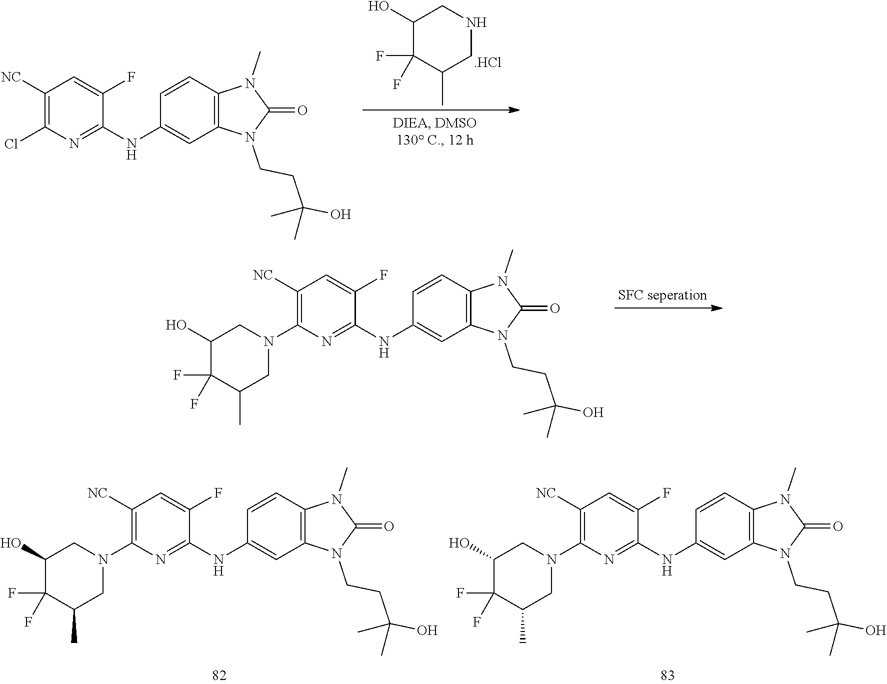

### 2-(4,4-Difluoro-3-hydroxy-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile

A flask with mixture of 2-chloro-5-fluoro-6-[[3-(3-hydroxy-3-methyl-butyl)-1-methyl-2-oxo-benzimidazol-5-yl]amino]pyridine-3-carbonitrile (300 mg, 742.87 μmol), (3R,5S)-4,4-difluoro-5-methyl-piperidin-3-ol (278.75 mg, 1.49 mmol, HCl), DIEA (480.06 mg, 3.71 mmol, 646.98 μL) in DMSO (3 mL) was degassed and purged with N₂ for 3 times, and then the mixture was stirred at 130° C. for 12 hr under N₂ atmosphere. The mixture was cooled to 15° C. and purified directly with prep-HPLC (column: Waters Xbridge Prep OBD C18 150*40 mm*10 μm; mobile phase: [water(NH₄HCO₃)-ACN]; B %: 35%-55%, 8 min) to give the title compound as a yellow solid (150 mg, 286.39 μmol, 39% yield, 99% purity). ¹H NMR (400 MHz, MeCN-d₃) δ 7.80 (br s, 1H), 7.45-7.50 (m, 2H), 7.20 (dd, J=8.40, 1.97 Hz, 1H), 7.01 (d, J=8.34 Hz, 1H), 4.19-4.28 (m, 1H), 3.99-4.06 (m, 1H), 3.90-3.98 (m, 3H), 3.76-3.90 (m, 1H), 3.34 (s, 3H), 2.96-3.05 (m, 1H), 2.94 (s, 1H), 2.88 (t, J=12.64 Hz, 1H), 2.14-2.26 (m, 1H), 1.77-1.85 (m, 2H), 1.22 (d, J=2.03 Hz, 6H), 0.99 (d, J=6.79 Hz, 3H).

### 2-((3S,5R)-4,4-difluoro-3-hydroxy-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile (82) and 2-((3R,5S)-4,4-difluoro-3-hydroxy-5-methylpiperidin-1-yl)-5-fluoro-6-((3-(3-hydroxy-3-methylbutyl)-1-methyl-2-oxo-2,3-dihydro-1H-benzo[d]imidazol-5-yl)amino)nicotinonitrile (83)

2-[(3R,5S)-4,4-difluoro-3-hydroxy-5-methyl-1-piperidyl]-5-fluoro-6-[[3-(3-hydroxy-3-methyl-butyl)-1-methyl-2-oxo-benzimidazol-5-yl]amino]pyridine-3-carbonitrile (150 mg, 289.28 μmol) was separated by SFC (column: DAICEL CHIRALPAK IG (250 mm*30 mm, 10 μm); mobile phase: [Heptane-EtOH]; B %: 20%-20%, 10 min) to give 2-[(3S,5R)-4,4-difluoro-3-hydroxy-5-methyl-1-piperidyl]-5-fluoro-6-[[3-(3-hydroxy-3-methyl-butyl)-1-methyl-2-oxo-benzimidazol-5-yl]amino]pyridine-3-carbonitrile as a white solid (45 mg, 85.92 μmol, 30% yield, 99% purity); ¹H NMR (400 MHz, MeOD-d₄) δ 7.34-7.43 (m, 2H), 7.26 (br d, J=8.44 Hz, 1H), 7.01 (br d, J=8.44 Hz, 1H), 4.16 (br d, J=12.47 Hz, 1H), 3.88-3.98 (m, 3H), 3.71 (ddd, J=15.28, 10.45, 5.20 Hz, 1H), 3.20 (br s, 3H), 2.91 (br t, J=11.98 Hz, 1H), 2.75 (br t, J=12.53 Hz, 1H), 1.96-2.12 (m, 1H), 1.71-1.79 (m, 2H), 1.17 (s, 6H), 0.89 (br d, J=6.72 Hz, 3H); and 2-[(3R,5S)-4,4-difluoro-3-hydroxy-5-methyl-1-piperidyl]-5-fluoro-6-[[3-(3-hydroxy-3-methyl-butyl)-1-methyl-2-oxo-benzimidazol-5-yl]amino]pyridine-3-carbonitrile as a white solid (45 mg, 85.92 μmol, 30% yield, 99% purity). ¹H NMR (400 MHz, MeOD-d₄) δ 7.35-7.43 (m, 2H), 7.25 (br d, J=8.31 Hz, 1H), 7.01 (br d, J=8.19 Hz, 1H), 4.16 (br d, J=12.10 Hz, 1H), 3.93 (br d, J=8.19 Hz, 3H), 3.64-3.78 (m, 1H), 3.20 (br s, 3H), 2.70-2.96 (m, 2H), 1.95-2.12 (m, 1H), 1.69-1.80 (m, 2H), 1.17 (s, 6H), 0.88 (br d, J=6.60 Hz, 3H).

The absolute configurations of compounds 82 & 83 were randomly assigned based on the hydroxyl group and methyl group being in cis-conformation.

### Example 41: Synthesis of 2-((3S,5R)-3-amino-4,4-difluoro-5-methylpiperidin-1-yl)-5-fluoro-6-((1-methyl-2-oxo-3-(((R)-2-oxooxazolidin-5-yl)methyl)-2,3-dihydro-1H-benzo[d]imidazol-5-yl)amino)nicotinonitrile (84) and 2-[(3R,5S)-3-amino-4,4-difluoro-5-methyl-1-piperidyl]-5-fluoro-6-[[1-methyl-2-oxo-3-[[(5S)-2-oxooxazolidin-5-yl]methyl]benzimidazol-5-yl] amino]pyridine-3-carbonitrile (85)

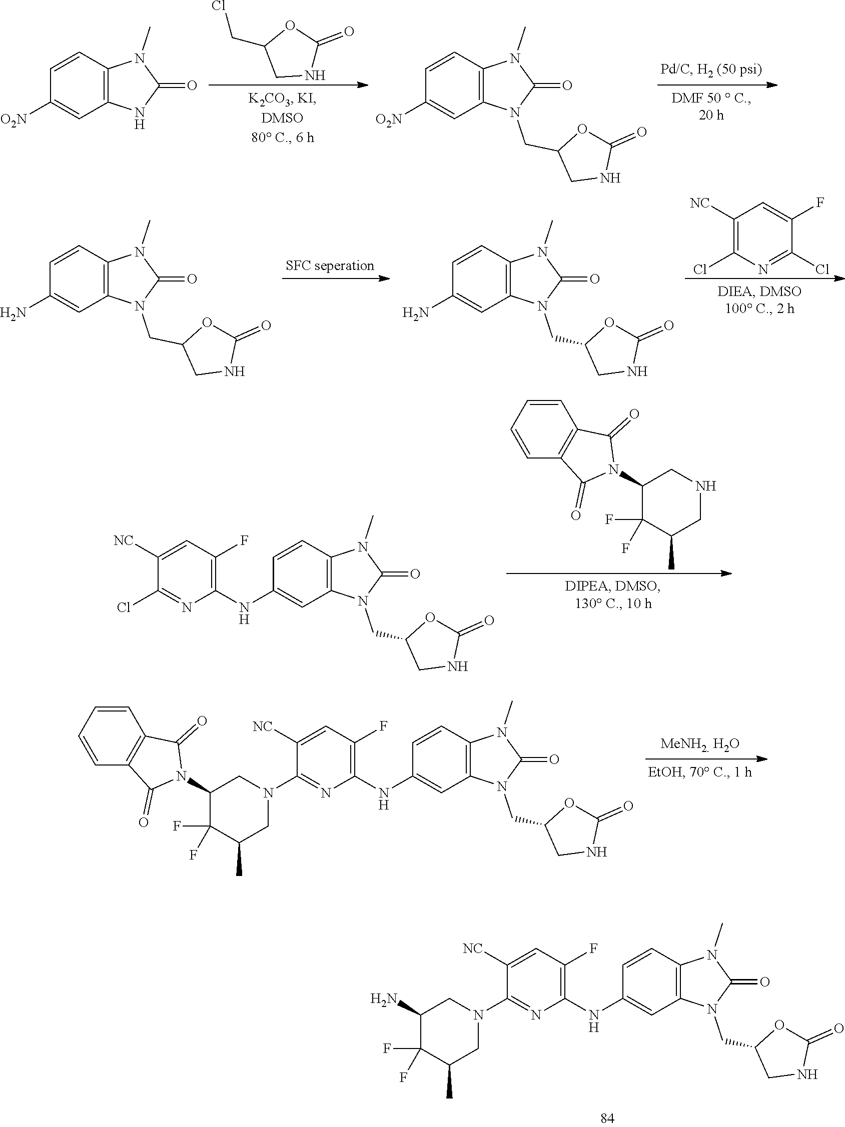

### 5-((3-Methyl-6-nitro-2-oxo-2,3-dihydro-1H-benzo[d]imidazol-1-yl)methyl)oxazolidin-2-one

A flask with mixture of 1-methyl-5-nitro-1H-benzo[d]imidazol-2(3H)-one (5 g, 25.89 mmol), 5-(chloromethyl)oxazolidin-2-one (5.26 g, 38.84 mmol), K₂CO₃ (7.16 g, 51.78 mmol) and KI (2.15 g, 12.95 mmol) in DMSO (50 mL) was degassed and purged with N₂ for 3 times, and then the mixture was stirred at 80° C. for 6 hr under N₂ atmosphere. Water (80 mL) was added to mixture, forming a precipitate which was filtered and washed with H₂O (20 mL) and petroleum ether/ethyl acetate=(1:1, 30 mL). The solid was dried under reduced pressure to give the title compound as a yellow solid (5.5 g, 18.07 mmol, 70% yield, 96% purity). LCMS: [M+H]⁺=293.1.

### 5-((6-Amino-3-methyl-2-oxo-2,3-dihydro-1H-benzo[d]imidazol-1-yl)methyl)oxazolidin-2-one

To a mixture of Pd/C (1 g, 10% purity) in DMF (300 mL) was added 5-((3-methyl-6-nitro-2-oxo-2,3-dihydro-1H-benzo[d]imidazol-1-yl)methyl)oxazolidin-2-one (5.5 g, 18.82 mmol). The mixture was stirred at 50° C. for 20 hr under H₂ (50 psi). Celite® was added to the mixture and filtered to give a filtrate which concentrated and purified by column chromatography (SiO₂, Ethyl acetate/EtOH=1/0 to 1/1) to give the title compound as a yellow solid (3.3 g, 11.83 mmol, 63% yield, 94% purity). ¹H NMR (400 MHz, DMSO-d₆) δ 7.53 (s, 1H), 6.80 (d, J=8.23 Hz, 1H), 6.46 (d, J=1.19 Hz, 1H), 6.33 (dd, J=8.23, 1.55 Hz, 1H), 4.75-4.89 (m, 3H), 3.89-4.00 (m, 2H), 3.57 (t, J=8.82 Hz, 1H), 3.31 (dd, J=8.58, 6.56 Hz, 1H), 3.23 (s, 3H).

### (R)-5-((6-Amino-3-methyl-2-oxo-2,3-dihydro-1H-benzo[d]imidazol-1-yl)methyl)oxazolidin-2-one

Racemic 5-((6-amino-3-methyl-2-oxo-2,3-dihydro-1H-benzo[d]imidazol-1-yl)methyl)oxazolidin-2-one (2.7 g, 10.29 mmol) was separated with SFC (column: DAICEL CHIRALPAK AD (250 mm*50 mm, 10 μm); mobile phase: [0.1% NH₃H₂O MeOH]; B %:50%-50%, 3.9 min) to give the title compound as a yellow solid (1.2 g, 4.26 mmol, 41% yield, 93% purity). ¹H NMR (400 MHz, DMSO-d₆) δ 7.55 (s, 1H), 6.80 (d, J=8.4 Hz, 1H), 6.47 (s, 1H), 6.35 (dd, J=8.0, 2.4 Hz, 1H), 4.83-4.88 (m, 2H), 3.93-4.00 (m, 2H), 3.58 (t, J=8.8 Hz, 1H), 3.32 (dd, J=8.8 Hz, 6.4 Hz, 1H), 3.23 (s, 3H).

### (R)-2-Chloro-5-fluoro-6-((1-methyl-2-oxo-3-((2-oxooxazolidin-5-yl)methyl)-2,3-dihydro-1H-benzo[d]imidazol-5-yl)amino)nicotinonitrile

A flask with mixture of 5-[(6-amino-3-methyl-2-oxo-benzimidazol-1-yl)methyl]oxazolidin-2-one (600 mg, 2.29 mmol), 2,6-dichloro-5-fluoro-pyridine-3-carbonitrile (436.94 mg, 2.29 mmol) and DIEA (591.34 mg, 4.58 mmol, 796.96 μL) in DMSO (6 mL) was degassed and purged with N₂ for 3 times, and then the mixture was stirred at 100° C. for 2 hr under N₂ atmosphere. Water (30 mL) was added to the mixture, forming a precipitate which was filtered and washed with H₂O (20 mL) and ethyl acetate (10 mL). The solid was dried under reduced pressure to give the title compound as a yellow solid (700 mg, 1.55 mmol, 68% yield, 92% purity). ¹H NMR (400 MHz, DMSO-d₆) δ 9.96 (s, 1H), 8.09 (d, J=10.27 Hz, 1H), 7.45-7.60 (m, 2H), 7.27 (d, J=7.70 Hz, 1H), 7.11 (d, J=7.95 Hz, 1H), 4.86 (s, 1H), 3.92-4.12 (m, 2H), 3.56 (t, J=7.83 Hz, 1H), 3.30 (s, 4H).

### 2-((3S,5R)-3-(1,3-dioxoisoindolin-2-yl)-4,4-difluoro-5-methylpiperidin-1-yl)-5-fluoro-6-((I-methyl-2-oxo-3-(((R)-2-oxooxazolidin-5-yl)methyl)-2,3-dihydro-1H-benzo[d]imidazol-5-yl)amino)nicotinonitrile

A flask with mixture of (R)-2-chloro-5-fluoro-6-((1-methyl-2-oxo-3-((2-oxooxazolidin-5-yl)methyl)-2,3-dihydro-1H-benzo[d]imidazol-5-yl)amino)nicotinonitrile (500 mg, 1.20 mmol), 2-[(3R,5S)-4,4-difluoro-5-methyl-3-piperidyl]isoindoline-1,3-dione (336.22 mg, 1.20 mmol) and DIEA (310.08 mg, 2.40 mmol, 417.90 μL) in DMSO (5 mL) was degassed and purged with N₂ for 3 times, and then the mixture was stirred at 130° C. for 10 hr under N₂ atmosphere. The mixture was concentrated in vacuo without further work-up. The residue was purified by prep-HPLC (column: Phenomenex C18 80*40 mm*3 μm; mobile phase: [water(NH₄HCO₃)-ACN]; B %: 25%-45%, 8 min) to give the title compound as a white solid (230 mg, 344.69 μmol, 29% yield, 99% purity). LCMS: [M+H]⁺=661.3.

### 2-((3S,5R)-3-amino-4,4-difluoro-5-methylpiperidin-1-yl)-5-fluoro-6-((I-methyl-2-oxo-3-(((R)-2-oxooxazolidin-5-yl)methyl)-2,3-dihydro-1H-benzo[d]imidazol-5-yl)amino)nicotinonitrile (84)

A flask with mixture of 2-((3S,5R)-3-(1,3-dioxoisoindolin-2-yl)-4,4-difluoro-5-methylpiperidin-1-yl)-5-fluoro-6-((1-methyl-2-oxo-3-(((R)-2-oxooxazolidin-5-yl)methyl)-2,3-dihydro-1H-benzo[d]imidazol-5-yl)amino)nicotinonitrile (230 mg, 348.17 μmol) in EtOH (15 mL) and MeNH₂ (15 mL, 40% purity) was degassed and purged with N₂ for 3 times, and then the mixture was stirred at 70° C. for 1 hr under N₂ atmosphere. The mixture was concentrated in vacuo and purified directly with prep-HPLC (column: Phenomenex C18 80*40 mm*3 μm; mobile phase: [water(NH₄HCO₃)-ACN]; B %: 20%-40%, 8 min) to give the title compound as a white solid (40 mg, 74.65 μmol, 21% yield, 99% purity). ¹H NMR (400 MHz, DMSO-d₆, 25° C.) δ 9.46 (s, 1H), 7.82 (d, J=10.85 Hz, 1H), 7.63 (d, J=1.31 Hz, 1H), 7.53 (s, 1H), 7.34 (d, J=7.03 Hz, 1H), 7.13 (d, J=8.34 Hz, 1H), 4.90 (d, J=2.38 Hz, 1H), 4.03-4.22 (m, 3H), 3.95-4.02 (m, 1H), 3.59 (t, J=8.94 Hz, 1H), 3.35 (s, 3H), 2.90-3.07 (m, 1H), 2.78 (q, J=12.12 Hz, 2H), 2.08-2.22 (m, 1H), 1.69 (s, 2H), 0.90 (d, J=6.68 Hz, 3H).

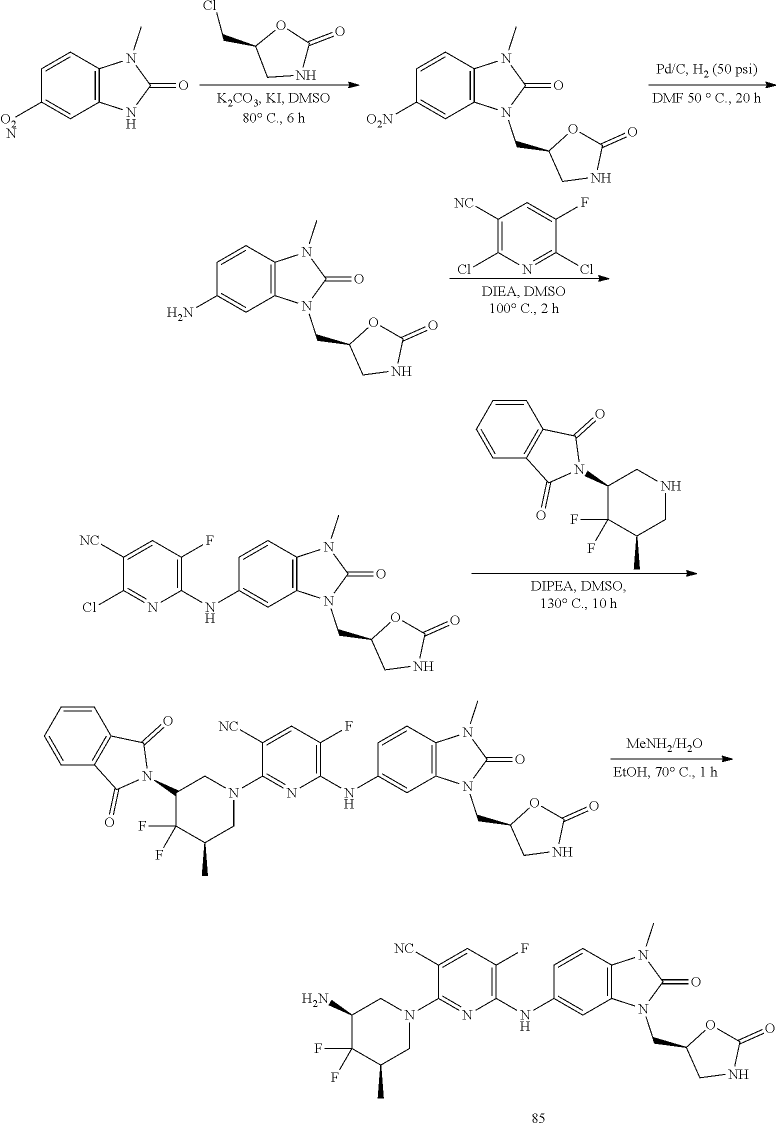

### (S)-5-((3-Methyl-6-nitro-2-oxo-2,3-dihydro-1H-benzo[d]imidazol-1-yl)methyl) oxazolidin-2-one

A flask with mixture of 3-methyl-6-nitro-1H-benzimidazol-2-one (5 g, 25.89 mmol), (5S)-5-(chloromethyl)oxazolidin-2-one (4.21 g, 31.06 mmol), K₂CO₃ (7.16 g, 51.77 mmol) and KI (2.15 g, 12.94 mmol) in DMSO (50 mL) was degassed and purged with N₂ for 3 times, and then the mixture was stirred at 80° C. for 6 hrs under N₂ atmosphere. Water (40 mL) was added to the mixture, forming a precipitate which was filtered and washed with H₂O (15 mL) and ethyl acetate (30 mL). The solid was dried under reduced pressure to give the title compound as a yellow solid (4 g, 12.87 mmol, 50% yield, 94% purity). ¹H NMR (400 MHz, DMSO-d₆) δ 8.22 (d, J=2.0 Hz, 1H), 8.08 (dd, J=8.4, 2.0 Hz, 1H), 7.55 (s, 1H), 7.38 (d, J=8.8 Hz, 1H), 4.88-4.98 (m, 1H), 4.15-4.34 (m, 2H), 3.59-3.68 (m, 1H), 3.43 (s, 3H), 3.33 (dd, J=9.05, 6.24 Hz, 1H).

### (S)-5-((6-Amino-3-methyl-2-oxo-2,3-dihydro-1H-benzo[d]imidazol-1-yl)methyl) oxazolidin-2-one

To a suspension of Pd/C (300 mg, 10% purity) in DMF (100 mL) was added (S)-5-((3-methyl-6-nitro-2-oxo-2,3-dihydro-1H-benzo[d]imidazol-1-yl)methyl)oxazolidin-2-one (4 g, 13.69 mmol). The mixture was stirred at 50° C. for 20 hr under H₂ (50 psi). The mixture was filtered with Celite® and the filtrate was concentrated to give a residue which was purified by silica gel column chromatography (silica gel, Ethyl acetate/EtOH=1/0 to 1/1) to give the title compound as a yellow solid (2 g, 6.71 mmol, 49% yield, 88% purity). ¹H NMR (400 MHz, DMSO-d₆) δ 7.53 (s, 1H), 6.80 (br d, J=8.0 Hz, 1H), 6.47 (s, 1H), 6.34 (br d, J=8.0 Hz, 1H), 4.65-5.07 (m, 3H), 3.87-4.03 (m, 2H), 3.57 (br t, J=8.8 Hz, 1H), 3.28-3.35 (m, 1H), 3.24 (s, 3H).

### (S)-2-Chloro-5-fluoro-6-((I-methyl-2-oxo-3-((2-oxooxazolidin-5-yl)methyl)-2,3-dihydro-1H-benzo[d]imidazol-5-yl)amino)nicotinonitrile

A flask with mixture of (S)-5-((6-amino-3-methyl-2-oxo-2,3-dihydro-1H-benzo[d]imidazol-1-yl)methyl)oxazolidin-2-one (1 g, 3.81 mmol), 2,6-dichloro-5-fluoro-pyridine-3-carbonitrile (728.24 mg, 3.81 mmol) and DIEA (985.59 mg, 7.63 mmol, 1.33 mL) in DMSO (10 mL) was degassed and purged with N₂ for 3 times, then the mixture was stirred at 100° C. for 2 hr under N₂ atmosphere. Water (25 mL) was added to the mixture, forming a precipitate which was filtered and washed with H₂O (10 mL) and ethyl acetate (15 mL). The solid was dried under reduced pressure to give the title compound as a yellow solid (1.1 g, 2.51 mmol, 66% yield, 95% purity). LCMS: [M+H]⁺=417.1.

### 2-((3R,5S)-3-(1,3-Dioxoisoindolin-2-yl)-4,4-difluoro-5-methylpiperidin-1-yl)-5-fluoro-6-((I-methyl-2-oxo-3-(((S)-2-oxooxazolidin-5-yl)methyl)-2,3-dihydro-1H-benzo[d]imidazol-5-yl)amino)nicotinonitrile

A flask with mixture of (S)-2-chloro-5-fluoro-6-((1-methyl-2-oxo-3-((2-oxooxazolidin-5-yl) methyl)-2,3-dihydro-1H-benzo[d]imidazol-5-yl)amino)nicotinonitrile (600 mg, 1.44 mmol), 2-((3R,5S)-4,4-difluoro-5-methylpiperidin-3-yl)isoindoline-1,3-dione (403.47 mg, 1.44 mmol) and DIEA (372.11 mg, 2.88 mmol, 501.49 μL) in DMSO (6 mL) was degassed and purged with N₂ for 3 times, then the mixture was stirred at 130° C. for 10 hrs under N₂ atmosphere. The mixture was concentrated in vacuo without further work-up. The residue was purified by prep-HPLC (column: Waters Xbridge BEH C18 100*30 mm*10 μm; mobile phase: [water(NH₄HCO₃)-ACN]; B %: 40%-60%, 8 min) to give the title compound as a white solid (270 mg, 396.46 μmol, 28% yield, 97% purity). ¹H NMR (400 MHz, DMSO-d₆) δ 9.62 (s, 1H), 7.87-7.95 (m, 4H), 7.52 (s, 1H), 7.46 (d, J=2.0 Hz, 1H), 7.18 (dd, J=8.0, 2.0 Hz, 1H), 6.94 (d, J=8.4 Hz, 1H), 4.81-4.91 (m, 1H), 4.44-4.57 (m, 1H), 4.20-4.39 (m, 3H), 3.87-3.94 (m, 1H), 3.76-3.84 (m, 1H), 3.57 (t, J=8.0 Hz, 1H), 3.15-3.30 (m, 2H), 3.05 (s, 3H), 2.18-2.40 (m, 1H), 0.96 (d, J=6.8 Hz, 3H).

### 2-((3R,5S)-3-Amino-4,4-difluoro-5-methylpiperidin-1-yl)-5-fluoro-6-((I-methyl-2-oxo-3-(((S)-2-oxooxazolidin-5-yl)methyl)-2,3-dihydro-1H-benzo[d]imidazol-5-yl)amino)nicotinonitrile (85)

To a solution of 2-((3R,5S)-3-(1,3-dioxoisoindolin-2-yl)-4,4-difluoro-5-methylpiperidin-1-yl)-5-fluoro-6-((1-methyl-2-oxo-3-(((S)-2-oxooxazolidin-5-yl)methyl)-2,3-dihydro-1H-benzo[d]imidazol-5-yl)amino)nicotinonitrile (270 mg, 408.72 μmol) in EtOH (20 mL) was added MeNH₂/H₂O (31.73 mg, 408.72 μmol, 20 mL, 40% purity). The mixture was stirred at 70° C. for 1 hr. The mixture was concentrated in vacuo and purified directly with prep-HPLC (column: Phenomenex C18 75*30 mm*3 μm; mobile phase: [water (NH₄HCO₃)-ACN]; B %: 25%-45%, 8 min) to give the title compound as a white solid (40 mg, 75.40 μmol, 18% yield, 100% purity). ¹H NMR (400 MHz, DMSO-d₆) δ 9.48 (s, 1H), 7.83 (d, J=10.8 Hz, 1H), 7.65 (d, J=2.0 Hz, 1H), 7.54 (s, 1H), 7.35 (dd, J=8.0, 4.0 Hz, 1H), 7.14 (d, J=8.0 Hz, 1H), 4.86-4.99 (m, 1H), 4.06-4.19 (m, 3H), 3.99 (br d, J=16.0 Hz, 1H), 3.61 (t, J=8.0 Hz, 1H), 3.36 (s, 3H), 2.93-3.11 (m, 1H), 2.70-2.88 (m, 2H), 2.07-2.23 (m, 1H), 1.70 (br s, 2H), 0.91 (d, J=8.0 Hz, 3H).

The absolute configurations of compounds 84 & 85 were randomly assigned based on the amino group and methyl group being in cis-conformation.

### Example 42: Synthesis of 5-fluoro-2-((3S,4S,5R)-4-fluoro-3,5-dimethylpiperidin-1-yl)-6-((1-methyl-2-oxo-3-(((R)-2-oxooxazolidin-5-yl)methyl)-2,3-dihydro-1H-benzo[d]imidazol-5-yl)amino)nicotinonitrile (86) and 5-fluoro-2-[(3S,4S,5R)-4-fluoro-3,5-dimethyl-1-piperidyl]-6-[[1-methyl-2-oxo-3-[[(5S)-2-oxooxazolidin-5-yl]methyl]benzimidazol-5-yl]amino]pyridine-3-carbonitrile (87)

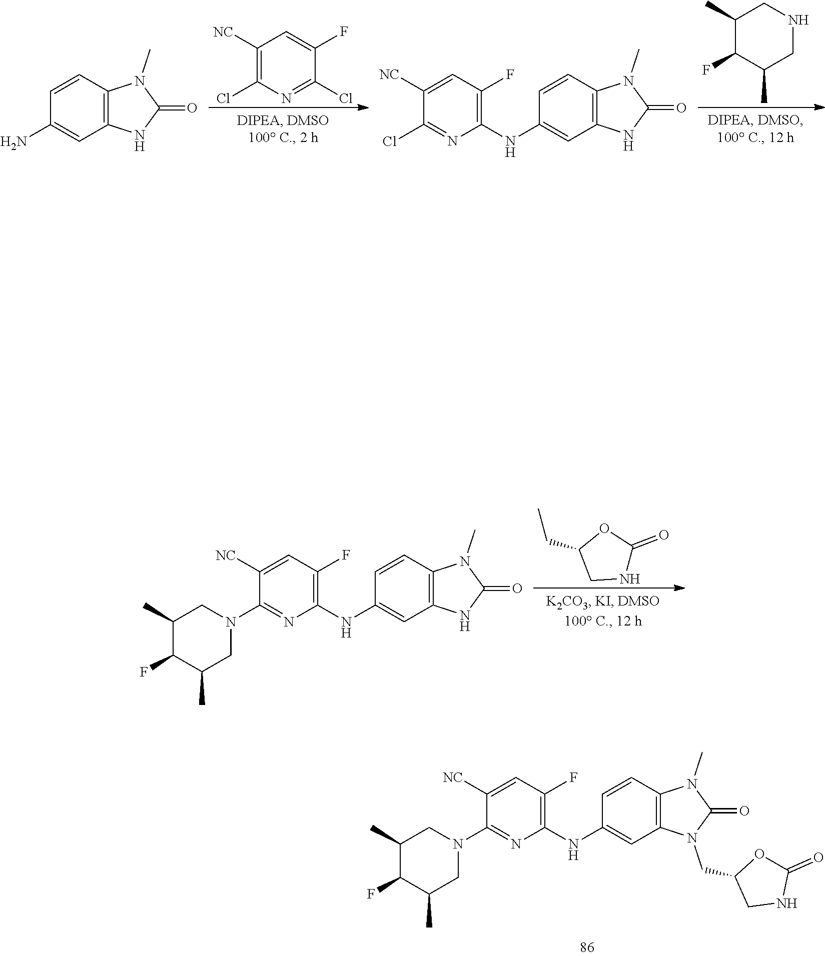

### 2-Chloro-5-fluoro-6-((1-methyl-2-oxo-2,3-dihydro-1H-benzo[d]imidazol-5-yl)amino)nicotinonitrile

To a solution of 6-amino-3-methyl-1H-benzimidazol-2-one (1 g, 6.13 mmol) and 2,6-dichloro-5-fluoro-pyridine-3-carbonitrile (1.17 g, 6.13 mmol) in DMSO (10 mL) was added DIPEA (1.58 g, 12.26 mmol, 2.13 mL). The mixture was stirred at 100° C. for 2 hr. The mixture was cooled to 15° C. and water (10 mL) was added, forming a precipitate which was filtered and the filter cake was washed with EtOAc (50 mL). The filter cake was dried in vacuo to give the title compound as a yellow solid (1.8 g, 5.10 mmol, 83% yield, 90% purity). ¹H NMR (400 MHz, DMSO-d₆) δ 10.89 (s, 1H), 9.86 (s, 1H), 8.12 (d, J=10.8 Hz, 1H), 7.38 (d, J=1.6 Hz, 1H), 7.26 (dd, J=8.4, 1.6 Hz, 1H), 7.06 (d, J=8.4 Hz, 1H), 2.54 (s, 3H).

### 5-Fluoro-2-((3S,4S,5R)-4-fluoro-3,5-dimethylpiperidin-1-yl)-6-((1-methyl-2-oxo-2,3-dihydro-1H-benzo[d]imidazol-5-yl)amino)nicotinonitrile

To a solution of 2-chloro-5-fluoro-6-[(1-methyl-2-oxo-3H-benzimidazol-5-yl)amino]pyridine-3-carbonitrile (1 g, 3.15 mmol) and (3S,4S,5R)-4-fluoro-3,5-dimethyl-piperidine (1.54 g, 6.30 mmol, TFA) in DMSO (10 mL) was added DIPEA (2.03 g, 15.74 mmol, 2.74 mL). The mixture was stirred at 100° C. for 12 hr. Water (5 mL) was added to the mixture, forming a precipitate which was filtered and the filter cake was washed by water (10 mL) and EtOAc (20 mL) and then the filter cake was dried in vacuo to give the title compound as a yellow solid (1 g, 2.34 mmol, 74% yield, 96% purity). ¹H NMR (400 MHz, DMSO-d₆) δ 10.85 (s, 1H), 9.31 (s, 1H), 7.73 (d, J=10.4 Hz, 1H), 7.32 (s, 2H), 7.01 (d, J=7.6 Hz, 1H), 4.42-4.65 (m, 1H), 3.90 (d, J=11.6 Hz, 2H), 3.26 (s, 3H), 2.82 (t, J=12 Hz, 2H), 1.73-1.97 (m, 2H), 0.91 (d, J=5.2 Hz, 6H).

### 5-Fluoro-2-((3S,4S,5R)-4-fluoro-3,5-dimethylpiperidin-1-yl)-6-((1-methyl-2-oxo-3-(((R)-2-oxooxazolidin-5-yl)methyl)-2,3-dihydro-1H-benzo[d]imidazol-5-yl)amino)nicotinonitrile (86)

To a mixture of 5-fluoro-2-[(3S,4S,5R)-4-fluoro-3,5-dimethyl-1-piperidyl]-6-[(1-methyl-2-oxo-3H-benzimidazol-5-yl)amino]pyridine-3-carbonitrile (100 mg, 242.46 μmol) and (R)-5-(chloromethyl)oxazolidin-2-one (49.30 mg, 363.69 μmol) in DMSO (1 mL) was added K₂CO₃ (67.02 mg, 484.92 μmol) and KI (20.12 mg, 121.23 μmol), then the reaction mixture was stirred at 100° C. for 12 hr. The reaction mixture was filtered and the filtrate was then purified by prep-HPLC (column: Waters Xbridge Prep OBD C18 150*40 mm*10 μm; mobile phase: [water(NH₄HCO₃)-ACN]; B %: 35%-55%, 8 min) to give the title compound as a white solid (40 mg, 77.96 μmol, 32% yield, 99% purity). ¹H NMR (400 MHz, DMSO-d₆) δ 9.40 (s, 1H), 7.77 (d, J=10.8 Hz, 1H), 7.59-7.51 (m, 2H), 7.38 (dd, J=1.6, 8.4 Hz, 1H), 7.11 (d, J=8.4 Hz, 1H), 4.96-4.83 (m, 1H), 4.61-4.40 (m, 1H), 4.12-4.04 (m, 1H), 4.02-3.95 (m, 1H), 3.87 (dd, J=4.0, 12.8 Hz, 2H), 3.59 (t, J=8.8 Hz, 1H), 3.34 (s, 3H), 3.31-3.29 (m, 1H), 2.85-2.74 (m, 2H), 2.00-1.75 (m, 2H), 0.89 (d, J=6.8 Hz, 6H).

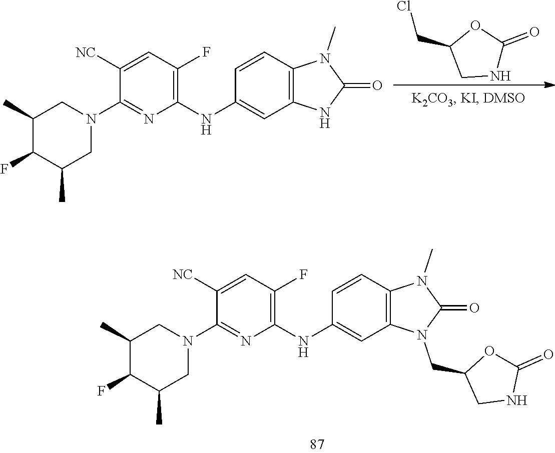 [SMILES: C[C@@H]1CN(c2nc(Nc3ccc4c(c3)[nH]c(=O)n4C)c(F)cc2C#N)C[C@H](C)[C@@H]1F ; O=C1NC[C@@H](CCl)O1 ; C[C@@H]1CN(c2nc(Nc3ccc4c(c3)n(C[C@@H]3CNC(=O)O3)c(=O)n4C)c(F)cc2C#N)C[C@H](C)[C@@H]1F]

### 5-Fluoro-2-[(3S,4S,5R)-4-fluoro-3,5-dimethyl-1-piperidyl]-6-[[1-methyl-2-oxo-3-[[(5S)-2-oxooxazolidin-5-yl]methyl]benzimidazol-5-yl]amino]pyridine-3-carbonitrile (87)

To a solution of 5-fluoro-2-[(3S,4S,5R)-4-fluoro-3,5-dimethyl-1-piperidyl]-6-[(1-methyl-2-oxo-3H-benzimidazol-5-yl)amino]pyridine-3-carbonitrile (150 mg, 363.69 μmol) in DMSO (1.5 mL) was added K₂CO₃ (100.53 mg, 727.39 μmol, 2 eq), KI (30.19 mg, 181.85 μmol) and (S)-5-(chloromethyl)oxazolidin-2-one (49.30 mg, 363.69 μmol). The mixture was stirred at 60° C. for 2 hr. The reaction mixture was filtered and the filtrate was purified by prep-HPLC (column: Waters Xbridge Prep OBD C18 150*40 mm*10 μm; mobile phase: [water(NH₄HCO₃)-ACN]; B %: 35%-50%, 8 min) to give the title compound as a white solid (30 mg, 58.65 μmol, 48% yield). ¹H NMR (400 MHz, DMSO-d₆) δ 9.40 (s, 1H), 7.77 (d, J=10.97 Hz, 1H), 7.50-7.61 (m, 2H), 7.38 (dd, J=8.46, 1.79 Hz, 1H), 7.11 (d, J=8.46 Hz, 1H). 4.82-4.97 (m, 1H), 4.42-4.62 (m, 1H), 4.03-4.15 (m, 1H), 3.96-4.02 (m, 1H), 3.87 (br dd, J=12.99, 4.05 Hz, 2H), 3.59 (t, J=8.82 Hz, 1H), 3.34 (s, 3H), 3.31-3.29 (m, 1H), 2.74-2.85 (m, 2H), 2.00-1.75 (m, 2H), 0.89 (d, J=6.91 Hz, 6H).

The absolute configurations of compounds 86 & 87 were randomly assigned based on all the substituents of the piperidine ring being in cis-conformation.

### Example 43: Synthesis of 5-fluoro-2-((3S,4S,5R)-4-fluoro-3,5-dimethylpiperidin-1-yl)-6-((1-methyl-2-oxo-3-(((R)-2-oxooxazolidin-4-yl)methyl)-2,3-dihydro-1H-benzo[d]imidazol-5-yl)amino)nicotinonitrile (88) and 5-fluoro-2-((3S,4S,5R)-4-fluoro-3,5-dimethylpiperidin-1-yl)-6-((1-methyl-2-oxo-3-(((S)-2-oxooxazolidin-4-yl)methyl)-2,3-dihydro-1H-benzo[d]imidazol-5-yl)amino)nicotinonitrile (89)

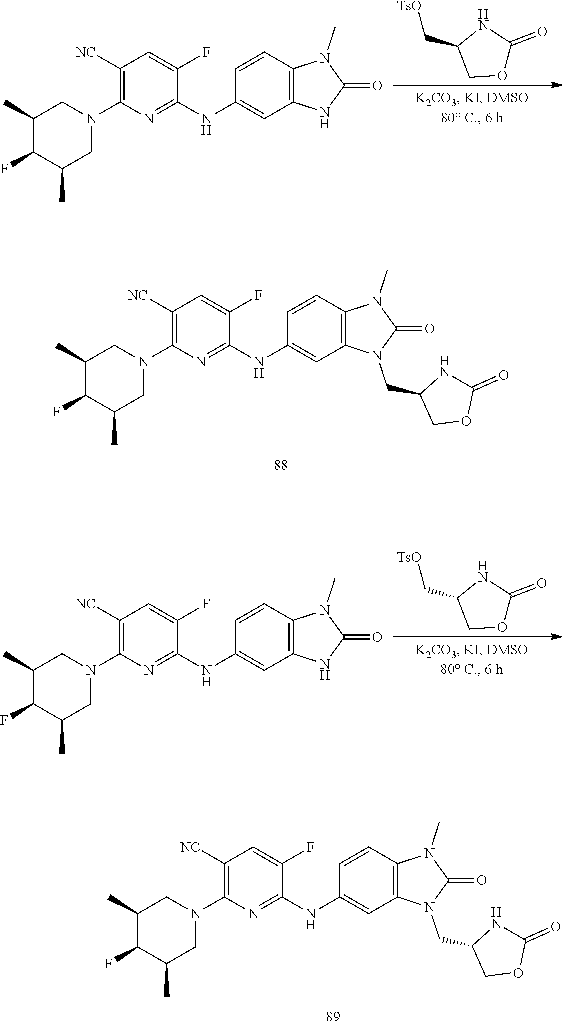 [SMILES: C[C@@H]1CN(c2nc(Nc3ccc4c(c3)[nH]c(=O)n4C)c(F)cc2C#N)C[C@H](C)[C@@H]1F ; Cc1ccc(S(=O)(=O)OC[C@@H]2COC(=O)N2)cc1 ; C[C@@H]1CN(c2nc(Nc3ccc4c(c3)n(C[C@@H]3COC(=O)N3)c(=O)n4C)c(F)cc2C#N)C[C@H](C)[C@@H]1F ; C[C@@H]1CN(c2nc(Nc3ccc4c(c3)[nH]c(=O)n4C)c(F)cc2C#N)C[C@H](C)[C@@H]1F ; Cc1ccc(S(=O)(=O)OC[C@H]2COC(=O)N2)cc1 ; C[C@@H]1CN(c2nc(Nc3ccc4c(c3)n(C[C@H]3COC(=O)N3)c(=O)n4C)c(F)cc2C#N)C[C@H](C)[C@@H]1F]

### 5-Fluoro-2-((3S,4S,5R)-4-fluoro-3,5-dimethylpiperidin-1-yl)-6-((1-methyl-2-oxo-3-(((R)-2-oxooxazolidin-4-yl)methyl)-2,3-dihydro-1H-benzo[d]imidazol-5-yl)amino)nicotinonitrile (88)

A flask with mixture of 5-fluoro-2-[(3S,5R)-4-fluoro-3,5-dimethyl-1-piperidyl]-6-[(1-methyl-2-oxo-3H-benzimidazol-5-yl)amino]pyridine-3-carbonitrile (100 mg, 242.46 μmol), [(4S)-2-oxooxazolidin-4-yl]methyl 4-methylbenzenesulfonate (98.67 mg, 363.69 μmol), K₂CO₃ (67.02 mg, 484.92 μmol) and KI (20.12 mg, 121.23 μmol) in DMSO (2 mL) was degassed and purged with N₂ for 3 times, and then the mixture was stirred at 80° C. for 6 hr under N₂ atmosphere. The reaction mixture was filtered and the filtrate was purified by prep-HPLC (column: Phenomenex C18 75*30 mm*3 μm; mobile phase: [water(NH₄HCO₃)-ACN]; B %₀: 35%-55%, 8 min) to give the title compound as a white solid (37 mg, 72.33 μmol, 30% yield, 1000% purity). ¹H NMR (400 MHz, DMSO-d₆) δ 9.38 (s, 1H), 7.83 (s, 1H), 7.77 (d, J=10.88 Hz, 1H), 7.49 (d, J=1.34 Hz, 1H), 7.34 (dd, J=8.38, 1.65 Hz, 1H), 7.10 (d, J=8.44 Hz, 1H), 4.42-4.59 (m, 1H), 4.33-4.40 (m, 1H), 4.18 (q, J=4.0.69 Hz, 2H), 3.80-3.95 (m, 4H), 3.33 (s, 3H), 2.78 (br t, J=12.41 Hz, 2H), 1.77-1.98 (m, 2H), 0.88 (dd, J=6.85, 3.42 Hz, 6H).

### 5-Fluoro-2-((3S,4S,5R)-4-fluoro-3,5-dimethylpiperidin-1-yl)-6-((1-methyl-2-oxo-3-(((S)-2-oxooxazolidin-4-yl)methyl)-2,3-dihydro-1H-benzo[d]imidazol-5-yl)amino)nicotinonitrile (89)

A flask with mixture of 5-fluoro-2-[(3S,5R)-4-fluoro-3,5-dimethyl-1-piperidyl]-6-[(1-methyl-2-oxo-3H-benzimidazol-5-yl)amino]pyridine-3-carbonitrile (100 mg, 242.46 μmol), [(4R)-2-oxooxazolidin-4-yl]methyl 4-methylbenzenesulfonate (98.67 mg, 363.69 μmol), K₂CO₃ (67.02 mg, 484.93 μmol) and KI (20.12 mg, 121.23 μmol) in DMSO (2 mL) was degassed and purged with N₂ for 3 times, and then the mixture was stirred at 80° C. for 6 hr under N₂ atmosphere. The reaction mixture was filtered and the filtrate was purified by prep-HPLC (column: Phenomenex C18 75*30 mm*3 μm; mobile phase: [water(NH₄HCO₃)-ACN]; B %: 35%-60%, 8 min) to give the title compound as a white solid (25 mg, 48.87 μmol, 20% yield, 100% purity). ¹H NMR (400 MHz, DMSO-d₆) δ 9.39 (s, 1H), 7.84 (s, 1H), 7.78 (d, J=10.88 Hz, 1H), 7.49 (d, J=1.71 Hz, 1H), 7.35 (dd, J=8.44, 1.71 Hz, 1H), 7.11 (d, J=8.44 Hz, 1H), 4.44-4.59 (m, 1H), 4.34-4.41 (m, 1H), 4.16-4.21 (m, 2H), 3.80-3.95 (m, 4H), 3.34 (s, 3H), 2.74-2.83 (m, 2H), 1.79-1.97 (m, 2H), 0.89 (dd, J=6.85, 3.42 Hz, 6H).

The absolute configurations of compounds 88 & 89 were randomly assigned based on all the substituents of the piperidine ring being in cis-conformation.

### Example 44: Synthesis of 2-((3R,5S)-3-amino-4,4-difluoro-5-methylpiperidin-1-yl)-5-fluoro-6-((3-(3-hydroxy-3-methylbutyl)-1-((S)-2-hydroxypropyl)-2-oxo-2,3-dihydro-1H-benzo[d]imidazol-5-yl)amino)nicotinonitrile (90) and 2-((3R,5S)-3-amino-4,4-difluoro-5-methylpiperidin-1-yl)-5-fluoro-6-((3-(3-hydroxy-3-methylbutyl)-1-((S)-2-hydroxypropyl)-2-oxo-2,3-dihydro-1H-benzo[d]imidazol-5-yl)amino)nicotinonitrile (91)

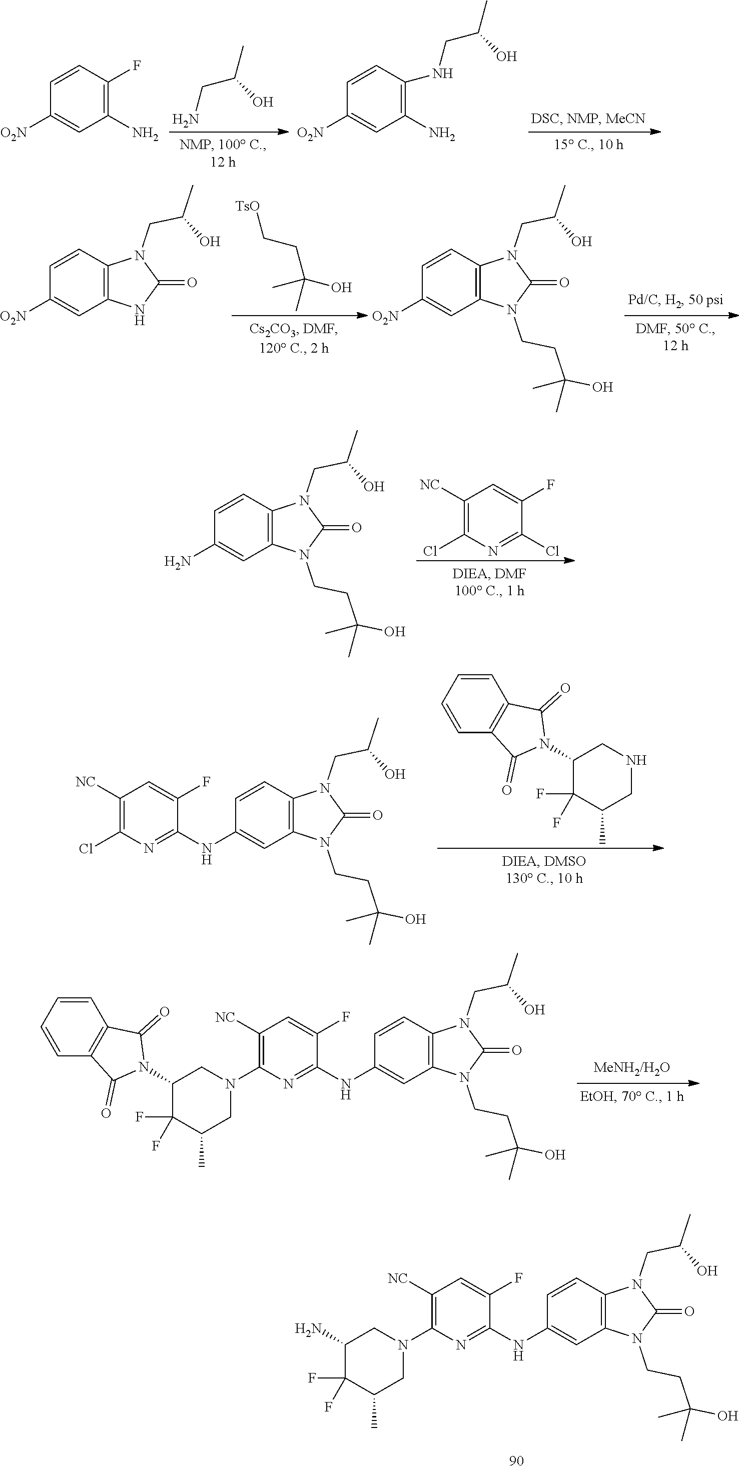

### (S)-1-((2-Amino-4-nitrophenyl)amino)propan-2-ol

A mixture of 2-fluoro-5-nitroaniline (6 g, 38.43 mmol) and (S)-1-aminopropan-2-ol (11.55 g, 153.73 mmol, 12.10 mL) in NMP (120 mL) was stirred at 100° C. for 12 hr. The reaction mixture (combined with another batch at same scale) was treated with H₂O (300 mL), forming a precipitate which was filtered and the solid was washed with solvent (Petroleum ether/Ethyl acetate=2:1, 500 mL). The filter cake was collected and dried in vacuo to give the title compound as a yellow solid (15 g, 65.34 mmol, 85% yield, 92% purity). ¹H NMR (400 MHz, DMSO-d₆) δ 7.51 (d, J=8.8 Hz, 1H), 7.42 (d, J=2.0 Hz, 1H), 6.50 (d, J=8.8 Hz, 1H), 5.90 (s, 1H), 5.17 (s, 2H), 4.86 (s, 1H), 3.81-3.93 (m, 1H), 3.05-3.21 (m, 2H), 1.13 (d, J=6.0 Hz, 3H). LCMS: [M+H]⁺=212.3.

### (S)-1-(2-Hydroxypropyl)-5-nitro-1H-benzo[d]imidazol-2(3H)-one

To a solution of (S)-1-((2-amino-4-nitrophenyl)amino)propan-2-ol (15 g, 71.02 mmol) in MeCN (150 mL) and NMP (15 mL) was added DSC (19.10 g, 74.57 mmol). The mixture was stirred at 15° C. for 12 hr. The reaction mixture was filtered to give a filter cake which was washed with solvent (Petroleum ether/Ethyl acetate=1:1, 80 mL), collected and dried in vacuo to give the title compound as a yellow solid (9 g, 37.18 mmol, 52% yield, 98% purity). ¹H NMR (400 MHz, DMSO-d₆) δ 11.40 (s, 1H), 7.99 (dd, J=8.8, 2.0 Hz, 1H), 7.74 (d, J=2.0 Hz, 1H), 7.35 (d, J=8.8 Hz, 1H), 4.91 (d, J=4.8 Hz, 1H), 3.96 (m, 1H), 3.71-3.79 (m, 2H), 1.10 (d, J=6.0 Hz, 3H).

### (S)-3-(3-Hydroxy-3-methylbutyl)-1-(2-hydroxypropyl)-5-nitro-1H-benzo[d]imidazol-2(3H)-one

To a solution of (S)-1-(2-hydroxypropyl)-5-nitro-1H-benzo[d]imidazol-2(3H)-one (4 g, 16.86 mmol) and 3-hydroxy-3-methylbutyl 4-methylbenzenesulfonate (6.97 g, 26.98 mmol) in DMF (50 mL) was added Cs₂CO₃ (10.99 g, 33.73 mmol). The mixture was stirred at 120° C. for 2 hr. Then water (80 mL) was added to the mixture (combined with another batch of 1 g scale), and extracted with EtOAc (100 mL×3). The combined organic phase was washed with brine (150 mL), dried with anhydrous Na₂SO₄, filtered and concentrated in vacuo to give the title compound as a yellow oil (6.5 g, 91% purity). LCMS: [M+H]⁺=324.3.

### (S)-5-Amino-3-(3-hydroxy-3-methylbutyl)-1-(2-hydroxypropyl)-1H-benzo[d]imidazol-2(3H)-one

To a solution of (S)-3-(3-hydroxy-3-methylbutyl)-1-(2-hydroxypropyl)-5-nitro-1H-benzo[d]imidazol-2(3H)-one (6.5 g, 20.10 mmol) in DMF (100 mL) was added Pd/C (2 g, 10% purity) under Ar. The flask was degassed under vacuum and purged with H₂ for three times. The mixture was stirred under H₂ (50 psi) at 50° C. for 12 hr. The reaction mixture was filtered, and the filtrate was concentrated in vacuo to give the title compound as a yellow oil (5 g, 15.00 mmol, 75% yield, 88% purity). LCMS: [M+H]⁺=294.1.

### (S)-2-Chloro-5-fluoro-6-((3-(3-hydroxy-3-methylbutyl)-1-(2-hydroxypropyl)-2-oxo-2,3-dihydro-1H-benzo[d]imidazol-5-yl)amino)nicotinonitrile

To a solution of (S)-5-amino-3-(3-hydroxy-3-methylbutyl)-1-(2-hydroxypropyl)-1H-benzo[d]imidazol-2(3H)-one (1 g, 3.41 mmol) and 2,6-dichloro-5-fluoronicotinonitrile (651.04 mg, 3.41 mmol) in DMF (10 mL) was added DIEA (881.12 mg, 6.82 mmol, 1.19 mL). The mixture was stirred at 100° C. for 1 hr. The reaction mixture was treated with H₂O (50 mL), forming a precipitate which was filtered and the solid was washed with solvent (Petroleum ether/Ethyl acetate=2:1, 50 mL). The filter cake was collected and dried in vacuo to give the title compound as a yellow solid (1.3 g, 2.67 mmol, 78% yield, 92% purity). LCMS: [M+H]⁺=448.1.

### 2-((3R,5S)-3-(1,3-Dioxoisoindolin-2-yl)-4,4-difluoro-5-methylpiperidin-1-yl)-5-fluoro-6-((3-(3-hydroxy-3-methylbutyl)-1-((S)-2-hydroxypropyl)-2-oxo-2,3-dihydro-1H-benzo[d]imidazol-5-yl)amino)nicotinonitrile

To a solution of (S)-2-chloro-5-fluoro-6-((3-(3-hydroxy-3-methylbutyl)-1-(2-hydroxypropyl)-2-oxo-2,3-dihydro-1H-benzo[d]imidazol-5-yl)amino)nicotinonitrile (300 mg, 669.81 μmol) and 2-((3R,5S)-4,4-difluoro-5-methylpiperidin-3-yl)isoindoline-1,3-dione (187.73 mg, 669.81 μmol) in DMSO (3 mL) was added DIEA (432.84 mg, 3.35 mmol, 583.34 μL). The mixture was stirred at 130° C. for 10 hr. The mixture (combined with another batch at 50 mg scale) was purified directly without further work-up. The residue was purified by prep-HPLC (column: Waters Xbridge Prep OBD C18 150*40 mm*10 μm; mobile phase: [water (NH₄HCO₃)-ACN]; B %: 35%-65%, 8 min) to give the title compound as a yellow solid (90 mg, 123.61 μmol, 18% yield, 95% purity). LCMS: [M+H]⁺=692.3.

### 2-((3R,5S)-3-Amino-4,4-difluoro-5-methylpiperidin-1-yl)-5-fluoro-6-((3-(3-hydroxy-3-methylbutyl)-1-((S)-2-hydroxypropyl)-2-oxo-2,3-dihydro-1H-benzo[d]imidazol-5-yl)amino)nicotinonitrile (90)

To a solution of 2-((3R,5S)-3-(1,3-dioxoisoindolin-2-yl)-4,4-difluoro-5-methylpiperidin-1-yl)-5-fluoro-6-((3-(3-hydroxy-3-methylbutyl)-1-((S)-2-hydroxypropyl)-2-oxo-2,3-dihydro-1H-benzo[d]imidazol-5-yl)amino)nicotinonitrile (70 mg, 101.20 μmol) in EtOH (3.5 mL) was added MeNH₂ (7.86 mg, 101.20 μmol, 3.5 mL, 40% purity) (40% in water) under N₂. The mixture was stirred at 70° C. for 1 hr. The mixture (combined with another batch at 20 mg scale) was concentrated in vacuo. The residue was purified by prep-HPLC (column: Phenomenex C18 75*30 mm*3 μm; mobile phase: [water (NH₄HCO₃)-ACN]; B %: 35%-65%, 8 min) to give the title compound as a white solid (20 mg, 35.35 μmol, 35% yield, 99% purity). ¹H NMR (400 MHz, DMSO-d₆) δ 9.44 (s, 1H), 7.81 (d, J=10.85 Hz, 1H), 7.40 (d, J=1.91 Hz, 1H), 7.34 (dd, J=8.46, 1.91 Hz, 1H), 7.15 (d, J=8.46 Hz, 1H), 4.86 (d, J=5.01 Hz, 1H), 4.51 (s, 1H), 4.11 (d, J=12.16 Hz, 1H), 3.93-4.01 (m, 2H), 3.89 (dd, J=10.55, 6.62 Hz, 2H), 3.71 (dd, J=5.84, 2.03 Hz, 2H), 2.88-3.02 (m, 1H), 2.69-2.87 (m, 2H), 2.03-2.19 (m, 1H), 1.70 (t, J=8.05 Hz, 4H), 1.16 (d, J=2.62 Hz, 6H), 1.06 (d, J=6.20 Hz, 3H), 0.90 (d, J=6.68 Hz, 3H).

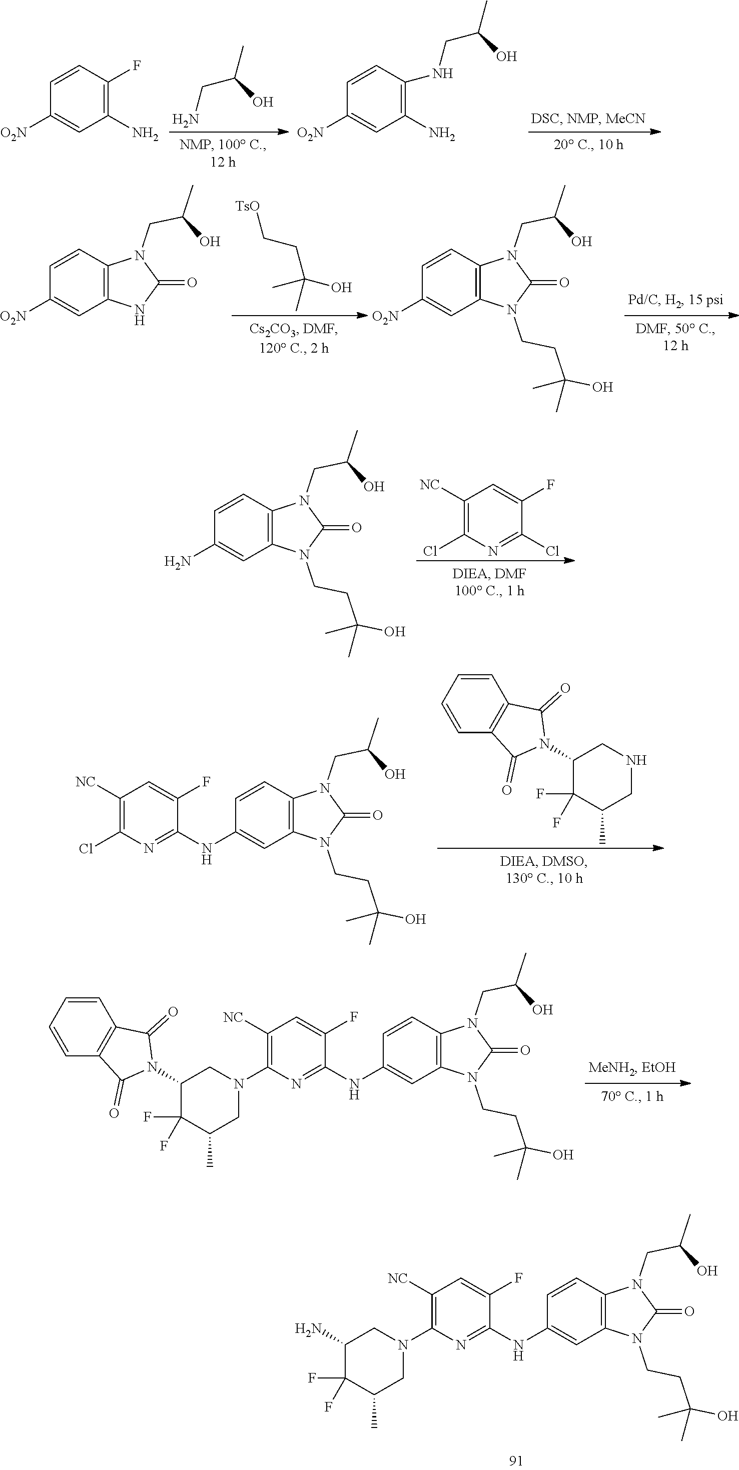

### (R)-1-((2-Amino-4-nitrophenyl)amino)propan-2-ol

A mixture of 2-fluoro-5-nitro-aniline (10 g, 64.06 mmol) and (R)-1-aminopropan-2-ol (19.25 g, 256.24 mmol, 20.17 mL) in NMP (100 mL) was stirred at 100° C. for 12 hr. The reaction mixture was treated with H₂O (300 mL), forming a precipitate which was filtered and the solid was washed with solvent (EtOAc:PE=1:2, 500 mL), the filter cake was collected and dried in vacuo to give the title compound as a yellow solid (12 g, 55.68 mmol, 87% yield, 98% purity). LCMS: [M+H]⁺=212.3.

### (R)-1-(2-Hydroxypropyl)-5-nitro-1H-benzo[d]imidazol-2(3H)-one

To a solution of (R)-1-(2-amino-4-nitro-anilino)propan-2-ol (10 g, 47.34 mmol) in ACN (100 mL) was added DSC (12.13 g, 47.34 mmol) followed with NMP (10 mL). The reaction mixture was stirred at 20° C. for 12 hr. The reaction mixture was filtered and then the solid was washed with (PE:EtOAc=1:1, 80 mL), collected and dried in vacuo to give the title compound as a yellow solid (9.3 g, 38.81 mmol, 82% yield, 99% purity). ¹H NMR (400 MHz, DMSO-d₆) δ 11.40 (s, 1H), 7.99 (dd, J=2.4, 8 Hz, 1H), 7.74 (d, J=2.4 Hz, 1H), 7.35 (d, J=8.8 Hz, 1H), 4.91 (d, J=4.8 Hz, 1H), 4.02-3.91 (m, 1H), 3.81-3.69 (m, 2H), 1.10 (d, J=6.4 Hz, 3H).

### (R)-3-(3-Hydroxy-3-methylbutyl)-1-(2-hydroxypropyl)-5-nitro-1H-benzo[d]imidazol-2(3H)-one

To a mixture of 3-[(R)-2-hydroxypropyl]-6-nitro-1H-benzimidazol-2-one (4.3 g, 18.13 mmol) and (3-hydroxy-3-methyl-butyl) 4-methylbenzenesulfonate (7.49 g, 29.00 mmol) in DMF (50 mL) was added Cs₂CO₃ (11.81 g, 36.25 mmol) and stirred at 120° C. for 2 hr. The reaction mixture was treated with H₂O (80 mL) and extracted with EtOAc (100 mL×3), then the combined organic phase was washed with brine (150 mL), dried with anhydrous Na₂SO₄, filtered and concentrated in vacuo to give the title compound as a yellow oil (6.5 g, crude). ¹H NMR (400 MHz, CDCl₃) δ 8.06 (dd, J=2.0, 8.8 Hz, 1H), 7.92 (d, J=2.0 Hz, 1H), 7.19 (d, J=8.8 Hz, 1H), 4.26-4.17 (m, 1H), 4.14-4.06 (m, 2H), 4.02-3.93 (m, 1H), 3.91-3.82 (m, 1H), 2.51 (s, 1H), 1.91 (t, J=7.6 Hz, 2H), 1.48 (s, 1H), 1.30 (s, 6H), 1.28 (d, J=6.0 Hz, 3H).

### (R)-5-Amino-3-(3-hydroxy-3-methylbutyl)-1-(2-hydroxypropyl)-1H-benzo[d]imidazol-2(3H)-one

To a solution of 3-(3-hydroxy-3-methyl-butyl)-1-[(R)-2-hydroxypropyl]-5-nitro-benzimidazol-2-one (6.5 g, 20.10 mmol) in DMF (100 mL) was added Pd/C (3 g, 21.65 mmol, 10% purity) under Ar. The flask was degassed under vacuum and purged with H₂ for three times. The mixture was stirred under H₂ (50 psi) at 50° C. for 12 hours. The reaction mixture was filtered, and the filtrate was concentrated in vacuo and purified by column chromatography (SiO₂, EtOAc/EtOH=1/0 to 3/1) to give the title compound as a yellow solid (3.6 g, 11.54 mmol, 57% yield, 94% purity). LCMS: [M+H]⁺=294.1.

### (R)-2-Chloro-5-fluoro-6-((3-(3-hydroxy-3-methylbutyl)-1-(2-hydroxypropyl)-2-oxo-2,3-dihydro-1H-benzo[d]imidazol-5-yl)amino)nicotinonitrile

To a mixture of 5-amino-3-(3-hydroxy-3-methyl-butyl)-1-[(R)-2-hydroxypropyl]benzimidazol-2-one (1 g, 3.41 mmol) and 2,6-dichloro-5-fluoro-pyridine-3-carbonitrile (651.04 mg, 3.41 mmol) in DMF (10 mL) was added DIPEA (881.12 mg, 6.82 mmol, 1.19 mL), and the reaction mixture was stirred at 100° C. for 1 hr. The reaction mixture was treated with H₂O (20 mL), forming a precipitate which was filtered and the solid was washed with (PE:EtOAc=2:1, 40 mL). The solid was dried in vacuo to give the title compound as a brownish red solid (1.3 g, 2.61 mmol, 77% yield, 90% purity). LCMS: [M+H]⁺=448.2.

### 2-((3R,5S)-3-(1,3-Dioxoisoindolin-2-yl)-4,4-difluoro-5-methylpiperidin-1-yl)-5-fluoro-6-((3-(3-hydroxy-3-methylbutyl)-1-((R)-2-hydroxypropyl)-2-oxo-2,3-dihydro-1H-benzo[d]imidazol-5-yl)amino)nicotinonitrile

To a mixture of 2-chloro-5-fluoro-6-[[3-(3-hydroxy-3-methyl-butyl)-1-[(R)-2-hydroxypropyl]-2-oxo-benzimidazol-5-yl]amino]pyridine-3-carbonitrile (319.61 mg, 713.60 μmol) and 2-[(3R,5S)-4,4-difluoro-5-methyl-3-piperidyl]isoindoline-1,3-dione (200 mg, 713.60 μmol) in DMSO (3 mL) was added DIPEA (184.46 mg, 1.43 mmol, 248.59 μL), and the reaction mixture was stirred at 130° C. for 10 hr. The reaction mixture was filtered (combined with one batch of 100 mg scale and another batch of 200 mg scale) and was purified by prep-HPLC (column: C18 (250*50 mm*10 μm); mobile phase: [water(NH₄HCO₃)-ACN]; B %: 40%-65%, 10 min) to give the title compound as a yellow solid (250 mg, 90% purity). LCMS: [M+H]⁺=692.3.

### 2-((3R,5S)-3-Amino-4,4-difluoro-5-methylpiperidin-1-yl)-5-fluoro-6-((3-(3-hydroxy-3-methylbutyl)-1-((R)-2-hydroxypropyl)-2-oxo-2,3-dihydro-1H-benzo[d]imidazol-5-yl)amino)nicotinonitrile (91)

To a solution of 2-[(3R,5S)-3-(1,3-dioxoisoindolin-2-yl)-4,4-difluoro-5-methyl-1-piperidyl]-5-fluoro-6-[[3-(3-hydroxy-3-methyl-butyl)-1-[(R)-2-hydroxypropyl]-2-oxo-benzimidazol-5-yl]amino]pyridine-3-carbonitrile (100 mg, 144.57 μmol) in EtOH (7.5 mL) was added MeNH₂ (11.22 mg, 144.57 μmol, 7.5 mL, 40% purity) and was stirred at 70° C. for 1 hr. The reaction mixture was concentrated in vacuo and then purified by prep-HPLC (column: Phenomenex C18 75*30 mm*3 μm; mobile phase: [water(NH₄HCO₃)-ACN]; B %: 15%-60%, 8 min) to give the title compound as a white solid (30 mg, 53.42 μmol, 37% yield, 100% purity). ¹H NMR (400 MHz, DMSO-d₆) δ 9.44 (s, 1H), 7.81 (d, J=10.8 Hz, 1H), 7.40 (d, J=2.0 Hz, 1H), 7.33 (dd, J=1.6, 8.8 Hz, 1H), 7.15 (d, J=8.4 Hz, 1H), 4.86 (d, J=5.2 Hz, 1H), 4.52 (s, 1H), 4.12 (d, J=12.0 Hz, 1H), 4.03-3.85 (m, 4H), 3.75-3.66 (m, 2H), 3.00-2.87 (m, 1H), 2.87-2.79 (m, 1H), 2.74 (t, J=12.4 Hz, 1H), 2.22-2.02 (m, 1H), 1.80-1.62 (m, 4H), 1.16 (d, J=2.0 Hz, 6H), 1.06 (d, J=6.4 Hz, 3H), 0.89 (d, J=6.4 Hz, 3H).

The absolute configurations of compounds 90 & 91 were randomly assigned based on the amino group and methyl group being in cis-conformation.

### Example 45: Degradation Activity

TABLE 1
Degradation activity of compounds

| Compound # | HiBiT SU-DHL-4 DC₅₀ (μM) |
|---|---|
| 1 | 0.006 |
| 2 | 0.006 |
| 3 | 0.003 |
| 6 | 0.013 |
| 7 | 0.005 |
| 9 | >0.4 |
| 11 | 0.002 |
| 14 & 15 (tested as mixture) | 0.060 |
| 18 | 0.005 |
| 24 | 0.030 |
| 25 | >0.4 |
| 35 | >0.4 |
| 36 | >0.4 |
| 37 | 0.062 |
| 38 | >0.4 |
| 39 | 0.041 |
| 40 | >0.4 |
| 41 | 0.010 |
| 42 | 0.071 |
| 43 | 0.010 |
| 44 | 0.070 |
| 45 | 0.003 |
| 46 | 0.006 |
| 47 | 0.011 |
| 48 | 0.031 |
| 49 | >0.4 |
| 50 | 0.028 |
| 51 | >0.4 |
| 52 | 0.017 |
| 53 | >0.4 |
| 54 | 0.038 |
| 55 | 0.033 |
| 56 | >0.4 |
| 57 | >0.4 |
| 58 | >0.4 |
| 59 | 0.048 |
| 60 | >0.4 |
| 61 | >0.4 |
| 62 | 0.008 |
| 63 | 0.039 |
| 64 | 0.007 |
| 65 | 0.056 |
| 66 | 0.006 |
| 67 | 0.004 |
| 68 | 0.004 |
| 69 | 0.015 |
| 70 | >0.4 |
| 71 | >0.4 |
| 72 | 0.002 |
| 73 | 0.009 |
| 74 | 0.022 |
| 75 | >0.4 |
| 76 | 0.004 |
| 77 | 0.004 |
| 78 | >0.4 |
| 79 | 0.012 |
| 80 | >0.4 |
| 81 | 0.031 |
| 82 | 0.033 |
| 83 | >0.4 |
| 84 | >0.4 |
| 85 | >0.4 |
| 86 | 0.083 |
| 87 | 0.101 |
| 88 | 0.080 |
| 89 | 0.102 |
| 90 | 0.073 |
| 91 | 0.118 |

### HiBiT Protocol

DC₅₀ (concentration to reach 50% degradation) values were determined from a cellular degradation assay (HiBiT, Promega™) in Su-DHL-4 cells (Table 1). Endogenous BCL6 was tagged with the 11-amino acid SmBiT through CRISPR/Cas9 gene editing and single cell clone selection. After 24 hours of compound treatment, cells were lysed and incubated with LgBiT protein to reconstitute intact nanoluciferase. Substrate was then added and relative luciferase units were measured. Degradation levels for each treatment were taken as a percentage compared to the control, 100% DMSO (Prism).

### Example 46: Synergistic Effects of BCL6 Degradation and EZH2 Inhibition

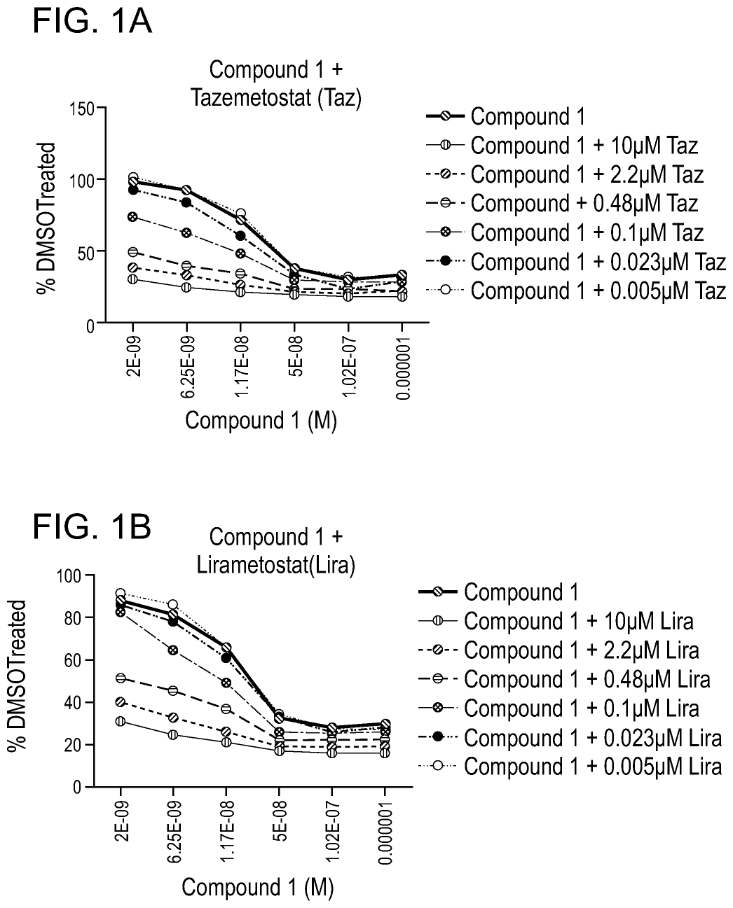

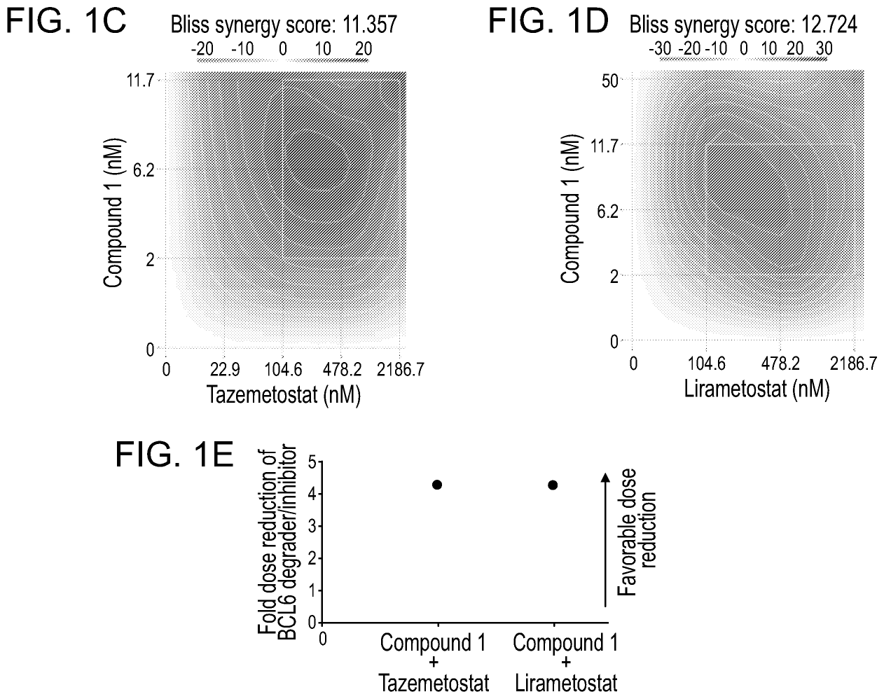

Enhancer of zeste homolog 2 (EZH2) is a member of the polycomb repressive complex 2 (PRC2) that methylates histone 3 lysine 27, a mark that is associated with gene repression. EZH2 expression is up-regulated in both normal germinal center B cell development and in the germinal center (GC) subtype of B cell lymphomas, much like BCL6 (Caganova et al., J. Clin. Invest. 123(12):5009-5022 (2013); Beguelin et al., Cancer Cell 23(5):677-692 (2013)). Cooperation between BCL6 and PRC2 in negatively regulating transcription has been described in both normal B-cell development and lymphomagenesis (Beguelin et al., Cancer Cell 30(2):197-213 (2016)). Su-DHL-4 cells were treated with various concentrations of Tazemetostat (Taz) or Lirametostat (Lira) (ranging from 10 μM to 5 nM). After 48 hours, the cells were treated with various concentrations of compound 1 (ranging from 50 nM to 2 nM). Cell proliferation was monitored via CellTiter-Glo® assay (Promega™) five days after the treatment of compound 1. The anti-proliferative effects were plotted for both Taz (FIG. 1A) and Lira treatments (FIG. 1B). The excess over Bliss (eob) scores were calculated over a range of at least three concentrations (FIG. 1C-FIG. 1D), and a significant synergistic effect (eob>1, average synergy score of over 10, SynergyFinder (Ianevski et al., Bioinformatics 33(15):2413-2415 (2017)), was observed over most of the concentration range. Since anti-proliferation effect was only observed at DC₉₉ of BCL6 degrader treatment in single-compound experiment, the dose reduction at 60% of growth inhibition (GI₆₀) was calculated through the addition of the two EZH2 inhibitors. BCL6 degraders for both Taz and Lira treatments at 480 nM reduced the BCL6 degrader dose requirement to achieve GI₆₀ by around 4.3 fold (FIG. 1E). Taken together, these results demonstrated that EZH2 inhibition exerted synergistic anti-proliferative effects with BCL6 degraders.

### Example 47: Proliferation Protocol

To ensure HiBiT signal decrease was due to BCL6 degradation and not from dying cells, CellTiter-Glo® (CTG, Promega™) was performed on the same number of cells that were treated for 24 hours with BCL6 degraders. To study the anti-proliferative effects of BCL6 degraders and its synergistic effect with EZH2 inhibitors, Su-DHL-4 and other DLBCL cells were treated with the compounds at DC₉₉, DC₇₅, DC₅₀, or 1 μM followed by 5 days of culture, at which point CTG values and/or Caspase3/7 activities were measured as percent DMSO treated.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound having a structure represented by formula I: 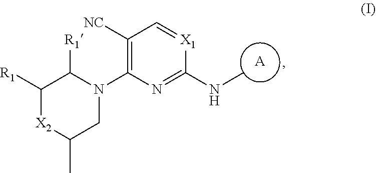
or a pharmaceutically acceptable salt or stereoisomer thereof,
wherein:
X₁ is N, CH, CCl, CF, or CCN;
each X₂ is independently CH₂, S, CHF, CHCl, CHOH, or CF₂;
R₁ is hydrogen, ═O, —CN, —C≡CH, —OH, —SH, —NH₂, —COOH, halo, (C₁-C₆)alkyl, —O—(C₁-C₆)alkyl, (C₁-C₆)haloalkyl, amido, carboxy, carbamoyl, sulfamoyl, phenyl, 5- to 8-membered heterocyclyl, —NR₇R₈, —C(O)R₉, —C(O)NR₁₀R₁₁, or L₁Y₁, wherein said alkyl, phenyl, or heterocyclyl is optionally substituted with one or more groups selected from halo, —COOH, —OH, —NH₂, (C₁-C₆)alkyl, —C(O)O—(C₁-C₆)alkyl, —C(O)N(C₁-C₆ alkyl)₂, —O—(C₁-C₆)alkyl, —N(C₁-C₃ alkyl)₂, phenyl, and 4- to 6-membered heterocyclyl, optionally substituted with one or more groups selected from halo and (C₁-C₆)alkyl;
R₇ is hydrogen, (C₁-C₄)alkyl, or (C₃-C₆)cycloalkyl;
R₈ is hydrogen, (C₁-C₄)alkyl, (C₁-C₄)haloalkyl, (C₃-C₆)cycloalkyl, or 6-membered heterocyclyl;
R₉ is —(C₁-C₃)alkyl-N(C₁-C₃ alkyl)₂, (C₃-C₆)cycloalkyl, or 5- to 6-membered heterocyclyl, wherein said heterocyclyl is optionally substituted with (C₁-C₃)alkyl;
R₁₀ is hydrogen, (C₁-C₃)alkyl, or (C₃-C₆)cycloalkyl;
R₁₁ is (C₃-C₆)cycloalkyl or (C₁-C₆)alkyl optionally substituted with —NH₂, —O—(C₁-C₆)alkyl, —O—(C₁-C₆)alkyl-NH₂, or —O—(C₁-C₆)alkyl-O—(C₁-C₆)alkyl-NH₂;
L₁ is absent, (C₁-C₆)alkylene or (C₃-C₇)carbocyclyl; wherein said alkylene or carbocyclyl is further optionally substituted by one or more, identical or different R_{A} groups;
each R_{A} is independently oxo, alkyl, alkenyl, alkynyl, halo, haloalkyl, carbocyclyl, heterocyclyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, or phosphinyl;
Y₁ is —CN, —OH, halo, (C₁-C₄)alkoxy, (C₁-C₄)haloalkyl, (C₁-C₄)haloalkoxy, 4- to 7-membered heterocyclyl, (C₃-C₆)carbocyclyl, —NR_{7′}R_{8′}, —C(O)R₉, —C(O)NR₁₀R₁₁; wherein said alkyl, carbocyclyl, or heterocyclyl is further optionally substituted by one or more, identical or different groups selected from (C₁-C₄)alkyl, halo, (C₁-C₄)haloalkyl, —CN, —OH, and —NH₂;
R_{7′} and R_{8′} are each independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl; wherein said alkyl, carbocyclyl, heterocyclyl, aryl or heteroaryl is further optionally substituted by one or more, identical or different R_{A} groups, or
R_{7′} and R_{8′} together with the nitrogen atom to which they are attached form a 3- to 7-membered heterocyclyl, wherein said heterocyclyl is further optionally substituted by one or more, identical or different R_{A} groups, or L₁ is (C₂-C₄)alkylene which is bound to R_{7′} to form a 4- to 6-membered heterocyclyl group;
R_{1′} is absent, hydrogen, —CN, —C≡CH, —OH, —SH, —NH₂, —COOH, halo, (C₁-C₆)alkyl, —O—(C₁-C₆)alkyl, (C₁-C₆)haloalkyl, amido, carboxy, carbamoyl, sulfamoyl, phenyl, 5- to 8-membered heterocyclyl, —NR₇R₈, —C(O)R₉, or —C(O)NR₁₀R₁₁; wherein said alkyl, phenyl, or heterocyclyl is further optionally substituted by one or more, identical or different R_{A} groups, or
R_{1′} and L₁ together with the same carbon atom to which they are attached form a spiro (C₃-C₇)carbocyclyl group or a 4- to 7-membered heterocyclyl group; wherein said carbocyclyl or heterocyclyl, is further optionally substituted by one or more, identical or different R_{A} groups;
\textcircled{A} is 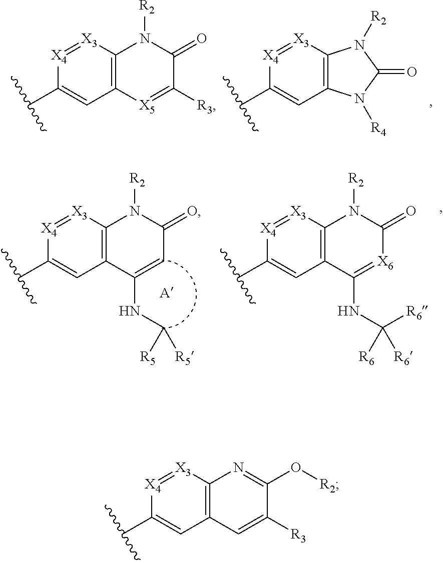
X₃ and X₄ are independently CR₁₂ or N;
X₅ is CH or N;
R₁₂ is hydrogen, (C₁-C₄)alkyl, halo, hydroxy, amino, (C₁-C₄)alkoxy, (C₁-C₄)haloalkyl, (C₁-C₄)haloalkoxy, (C₂-C₄)alkenyl, (C₂-C₄)alkynyl, nitro, cyano, NH(C₁-C₄)alkyl, or N(C₁-C₄ alkyl)₂;
R₂ is hydrogen, (C₁-C₆)alkyl, (C₃-C₆)carbocyclyl, 4- to 7-membered heterocyclyl, (C₃-C₇)carbocyclyl(C₁-C₆)alkyl, or 4- to 7-membered heterocyclyl(C₁-C₆)alkyl; wherein said alkyl, carbocyclyl, or heterocyclyl is further optionally substituted by one or more, identical or different R₁₃ groups, wherein R₁₃ is (C₁-C₆)alkyl, (C₁-C₆)alkoxy, halo, amino, hydroxyl, haloalkyl, NH(C₁-C₆)alkyl, or N((C₁-C₆)alkyl)₂, (C₃-C₆)carbocyclyl, or 4- to 7-membered heterocyclyl, or
R₂ is -L₂-Y₂—Z;
L₂ is absent or (C₁-C₅)alkylene optionally substituted by one or more substituents selected from (C₁-C₂)alkyl and oxo;
Y₂ is absent, O, S, S(O), S(O)₂, NR′, C(O), C(O)O, OC(O), C(O)N(R′), N(R′)C(O), N(R′)C(O)N(R′), N(R′)C(O)O, OC(O)N(R′), S(O)₂N(R′), or N(R′)S(O)₂;
each R′ is independently hydrogen or (C₁-C₄)alkyl;
Z is hydrogen, (C₁-C₆)alkyl, (C₂-C₆)alkenyl, (C₂-C₆)alkynyl, (C₃-C₁₀)carbocyclyl, or 3- to 10-membered heterocyclyl; wherein Z is optionally substituted by one or more substituents independently selected from (C₁-C₄)alkyl, halo, (C₁-C₄)haloalkyl, (C₁-C₄)haloalkoxy, amino, (C₁-C₄)aminoalkyl, cyano, hydroxy, carboxy, carbamoyl, sulphamoyl, mercapto, ureido, NR^{r}R^{s}, OR^{r}, C(O)R^{r}, C(O)OR^{r}, OC(O)R^{r}, C(O)NR^{r}R^{s}, N(R^{r})C(O)R^{r}, S(O)₀₋₂R^{r}, S(O)₂NR^{r}R^{s}, N(R^{r})SO₂R^{r}, Si(R^{r})(R^{s})R^{t} and (CH₂)₁₋₃NR^{r}R^{s}; wherein R^{r}, R^{s}, and R^{t} are each independently hydrogen, (C₁-C₆)alkyl, or (C₃-C₆)cycloalkyl; or R^{r} and R^{s} together with the nitrogen atom to which they are attached form a 4- to 9-membered heterocyclyl which is optionally substituted by one or more substituents selected from (C₁-C₄)alkyl, halo, (C₁-C₄)haloalkyl, (C₁-C₄)haloalkoxy, (C₁-C₄)alkoxy, (C₁-C₄)alkylamino, amino, cyano, and hydroxy;
R₃ is -L₃CR₁₄R₁₅R₁₆, or —CH═CH—R₁₆;
L₃ is absent, O, S, (C₁-C₄)alkylene, —O—(C₁-C₄)alkylene, or —S—(C₁-C₄)alkylene;
R₁₄ is hydrogen or (C₁-C₄)alkyl;
R₁₅ is hydrogen or (C₁-C₄)alkyl, or
R₁₄ and R₁₅ together with the carbon atom to which they are attached form a (C₃-C₅)carbocyclyl, 4- to 7-membered heterocyclyl, or C═O;
R₁₆ is (C₁-C₆)alkyl, —NR₁₇R₁₈, —OR₁₇, —C(O)R₁₇, —C(O)OR₁₇, —N(R₁₈)C(O)R₁₇, —C(O)NR₁₇R₁₈, —S(O)—(C₁-C₆)alkyl, —S(O)₂—(C₁-C₆)alkyl, —P(O)—(C₁-C₆ alkyl)₂, —C(NH)NH₂, —(C₁-C₄)alkyl-NR₁₈C(O)R₁₇, or 4- to 7-membered heterocyclyl;
R₁₇ is hydrogen, 3- to 6-membered heterocyclyl, or (C₁-C₄)alkyl optionally substituted by one or more, identical or different groups selected from OH, Cl, F, CF₃, N(C₁-C₄ alkyl)₂, (C₃-C₆)carbocyclyl, 3- to 6-membered heterocyclyl, (C₂-C₄)alkenyl, and (C₂-C₄)alkynyl;
R₁₈ is hydrogen or (C₁-C₄)alkyl:
R₄ is hydrogen, methyl, —(CH₂)₁₋₃W₁W₂, or 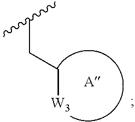
W₁ is CR₁₉R_{19′} or C(O);
R₁₉ and R_{19′} are independently hydrogen, (C₁-C₂)alkyl, fluoro, hydroxy, cyano, nitro, (C₁-C₂)alkoxy, (C₁-C₂)haloalkyl, (C₁-C₂)haloalkoxy, amino, NH(C₁-C₂)alkyl, or N(C₁-C₂ alkyl)₂, or
R₁₉ and R_{19′} together with the carbon atom to which they are attached form C(O), (C₃-C₆)carbocyclyl or 3- to 6-membered heterocyclyl, which is optionally substituted by one or more substituents independently selected from (C₁-C₂)alkyl, halo, (C₁-C₂)haloalkyl, (C₁-C₂)haloalkoxy, (C₁-C₂)alkoxy, (C₁-C₂)alkylamino, amino, cyano, and hydroxy;
W₂ is cyano, hydroxy, 5- or 6-membered heteroaryl, phenyl, C(O)—(C₁-C₂)alkyl, S(O)₂—(C₁-C₂)alkyl, C(O)OCH₃, C(O)NHCH₃, CR₂₀R₂₁R₂₂, amino, NH(C₁-C₂)alkyl, or N(C₁-C₂ alkyl)₂:
R₂₀ is hydrogen, (C₁-C₂)alkyl, fluoro, chloro, bromo, hydroxy, amino, cyano, nitro, (C₁-C₂)alkoxy, (C₁-C₂)haloalkyl, or (C₁-C₂)haloalkoxy;
R₂₁ is hydrogen, (C₁-C₂)alkyl, fluoro, chloro, bromo, hydroxy, cyano, nitro, (C₁-C₂)alkoxy, (C₁-C₂)haloalkyl, (C₁-C₂)haloalkoxy, or —Y₃-L₄-Z₂;
Y₃ is absent, O, S, S(O), S(O)₂, NR′, C(O), C(O)O, OC(O), C(O)N(R′), N(R′)C(O), S(O)₂N(R′), or N(R′)SO₂;
L₄ is absent or (C₁-C₂)alkylene;
Z₂ is hydrogen, (C₁-C₆)alkyl, (C₂-C₄)alkenyl, (C₂-C₄)alkynyl, phenyl, (C₃-C₆)carbocyclyl, or 4- to 6-membered heterocyclyl, wherein Z₂ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, amino, cyano, hydroxy, C(O)R′, C(O)OR′, OC(O)R′, C(O)NR′R′, and N(R′)C(O)R′, wherein each R′ is independently hydrogen or (C₁-C₄)alkyl;
or R₂₀ and R₂₁ together with the carbon atom to which they are attached form (C₃-C₆)carbocyclyl or 3- to 6-membered heterocyclyl, optionally substituted by one or more substituents selected from (C₁-C₂)alkyl, halo, (C₁-C₂)haloalkyl, (C₁-C₂)haloalkoxy, (C₁-C₂)alkoxy, (C₁-C₂)alkylamino, amino, cyano, and hydroxy;
R₂₂ is (C₁-C₂)alkyl, —C(O)OR″, OR″, —C(O)NR″, NR″R″, phenyl, or 5-membered heteroaryl, wherein each R″ is independently hydrogen or (C₁-C₂)alkyl;
A″ is (C₄-C₆)carbocyclyl or 4- to 6-membered heterocyclyl, optionally substituted with one or more substituents independently selected from (C₁-C₂)alkyl, halo, hydroxy, oxo, cyano, and (C₁-C₂)alkoxy;
W₃ is NR₂₃ or CR₂₄R_{24′};
R₂₃ is hydrogen, (C₁-C₂)alkyl, (C₁-C₄)haloalkyl, (C₁-C₄)hydroxyalkyl, —C(O)CH₃, or —C(O)O—(C₁-C₄)alkyl;
R₂₄ and R_{24′} are independently hydrogen, (C₁-C₂)alkyl, cyclopropyl, fluoro, chloro, bromo, hydroxy, amino, cyano, nitro, (C₁-C₂)haloalkyl, (C₁-C₂)haloalkoxy, (C₁-C₂)alkoxy, —C(O)OR″, NR″R″, phenyl, or 5-membered heteroaryl;
R₅ is hydrogen, (C₁-C₄)alkyl, (C₃-C₆)cycloalkyl, (C₁-C₄)haloalkyl, or cyano, wherein said alkyl or cycloalkyl is optionally substituted by one or more substituents selected from (C₁-C₄)alkyl, (C₃-C₆)cycloalkyl, hydroxy, (C₁-C₂)alkoxy, amino, NH(C₁-C₂)alkyl, N((C₁-C₂)alkyl)₂, (C₁-C₂)aminoalkyl, and halo;
R₅′ is hydrogen, (C₁-C₄)alkyl, cyano, (C₁-C₄)haloalkyl, or —Y₄-L₅-Z₃;
Y₄ is absent, C(O)O, or C(O)N(R″);
L₅ is absent or (C₁-C₂)alkylene;
Z₃ is hydrogen, (C₁-C₆)alkyl, phenyl, (C₃-C₆)cycloalkyl, or 4- to 6-membered heterocyclyl, wherein Z₃ is optionally substituted by one or more substituents independently selected from (C₁-C₂)alkyl, halo, (C₁-C₂)haloalkyl, (C₁-C₂)haloalkoxy, (C₁-C₂)alkoxy, amino, nitro, cyano, and hydroxy, or
R₅ and R₅′, together with the carbon atom to which they are attached, form a (C₄-C₆)carbocyclyl, or 4- to 6-membered heterocyclyl;
A′ is a 6- or 7-membered heterocyclyl, which in addition to R₅ and R₅′, is optionally further substituted by one more substituents independently selected from oxo, (C₁-C₂)alkyl, cyclopropyl, spiro-cyclopropyl, halo, (C₁-C₂)haloalkyl, (C₁-C₂)haloalkoxy, (C₁-C₂)alkoxy, amino, cyano, and hydroxy;
X₆ is CR₂₅ or N;
R₂₅ is hydrogen, fluor, chloro, or methyl;
R₆ is hydrogen, (C₁-C₂)alkyl, (C₃-C₄)cycloalkyl, (C₁-C₂)haloalkyl, cyano, (C₂-C₄)alkenyl, or (C₂-C₄)alkynyl;
R₆′ is (C₁-C₄)alkyl, cyano, (C₁-C₄)haloalkyl, or —Y₅-L₆-Z₄;
Y₅ is absent, C(O), C(O)O, OC(O), C(O)N(R″), or S(O)₂N(R″);
L₆ is absent or (C₁-C₂)alkylene optionally substituted by one or more substituents selected from (C₁-C₂)alkyl and oxo;
Z₄ is hydrogen, (C₁-C₆)alkyl, (C₂-C₄)alkenyl, (C₂-C₄)alkynyl, phenyl, (C₃-C₆)carbocyclyl, (C₃-C₆)cycloalkenyl, or 4- to 6-membered heterocyclyl, wherein Z₄ is optionally substituted by one or more substituents independently selected from oxo, (C₁-C₄)alkyl, (C₃-C₆)cycloalkyl, halo, (C₁-C₄)haloalkyl, (C₁-C₄)haloalkoxy, (C₁-C₄)alkoxy, (C₁-C₄)alkylamino, amino, nitro, cyano, hydroxy, C(O)R^{u}, C(O)OR^{u}, OC(O)R^{u}, C(O)NR^{u}R^{u}, and N(R^{u})C(O)R^{u}, wherein each R^{u} is independently hydrogen, (C₁-C₄)alkyl, or (C₃-C₆)cycloalkyl, or
Z₄ is -Q-L₇-W₄, wherein
Q is absent, O, NH, or N(C₁-C₂)alkyl;
L₇ is absent or (C₁-C₂)alkylene optionally substituted by one or more substituents selected from oxo and (C₁-C₂)alkyl;
W₄ is (C₁-C₄)alkyl, phenyl, (C₃-C₆)cycloalkyl, (C₃-C₆)cycloalkenyl, or 5- or 6-membered heterocyclyl, wherein W₄ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, amino, nitro, cyano, or hydroxy, or R₆ and R₆′, together with the carbon atom to which they are attached, for a (C₃-C₁₀)carbocyclyl or a 4- to 10-membered heterocyclyl, which is optionally substituted by one or more substituents independently selected from oxo, (C₁-C₂)alkyl, halo, (C₁-C₂)haloalkyl, (C₁-C₂)haloalkoxy, (C₁-C₂)alkoxy, (C₁-C₂)alkylamino, amino, nitro, cyano, or hydroxy; or the (C₃-C₁₀)carbocyclyl or 4- to 10-membered heterocyclyl is optionally fused to a 5- or 6-membered heteroaryl or phenyl ring, and the 5- or 6-membered heteroaryl or phenyl ring is optionally substituted by (C₁-C₂)alkyl, halo, (C₁-C₂)haloalkyl, (C₁-C₂)haloalkoxy, (C₁-C₂)alkoxy, (C₁-C₂)alkylamino, amino, nitro, cyano, or hydroxy; and
R₆″ is hydrogen, (C₁-C₄)alkyl, (C₁-C₂)haloalkyl, (C₁-C₂)alkoxy, (C₁-C₂)haloalkoxy, cyano, nitro, acetylenyl, phenyl, or 5- or 6-membered heteroaryl, wherein said alkyl, phenyl, or heteroaryl is optionally substituted by one or more substituents independently selected from halo, hydroxy, and amino.

2. The compound of claim 1, wherein R₁ is methyl, —OH, —NH₂, —CH₂CH₂OH, —CH₂CH₂NH₂,

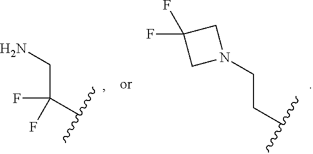 [SMILES: *C(F)(F)CN ; *CCN1CC(F)(F)C1]

3-5. (canceled)

6. The compound of claim 1, wherein X₁ is N, CH, CCl, or CF.

7-10. (canceled)

11. The compound of claim 1, wherein X₂ is CH₂, CHF, CHCl, or CF₂.

12.-15. (canceled)

16. The compound of claim 1, wherein \textcircled{A} is 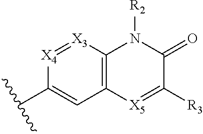
and the compound of formula (I) has the structure of formula I-1, 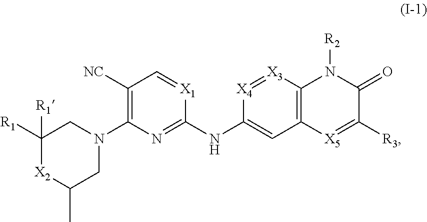
or a pharmaceutically acceptable salt or stereoisomer thereof.

17. The compound of claim 16, wherein X₃ is CH, N, CF, or COMe.

18.-20. (canceled)

21. The compound of claim 16, wherein X₄ is CH.

22. The compound of claim 16, wherein R₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different R₁₃ groups.

23. The compound of claim 22, wherein R₂ is methyl or wherein the heterocyclyl contains 1 heteroatom selected from N and O.

24. (canceled)

25. The compound of claim 16, wherein R₃ is -L₃CR₁₄R₁₅R₁₆.

26. The compound of claim 25, wherein L₃ is —O—(C₁)alkylene.

27. The compound of claim 25, wherein R₁₄ and R₁₅ together with the carbon atom to which they are attached form a (C₃-C₅)carbocyclyl, 4- to 7-membered heterocyclyl, or C═O.

28. The compound of claim 27, wherein R₁₄ and R₁₅ together with the same carbon atom to which they are attached form C═O, or
R₁₄ and R₁₅ together with the same carbon atom to which they are attached form an oxetane ring.

29. (canceled)

30. The compound of claim 25, wherein R₁₆ is (C₁-C₆)alkyl, —NR₁₇R₁₈, or —OR₁₇.

31. The compound of claim 30, wherein R₁₆ is methyl, hydroxyl, amino, or NHMe.

32. The compound of claim 16, wherein the compound is of formula I-1a, I-1b, I-1c, I-1d, I-1e, I-1f, I-1g, I-1h, I-1i, or I-1j: 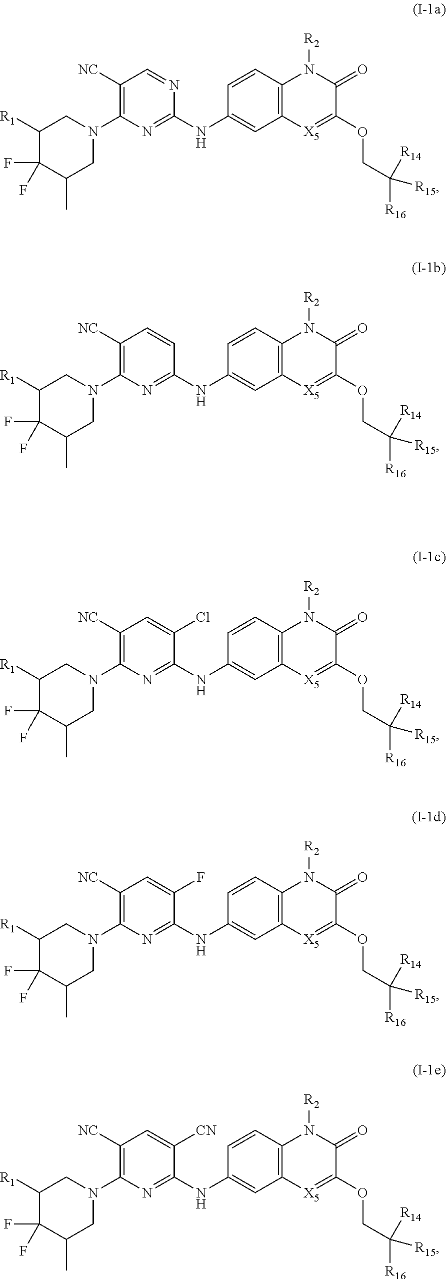 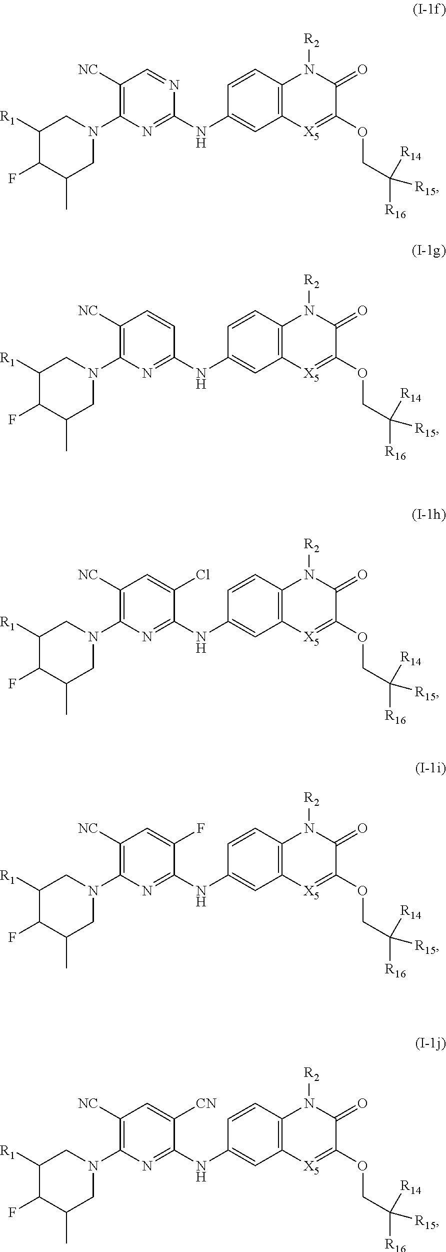
or a pharmaceutically acceptable salt or stereoisomer thereof.

33. The compound of claim 32, wherein the compound is of formula I-1a′, I-1b′, I-1c′, I-1d′, or I-1e′: 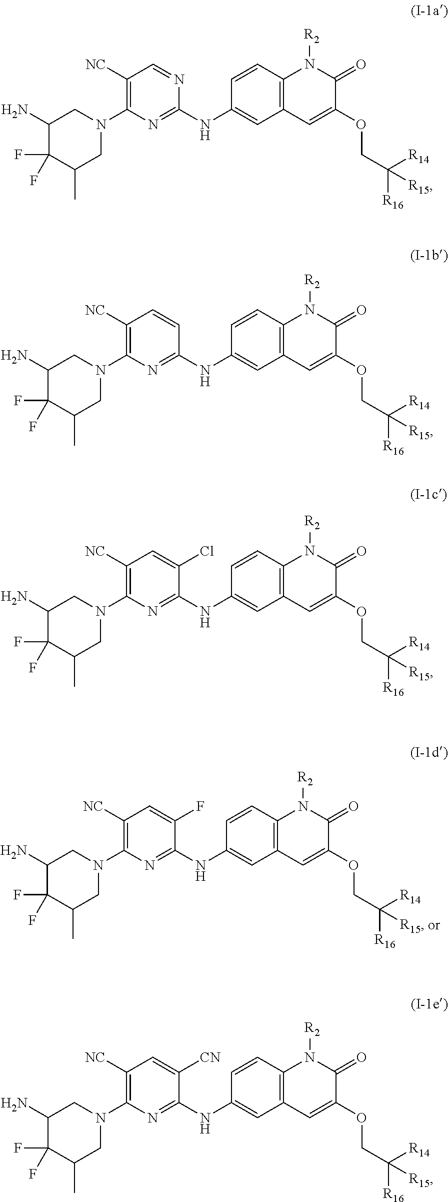
or a pharmaceutically acceptable salt or stereoisomer thereof.

34. The compound of claim 32, wherein the compound is of formula I-1k, I-1l, I-1m, I-1n, I-1o, I-1p, I-1q, I-r, I-1s, I-1t, I-1u, I-1V, I-1w, I-1x, I-1y, I-1z, I-1aa, I-1bb, I-1cc, or I-1dd: 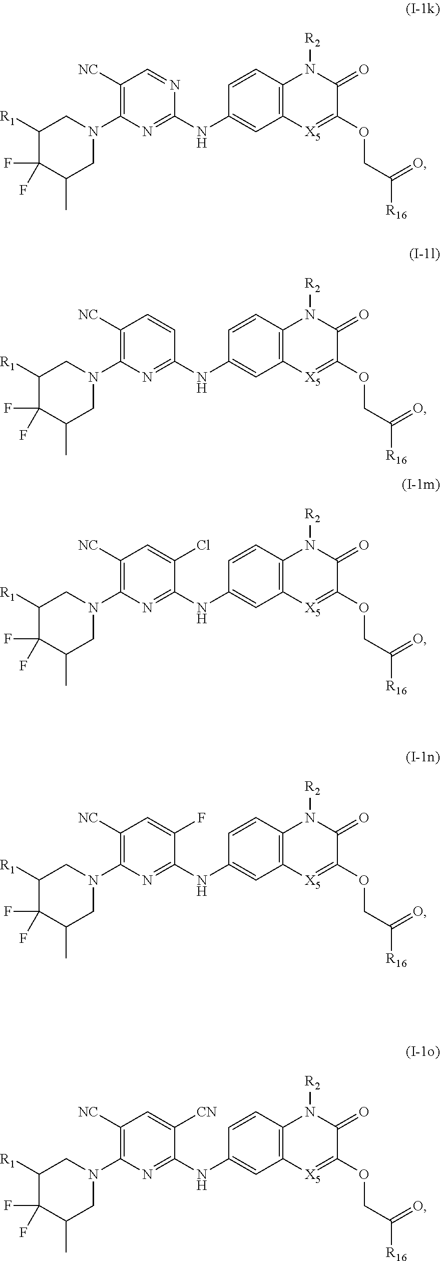 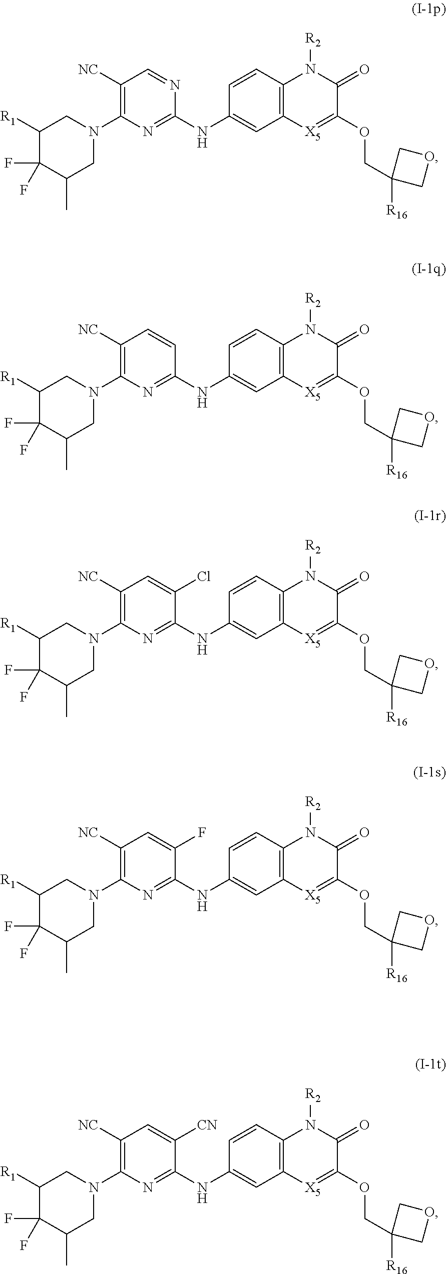 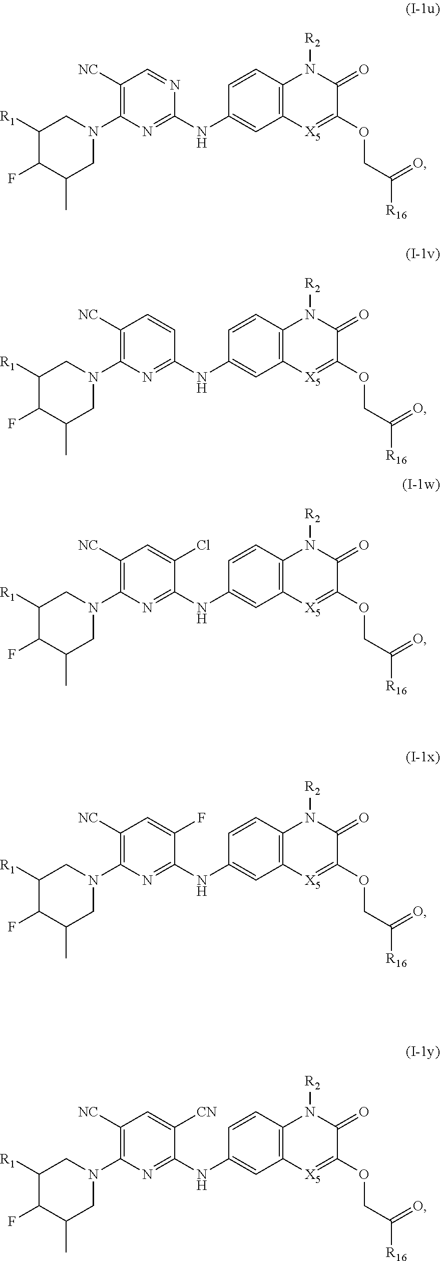 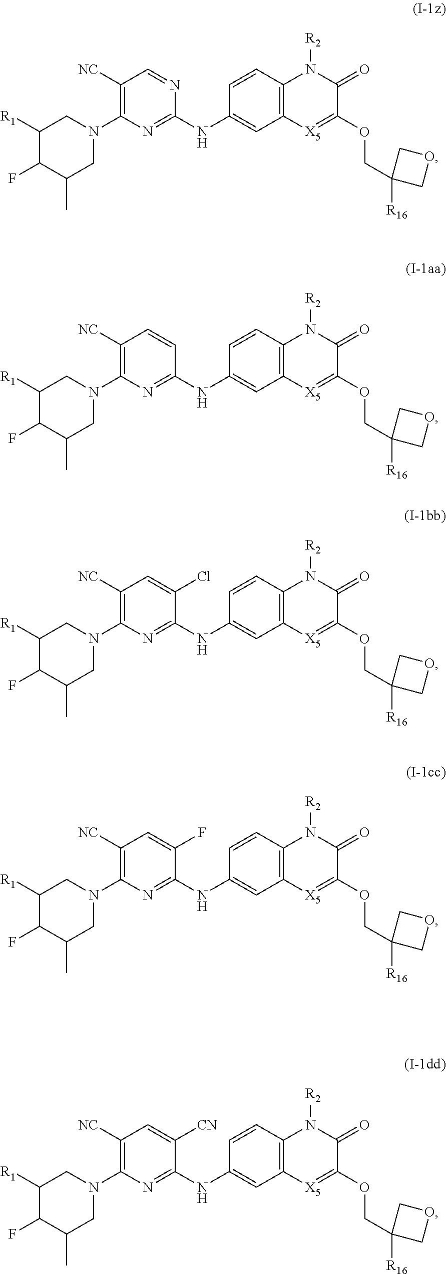
or a pharmaceutically acceptable salt or stereoisomer thereof.

35. The compound of claim 1, wherein \textcircled{A} is 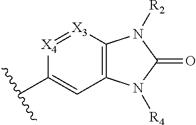
and the compound of formula (I) has the structure of formula I-2, 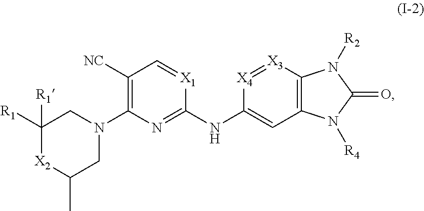
or a pharmaceutically acceptable salt or stereoisomer thereof.

36. The compound of claim 35, wherein X₃ is CH.

37. The compound of claim 35, wherein X₄ is CH.

38. The compound of claim 35, wherein R₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different R₁₃ groups.

39. The compound of claim 38, wherein R₂ is methyl or wherein the heterocyclyl contains 1 heteroatom selected from N and O.

40. (canceled)

41. The compound of claim 35, wherein R₄ is —(CH₂)₂W₁W₂.

42. The compound of claim 41, wherein W₁ is CR₂₁R₂₁.

43. The compound of claim 42, wherein R₂₁ and R_{21′} are both methyl.

44. The compound of claim 41, wherein W₂ is cyano, hydroxy, or amino.

45. (canceled)

46. The compound of claim 35, wherein the compound is of formula I-2a, I-2b, I-2c, I-2d, I-2e, I-2f, 1-2g I-2h, I-2i, or I-2j: 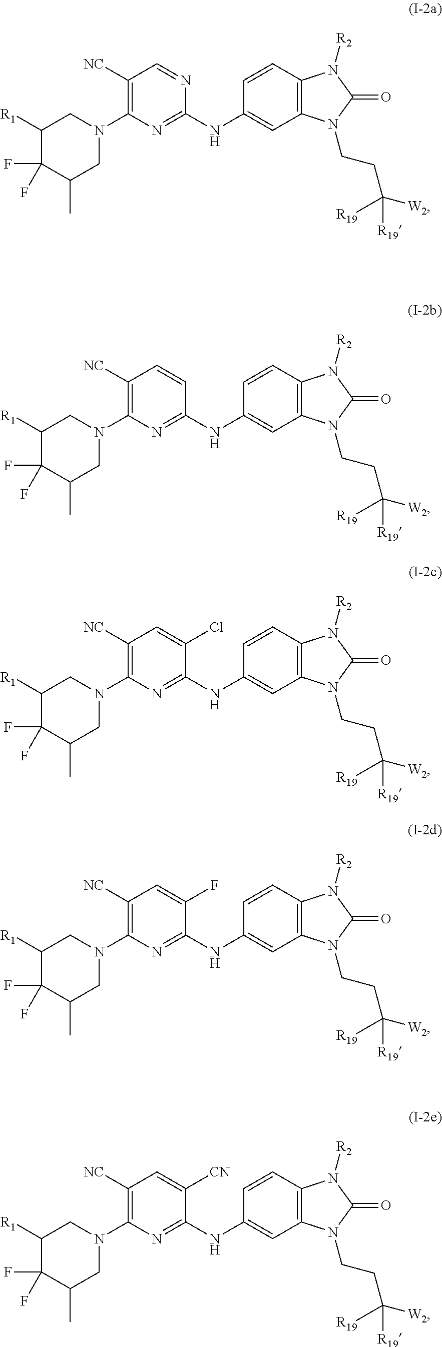 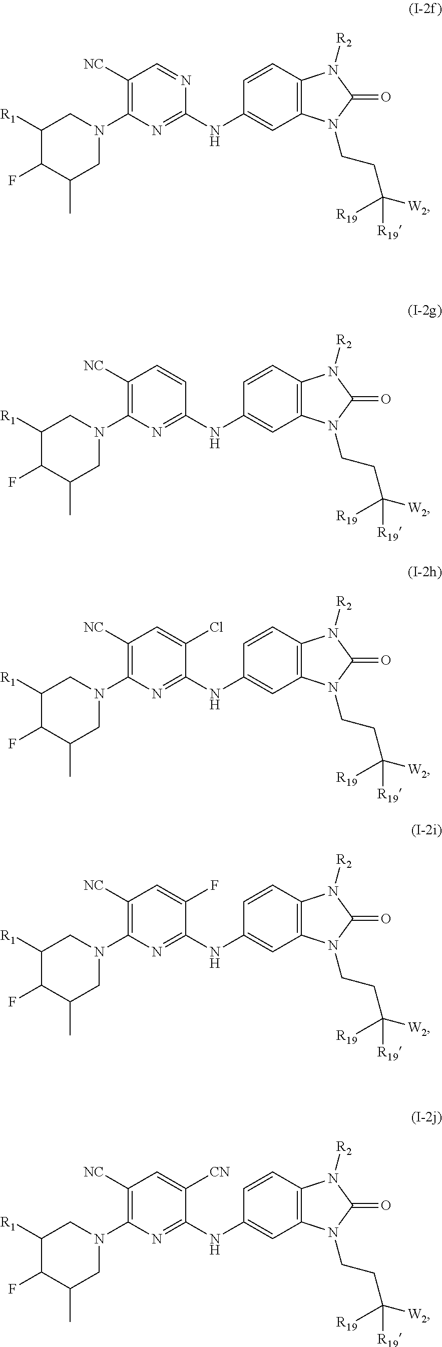
or a pharmaceutically acceptable salt or stereoisomer thereof.

47. The compound of claim 46, wherein the compound is of formula I-2k, I-2l, I-2m, I-2n, I-2o, I-2p, I-2q, I-2r, I-2s, I-2t, I-2u, I-2v, I-2w, I-2x, I-2y, I-2z, I-2aa, I-2bb, I-2cc, or I-2dd: 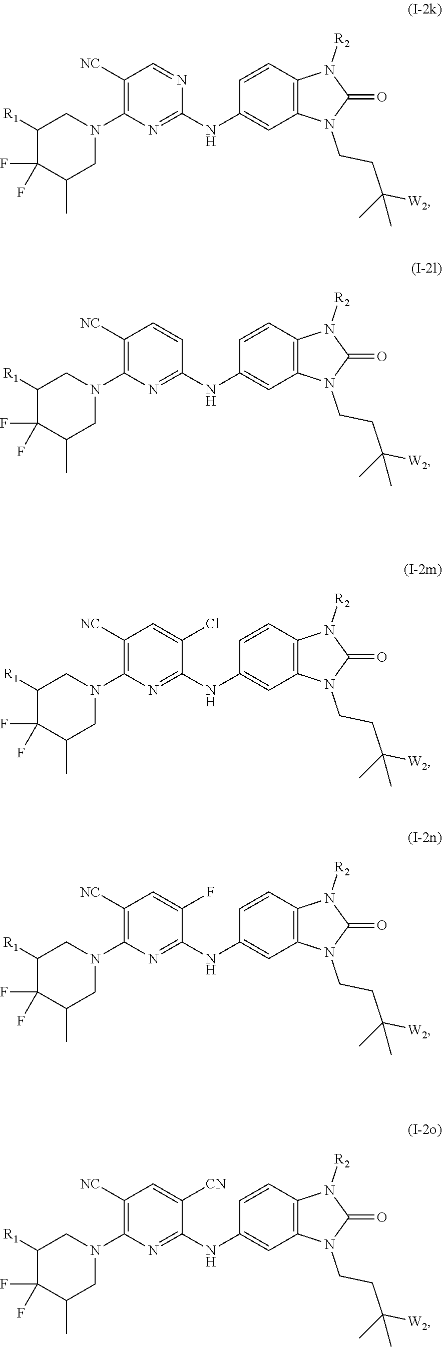 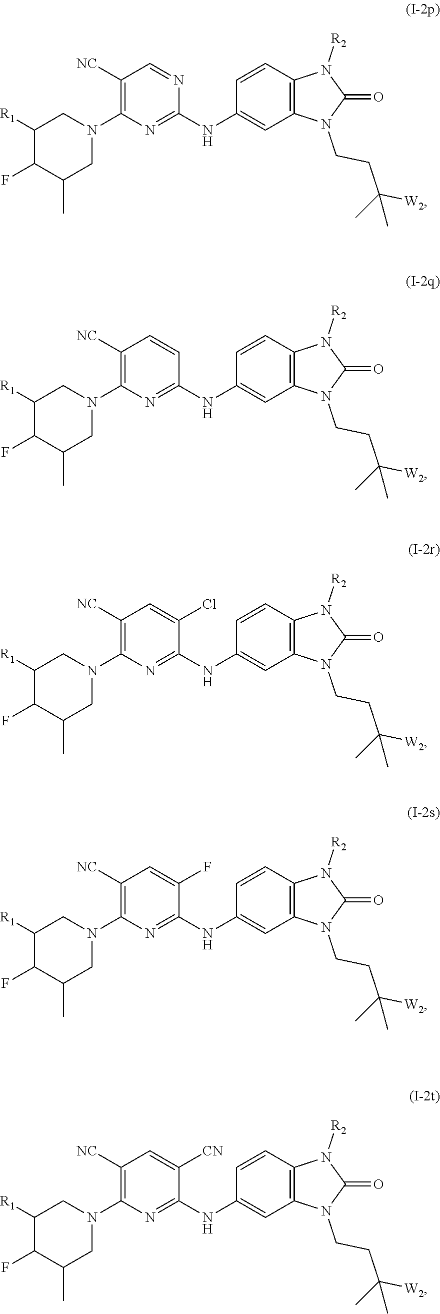 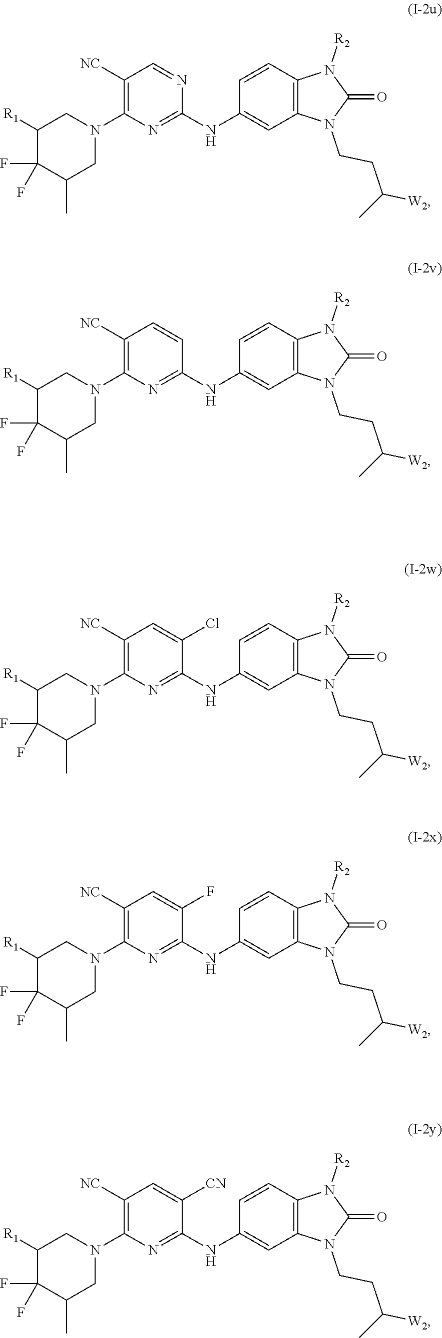 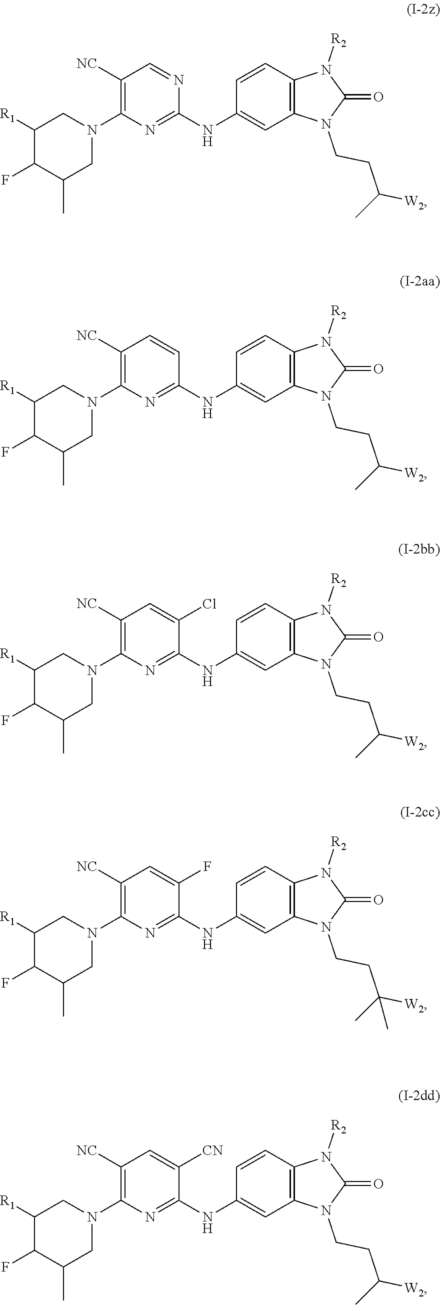
or a pharmaceutically acceptable salt or stereoisomer thereof.

48. The compound of claim 1, wherein \textcircled{A} is 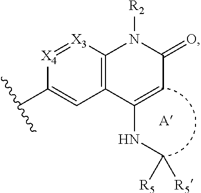
and the compound of formula (I) has the structure of formula I-3, 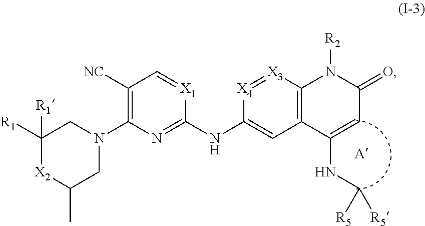
or a pharmaceutically acceptable salt or stereoisomer thereof.

49. The compound of claim 48, wherein X₃ is CH.

50. The compound of claim 48, wherein X₄ is CH.

51. The compound of claim 48, wherein R₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different R₁₃ groups.

52. The compound of claim 48, wherein A′ is a 7-membered heterocyclyl, wherein the heterocyclyl contains 2 heteroatoms selected from N and O, and which in addition to R₅ and R₅′, is optionally further substituted by one more substituents independently selected from oxo, (C₁-C₂)alkyl, cyclopropyl, spiro-cyclopropyl, halo, (C₁-C₂)haloalkyl, (C₁-C₂)alkoxy, amino, cyano, and hydroxy.

53. The compound of claim 48, wherein the compound is of formula I-3a, I-3b, I-3c, I-3d, I-3e, I-3f, I-3g, I-3h, I-3i, I-3j, I-3k, I-31, I-3m, I-3n, I-3o, I-3p, I-3q, I-3r, I-3s, or I-3t: 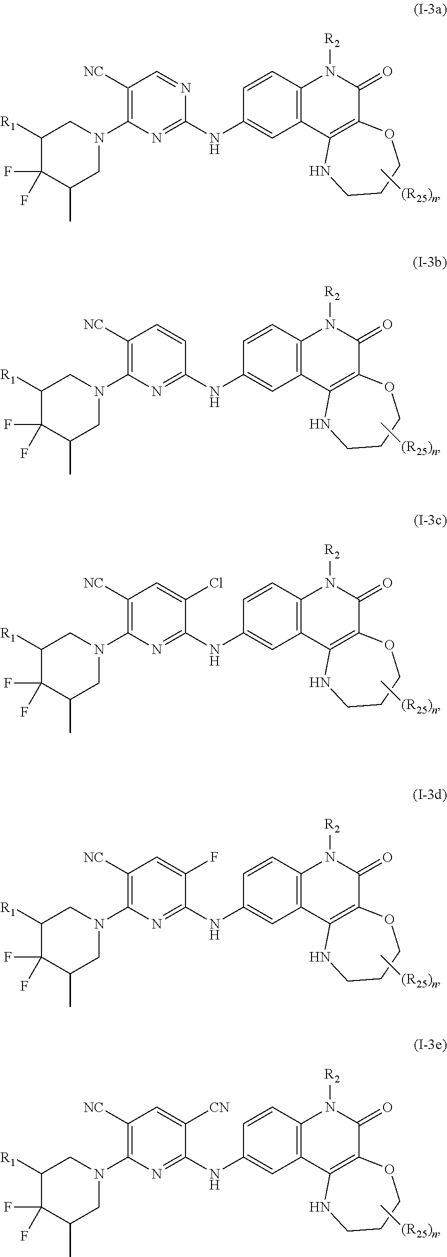 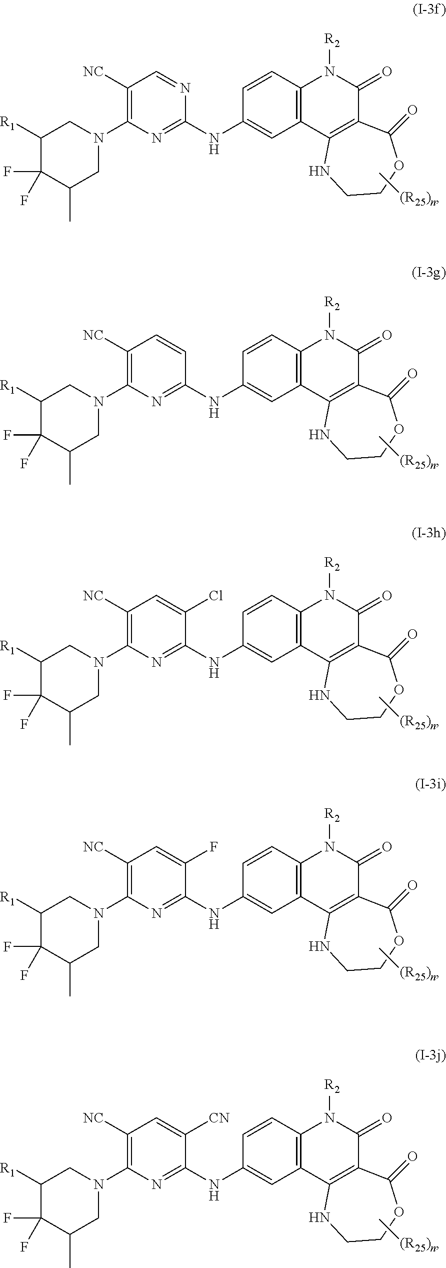 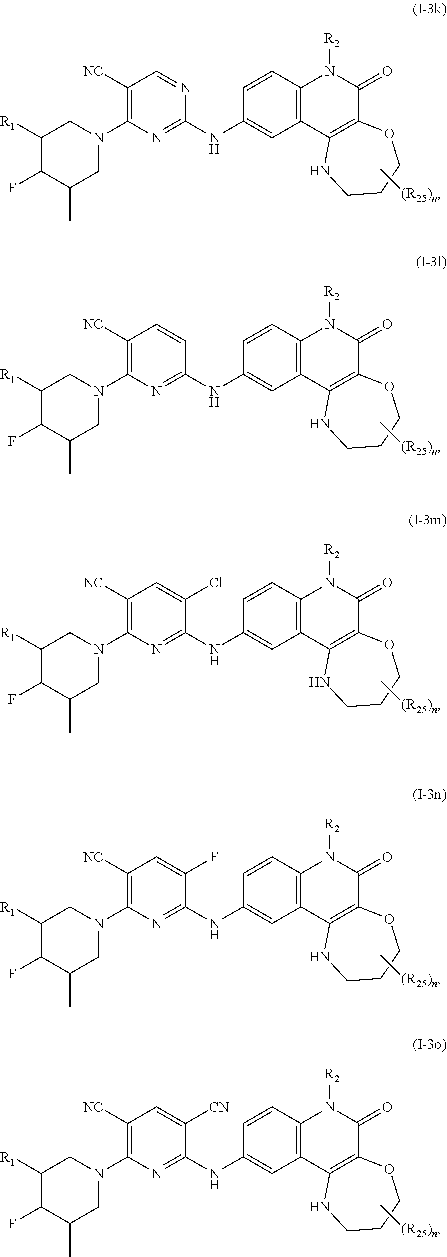 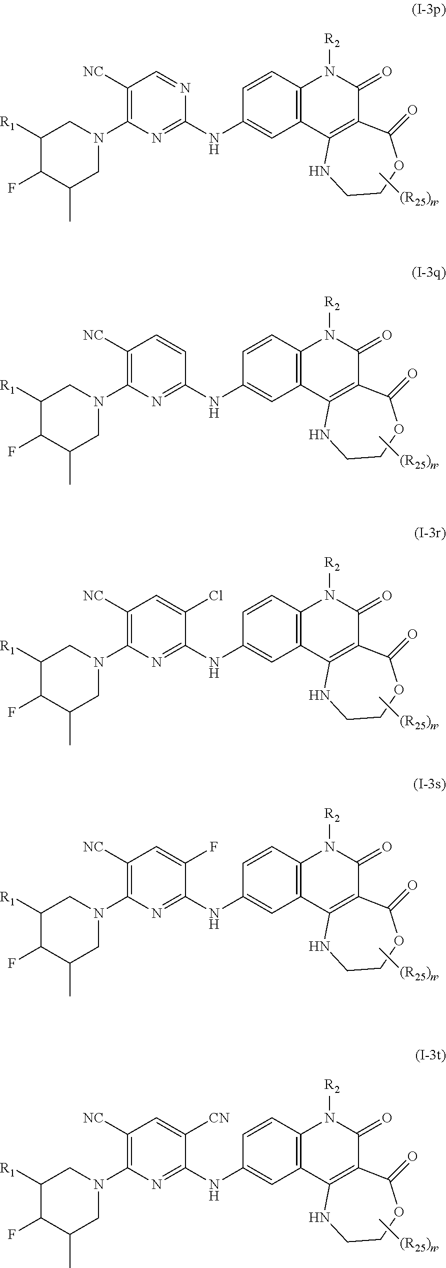
or a pharmaceutically acceptable salt or stereoisomer thereof, wherein each R₂₅ is independently oxo, (C₁-C₂)alkyl, cyclopropyl, spiro-cyclopropyl, halo, (C₁-C₂)haloalkyl, (C₁-C₂)alkoxy, amino, cyano, and hydroxy; and n is 0-3.

54. The compound of claim 48, wherein the compound is of formula I-3u or I-3v: 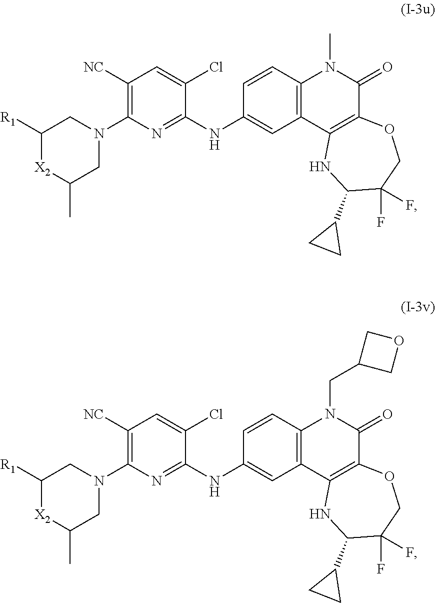
or a pharmaceutically acceptable salt or stereoisomer thereof.

55. The compound of claim 1, wherein \textcircled{A} is 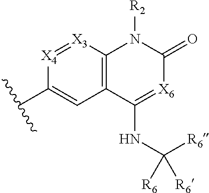
and the compound of formula (I) has the structure of formula I-4, 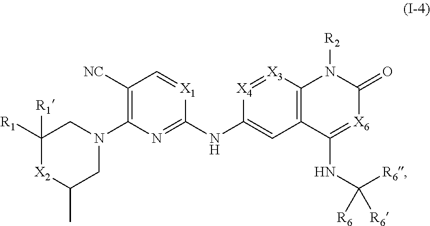
or a pharmaceutically acceptable salt or stereoisomer thereof.

56. The compound of claim 55, wherein X₃ is CH.

57. The compound of claim 55, wherein X₄ is CH.

58. The compound of claim 55, wherein R₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different R₁₃ groups.

59. The compound of claim 55, wherein the compound is of formula I-4a, I-4b, I-4c, I-4d, I-4e, I-4f, 1-4g, I-4h, I-4i, or I-4j: 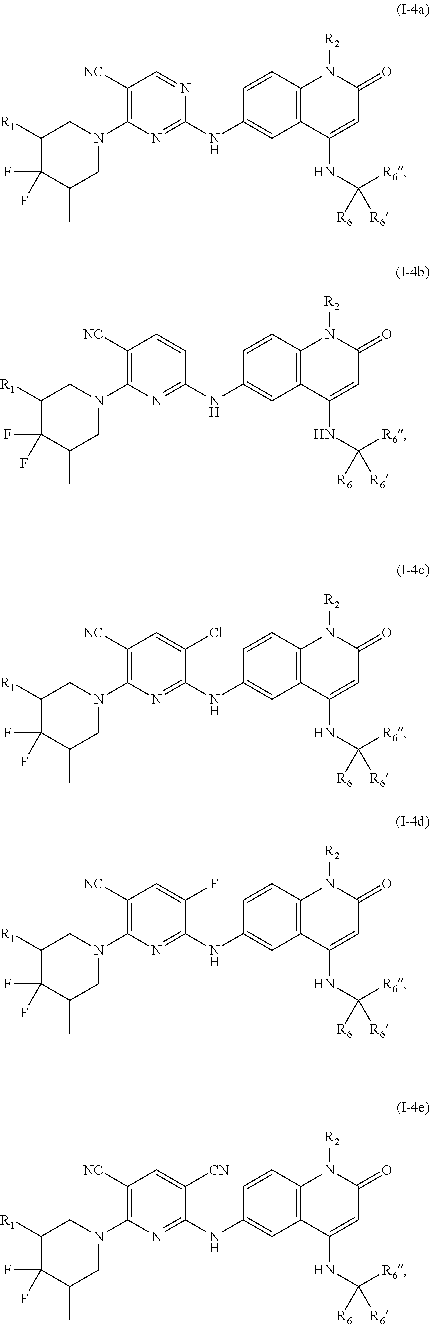 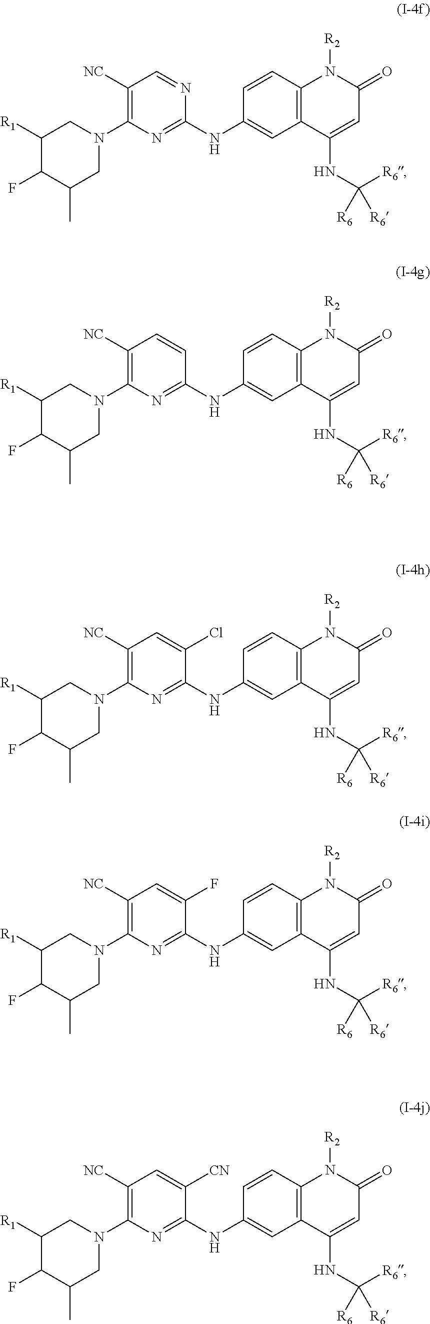
or a pharmaceutically acceptable salt or stereoisomer thereof.

60. The compound of claim 1, wherein \textcircled{A} is 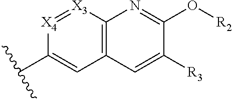
and the compound of formula (I) has the structure of formula I-5, 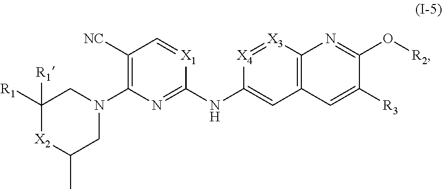
or a pharmaceutically acceptable salt or stereoisomer thereof.

61. The compound of claim 60, wherein X₃ is CH.

62. The compound of claim 60, wherein X₄ is CH.

63. The compound of claim 60, wherein R₂ is 4-membered heterocyclyl or 4-membered heterocyclyl(C₂)alkyl; wherein said alkyl, carbocyclyl, or heterocyclyl is further optionally substituted by one or more, identical or different R₁₃ groups.

64. The compound of claim 60, wherein the compound is of formula I-5a, I-5b, I-5c, I-5d, I-5e, I-5f, I-5g, I-5h, I-5i, or I-5j: 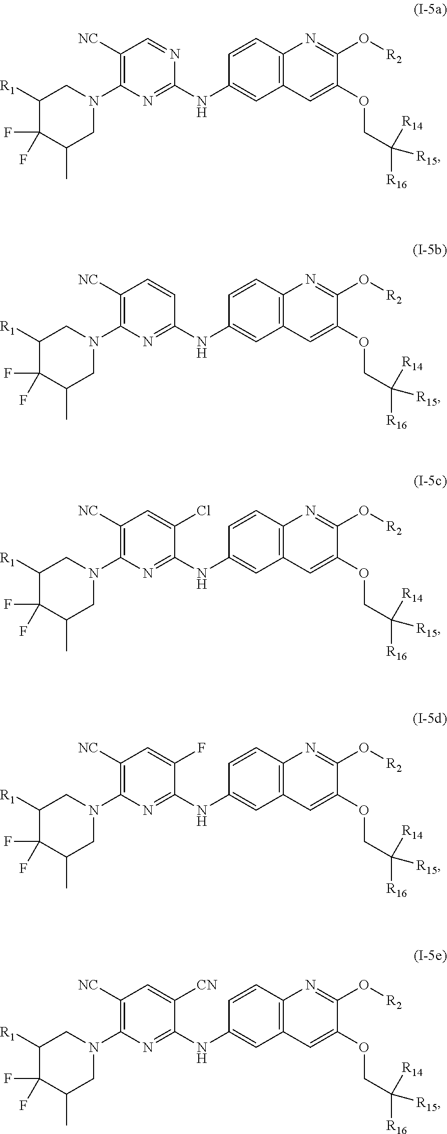 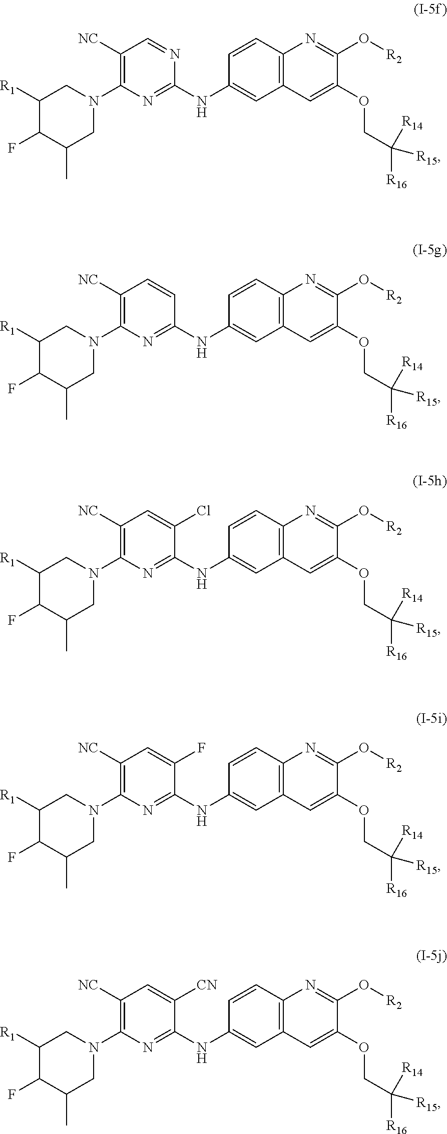
or a pharmaceutically acceptable salt or stereoisomer thereof.

65. The compound of claim 1, which is: 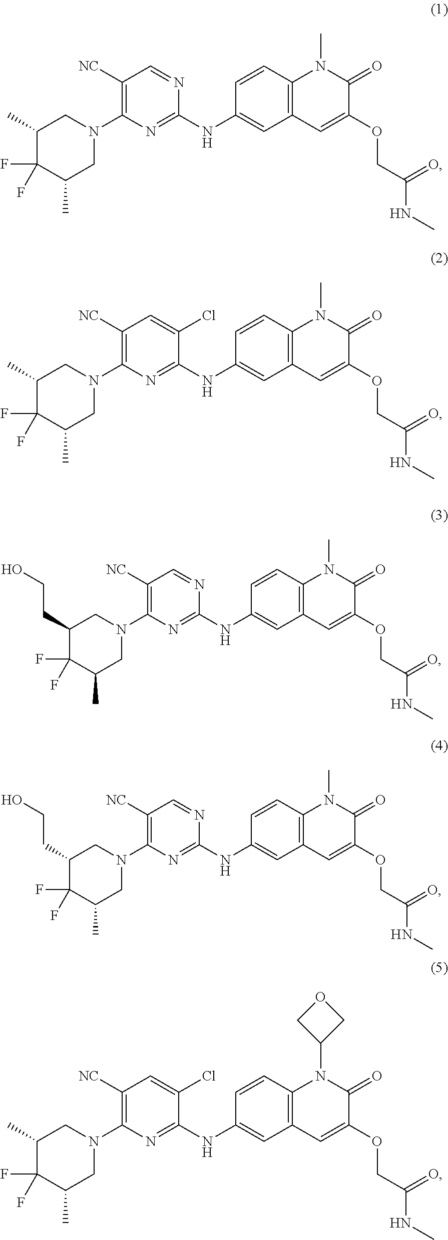 [SMILES: CNC(=O)COc1cc2cc(Nc3ncc(C#N)c(N4C[C@@H](C)C(F)(F)[C@@H](C)C4)n3)ccc2n(C)c1=O ; CNC(=O)COc1cc2cc(Nc3nc(N4C[C@@H](C)C(F)(F)[C@@H](C)C4)c(C#N)cc3Cl)ccc2n(C)c1=O ; CNC(=O)COc1cc2cc(Nc3ncc(C#N)c(N4C[C@H](CCO)C(F)(F)[C@H](C)C4)n3)ccc2n(C)c1=O ; CNC(=O)COc1cc2cc(Nc3ncc(C#N)c(N4C[C@@H](CCO)C(F)(F)[C@@H](C)C4)n3)ccc2n(C)c1=O ; CNC(=O)COc1cc2cc(Nc3nc(N4C[C@@H](C)C(F)(F)[C@@H](C)C4)c(C#N)cc3Cl)ccc2n(C2COC2)c1=O] 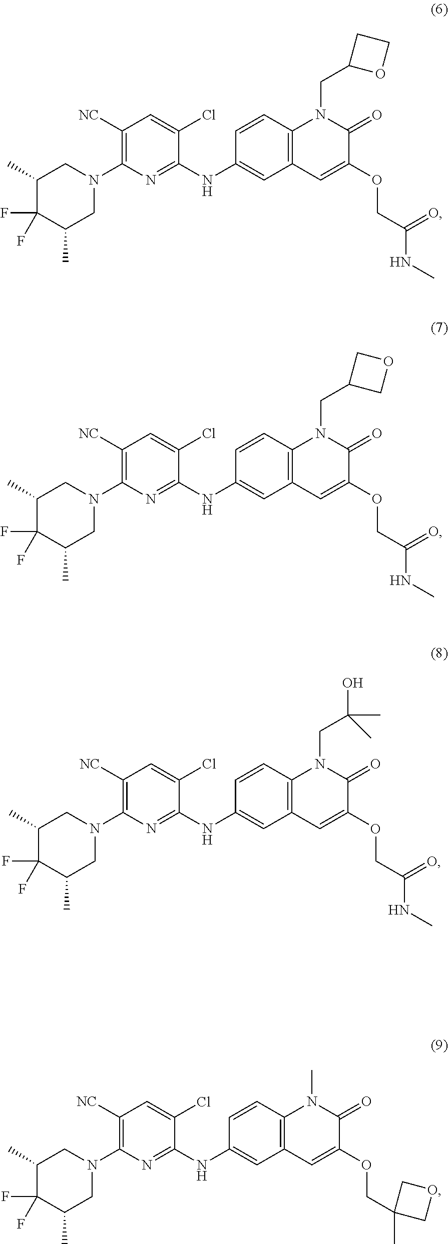 [SMILES: CNC(=O)COc1cc2cc(Nc3nc(N4C[C@@H](C)C(F)(F)[C@@H](C)C4)c(C#N)cc3Cl)ccc2n(CC2CCO2)c1=O ; CNC(=O)COc1cc2cc(Nc3nc(N4C[C@@H](C)C(F)(F)[C@@H](C)C4)c(C#N)cc3Cl)ccc2n(CC2COC2)c1=O ; CNC(=O)COc1cc2cc(Nc3nc(N4C[C@@H](C)C(F)(F)[C@@H](C)C4)c(C#N)cc3Cl)ccc2n(CC(C)(C)O)c1=O ; C[C@@H]1CN(c2nc(Nc3ccc4c(c3)cc(OCC3(C)COC3)c(=O)n4C)c(Cl)cc2C#N)C[C@H](C)C1(F)F] 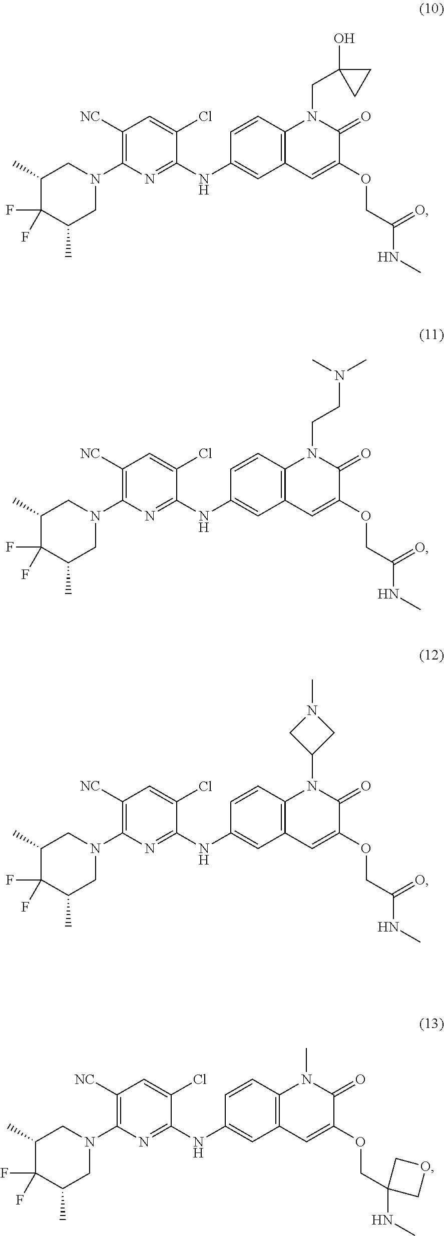 [SMILES: CNC(=O)COc1cc2cc(Nc3nc(N4C[C@@H](C)C(F)(F)[C@@H](C)C4)c(C#N)cc3Cl)ccc2n(CC2(O)CC2)c1=O ; CNC(=O)COc1cc2cc(Nc3nc(N4C[C@@H](C)C(F)(F)[C@@H](C)C4)c(C#N)cc3Cl)ccc2n(CCN(C)C)c1=O ; CNC(=O)COc1cc2cc(Nc3nc(N4C[C@@H](C)C(F)(F)[C@@H](C)C4)c(C#N)cc3Cl)ccc2n(C2CN(C)C2)c1=O ; CNC1(COc2cc3cc(Nc4nc(N5C[C@@H](C)C(F)(F)[C@@H](C)C5)c(C#N)cc4Cl)ccc3n(C)c2=O)COC1] 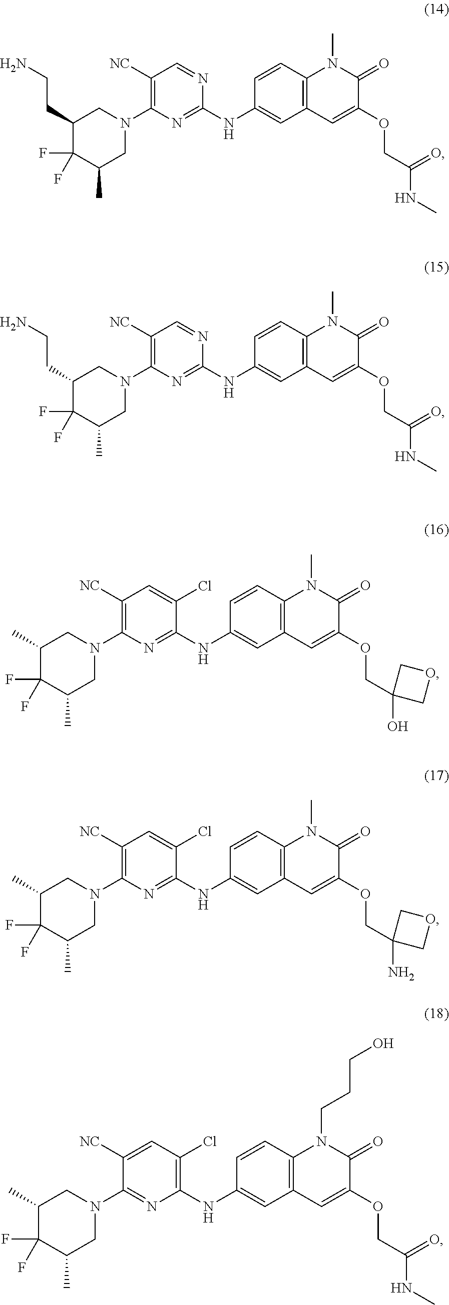 [SMILES: CNC(=O)COc1cc2cc(Nc3ncc(C#N)c(N4C[C@H](CCN)C(F)(F)[C@H](C)C4)n3)ccc2n(C)c1=O ; CNC(=O)COc1cc2cc(Nc3ncc(C#N)c(N4C[C@@H](CCN)C(F)(F)[C@@H](C)C4)n3)ccc2n(C)c1=O ; C[C@@H]1CN(c2nc(Nc3ccc4c(c3)cc(OCC3(O)COC3)c(=O)n4C)c(Cl)cc2C#N)C[C@H](C)C1(F)F ; C[C@@H]1CN(c2nc(Nc3ccc4c(c3)cc(OCC3(N)COC3)c(=O)n4C)c(Cl)cc2C#N)C[C@H](C)C1(F)F ; CNC(=O)COc1cc2cc(Nc3nc(N4C[C@@H](C)C(F)(F)[C@@H](C)C4)c(C#N)cc3Cl)ccc2n(CCCO)c1=O] 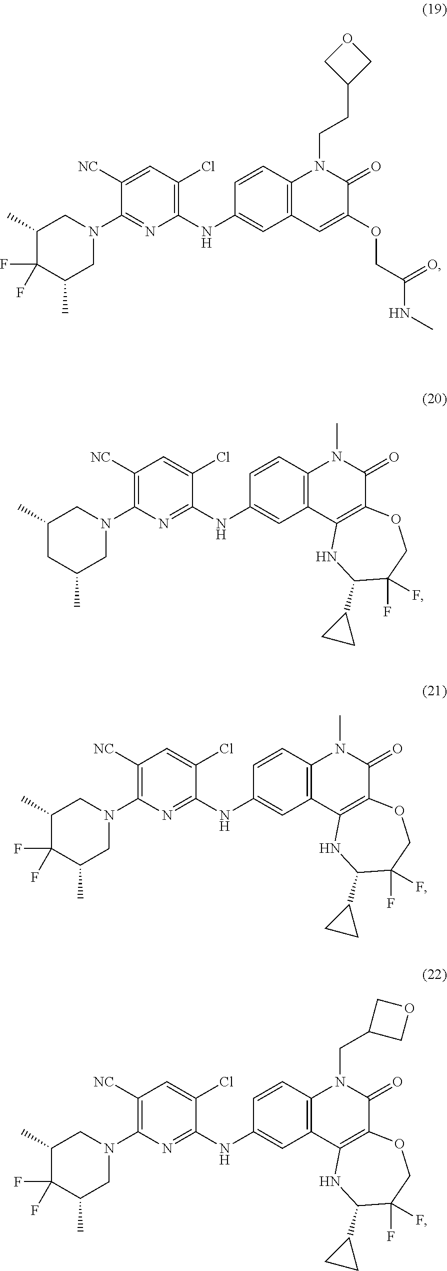 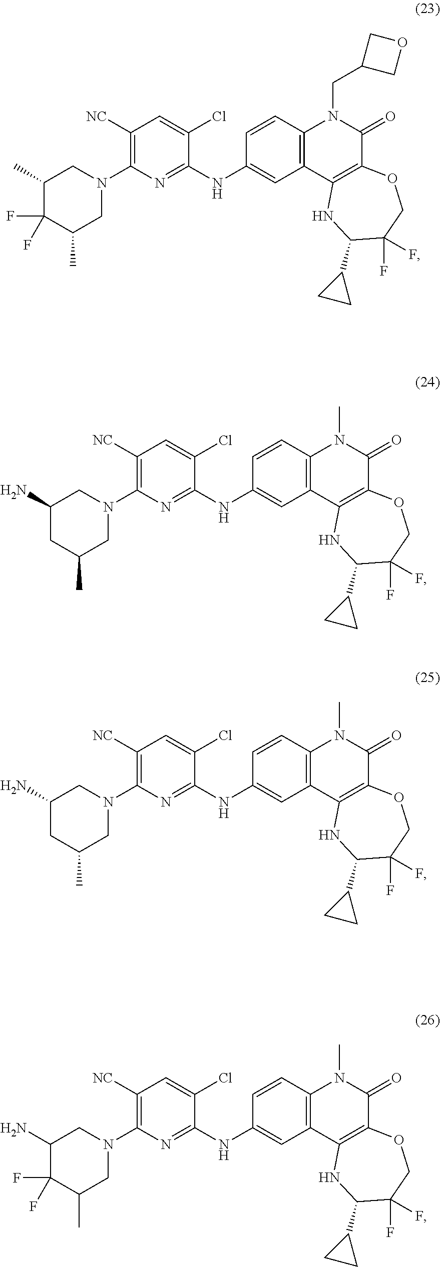 [SMILES: C[C@@H]1CN(c2nc(Nc3ccc4c(c3)c3c(c(=O)n4CC4COC4)OCC(F)(F)[C@H](C4CC4)N3)c(Cl)cc2C#N)C[C@H](C)C1(F)F ; C[C@H]1C[C@@H](N)CN(c2nc(Nc3ccc4c(c3)c3c(c(=O)n4C)OCC(F)(F)[C@H](C4CC4)N3)c(Cl)cc2C#N)C1 ; C[C@@H]1C[C@H](N)CN(c2nc(Nc3ccc4c(c3)c3c(c(=O)n4C)OCC(F)(F)[C@H](C4CC4)N3)c(Cl)cc2C#N)C1 ; CC1CN(c2nc(Nc3ccc4c(c3)c3c(c(=O)n4C)OCC(F)(F)[C@H](C4CC4)N3)c(Cl)cc2C#N)CC(N)C1(F)F] 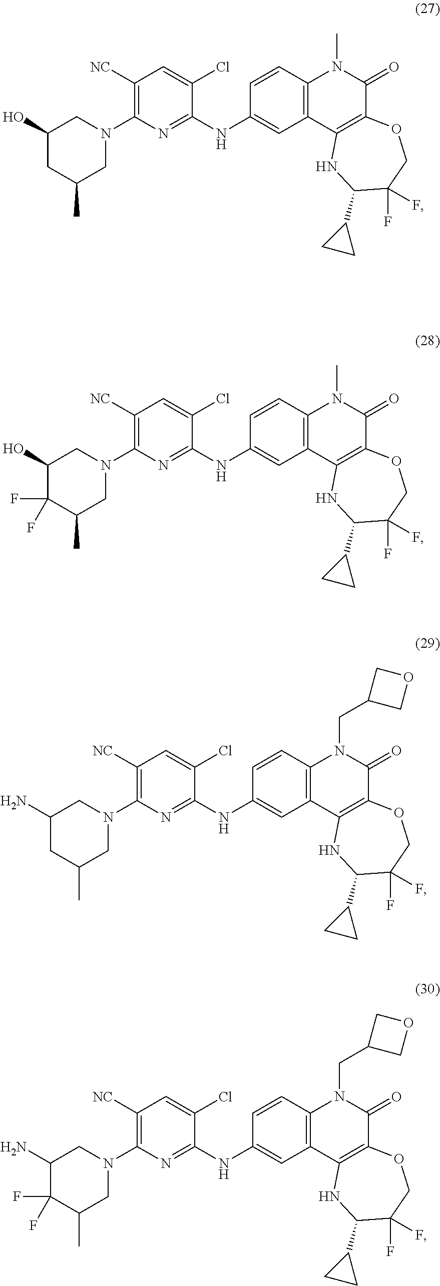 [SMILES: C[C@H]1C[C@@H](O)CN(c2nc(Nc3ccc4c(c3)c3c(c(=O)n4C)OCC(F)(F)[C@H](C4CC4)N3)c(Cl)cc2C#N)C1 ; C[C@@H]1CN(c2nc(Nc3ccc4c(c3)c3c(c(=O)n4C)OCC(F)(F)[C@H](C4CC4)N3)c(Cl)cc2C#N)C[C@H](O)C1(F)F ; CC1CC(N)CN(c2nc(Nc3ccc4c(c3)c3c(c(=O)n4CC4COC4)OCC(F)(F)[C@H](C4CC4)N3)c(Cl)cc2C#N)C1 ; CC1CN(c2nc(Nc3ccc4c(c3)c3c(c(=O)n4CC4COC4)OCC(F)(F)[C@H](C4CC4)N3)c(Cl)cc2C#N)CC(N)C1(F)F] 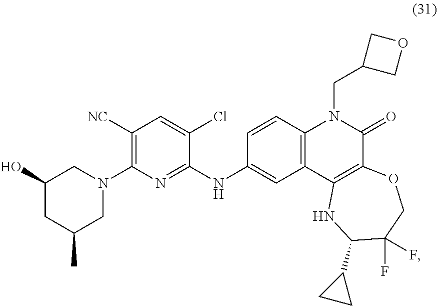 [SMILES: C[C@H]1C[C@@H](O)CN(c2nc(Nc3ccc4c(c3)c3c(c(=O)n4CC4COC4)OCC(F)(F)[C@H](C4CC4)N3)c(Cl)cc2C#N)C1] 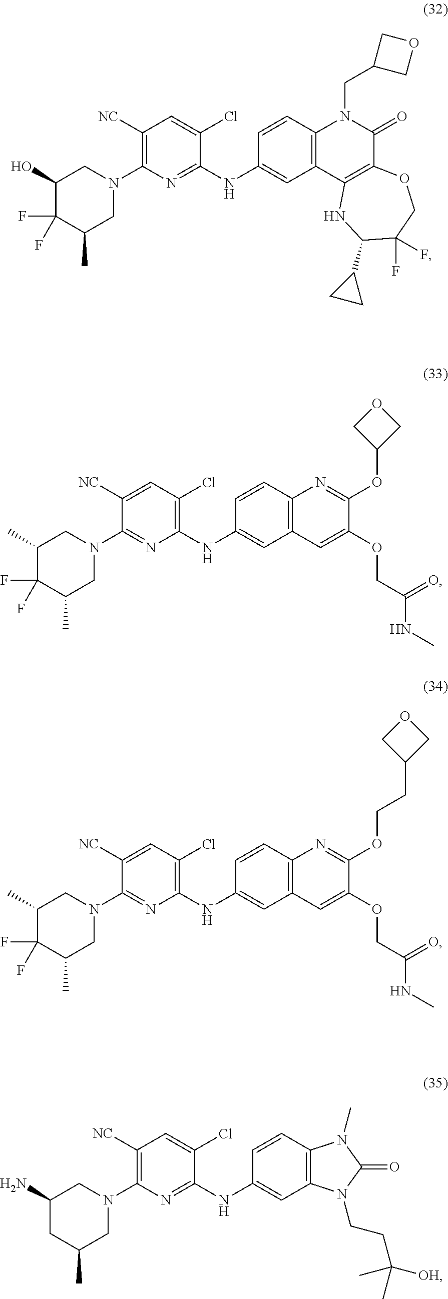 [SMILES: C[C@@H]1CN(c2nc(Nc3ccc4c(c3)c3c(c(=O)n4CC4COC4)OCC(F)(F)[C@H](C4CC4)N3)c(Cl)cc2C#N)C[C@H](O)C1(F)F ; CNC(=O)COc1cc2cc(Nc3nc(N4C[C@@H](C)C(F)(F)[C@@H](C)C4)c(C#N)cc3Cl)ccc2nc1OC1COC1 ; CNC(=O)COc1cc2cc(Nc3nc(N4C[C@@H](C)C(F)(F)[C@@H](C)C4)c(C#N)cc3Cl)ccc2nc1OCCC1COC1 ; C[C@H]1C[C@@H](N)CN(c2nc(Nc3ccc4c(c3)n(CCC(C)(C)O)c(=O)n4C)c(Cl)cc2C#N)C1] 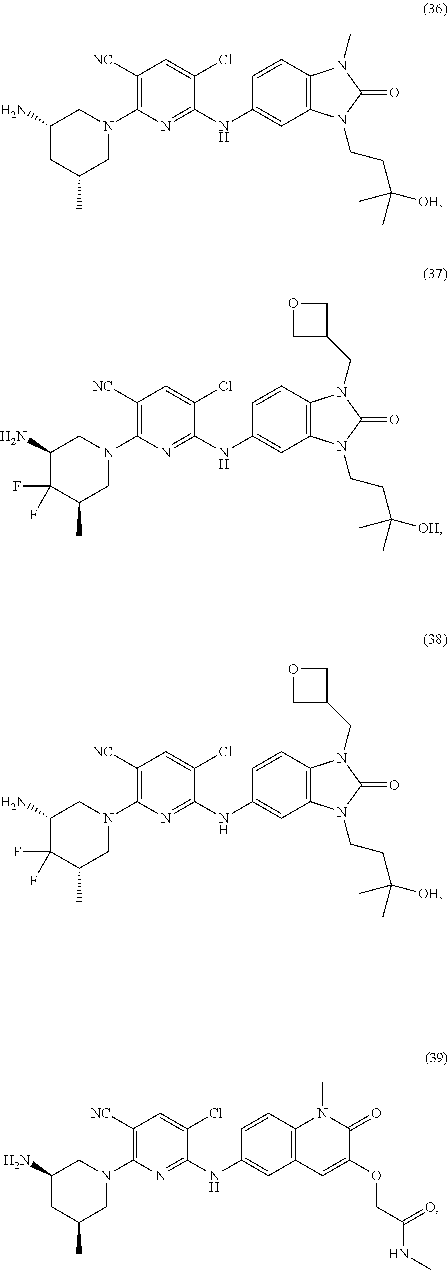 [SMILES: C[C@@H]1C[C@H](N)CN(c2nc(Nc3ccc4c(c3)n(CCC(C)(C)O)c(=O)n4C)c(Cl)cc2C#N)C1 ; C[C@@H]1CN(c2nc(Nc3ccc4c(c3)n(CCC(C)(C)O)c(=O)n4CC3COC3)c(Cl)cc2C#N)C[C@H](N)C1(F)F ; C[C@H]1CN(c2nc(Nc3ccc4c(c3)n(CCC(C)(C)O)c(=O)n4CC3COC3)c(Cl)cc2C#N)C[C@@H](N)C1(F)F ; CNC(=O)COc1cc2cc(Nc3nc(N4C[C@@H](C)C[C@@H](N)C4)c(C#N)cc3Cl)ccc2n(C)c1=O] 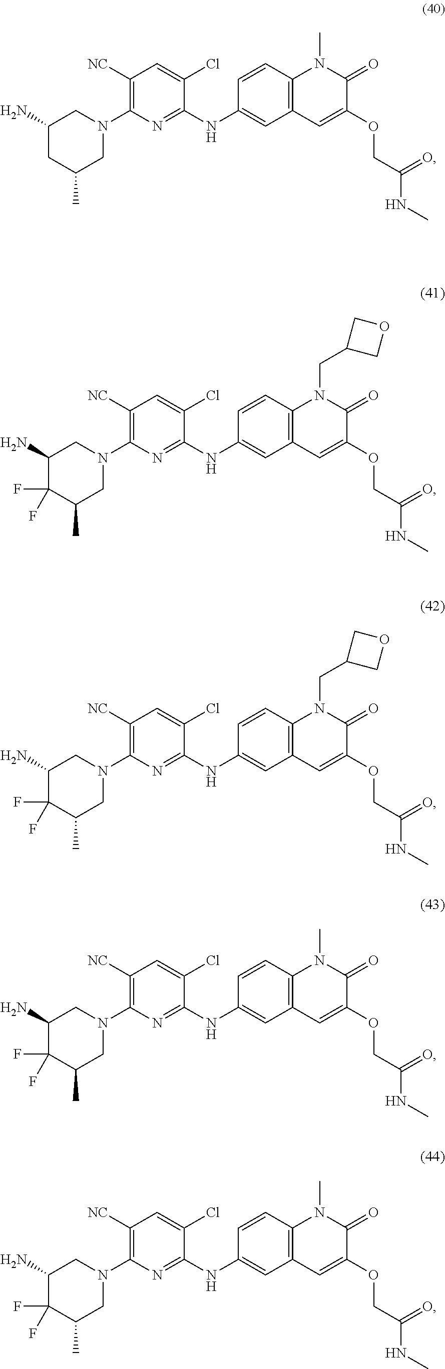 [SMILES: CNC(=O)COc1cc2cc(Nc3nc(N4C[C@H](C)C[C@H](N)C4)c(C#N)cc3Cl)ccc2n(C)c1=O ; CNC(=O)COc1cc2cc(Nc3nc(N4C[C@@H](C)C(F)(F)[C@@H](N)C4)c(C#N)cc3Cl)ccc2n(CC2COC2)c1=O ; CNC(=O)COc1cc2cc(Nc3nc(N4C[C@@H](N)C(F)(F)[C@@H](C)C4)c(C#N)cc3Cl)ccc2n(CC2COC2)c1=O ; CNC(=O)COc1cc2cc(Nc3nc(N4C[C@@H](C)C(F)(F)[C@@H](N)C4)c(C#N)cc3Cl)ccc2n(C)c1=O ; CNC(=O)COc1cc2cc(Nc3nc(N4C[C@@H](N)C(F)(F)[C@@H](C)C4)c(C#N)cc3Cl)ccc2n(C)c1=O] 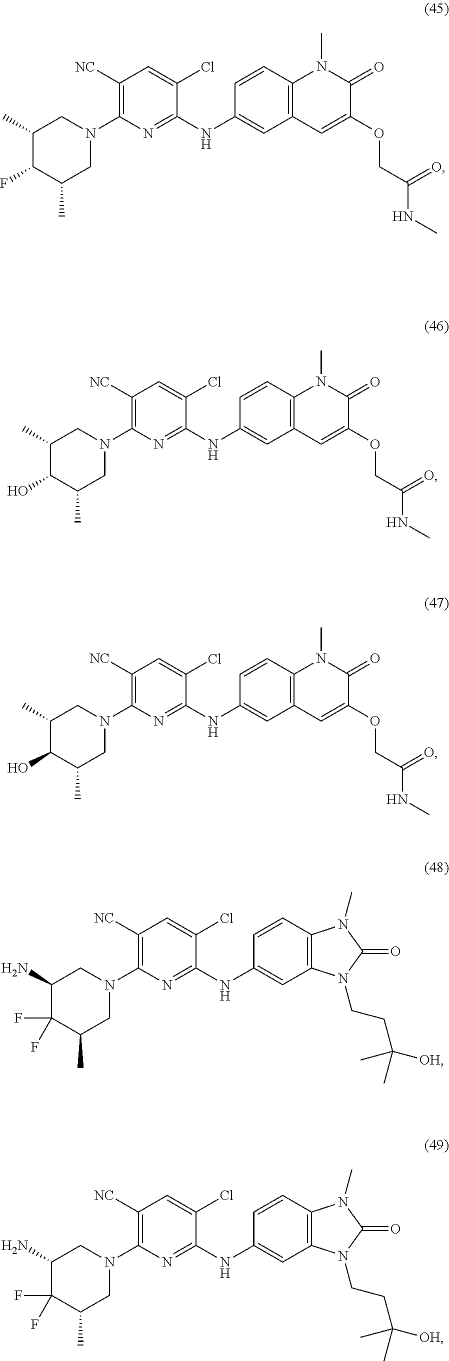 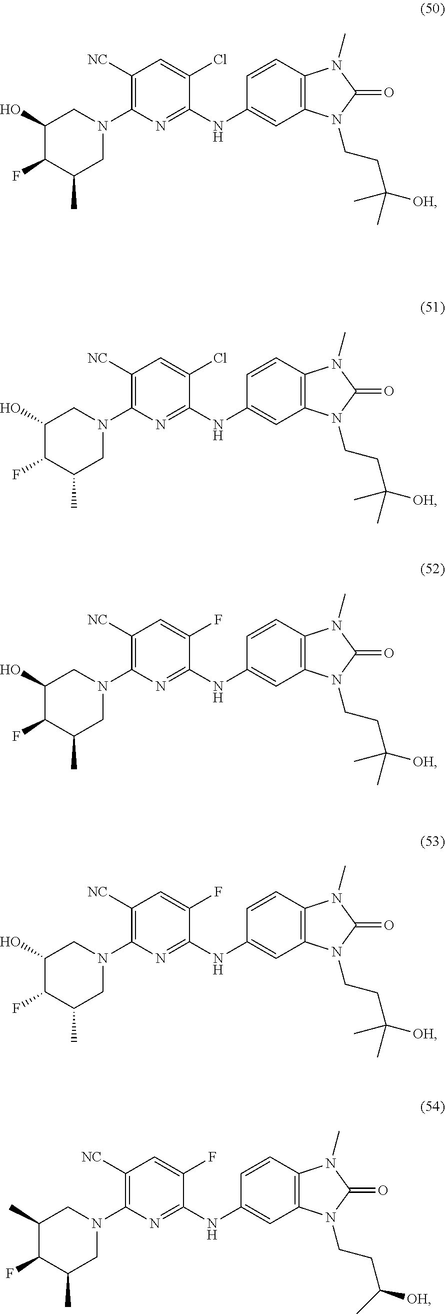 [SMILES: C[C@@H]1CN(c2nc(Nc3ccc4c(c3)n(CCC(C)(C)O)c(=O)n4C)c(Cl)cc2C#N)C[C@H](O)[C@@H]1F ; C[C@H]1CN(c2nc(Nc3ccc4c(c3)n(CCC(C)(C)O)c(=O)n4C)c(Cl)cc2C#N)C[C@@H](O)[C@H]1F ; C[C@@H]1CN(c2nc(Nc3ccc4c(c3)n(CCC(C)(C)O)c(=O)n4C)c(F)cc2C#N)C[C@H](O)[C@@H]1F ; C[C@H]1CN(c2nc(Nc3ccc4c(c3)n(CCC(C)(C)O)c(=O)n4C)c(F)cc2C#N)C[C@@H](O)[C@H]1F ; C[C@H](O)CCn1c(=O)n(C)c2ccc(Nc3nc(N4C[C@@H](C)[C@@H](F)[C@@H](C)C4)c(C#N)cc3F)cc21] 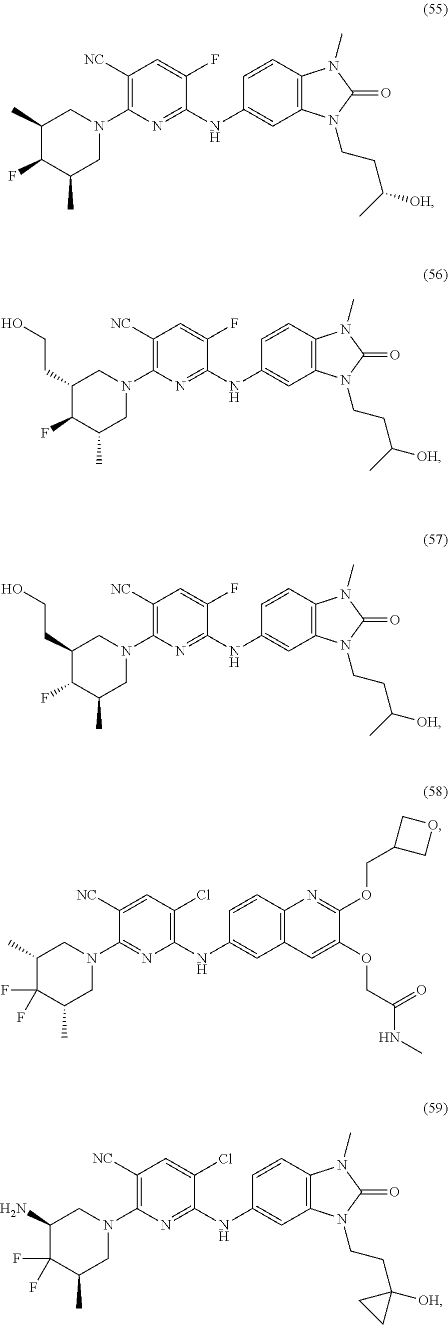 [SMILES: C[C@@H]1CN(c2nc(Nc3ccc4c(c3)n(CC[C@@H](C)O)c(=O)n4C)c(F)cc2C#N)C[C@H](C)[C@@H]1F ; CC(O)CCn1c(=O)n(C)c2ccc(Nc3nc(N4C[C@@H](CCO)[C@H](F)[C@@H](C)C4)c(C#N)cc3F)cc21 ; CC(O)CCn1c(=O)n(C)c2ccc(Nc3nc(N4C[C@H](CCO)[C@@H](F)[C@H](C)C4)c(C#N)cc3F)cc21 ; CNC(=O)COc1cc2cc(Nc3nc(N4C[C@@H](C)C(F)(F)[C@@H](C)C4)c(C#N)cc3Cl)ccc2nc1OCC1COC1 ; C[C@@H]1CN(c2nc(Nc3ccc4c(c3)n(CCC3(O)CC3)c(=O)n4C)c(Cl)cc2C#N)C[C@H](N)C1(F)F] 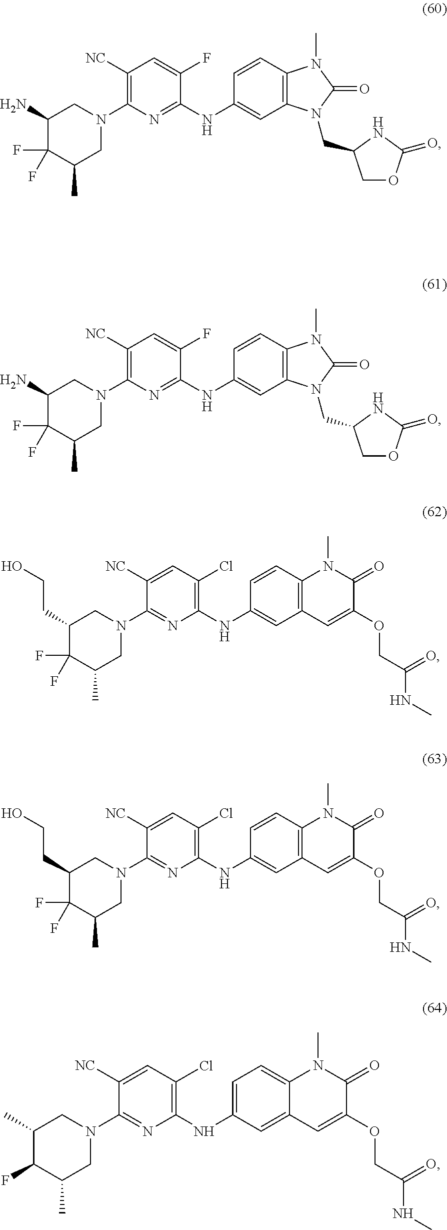 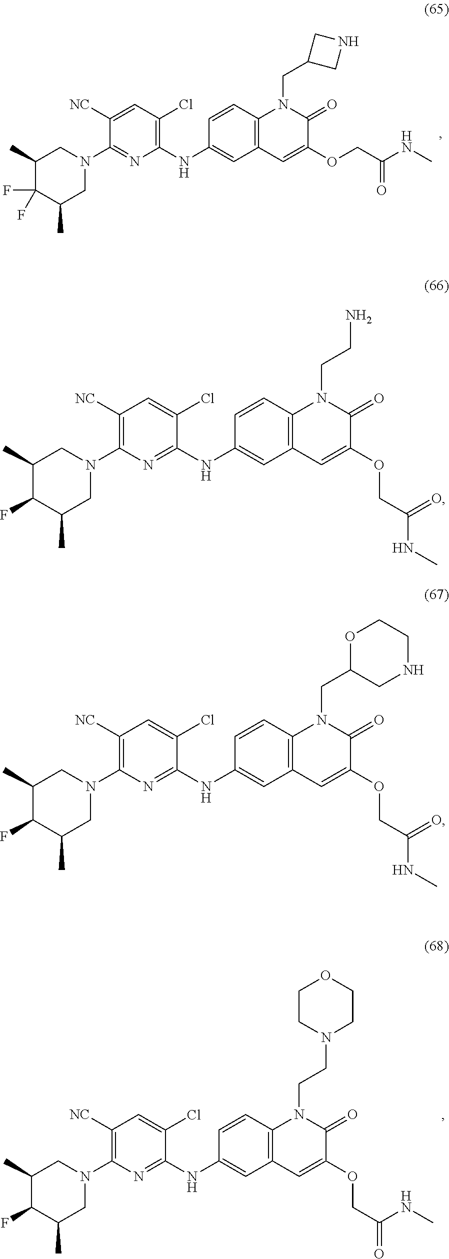 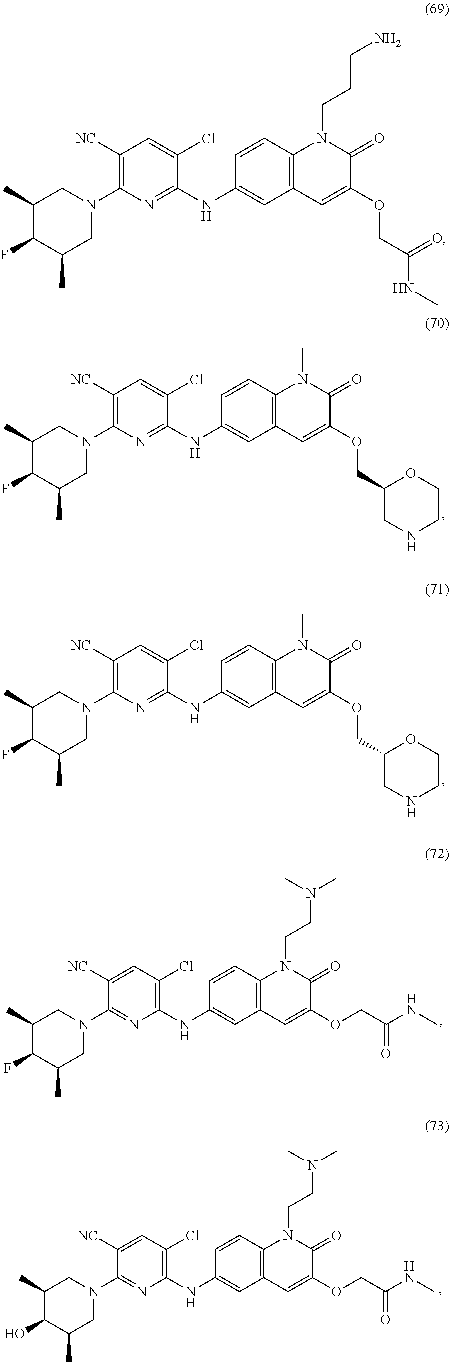 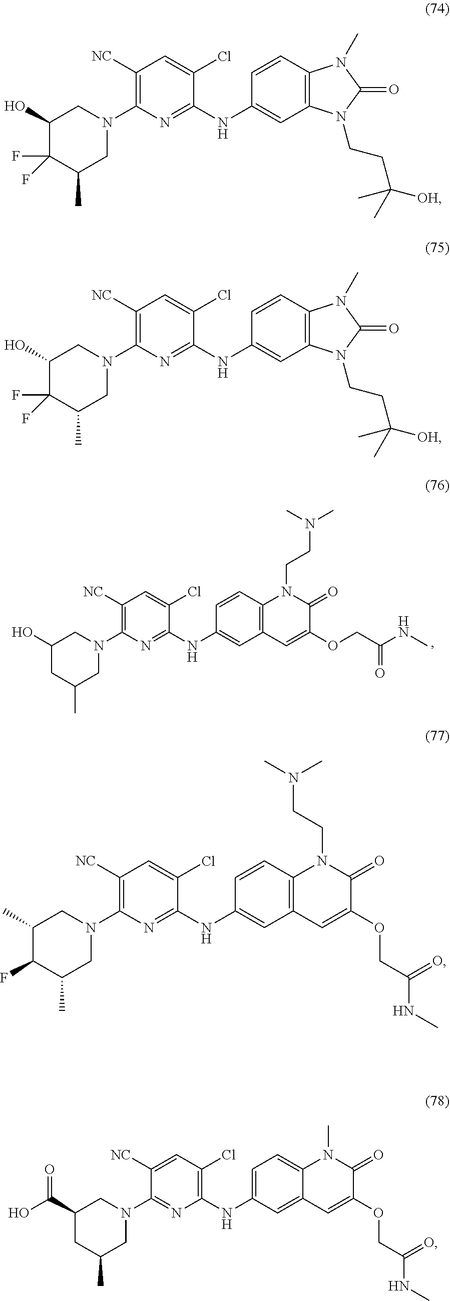 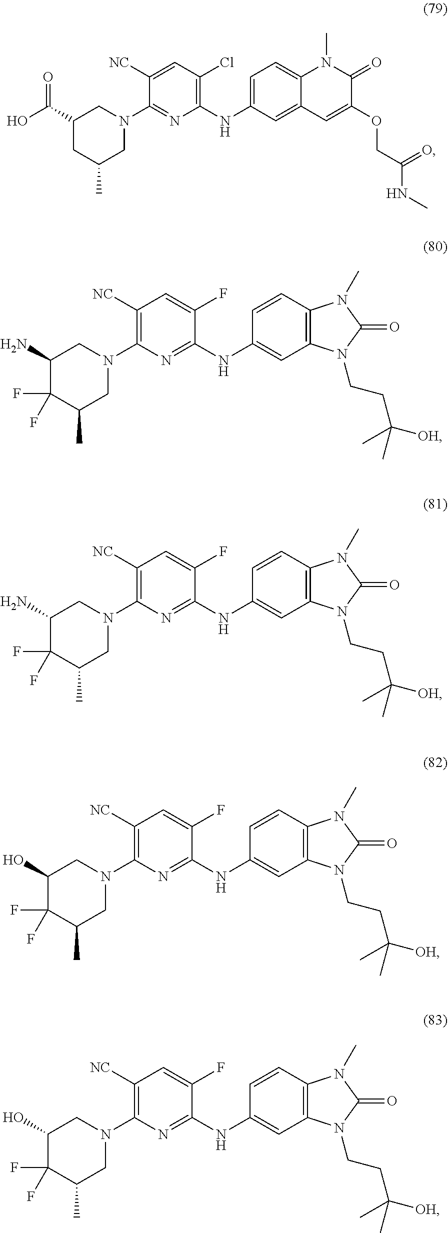 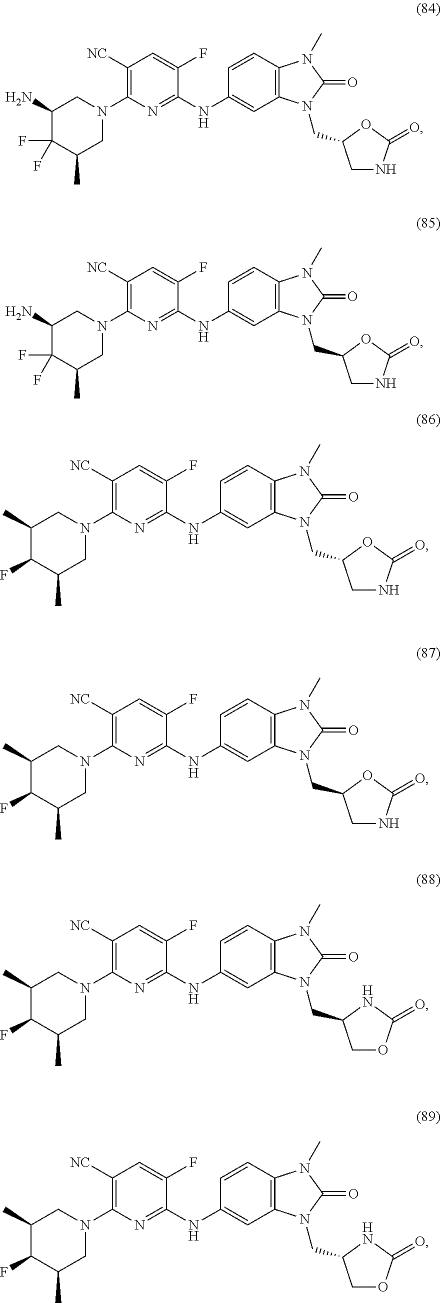 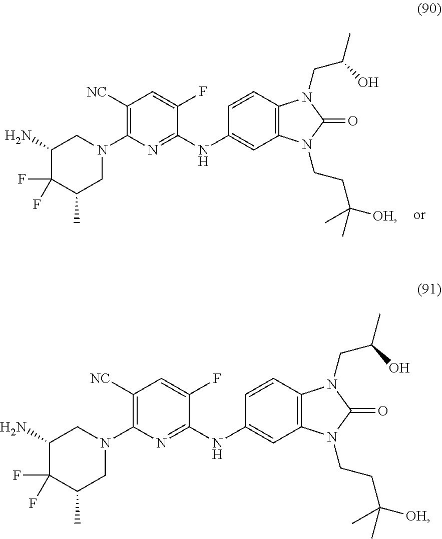 [SMILES: C[C@H](O)Cn1c(=O)n(CCC(C)(C)O)c2cc(Nc3nc(N4C[C@@H](N)C(F)(F)[C@@H](C)C4)c(C#N)cc3F)ccc21 ; "or" ; C[C@@H](O)Cn1c(=O)n(CCC(C)(C)O)c2cc(Nc3nc(N4C[C@@H](N)C(F)(F)[C@@H](C)C4)c(C#N)cc3F)ccc21]
or a pharmaceutically acceptable salt or stereoisomer thereof.

66. A pharmaceutical composition, comprising a therapeutically effective amount of the compound or pharmaceutically acceptable salt or stereoisomer of claim 1, and a pharmaceutically acceptable carrier.

67. The pharmaceutical composition of claim 66, which is in the form of a liquid or a solid.

68. A method of treating a disease or disorder characterized by aberrant B-cell lymphoma 6 (BCL6) activity, comprising administering to a subject in need thereof a therapeutically effective amount of the compound or a pharmaceutically acceptable salt or stereoisomer thereof of claim 1.

69. The method of claim 68, wherein the disease or disorder is a lymphoid malignancy.

70. The method of claim 69, wherein the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), or cutaneous T-cell lymphoma.

71. The method of claim 69, further comprising administering an additional anti-cancer agent.

72. The method of claim 71, wherein the additional anti-cancer agent is an enhancer of zeste homolog 2 (EZH2) inhibitor.
